(19) United States
(12) Patent Application Publication
(10) Pub. No.: US 2015/0364692 A1
(43) Pub. Date: Dec. 17, 2015

(54) COMPOUND, MATERIAL FOR ORGANIC ELECTROLUMINESCENT ELEMENTS, ORGANIC ELECTROLUMINESCENT ELEMENT, AND ELECTRONIC DEVICE

(71) Applicant: IDEMITSU KOSAN CO., LTD., Tokyo (JP)

(72) Inventors: Masahiro KAWAMURA, Chiba-shi (JP); Kazuki NISHIMURA, Sodegaura-shi (JP)

(73) Assignee: IDEMITSU KOSAN CO., LTD., Chiyoda-ku (JP)

(21) Appl. No.: 14/761,767

(22) PCT Filed: May 1, 2014
(86) PCT No.: PCT/JP2014/062116

§ 371 Date: Jul. 17, 2015

(30) Foreign Application Priority Data
May 2, 2013 (JP) 2013-096851
Dec. 26, 2013 (JP) 2013-269997

(51) Int. Cl. H01L 51/00; C07D 491/048; C07D 495/04; C07D 209/82; C07D 409/04; C07D 405/04; C07D 209/96; C07D 209/80; C07D 209/94

(52) U.S. Cl. CPC H01L 51/0054; C07D 209/80; H01L 51/0072; C07D 491/048; H01L 51/0071; C07D 495/04; C07D 209/94; C07D 409/04; H01L 51/0074; C07D 405/04; H01L 51/0073; C07D 209/96; H01L 51/0058; C07D 209/82; H01L 51/0085; H01L 51/5012

(57) ABSTRACT

The present invention provides an organic electroluminescence device with a high emission efficiency and a long lifetime, an electronic equipment including the organic electroluminescence device, and a compound which realizes them. The compound includes a carbazole ring having a specific structure and a fluoranthene skeleton, the organic electroluminescence device includes this compound, and the electronic equipment includes such an organic electroluminescence device.

### TECHNICAL FIELD

The present invention relates to compounds, materials for organic electroluminescence devices comprising the compounds, organic electroluminescence devices employing the compounds, and electronic equipment comprising the organic electroluminescence devices.

### BACKGROUND ART

An organic electroluminescence (EL) device is generally composed of an anode, a cathode, and one or more organic thin film layers sandwiched between the anode and the cathode. When a voltage is applied between the electrodes, electrons are injected from the cathode and holes are injected from the anode each into a light emitting region. The injected electrons recombine with the injected holes in the light emitting region to form excited states. When the excited states return to the ground state, the energy is released as light.

Many researches have been made on the applications of organic EL device to display, etc. because of its possibility of a wide selection of emission colors by using various emitting materials in a light emitting layer. Particularly, the research on the materials which emit three primary red, green, blue colors has been made most actively, and the intensive research has been made to improve their properties.

As such materials for organic electroluminescence devices, biscarbazole derivatives each having a fluoranthene ring are exemplified in Patent Documents 1 and 2. A fluoranthene derivative in which a fluoranthene ring is bonded to a carbazole ring, etc. via a nitrogen-containing heterocyclic ring is exemplified in Patent Document 3. A fluoranthene derivative having a fluoranthene ring and a carbazole ring is disclosed in Patent Document 4.

However, in view of further improving the device performance, it has been still demanded to develop a new material in the field of organic EL devices.

### PRIOR ART

### Patent Documents

Patent Document 1: WO 2012/108388
Patent Document 2: WO 2012/108389
Patent Document 3: WO 2012/030145
Patent Document 4: WO 2013/032278

### SUMMARY OF THE INVENTION

### Problems to be Solved by the Invention

It is an object of the present invention to provide an organic electroluminescence device with a high emission efficiency and a long lifetime, an electronic equipment comprising such an organic electroluminescence device, and a compound which realizes them.

### Means for Solving the Problem

As a result of extensive research, the inventors have found that the object is achieved by a compound having a carbazole ring with a specific structure and a fluoranthene skeleton. The present invention is based on this finding.

In an aspect of the present invention, the following items 1 to 4 are provided:

1. A compound represented by formula (1):

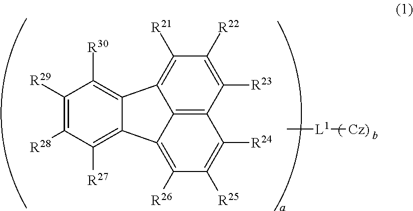

wherein each of R²¹ to R³⁰ independently represents a hydrogen atom or a substituent, provided that one of R²¹ to R³⁰ represents a direct bond to L¹ or Cz; L¹ represents a direct bond, a substituted or unsubstituted, divalent aromatic hydrocarbon group having 6 to 60 ring carbon atoms, a substituted or unsubstituted, divalent oxygen-containing heterocyclic group having 5 to 60 ring atoms, or a substituted or unsubstituted, divalent sulfur-containing heterocyclic group having 5 to 60 ring atoms; Cz represents a structure represented by formula (2); and each of a and b independently represents an integer of 1 to 3, provided that when at least one of a and b is 2 or 3, L¹ is not a direct bond,

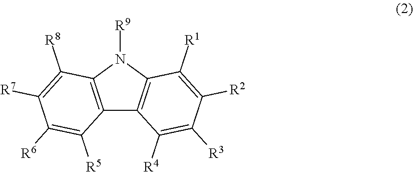

wherein each of R¹ to R⁹ independently represents a hydrogen atom or a substituent; and adjacent groups in R¹ to R⁸ may be bonded to each other to form a ring structure, provided that at least one adjacent pair in R¹ to R⁸ are bonded to each other to form a ring structure represented by formula (3) or (4):

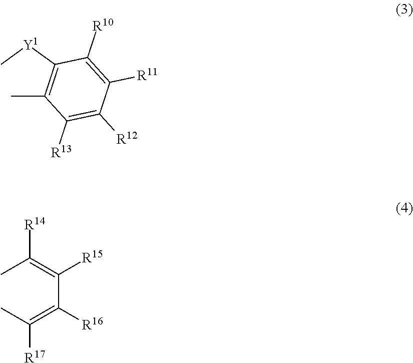

wherein each of R¹⁰ to R¹⁷ independently represents a hydrogen atom or a substituent; adjacent groups in R¹⁰ to R¹³ may be bonded to each other to form a ring structure; Y¹ and R¹⁰ may be bonded to each other to form a ring structure; adjacent groups in R¹⁴ to R¹⁷ may be bonded to each other to form a ring structure; and Y¹ represents an oxygen atom, a sulfur atom, or —CR³¹R³²—, wherein each of R³¹ and R³² independently represents a hydrogen atom or a substituent;

provided that one of R¹ to R¹⁷, R³¹, and R³² represents a direct bond to L¹ or one of R²¹ to R³⁰,

2. A material for organic electroluminescence devices comprising the compound of item 1,

3. An organic electroluminescence device comprising two or more organic thin film layers between a cathode and an anode, wherein the thin film layers comprise a light emitting layer and at least one layer of the thin film layers comprises the compound of item 1, and

4. An electronic equipment comprising the organic electroluminescence device of item 3.

### Effects of the Invention

According to the present invention, an organic electroluminescence device having a high emission efficiency and a long lifetime and an electronic equipment comprise such an organic electroluminescence device are provided, and a compound which realizes them is also provided.

### BRIEF DESCRIPTION OF THE DRAWINGS

FIG. 1 is a schematic illustration showing an example of the structure of an organic electroluminescence device (also referred to as “organic EL device”) according to one embodiment of the invention.

### MODE FOR CARRYING OUT THE INVENTION

The term of “XX to YY carbon atoms” referred to by “a substituted or unsubstituted group ZZ having XX to YY carbon atoms” used herein is the number of carbon atoms of the unsubstituted group ZZ and does not include any carbon atom in the substituent of the substituted group ZZ. The term of “XX to YY atoms” referred to by “a substituted or unsubstituted group ZZ having XX to YY atoms” used herein is the number of atoms of the unsubstituted group ZZ and does not include any atom in the substituent of the substituted group ZZ. In both cases, “YY” is larger than “XX” and each of “XX” and “YY” represents an integer of 1 or more.

The definition of “hydrogen atom” used herein includes isotopes different in the neutron numbers, i.e., light hydrogen (protium), heavy hydrogen (deuterium), and tritium.

The terms of “heteroaryl group”, “heteroarylene group” and “heterocyclic group” used herein means a group having at least one hetero atom as a ring atom. The hetero atom is preferably at least one selected from a nitrogen atom, an oxygen atom, a sulfur atom, a silicon atom, and a selenium atom.

The term of “unsubstituted” referred to by “substituted or unsubstituted” used herein means that no hydrogen atom in a group is substituted by a substituent.

The number of “ring carbon atoms” referred to herein means the number of the carbon atoms included in the atoms which are members forming the ring itself of a compound in which a series of atoms is bonded to form a ring (for example, a monocyclic compound, a fused ring compound, a cross-linked compound, a carbocyclic compound, and a heterocyclic compound). If the ring has a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. If a benzene ring or a naphthalene ring has, for example, an alkyl substituent, the carbon atom in the alkyl substituent is not counted as the ring carbon atom of the benzene or naphthalene ring. In case of a fluorene ring to which a fluorene substituent is bonded (inclusive of a spirofluorene ring), the carbon atom in the fluorene substituent is not counted as the ring carbon atom of the fluorene ring.

The number of “ring atom” referred to herein means the number of the atoms which are members forming the ring itself (for example, a monocyclic ring, a fused ring, and a ring assembly) of a compound in which a series of atoms is bonded to form the ring (for example, a monocyclic compound, a fused ring compound, a cross-linked compound, a carbocyclic compound, and a heterocyclic compound). The atom not forming the ring (for example, hydrogen atom(s) for saturating the valence of the atom which forms the ring) and the atom in a substituent, if the ring is substituted, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In case of a fluorene ring to which a fluorene substituent is bonded (inclusive of a spirofluorene ring), the atom in the fluorene substituent is not counted as the ring atom of the fluorene ring.

The optimal substituent referred to by “substituted or unsubstituted” used herein is preferably selected from the group consisting of an alkyl group having 1 to 50, preferably 1 to 18, more preferably 1 to 8 carbon atoms; a cycloalkyl group having 3 to 50, preferably 3 to 10, more preferably 3 to 8, still more preferably 5 or 6 ring carbon atoms; an aryl group having 6 to 50, preferably 6 to 25, more preferably 6 to 18 ring carbon atoms; an aralkyl group having 7 to 51, preferably 7 to 30, more preferably 7 to 20 carbon atoms which includes an aryl group having 6 to 50, preferably 6 to 25, more preferably 6 to 18 ring carbon atoms; an amino group; a mono- or di-substituted amino group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; an alkoxy group having an alkyl group having 1 to 50, preferably 1 to 18, more preferably 1 to 8 carbon atoms; an aryloxy group having an aryl group having 6 to 50, preferably 6 to 25, more preferably 6 to 18 ring carbon atoms; a mono-, di- or tri-substituted silyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a heteroaryl group having 5 to 50, preferably 5 to 24, more preferably 5 to 13 ring atoms; a haloalkyl group having 1 to 50, preferably 1 to 18, more preferably 1 to 8 carbon atoms; a halogen atom selected from a fluorine atom, a chlorine atom, a bromine atom and an iodine atom; a cyano group; a nitro group; a substituted sulfonyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a di-substituted phosphoryl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

The optional substituent may be further substituted with an optional substituent mentioned above. The optional substituents may be bonded to each other to form a ring.

In the present invention, those which are defined as being preferred can be selected arbitrarily and a combination thereof is a more preferred embodiment.

### [Compound]

The compound of the invention which is useful as a material for organic electroluminescence devices is represented by formula (1);

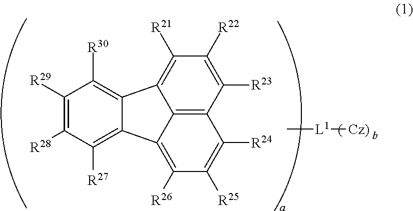

wherein each of R²¹ to R³⁰ independently represents a hydrogen atom or a substituent, provided that one of R²¹ to R³⁰ represents a direct bond to L¹ or Cz; L¹ represents a direct bond, a substituted or unsubstituted, divalent aromatic hydrocarbon group having 6 to 60 ring carbon atoms, a substituted or unsubstituted, divalent oxygen-containing heterocyclic group having 5 to 60 ring atoms, or a substituted or unsubstituted, divalent sulfur-containing heterocyclic group having 5 to 60 ring atoms; Cz represents a structure represented by formula (2); and each of a and b independently represents an integer of 1 to 3, provided that when at least one of a and b is 2 or 3, L¹ is not a direct bond,

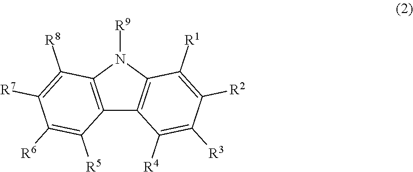

wherein each of R¹ to R⁹ independently represents a hydrogen atom or a substituent; and adjacent groups in R¹ to R⁸, for example, R¹ and R², R² and R³, R³ and R⁴, R⁴ and R⁵, R⁵ and R⁶, R⁶ and R⁷, and R⁷ and R⁸, may be bonded to each other to form a ring structure, provided that at least one adjacent pair in R¹ to R⁸ are bonded to each other to form a ring structure represented by formula (3) or (4):

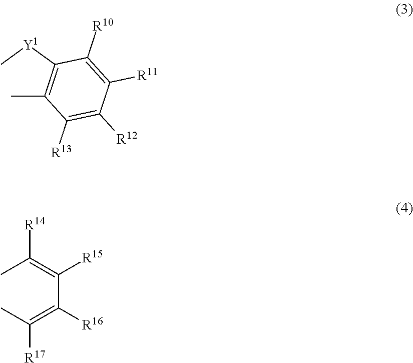

wherein each of R¹⁰ to R¹⁷ independently represents a hydrogen atom or a substituent; adjacent groups in R¹⁰ to R¹³, for example, R¹⁰ and R¹¹, R¹¹ and R¹², and R¹² and R¹³, may be bonded to each other to form a ring structure; Y¹ and R¹⁰ may be bonded to each other to form a ring structure; adjacent groups in R¹⁴ to R¹⁷, for example, R¹⁴ and R¹⁵, R¹⁵ and R¹⁶, and R¹⁶ and R¹⁷, may be bonded to each other to form a ring structure; and Y¹ represents an oxygen atom, a sulfur atom, or —CR³¹R³²—, wherein each of R³¹ and R³² independently represents a hydrogen atom or a substituent, preferably an alkyl group having 1 to 8 carbon atoms or an aryl group having 6 to 18 ring carbon atoms, more preferably a methyl group or a phenyl group;

provided that one of R¹ to R¹⁷, R³¹, and R³² represents a direct bond to L¹ or one of R²¹ to R³⁰.

The ring structure to be formed by the adjacent groups in R¹⁰ to R¹³ which are bonded to each other, the ring structure to be formed by Y¹ and R¹⁰ which are bonded to each other, and the ring structure to be formed by the adjacent groups in R¹⁴ to R¹⁷ which are bonded to each other may be, for example, an aromatic ring, such as a benzene ring and a naphthalene ring, and a ring in which the conjugated system is discontinued. Examples of such a ring structure include those described below.

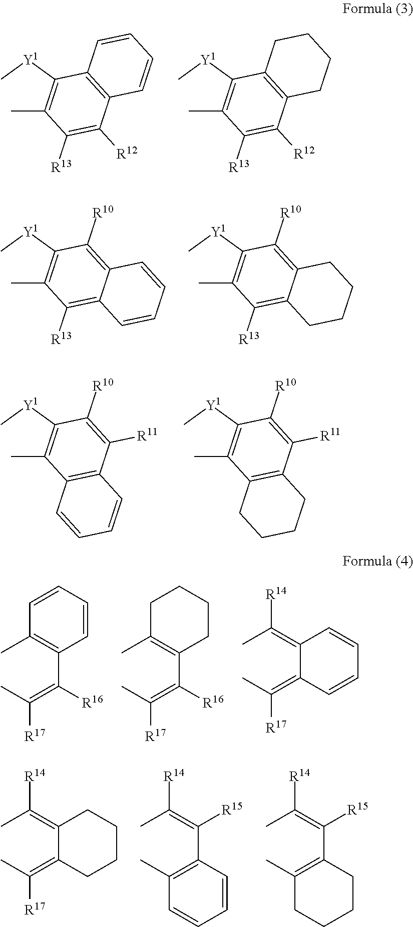

As described above, one of R²¹ to R³⁰ represents a direct bond to L¹ or Cz. Namely, when L¹ represents a direct bond, one of R²¹ to R³⁰ represents a direct bond to Cz, and when L¹ is other than a direct bond, one of R²¹ to R³⁰ represents a direct bond to L¹. The “direct bond” used herein is generally also referred to as “single bond.”

One of R¹ to R¹⁷, R³¹, and R³² represents a direct bond to L¹ or one of R²¹ to R³⁰. Namely, when L¹ represents a direct bond, one of R¹ to R¹⁷, R³¹, and R³² represents a direct bond to one of R²¹ to R³⁰, and when L¹ is other than a direct bond, one of R¹ to R¹⁷, R³¹, and R³² represents a direct bond to L¹. Note that when formula (2) includes formula (3), the phrase “one of R¹ to R¹⁷, R³¹, and R³²” shall read as “one of R¹ to R¹³, R³¹, and R³²,” and when formula (2) includes formula (4), the phrase “one of R¹ to R¹⁷, R³¹, and R³²” shall read as “one of R¹ to R⁹, R¹⁴ to R¹⁷, R³¹, and R³².”

Examples of the divalent aromatic hydrocarbon group having 6 to 60 ring carbon atoms for L¹ include divalent groups obtained by removing two hydrogen atoms from a benzene ring, a naphthalene ring, an anthracene ring, a benzanthracene ring, a phenanthrene ring, a benzophenanthrene ring, a fluorene ring, a benzofluorene ring, a dibenzofluorene ring, a picene ring, a tetracene ring, a pentacene ring, a pyrene ring, a chrysene ring, a benzochrysene ring, a s-indacene ring, an as-indacene ring, a fluoranthene ring, a benzofluoranthene ring, a triphenylene ring, a benzotriphenylene ring, a perylene ring, a coronene ring, or a dibenzanthracene ring. Preferred are divalent aromatic hydrocarbon group having 6 to 40 ring carbon atoms, more preferred are divalent aromatic hydrocarbon groups having 6 to 20 ring carbon atoms, still more preferred are divalent aromatic hydrocarbon groups having 6 to 14 ring carbon atoms, and further preferred are divalent groups obtained by removing two hydrogen atoms form a benzene ring and a naphthalene ring.

Examples of the divalent oxygen-containing heterocyclic group having 5 to 60 ring atoms for L¹ include divalent groups obtained by removing two hydrogen atoms from a furan ring, a benzofuran ring, an isobenzofuran ring, a dibenzofuran ring, a dioxane ring, a morpholine ring, an oxazole ring, an oxadiazole ring, a benzoxazole ring, a pyran ring, a benzonaphtofuran ring, or a dinaphtofuran ring. Preferred are divalent oxygen-containing heterocyclic groups having 5 to 40 ring atoms, more preferred are divalent oxygen-containing heterocyclic groups having 5 to 20 ring atoms, still more preferred are divalent oxygen-containing heterocyclic groups having 5 to 13 ring atoms, and further preferred is a divalent group obtained by removing two hydrogen atoms from a dibenzofuran ring.

Examples of the divalent sulfur-containing heterocyclic group having 5 to 60 ring atoms for L¹ include divalent groups obtained by removing two hydrogen atoms from a benzothiophene ring, a dibenzothiophene ring, a thiophene ring, a thiazole ring, a thiadiazole ring, a benzothiazole ring, a benzonaphthothiophene ring, or a dinaphthothiophene ring. Preferred are divalent sulfur-containing heterocyclic groups having 5 to 40 ring atoms, divalent sulfur-containing heterocyclic groups having 5 to 20 ring atoms, divalent sulfur-containing heterocyclic groups having 5 to 13 ring atoms, and further preferred is a divalent group obtained by removing two hydrogen atoms from a dibenzothiophene ring.

Of the above, the divalent aromatic hydrocarbon group having 6 to 60 ring carbon atoms is preferred for L¹.

As described above, at least one adjacent pair of R¹ to R⁸ in formula (2) are bonded to each other to form a ring structure represented by formula (3) or (4). For example, formula (2) is represented by formulae (5) to (14). Thus, Cz is preferably represented by any one of formulae (5) to (14):

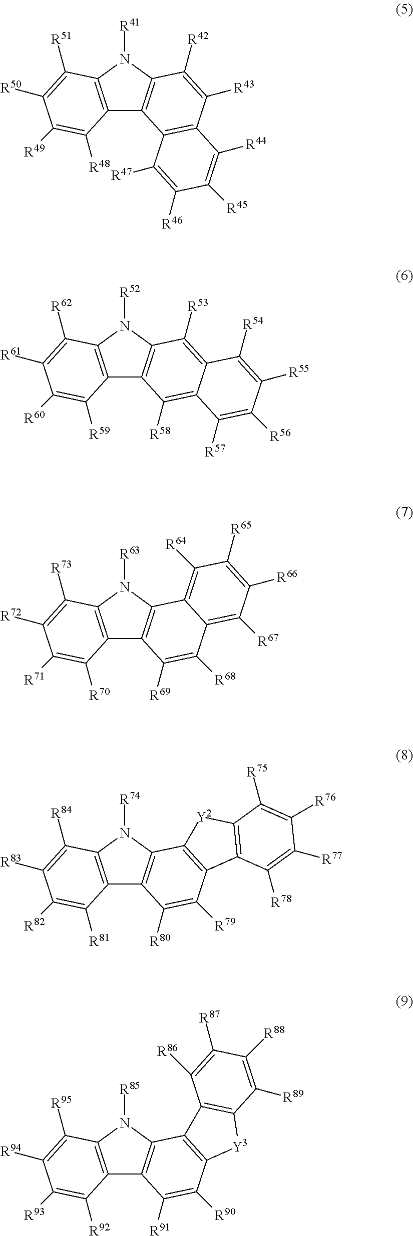

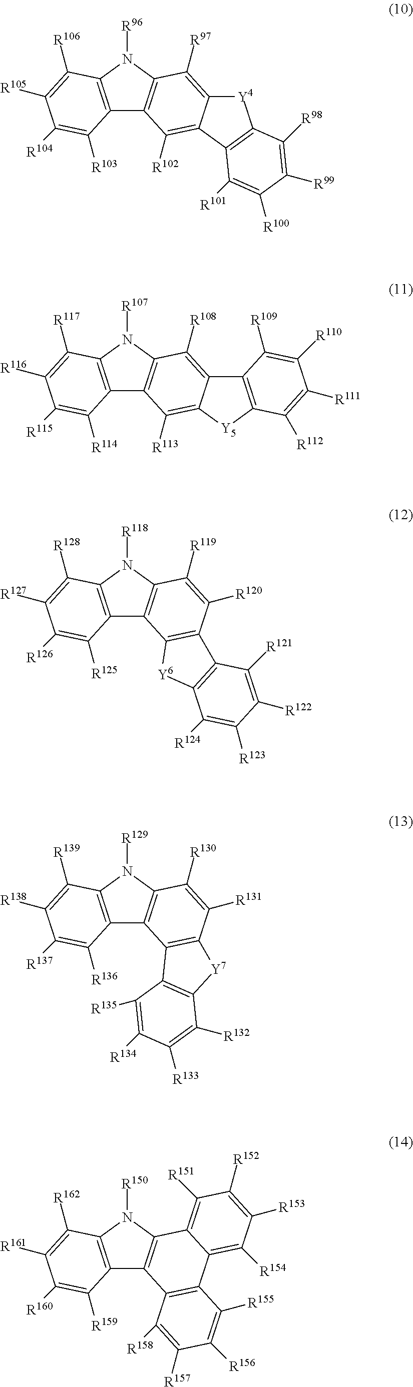

In formulae (5) to (14), each of R⁴¹ to R¹³⁹ and R¹⁵⁰ to R¹⁶² independently represents a hydrogen atom or a substituent. Adjacent groups in R⁴² to R⁵¹, R⁵³ to R⁶², R⁶⁴ to R⁷³, R⁷⁵ to R⁸⁴, R⁸⁶ to R⁹⁵, R⁹⁸ to R¹⁰⁶, R¹⁰⁸ to R¹¹⁷, R¹¹⁹ to R¹²⁸, R¹³⁰ to R¹³⁹, and R¹⁵¹ to R¹⁶² may be bonded to each other to form a ring structure. Each of Y² to Y⁷ represents an oxygen atom, a sulfur atom, or —CR¹⁴⁰R¹⁴¹—. Each of R¹⁴⁰ and R¹⁴¹ independently represents a hydrogen atom or a substituent, and preferably R¹⁴⁰ and R¹⁴¹ are both methyl groups.

One of R⁴¹ to R⁵¹, one of R⁵² to R⁶², one of R⁶³ to R⁷³, one of R⁷⁴ to R⁸⁴, one of R⁸⁵ to R⁹⁵, one of R⁹⁶ to R¹⁰⁶, one of R¹⁰⁷ to R¹¹⁷, one of R¹¹⁸ to R¹²⁸, one of R¹²⁹ to R¹³⁹, and one of R¹⁵⁰ to R¹⁶² each represent a direct bond to L¹ or one of R²¹ to R³⁰.

The ring structure to be formed by the adjacent groups in R⁴² to R⁵¹, R⁵³ to R⁶², R⁶⁴ to R⁷³, R⁷⁵ to R⁸⁴, R⁸⁶ to R⁹⁵, R⁹⁸ to R¹⁰⁶, R¹⁰⁸ to R¹¹⁷, R¹¹⁹ to R¹²⁸, R¹³⁰ to R¹³⁹, and R¹⁵¹ to R¹⁶² which are bonded to each other may be, for example, an aromatic ring, such as a benzene ring and a naphthalene ring, and a ring in which the conjugated system is discontinued.

As described above, the adjacent groups in R⁴² to R⁵¹, R⁵³ to R⁶², and R⁶⁴ to R⁷³ may be bonded to each other to form a ring structure. Examples of such adjacent groups are R⁴² and R⁴³, R⁴³ and R⁴⁴, R⁴⁴ and R⁴⁵, R⁴⁵ and R⁴⁶, R⁴⁶ and R⁴⁷, R⁴⁸ and R⁴⁹, R⁴⁹ and R⁵⁰, and R⁵⁰ and R⁵¹ in formula (5); R⁵³ and R⁵⁴, R⁵⁴ and R⁵⁵, R⁵⁵ and R⁵⁶, R⁵⁶ and R⁵⁷, R⁵⁷ and R⁵⁸, R⁵⁸ and R⁵⁹, R⁵⁹, and R⁶⁰, R⁶⁰ and R⁶¹, and R⁶¹ and R⁶² in formula (6); and R⁶⁴ and R⁶⁵, R⁶⁵ and R⁶⁶, R⁶⁶ and R⁶⁷, R⁶⁷ and R⁶⁸, R⁶⁸ and R⁶⁹, R⁶⁹ and R⁷⁰, R⁷⁰ and R⁷¹, R⁷¹ and R⁷², and R⁷² and R⁷³ in formula (7).

As described above, the adjacent groups in R⁷⁵ to R⁸⁴, R⁸⁶ to R⁹⁵, and R⁹⁸ to R¹⁰⁶ may be bonded to each other to form a ring structure. Examples of such adjacent groups are R⁷⁵ and R⁷⁶, R⁷⁶ and R⁷⁷, R⁷⁷ and R⁷⁸, R⁷⁸ and R⁷⁹, R⁷⁹ and R⁸⁰, R⁸⁰ and R⁸¹, R⁸¹ and R⁸², R⁸² and R⁸³, and R⁸³ and R⁸⁴ in formula (8); R⁸⁶ and R⁸⁷, R⁸⁷ and R⁸⁸, R⁸⁸ and R⁸⁹, R⁹⁰ and R⁹¹, R⁹¹ and R⁹², R⁹² and R⁹³, R⁹³ and R⁹⁴, and R⁹⁴ and R⁹⁵ in formula (9); and R⁹⁸ and R⁹⁹, R⁹⁹ and R¹⁰⁰, R¹⁰⁰ and R¹⁰¹, R¹⁰¹ and R¹⁰², R¹⁰² and R¹⁰³, R¹⁰³ and R¹⁰⁴, R¹⁰⁴ and R¹⁰⁵, and R¹⁰⁵ and R¹⁰⁶ in formula (10), provided that a pair of R¹⁰¹ and R¹⁰² and a pair of R¹⁰² and R¹⁰³ do not form ring structures at the same time.

As described above, the adjacent groups in R¹⁰⁸ to R¹¹⁷, R¹¹⁹ to R¹²⁸, R¹³⁰ to R¹³⁹, and R¹⁵¹ to R¹⁶² may be bonded to each other to form a ring structure. Examples of such adjacent groups are R¹⁰⁸ and R¹⁰⁹, R¹⁰⁹ and R¹¹⁰, R¹¹⁰ and R¹¹¹, R¹¹¹ and R¹¹², R¹¹³ and R¹¹⁴, R¹¹⁴ and R¹¹⁵, R¹¹⁵ and R¹¹⁶, and R¹¹⁶ and R¹¹⁷ in formula (11); R¹¹⁹ and R¹²⁰, R¹²⁰ and R¹²¹, R¹²¹ and R¹²², R¹²² and R¹²³, R¹²³ and R¹²⁴, R¹²⁵ and R¹²⁶, R¹²⁶ and R¹²⁷, and R¹²⁷ and R¹²⁸ in formula (12); R¹³⁰ and R¹³¹, R¹³² and R¹³³, R¹³³ and R¹³⁴, R¹³⁴ and R¹³⁵, R¹³⁶ and R¹³⁷, R¹³⁷ and R¹³⁸, and R¹³⁸ and R¹³⁹ in formula (13); and R¹⁵¹ and R¹⁵², R¹⁵² and R¹⁵³, R¹⁵³ and R¹⁵⁴, R¹⁵⁴ and R¹⁵⁵, R¹⁵⁵ and R¹⁵⁶, R¹⁵⁶ and R¹⁵⁷, R¹⁵⁷ and R¹⁵⁸, R¹⁵⁹ and R¹⁶⁰, R¹⁶⁰ and R¹⁶¹, and R¹⁶¹ and R¹⁶² in formula (14).

The substituent recited in each definition of formulae (1) to (14) is not particularly limited and it may be a conventionally known organic group. Examples thereof include a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted aralkyl group having 7 to 51 carbon atoms, an amino group, a mono- or di-substituted amino group wherein the substituent is selected from a substituted or unsubstituted alkyl group having 1 to 50 carbon atoms and a substituted or unsubstituted aryl group having 6 to 50 ring carbon atoms, a substituted or unsubstituted alkoxy group having 1 to 50 carbon atoms, a substituted or unsubstituted aryloxy group having 6 to 50 ring carbon atoms, a mono-, di- or tri-substituted silyl group wherein the substituent is selected from a substituted or unsubstituted alkyl group having 1 to 50 carbon atoms and a substituted or unsubstituted aryl group having 6 to 50 ring carbon atoms, a substituted or unsubstituted heteroaryl group having 5 to 50 ring atoms, a substituted or unsubstituted haloalkyl group having 1 to 50 carbon atoms, a halogen atom, a cyano group, a nitro group, and a sulfonyl group substituted by a substituent selected from a substituted or unsubstituted alkyl group having 1 to 50 carbon atoms and a substituted or unsubstituted aryl group having 6 to 50 ring carbon atoms.

Of the above, preferred are a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted aralkyl group having 7 to 51 carbon atoms, an amino group, a mono- or di-substituted amino group wherein the substituent is selected from a substituted or unsubstituted alkyl group having 1 to 50 carbon atoms and a substituted or unsubstituted aryl group having 6 to 50 ring carbon atoms, a substituted or unsubstituted alkoxy group having 1 to 50 carbon atoms, a substituted or unsubstituted aryloxy group having 6 to 50 ring carbon atoms, a mono-, di- or tri-substituted silyl group wherein the substituent is selected from a substituted or unsubstituted alkyl group having 1 to 50 carbon atoms and a substituted or unsubstituted aryl group having 6 to 50 ring carbon atoms, a substituted or unsubstituted heteroaryl group having 5 to 50 ring atoms, a substituted or unsubstituted haloalkyl group having 1 to 50 carbon atoms, a halogen atom, a cyano group, and a nitro group.

More preferred are a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a mono- or di-substituted amino group wherein the substituent is selected from a substituted or unsubstituted alkyl group having 1 to 50 carbon atoms and a substituted or unsubstituted aryl group having 6 to 50 ring carbon atoms, a substituted or unsubstituted heteroaryl group having 5 to 50 ring atoms, a halogen atom, and a cyano group.

Examples of the alkyl group having 1 to 50, preferably 1 to 18, more preferably 1 to 8 carbon atoms include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), a dodecyl group (inclusive of isomeric groups), a tridecyl group, a tetradecyl group, an octadecyl group, a tetracosanyl group, and a tetracontanyl group. Preferred examples include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), a dodecyl group (inclusive of isomeric groups), a tridecyl group, a tetradecyl group, and an octadecyl group. More preferred examples include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), and an octyl group (inclusive of isomeric groups).

Examples of the cycloalkyl group having 3 to 50, preferably 3 to 10, more preferably 3 to 8, still more preferably 5 or 6 ring carbon atoms include a cyclopropyl group, a cyclobutyl group, a cyclopentyl group, a cyclohexyl group, a cycloheptyl group, a cyclooctyl group, and an adamantyl group, with a cyclopentyl group and a cyclohexyl group being preferred.

Examples of the aryl group having 6 to 50, preferably 6 to 25, more preferably 6 to 18 ring carbon atoms include a phenyl group, a naphthyl group, a naphthylphenyl group, a biphenylyl group, a terphenylyl group, an acenaphthylenyl group, an anthryl group, a benzanthryl group, an aceanthryl group, a phenanthryl group, a benzophenanthryl group, a phenalenyl group, a fluorenyl group, a 9,9′-spirobifluorenyl group, a benzofluorenyl group, a dibenzofluorenyl group, a picenyl group, a pentaphenyl group, a pentacenyl group, a pyrenyl group, a chrysenyl group, a benzochrysenyl group, a s-indacenyl group, an as-indacenyl group, a fluoranthenyl group, a benzofluoranthenyl group, a tetracenyl group, a triphenylenyl group, a benzotriphenylenyl group, a perylenyl group, a coronyl group, and a dibenzanthryl group.

Examples of the arylene group having 6 to 50, preferably 6 to 25, and more preferably 6 to 18 ring carbon atoms include those derived from the aryl group mentioned above by removing one hydrogen atom.

The heteroaryl group having 5 to 50, preferably 5 to 24, more preferably 5 to 13 ring atoms include at least one, preferably 1 to 5, more preferably 1 to 3, and still more preferably 1 or 2 hetero atoms, for example, a nitrogen atom, a sulfur atom, an oxygen atom, and a phosphorus atom. Examples thereof include a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isooxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a carbazolyl group, a phenanthridinyl group, an acridinyl group, a phenanthrolinyl group, a phenazinyl group, a phenothiazinyl group, a phenoxazinyl group, an azatriphenylenyl group, a diazatriphenylenyl group, a xanthenyl group, an azacarbazolyl group, an azadibenzofuranyl group, an azadibenzothiophenyl group, a benzofuranobenzothiophenyl group, a benzothienobenzothiophenyl group, a dibenzofuranonaphthyl group, a dibenzothienonaphthyl group, and a dinaphthothienothiophenyl group. Preferred are a pyridyl group, a pyrimidinyl group, a triazinyl group, a pyrazinyl group, a quinolinyl group, a isoquinolinyl group, a quinoxalinyl group, and a quinazolinyl group.

In addition, examples of the heteroaryl group having 5 to 50 ring atoms further include mono-valent groups derived from the following compounds by removing one hydrogen atom;

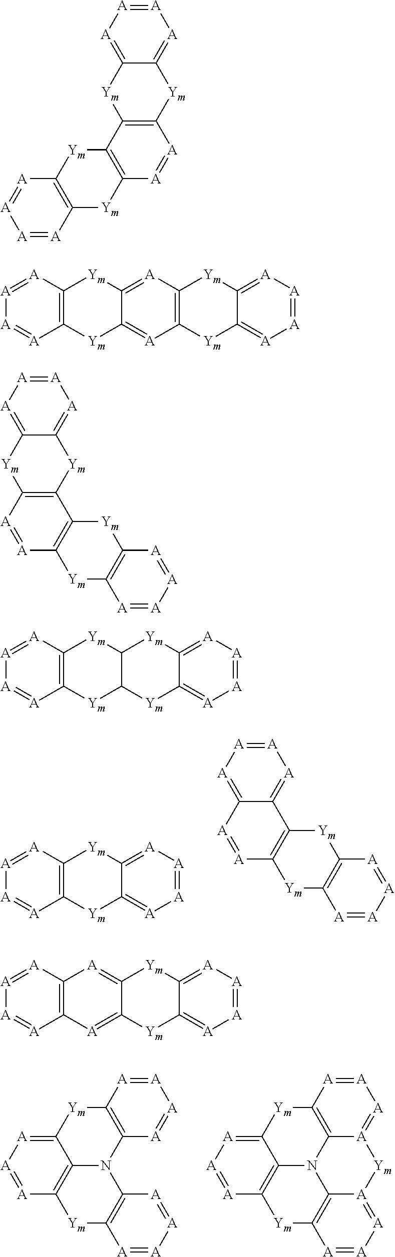

wherein;

each A independently represents CR¹⁰⁰ or a nitrogen atom;

each R¹⁰⁰ independently represents a hydrogen atom or a substituent;

each Y independently represents a single bond, C(R¹⁰¹)(R¹⁰²), an oxygen atom, a sulfur atom, or N(R¹⁰³);

each of R¹⁰¹, R¹⁰² and R¹⁰³ independently represents a hydrogen atom or a substituent; and

m independently represents 0 or 1.

The substituent referred to above is selected from those mentioned above.

Examples of the aralkyl group having 7 to 51 total carbon atoms which includes an aryl group having 6 to 50, preferably 6 to 25, more preferably 6 to 18 ring carbon atoms include those having the aryl group mentioned above.

Examples of the mono- or di-substituted amino group having a substituent selected from an alkyl group having 1 to 50, preferably 1 to 18, more preferably 1 to 8 carbon atoms and an aryl group having 6 to 50, preferably 6 to 25, more preferably 6 to 18 ring carbon atoms include those having a substituent selected from the alkyl group and the aryl group each mentioned above.

Examples of the alkoxy group having an alkyl group having 1 to 50, preferably 1 to 18, and more preferably 1 to 8 carbon atoms include those having the alkyl group mentioned above.

Examples of the aryloxy group having an aryl group having 6 to 50, preferably 6 to 25, and more preferably 6 to 18 ring carbon atoms include those having the aryl group mentioned above.

Examples of the mono-, di- or tri-substituted silyl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms include those having a substituent selected from the alkyl group and the aryl group each mentioned above.

Examples of the haloalkyl group having 1 to 50, preferably 1 to 18, and more preferably 1 to 8 carbon atoms include those derived from the alkyl group mentioned above by replacing one hydrogen atom with a halogen atom, such as a fluorine atom, a chlorine atom, a bromine atom, and an iodine atom.

Examples of the sulfonyl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms include those having a substituent selected from the alkyl group and the aryl group each mentioned above.

Examples of the di-substituted phosphoryl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms include those having substituents selected from the alkyl group and the aryl group each mentioned above.

In the compound of the invention, R⁹ of formula (2) is preferably a direct bond to L¹ or one of R²¹ to R³⁰, although not particularly limited thereto.

In addition, each of a and b in the compound of the invention is preferably 1 or 2 and more preferably 1.

Thus, the compound of the invention is preferably represented by any of formulae (1-5) to (1-14).

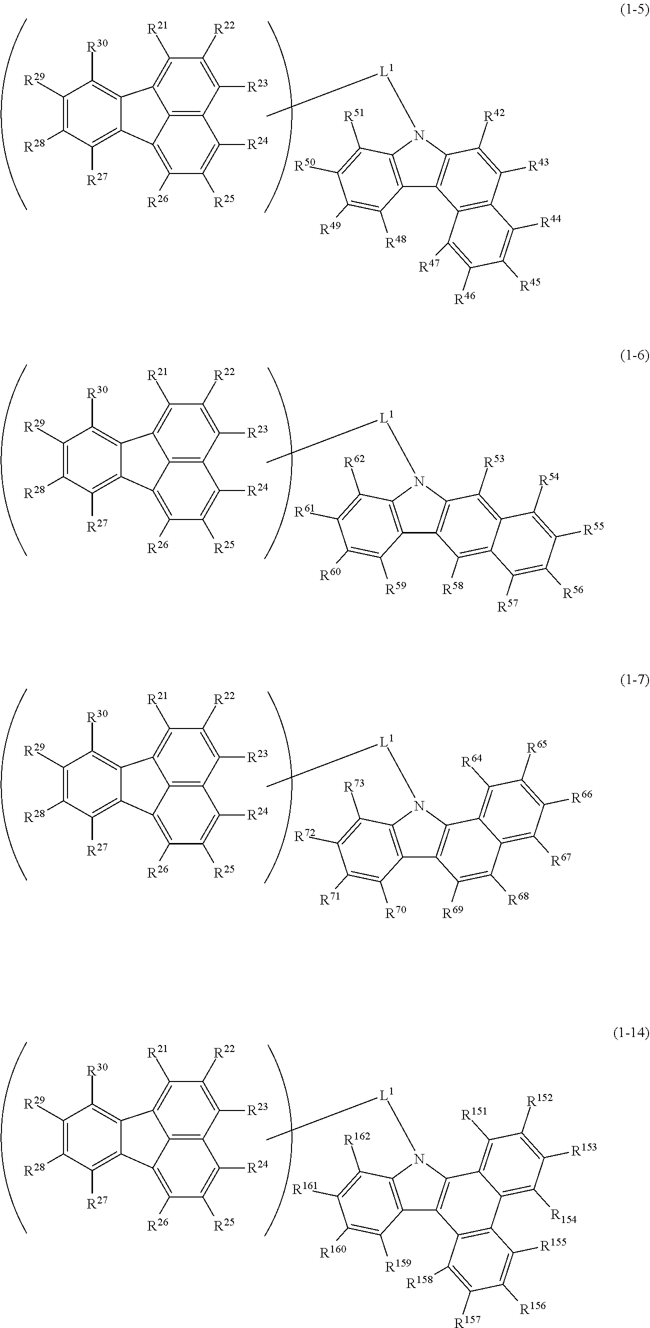

L¹, R²¹ to R³⁰, R⁴² to R⁵¹, R⁵³ to R⁶², R⁶⁴ to R⁷³, R¹⁵¹ to R¹⁶², and their preferred examples are as defined above.

Therefore, the adjacent groups in R⁴² to R⁵¹, R⁵³ to R⁶², R⁶⁴ to R⁷³, and R¹⁵¹ to R¹⁶² may be bonded to each other to form a ring structure. Examples of such adjacent groups are R⁴² and R⁴³, R⁴³ and R⁴⁴, R⁴⁴ and R⁴⁵, R⁴⁵ and R⁴⁶, R⁴⁶ and R⁴⁷, R⁴⁸ and R⁴⁹, R⁴⁹ and R⁵⁰, and R⁵⁰ and R⁵¹ in formula (1-5); R⁵³ and R⁵⁴, R⁵⁴ and R⁵⁵, R⁵⁵ and R⁵⁶, R⁵⁶ and R⁵⁷, R⁵⁷ and R⁵⁸, R⁵⁸ and R⁵⁹, R⁵⁹ and R⁶⁰, R⁶⁰ and R⁶¹, and R⁶¹ and R⁶² in formula (1-6); R⁶⁴ and R⁶⁵, R⁶⁵ and R⁶⁶, R⁶⁶ and R⁶⁷, R⁶⁷ and R⁶⁸, R⁶⁸ and R⁶⁹, R⁶⁹ and R⁷⁰, R⁷⁰ and R⁷¹, R⁷¹ and R⁷², and R⁷² and R⁷³ in formula (1-7); and R¹⁵¹ and R¹⁵², R¹⁵² and R¹⁵³, R¹⁵³ and R¹⁵⁴, R¹⁵⁴ and R¹⁵⁵, R¹⁵⁵ and R¹⁵⁶, R¹⁵⁶ and R¹⁵⁷, R¹⁵⁷ and R¹⁵⁸, R¹⁵⁹ and R¹⁶⁹, R¹⁶⁹ and R¹⁶¹, and R¹⁶¹ and R¹⁶² in formula (1-14).

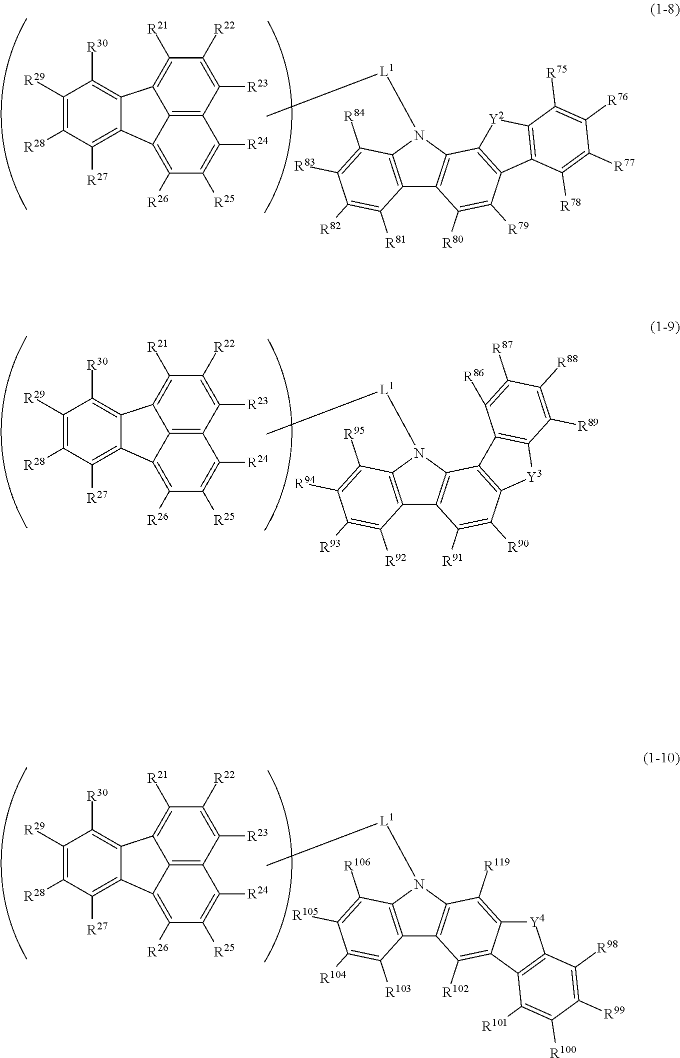

L¹, R²¹ to R³⁰, R⁷⁵ to R⁸⁴, R⁸⁶ to R⁹⁵, R⁹⁷ to R¹⁰⁶, Y² to Y⁴, and their preferred examples are as defined above.

Therefore, the adjacent groups in R⁷⁵ to R⁸⁴, R⁸⁶ to R⁹⁵, and R⁹⁸ to R¹⁰⁶ may be bonded to each other to form a ring structure. Examples of such adjacent groups are R⁷⁵ and R⁷⁶, R⁷⁶ and R⁷⁷, R⁷⁷ and R⁷⁸, R⁷⁸ and R⁷⁹, R⁷⁹ and R⁸⁰, R⁸⁰ and R⁸¹, R⁸¹ and R⁸², R⁸² and R⁸³, and R⁸³ and R⁸⁴ in formula (1-8); R⁸⁶ and R⁸⁷, R⁸⁷ and R⁸⁸, R⁸⁸ and R⁸⁹, R⁹⁰ and R⁹¹, R⁹¹ and R⁹², R⁹² and R⁹³, R⁹³ and R⁹⁴, and R⁹⁴ and R⁹⁵ in formula (1-9); and R⁹⁸ and R⁹⁹, R⁹⁹ and R¹⁰⁰, R¹⁰⁰ and R¹⁰¹, R¹⁰¹ and R¹⁰², R¹⁰² and R¹⁰³, R¹⁰³ and R¹⁰⁴, R¹⁰⁴ and R¹⁰⁵, and R¹⁰⁵ and R¹⁰⁶ in formula (1-10), provided that a pair of R¹⁰¹ and R¹⁰² and a pair of R¹⁰² and R¹⁰³ do not form a ring structure at the same time.

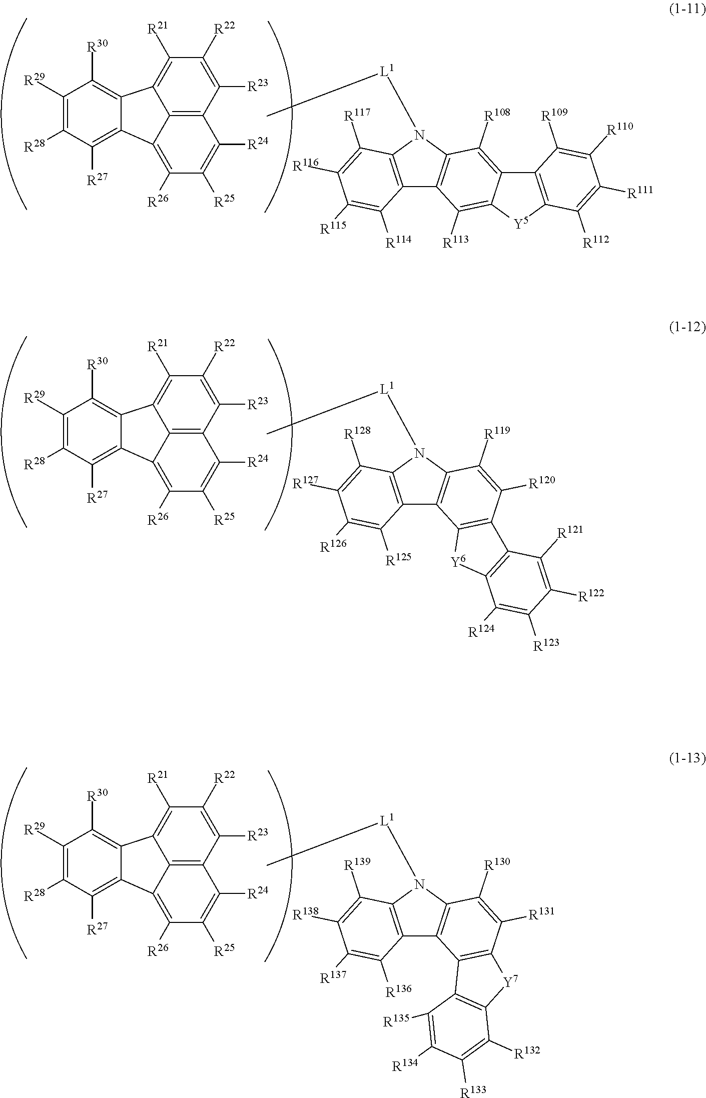

L¹, R²¹ to R³⁰, R¹⁰⁸ to R¹¹⁷, R¹¹⁹ to R¹²⁸, R¹³⁰ to R¹³⁹, Y⁵ to Y⁷, and their preferred examples are as defined above.

Therefore, the adjacent groups in R¹⁰⁸ to R¹¹⁷, R¹¹⁹ to R¹²⁸, and R¹³⁰ to R¹³⁹ may be bonded to each other to form a ring structure. Examples of such adjacent groups are R¹⁰⁸ and R¹⁰⁹, R¹⁰⁹ and R¹¹⁰, R¹¹⁰ and R¹¹¹, R¹¹¹ and R¹¹², R¹¹³ and R¹¹⁴, R¹¹⁴ and R¹¹⁵, R¹¹⁵ and R¹¹⁶, and R¹¹⁶ and R¹¹⁷ in formula (1-11); R¹¹⁹ and R¹²⁰, R¹²⁰ and R¹²¹, R¹²¹ and R¹²², R¹²² and R¹²³, R¹²³ and R¹²⁴, R¹²⁵ and R¹²⁶, R¹²⁶ and R¹²⁷, and R¹²⁷ and R¹²⁸ in formula (1-12); and R¹³⁰ and R¹³¹, R¹³² and R¹³³, R¹³³ and R¹³⁴, R¹³⁴ and R¹³⁵, R¹³⁶ and R¹³⁷, R¹³⁷ and R¹³⁸, and R¹³⁸ and R¹³⁹ in formula (1-13).

The compound of the invention represented by formula (1′) or (1″) is also preferred. Each group, a, b, and their preferred embodiments are as defined in formula (1).

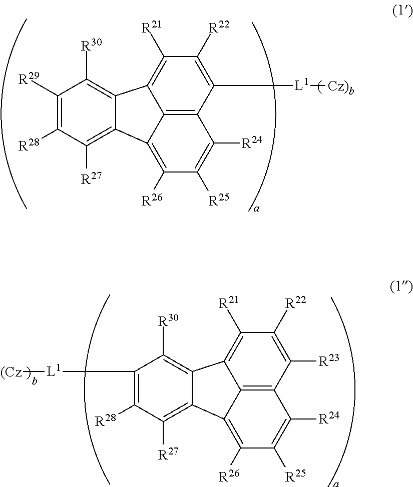

The compound represented by formula (1′) and the compound represented by formula (1″) are more preferably represented by any of formulae (1′-5) to (1′-14) and any of formulae (1″-5) to (1″-14), respectively.

Each of the groups in formulae (1′-5) to (1′-14) and their preferred examples are as defined in formula (1′). Each of the groups in formulae (1″-5) to (1″-14) and their preferred examples are as defined in formula (1″).

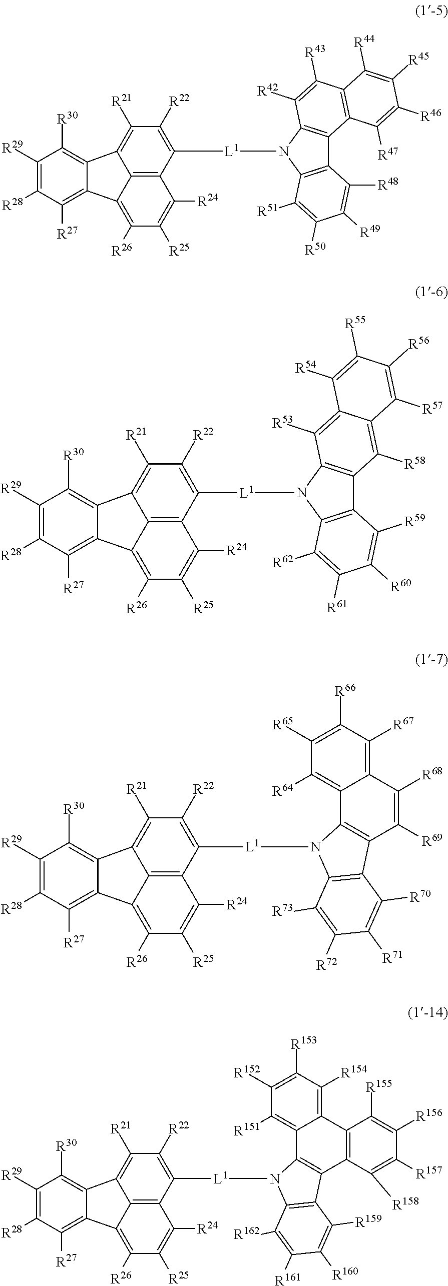

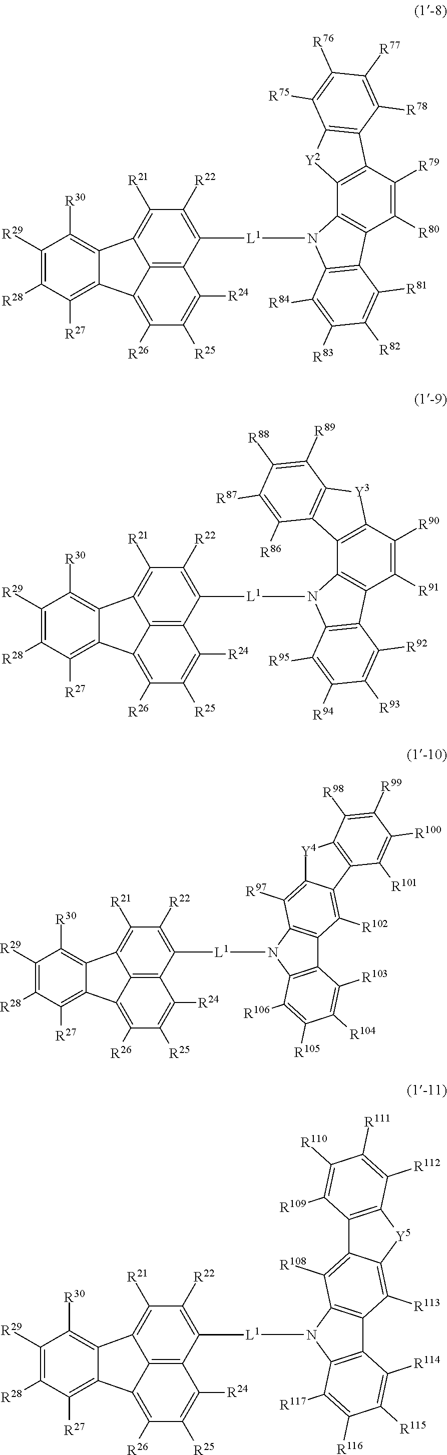

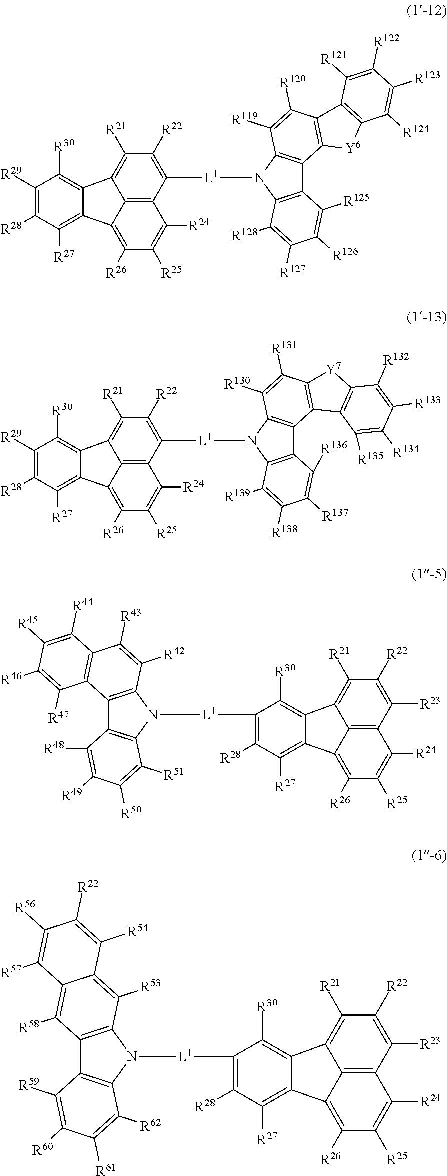

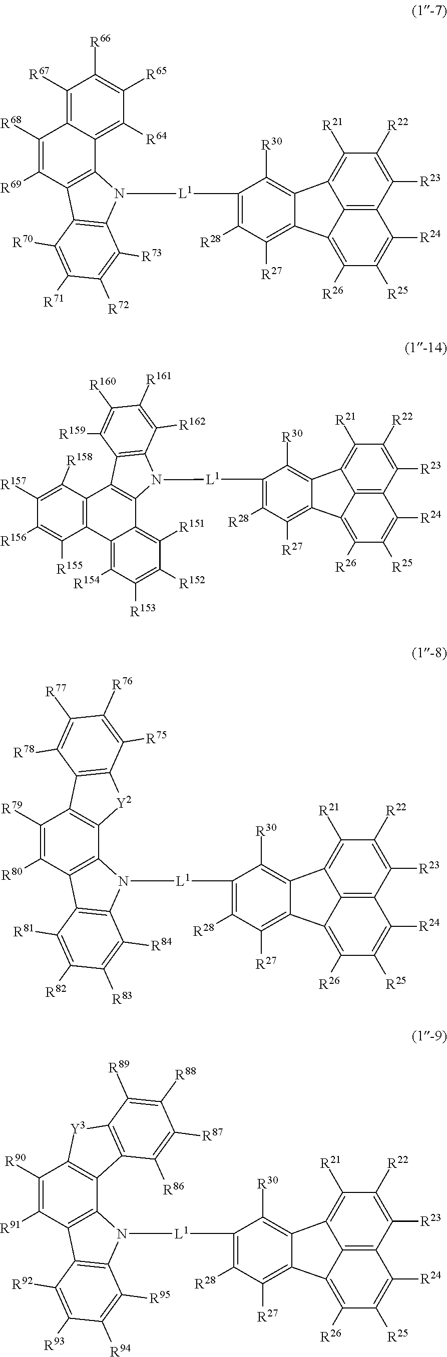

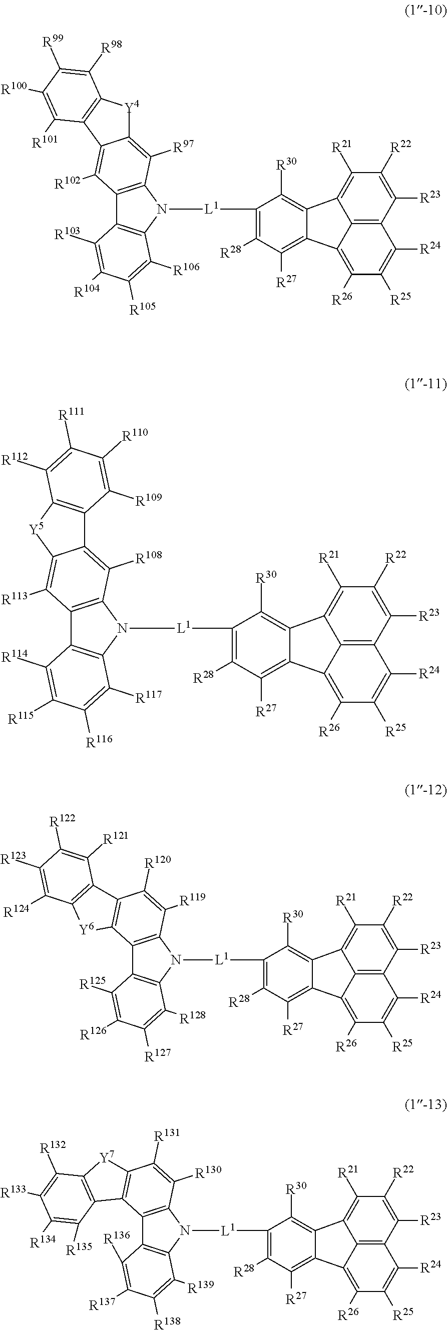

In view of the driving voltage of an organic EL device employing the compound of the invention, a fused structure in which two or more rings are fused to the carbazole skeleton is particularly preferred for Cz in formula (1), because the effect of reducing the voltage by the improved molecular packing is obtained. Namely, in the preferred fused structure, two or more pairs of adjacent groups in R¹ to R⁸ of formula (2) form the ring structures of formula (4), or one or more pairs of adjacent groups in R¹ to R⁸ form the structure(s) of formula (3). In addition, Cz is preferably a structure represented by any of formulae (8) to (14).

In view of the emission efficiency, the N-position of the fused carbazole skeleton represented by formulae (5) to (14) is preferably bonded to the fluoranthene-containing group directly or via L¹.

The compound of the invention wherein R²¹ to R³⁰ except for one which represents a direct bond to L¹ or Cz are all hydrogen atoms is preferred.

Examples of the compound of the invention are shown below, although not particularly limited thereto. The following examples may be said to be preferred compounds.

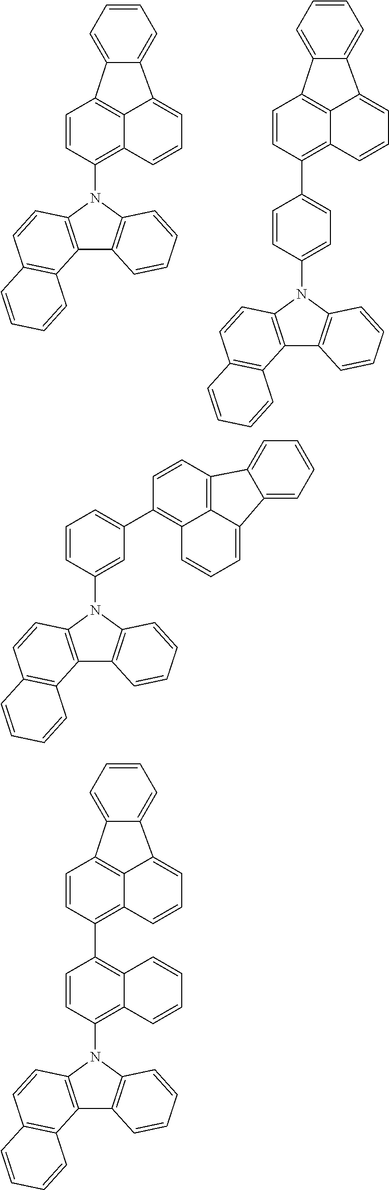

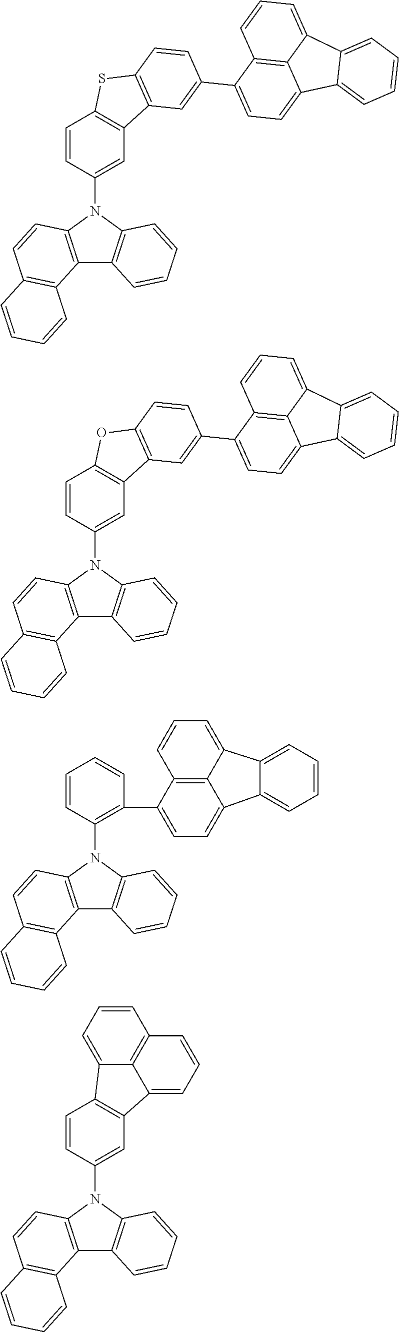 [SMILES: c1ccc2c(c1)-c1cccc3c(-c4ccc5sc6ccc(-n7c8ccccc8c8c9ccccc9ccc87)cc6c5c4)ccc-2c13 ; c1ccc2c(c1)-c1cccc3c(-c4ccc5oc6ccc(-n7c8ccccc8c8c9ccccc9ccc87)cc6c5c4)ccc-2c13 ; c1ccc2c(c1)-c1cccc3c(-c4ccccc4-n4c5ccccc5c5c6ccccc6ccc54)ccc-2c13 ; c1cc2c3c(cccc3c1)-c1cc(-n3c4ccccc4c4c5ccccc5ccc43)ccc1-2]

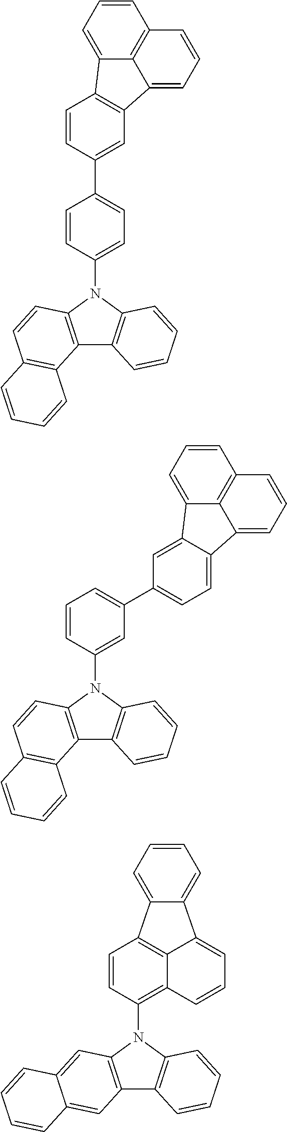 [SMILES: c1cc2c3c(cccc3c1)-c1cc(-c3ccc(-n4c5ccccc5c5c6ccccc6ccc54)cc3)ccc1-2 ; c1cc(-c2ccc3c(c2)-c2cccc4cccc-3c24)cc(-n2c3ccccc3c3c4ccccc4ccc32)c1 ; c1ccc2c(c1)-c1cccc3c(-n4c5ccccc5c5cc6ccccc6cc54)ccc-2c13]

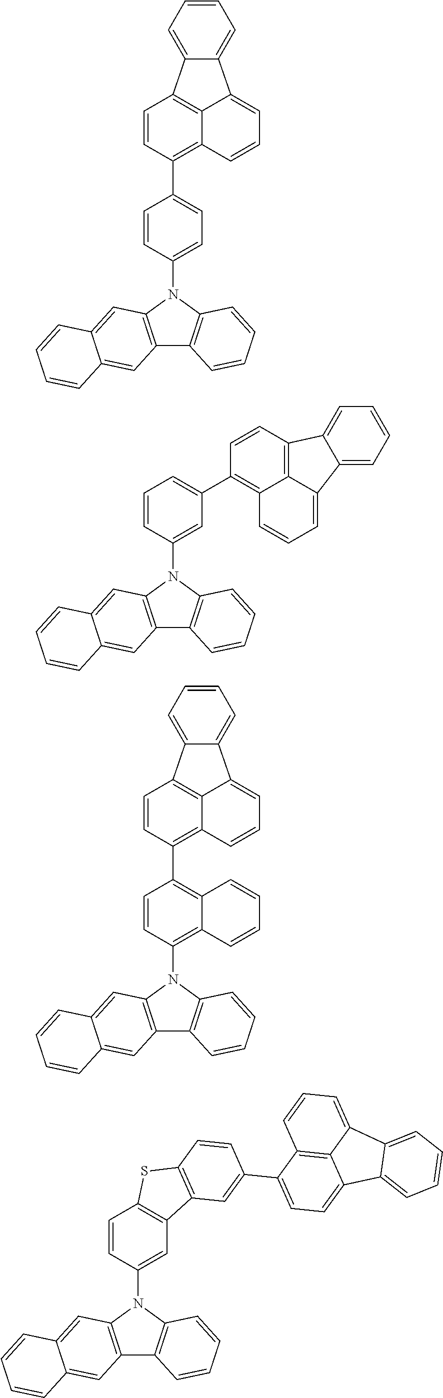

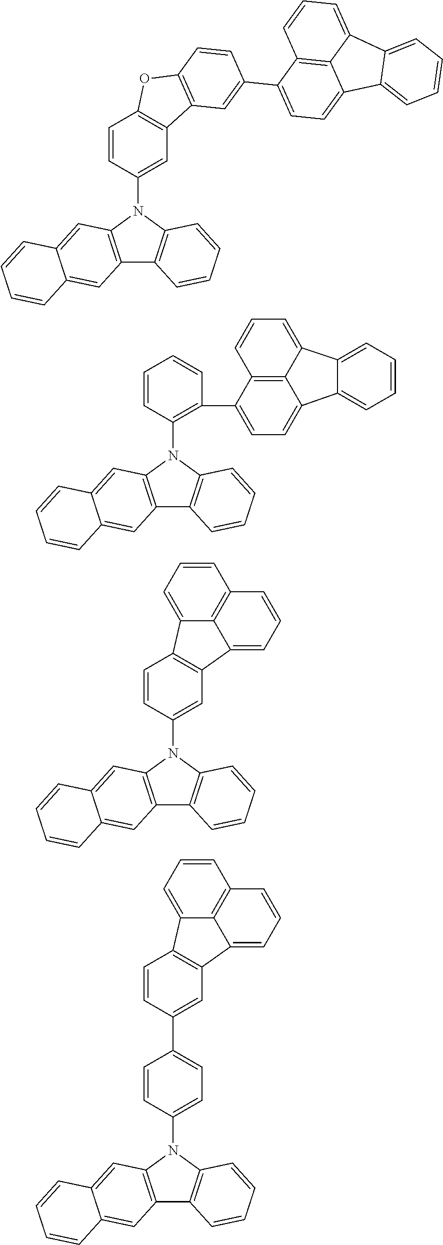

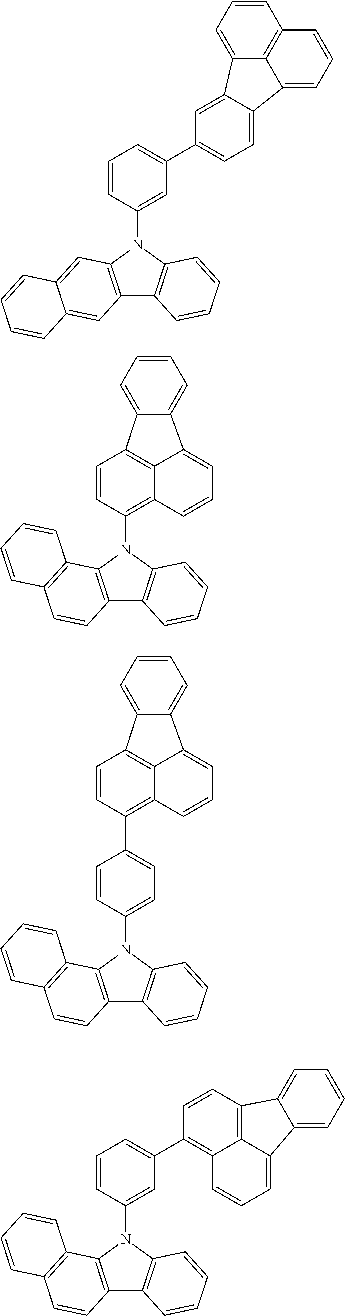

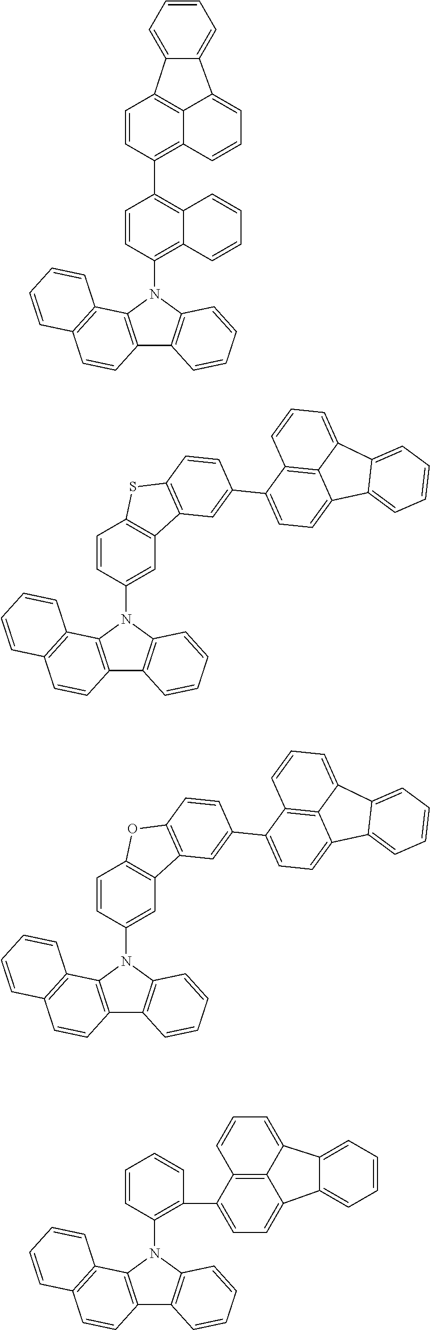 [SMILES: c1ccc2c(c1)-c1cccc3c(-c4ccc(-n5c6ccccc6c6ccc7ccccc7c65)c5ccccc45)ccc-2c13 ; c1ccc2c(c1)-c1cccc3c(-c4ccc5sc6ccc(-n7c8ccccc8c8ccc9ccccc9c87)cc6c5c4)ccc-2c13 ; c1ccc2c(c1)-c1cccc3c(-c4ccc5oc6ccc(-n7c8ccccc8c8ccc9ccccc9c87)cc6c5c4)ccc-2c13 ; c1ccc2c(c1)-c1cccc3c(-c4ccccc4-n4c5ccccc5c5ccc6ccccc6c54)ccc-2c13]

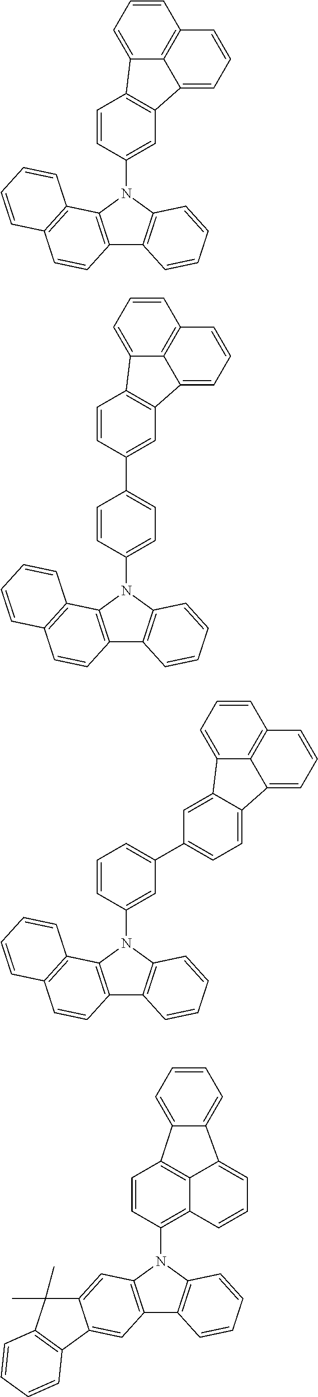

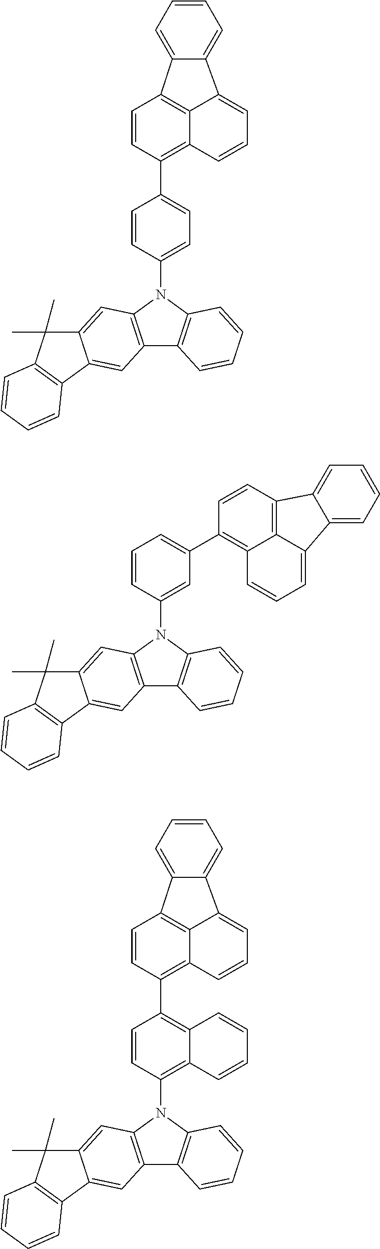 [SMILES: CC1(C)c2ccccc2-c2cc3c4ccccc4n(-c4ccc(-c5ccc6c7c(cccc57)-c5ccccc5-6)cc4)c3cc21 ; CC1(C)c2ccccc2-c2cc3c4ccccc4n(-c4cccc(-c5ccc6c7c(cccc57)-c5ccccc5-6)c4)c3cc21 ; CC1(C)c2ccccc2-c2cc3c4ccccc4n(-c4ccc(-c5ccc6c7c(cccc57)-c5ccccc5-6)c5ccccc45)c3cc21]

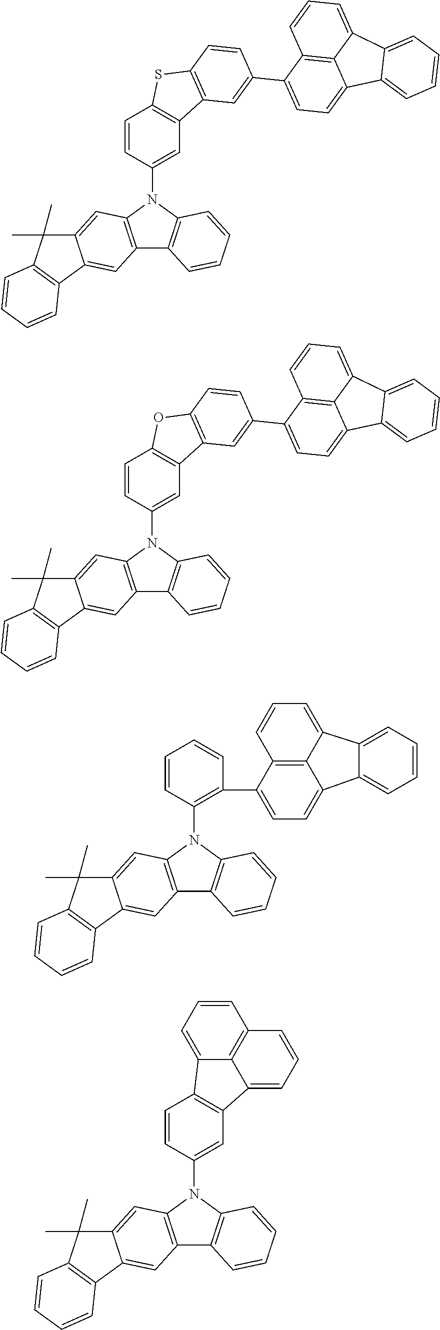 [SMILES: CC1(C)c2ccccc2-c2cc3c4ccccc4n(-c4ccc5sc6ccc(-c7ccc8c9c(cccc79)-c7ccccc7-8)cc6c5c4)c3cc21 ; CC1(C)c2ccccc2-c2cc3c4ccccc4n(-c4ccc5oc6ccc(-c7ccc8c9c(cccc79)-c7ccccc7-8)cc6c5c4)c3cc21 ; CC1(C)c2ccccc2-c2cc3c4ccccc4n(-c4ccccc4-c4ccc5c6c(cccc46)-c4ccccc4-5)c3cc21 ; CC1(C)c2ccccc2-c2cc3c4ccccc4n(-c4ccc5c(c4)-c4cccc6cccc-5c46)c3cc21]

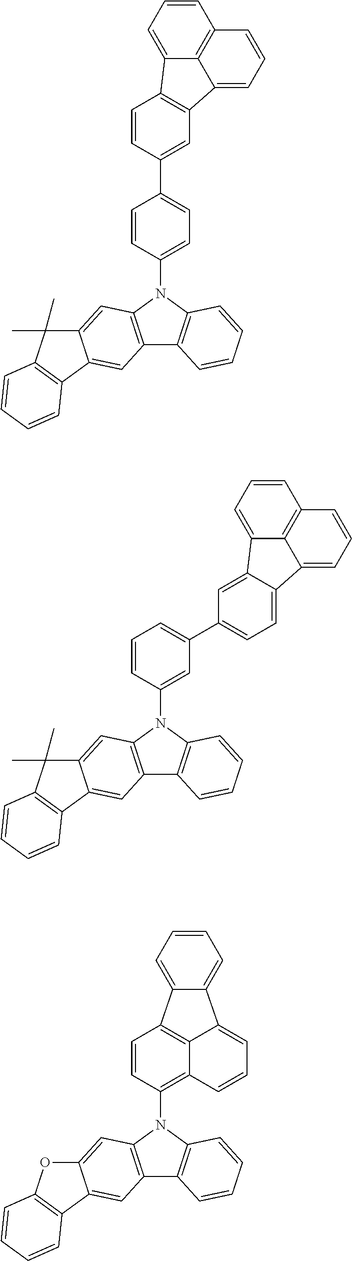 [SMILES: CC1(C)c2ccccc2-c2cc3c4ccccc4n(-c4ccc(-c5ccc6c(c5)-c5cccc7cccc-6c57)cc4)c3cc21 ; CC1(C)c2ccccc2-c2cc3c4ccccc4n(-c4cccc(-c5ccc6c(c5)-c5cccc7cccc-6c57)c4)c3cc21 ; c1ccc2c(c1)-c1cccc3c(-n4c5ccccc5c5cc6c(cc54)oc4ccccc46)ccc-2c13]

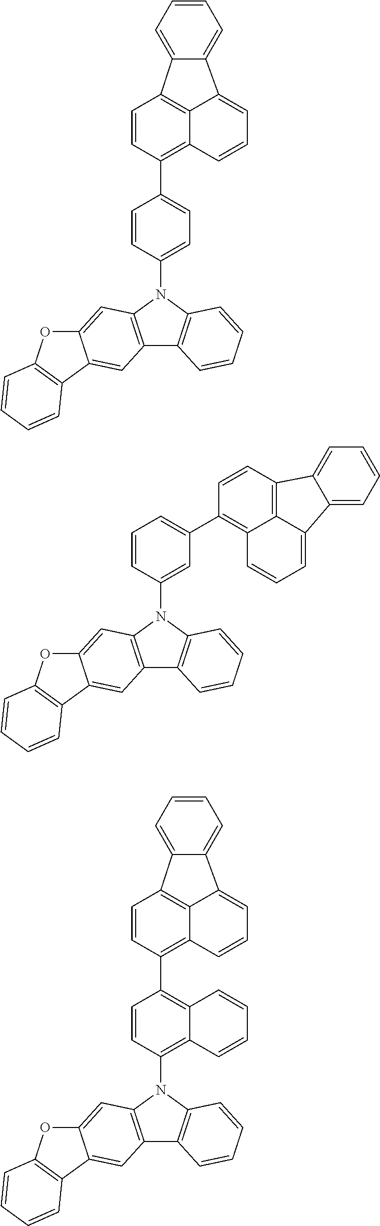 [SMILES: c1ccc2c(c1)-c1cccc3c(-c4ccc(-n5c6ccccc6c6cc7c(cc65)oc5ccccc57)cc4)ccc-2c13 ; c1cc(-c2ccc3c4c(cccc24)-c2ccccc2-3)cc(-n2c3ccccc3c3cc4c(cc32)oc2ccccc24)c1 ; c1ccc2c(c1)-c1cccc3c(-c4ccc(-n5c6ccccc6c6cc7c(cc65)oc5ccccc57)c5ccccc45)ccc-2c13]

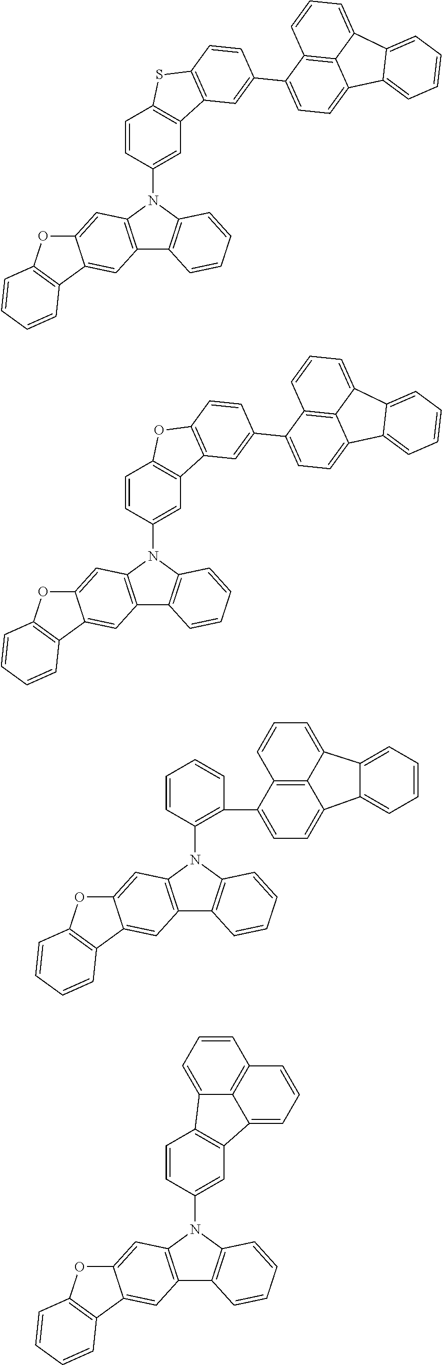 [SMILES: c1ccc2c(c1)-c1cccc3c(-c4ccc5sc6ccc(-n7c8ccccc8c8cc9c(cc87)oc7ccccc79)cc6c5c4)ccc-2c13 ; c1ccc2c(c1)-c1cccc3c(-c4ccc5oc6ccc(-n7c8ccccc8c8cc9c(cc87)oc7ccccc79)cc6c5c4)ccc-2c13 ; c1ccc2c(c1)-c1cccc3c(-c4ccccc4-n4c5ccccc5c5cc6c(cc54)oc4ccccc46)ccc-2c13 ; c1cc2c3c(cccc3c1)-c1cc(-n3c4ccccc4c4cc5c(cc43)oc3ccccc35)ccc1-2]

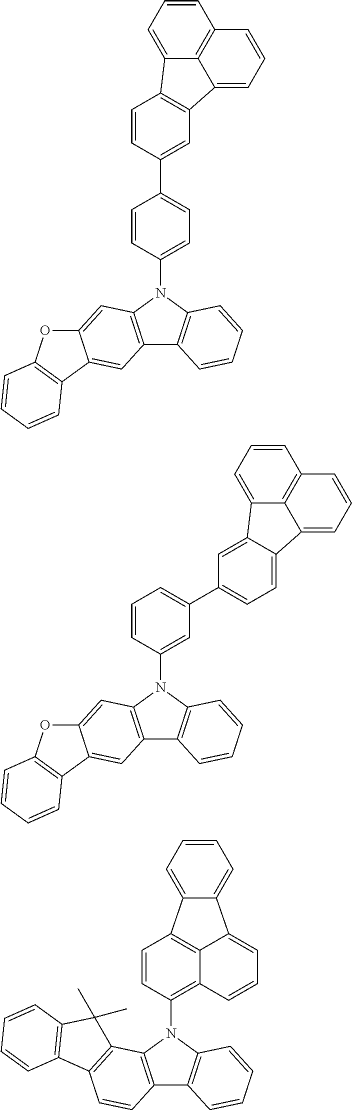 [SMILES: c1cc2c3c(cccc3c1)-c1cc(-c3ccc(-n4c5ccccc5c5cc6c(cc54)oc4ccccc46)cc3)ccc1-2 ; c1cc(-c2ccc3c(c2)-c2cccc4cccc-3c24)cc(-n2c3ccccc3c3cc4c(cc32)oc2ccccc24)c1 ; CC1(C)c2ccccc2-c2ccc3c4ccccc4n(-c4ccc5c6c(cccc46)-c4ccccc4-5)c3c21]

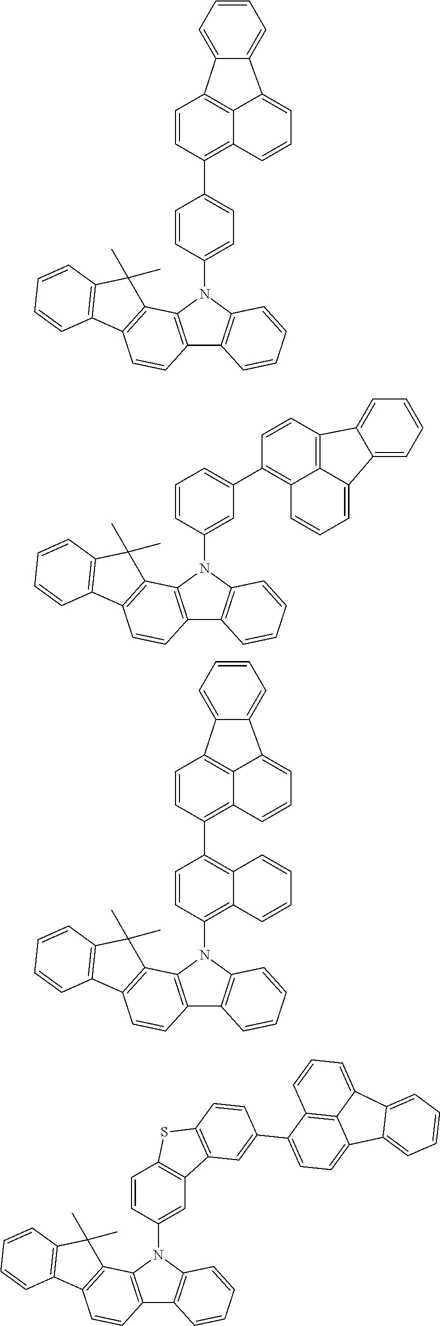

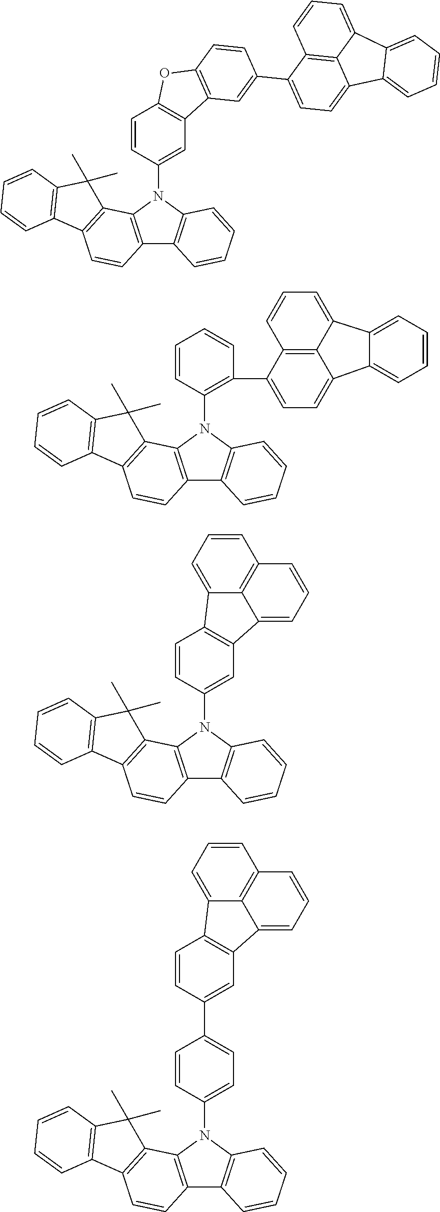

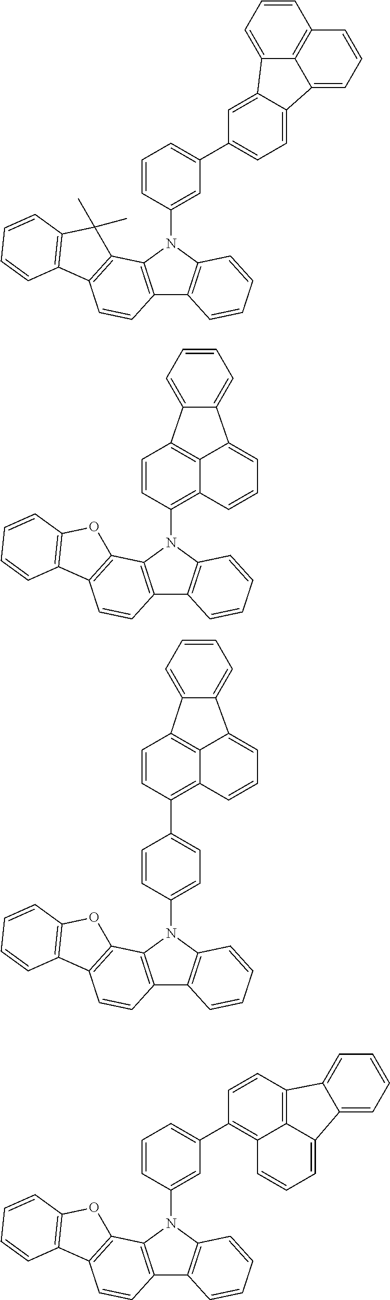

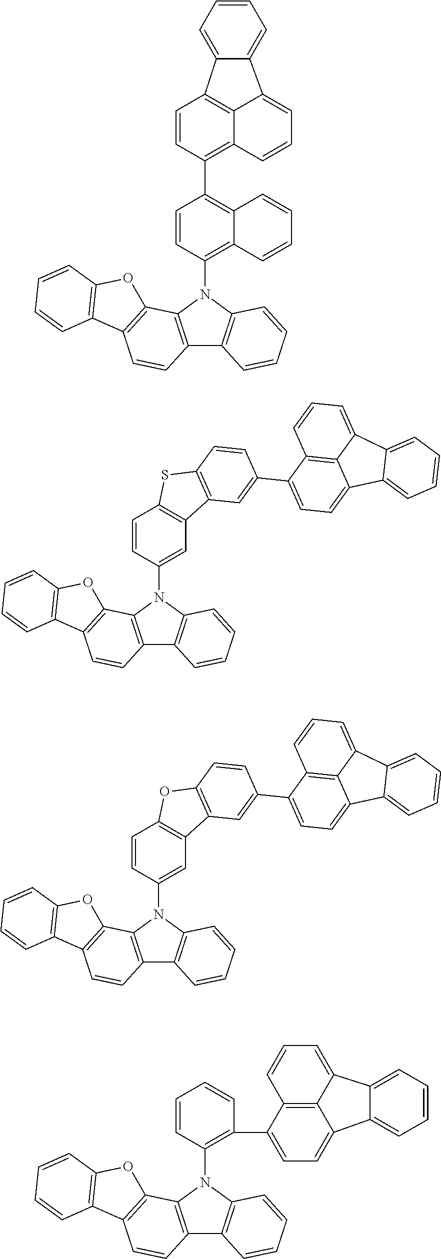 [SMILES: c1ccc2c(c1)-c1cccc3c(-c4ccc(-n5c6ccccc6c6ccc7c8ccccc8oc7c65)c5ccccc45)ccc-2c13 ; c1ccc2c(c1)-c1cccc3c(-c4ccc5sc6ccc(-n7c8ccccc8c8ccc9c%10ccccc%10oc9c87)cc6c5c4)ccc-2c13 ; c1ccc2c(c1)-c1cccc3c(-c4ccc5oc6ccc(-n7c8ccccc8c8ccc9c%10ccccc%10oc9c87)cc6c5c4)ccc-2c13 ; c1ccc2c(c1)-c1cccc3c(-c4ccccc4-n4c5ccccc5c5ccc6c7ccccc7oc6c54)ccc-2c13]

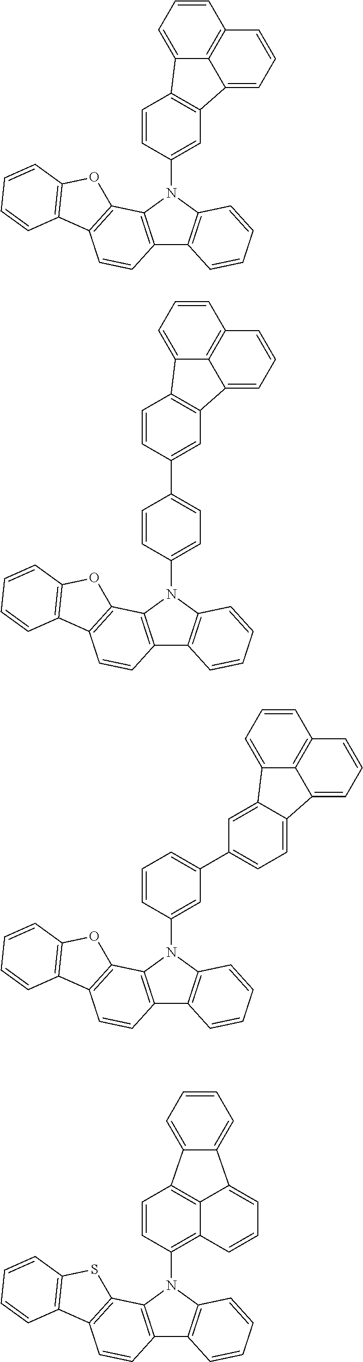

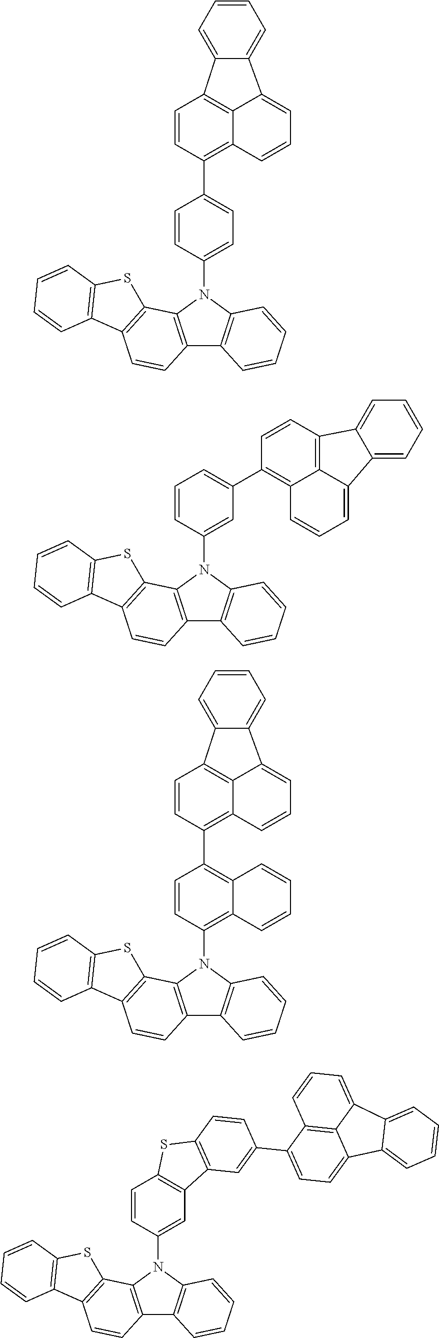

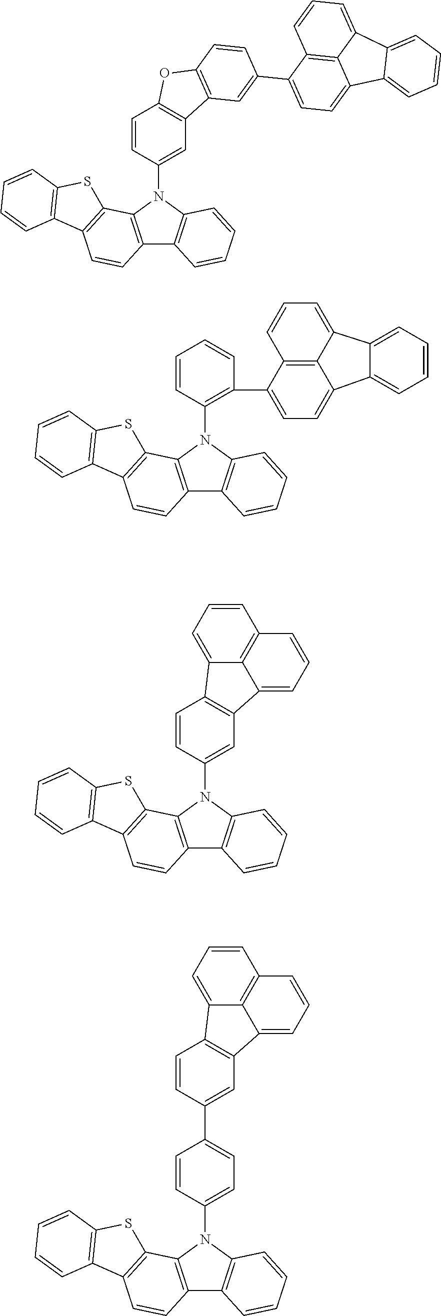 [SMILES: c1ccc2c(c1)-c1cccc3c(-c4ccc5oc6ccc(-n7c8ccccc8c8ccc9c%10ccccc%10sc9c87)cc6c5c4)ccc-2c13 ; c1ccc2c(c1)-c1cccc3c(-c4ccccc4-n4c5ccccc5c5ccc6c7ccccc7sc6c54)ccc-2c13 ; c1cc2c3c(cccc3c1)-c1cc(-n3c4ccccc4c4ccc5c6ccccc6sc5c43)ccc1-2 ; c1cc2c3c(cccc3c1)-c1cc(-c3ccc(-n4c5ccccc5c5ccc6c7ccccc7sc6c54)cc3)ccc1-2]

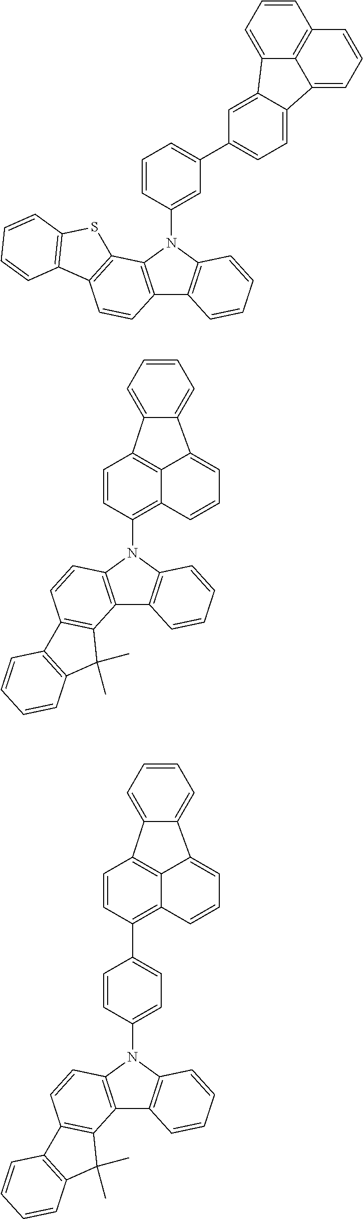

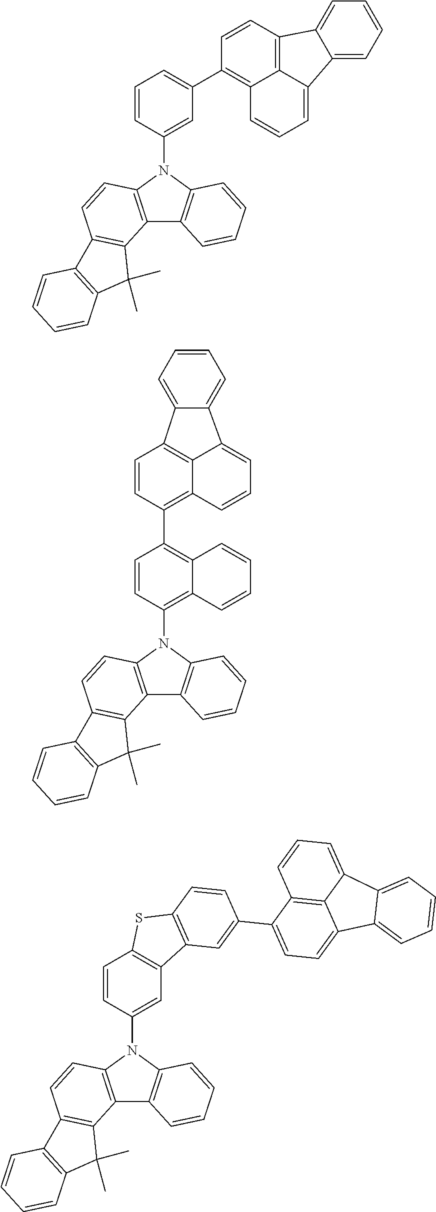 [SMILES: CC1(C)c2ccccc2-c2ccc3c(c21)c1ccccc1n3-c1cccc(-c2ccc3c4c(cccc24)-c2ccccc2-3)c1 ; CC1(C)c2ccccc2-c2ccc3c(c21)c1ccccc1n3-c1ccc(-c2ccc3c4c(cccc24)-c2ccccc2-3)c2ccccc12 ; CC1(C)c2ccccc2-c2ccc3c(c21)c1ccccc1n3-c1ccc2sc3ccc(-c4ccc5c6c(cccc46)-c4ccccc4-5)cc3c2c1]

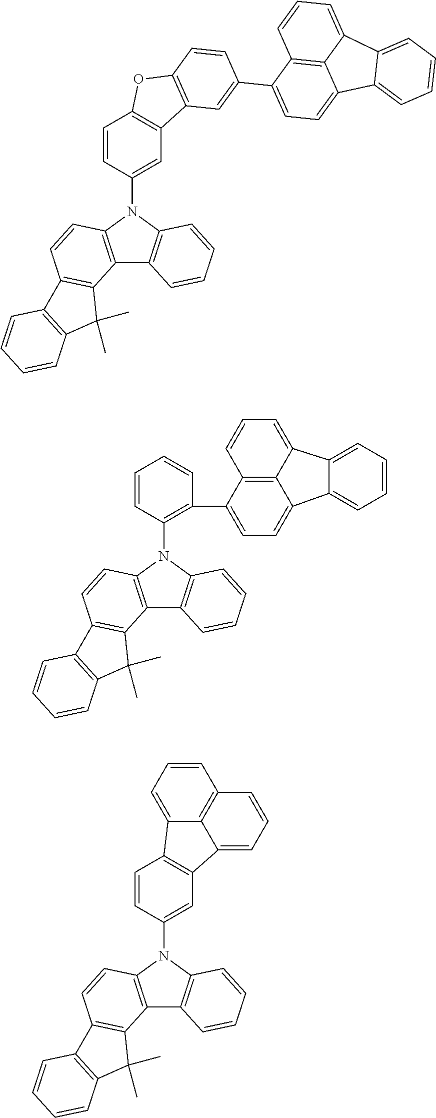 [SMILES: CC1(C)c2ccccc2-c2ccc3c(c21)c1ccccc1n3-c1ccc2oc3ccc(-c4ccc5c6c(cccc46)-c4ccccc4-5)cc3c2c1 ; CC1(C)c2ccccc2-c2ccc3c(c21)c1ccccc1n3-c1ccccc1-c1ccc2c3c(cccc13)-c1ccccc1-2 ; CC1(C)c2ccccc2-c2ccc3c(c21)c1ccccc1n3-c1ccc2c(c1)-c1cccc3cccc-2c13]

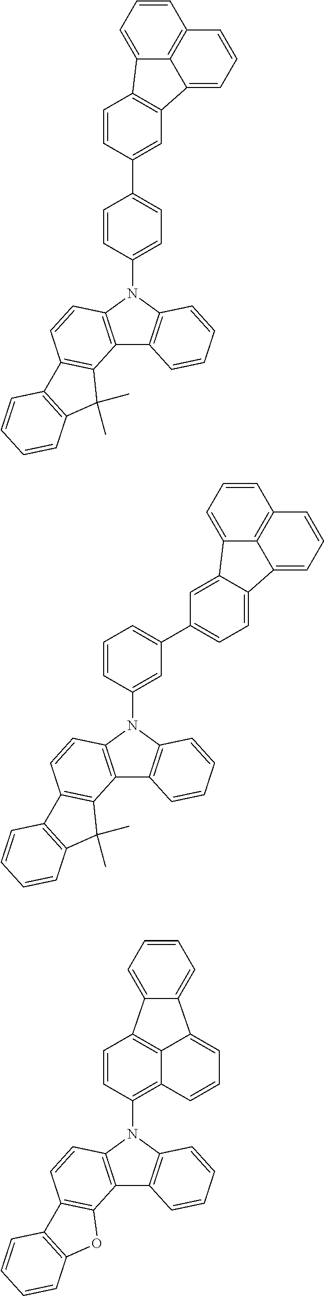 [SMILES: CC1(C)c2ccccc2-c2ccc3c(c21)c1ccccc1n3-c1ccc(-c2ccc3c(c2)-c2cccc4cccc-3c24)cc1 ; CC1(C)c2ccccc2-c2ccc3c(c21)c1ccccc1n3-c1cccc(-c2ccc3c(c2)-c2cccc4cccc-3c24)c1 ; c1ccc2c(c1)-c1cccc3c(-n4c5ccccc5c5c6oc7ccccc7c6ccc54)ccc-2c13]

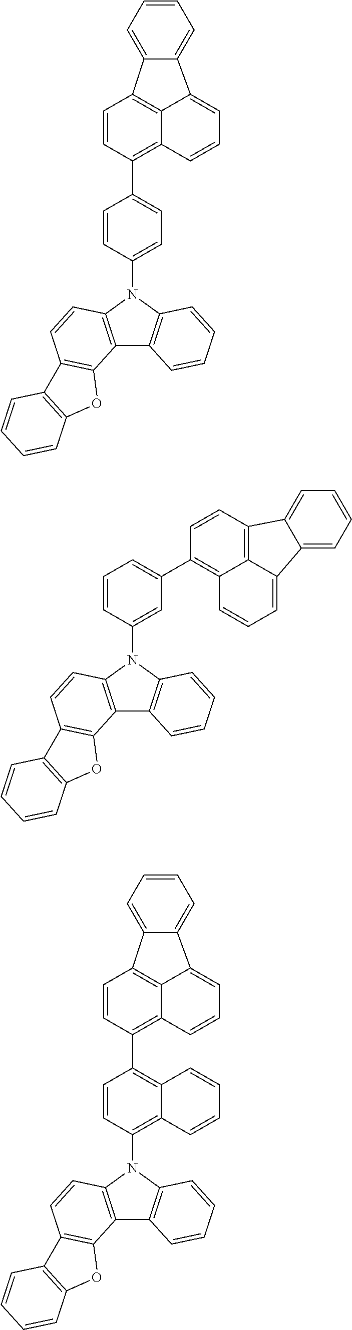 [SMILES: c1ccc2c(c1)-c1cccc3c(-c4ccc(-n5c6ccccc6c6c7oc8ccccc8c7ccc65)cc4)ccc-2c13 ; c1cc(-c2ccc3c4c(cccc24)-c2ccccc2-3)cc(-n2c3ccccc3c3c4oc5ccccc5c4ccc32)c1 ; c1ccc2c(c1)-c1cccc3c(-c4ccc(-n5c6ccccc6c6c7oc8ccccc8c7ccc65)c5ccccc45)ccc-2c13]

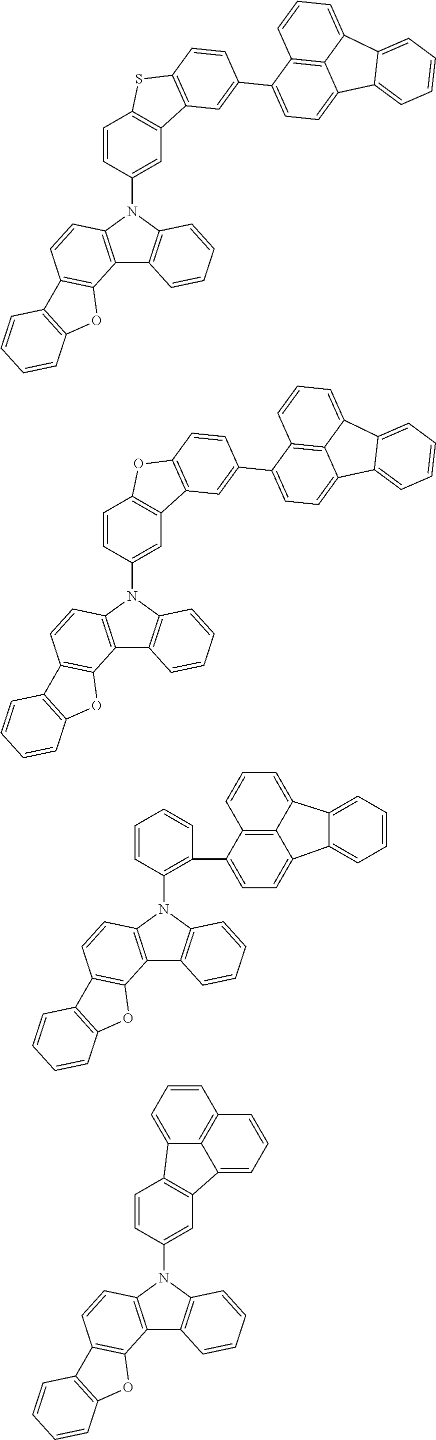 [SMILES: c1ccc2c(c1)-c1cccc3c(-c4ccc5sc6ccc(-n7c8ccccc8c8c9oc%10ccccc%10c9ccc87)cc6c5c4)ccc-2c13 ; c1ccc2c(c1)-c1cccc3c(-c4ccc5oc6ccc(-n7c8ccccc8c8c9oc%10ccccc%10c9ccc87)cc6c5c4)ccc-2c13 ; c1ccc2c(c1)-c1cccc3c(-c4ccccc4-n4c5ccccc5c5c6oc7ccccc7c6ccc54)ccc-2c13 ; c1cc2c3c(cccc3c1)-c1cc(-n3c4ccccc4c4c5oc6ccccc6c5ccc43)ccc1-2]

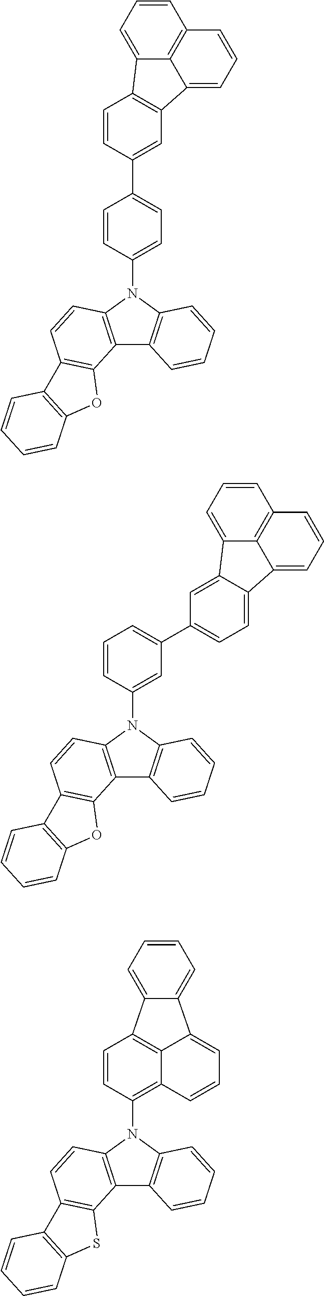 [SMILES: c1cc2c3c(cccc3c1)-c1cc(-c3ccc(-n4c5ccccc5c5c6oc7ccccc7c6ccc54)cc3)ccc1-2 ; c1cc(-c2ccc3c(c2)-c2cccc4cccc-3c24)cc(-n2c3ccccc3c3c4oc5ccccc5c4ccc32)c1 ; c1ccc2c(c1)-c1cccc3c(-n4c5ccccc5c5c6sc7ccccc7c6ccc54)ccc-2c13]

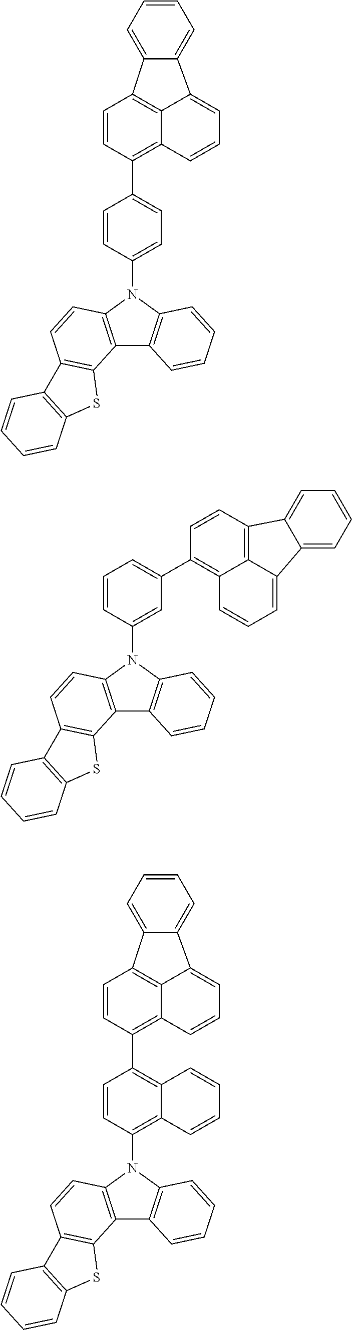 [SMILES: c1ccc2c(c1)-c1cccc3c(-c4ccc(-n5c6ccccc6c6c7sc8ccccc8c7ccc65)cc4)ccc-2c13 ; c1cc(-c2ccc3c4c(cccc24)-c2ccccc2-3)cc(-n2c3ccccc3c3c4sc5ccccc5c4ccc32)c1 ; c1ccc2c(c1)-c1cccc3c(-c4ccc(-n5c6ccccc6c6c7sc8ccccc8c7ccc65)c5ccccc45)ccc-2c13]

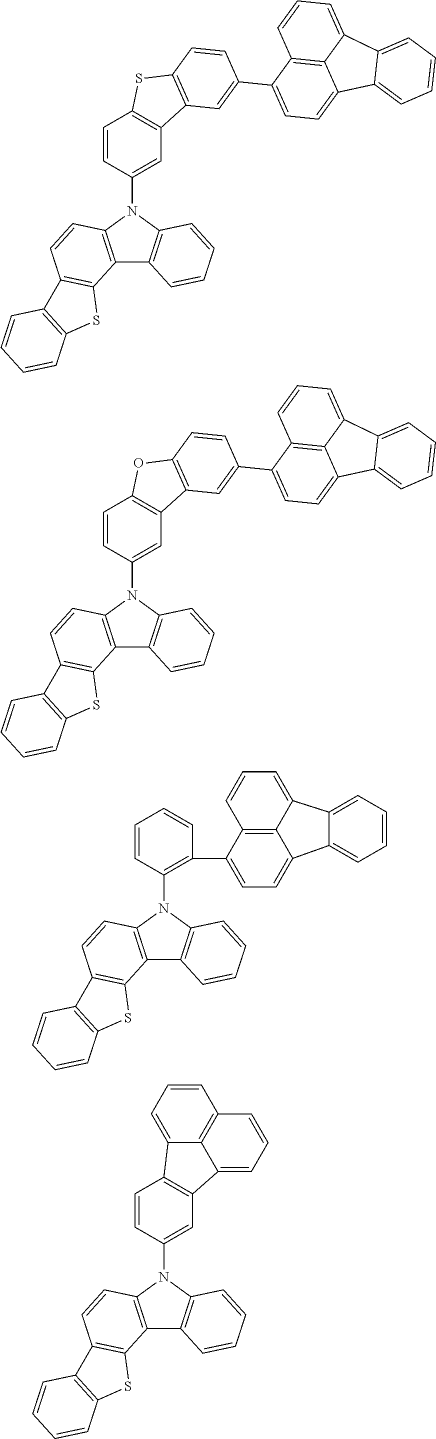 [SMILES: c1ccc2c(c1)-c1cccc3c(-c4ccc5sc6ccc(-n7c8ccccc8c8c9sc%10ccccc%10c9ccc87)cc6c5c4)ccc-2c13 ; c1ccc2c(c1)-c1cccc3c(-c4ccc5oc6ccc(-n7c8ccccc8c8c9sc%10ccccc%10c9ccc87)cc6c5c4)ccc-2c13 ; c1ccc2c(c1)-c1cccc3c(-c4ccccc4-n4c5ccccc5c5c6sc7ccccc7c6ccc54)ccc-2c13 ; c1cc2c3c(cccc3c1)-c1cc(-n3c4ccccc4c4c5sc6ccccc6c5ccc43)ccc1-2]

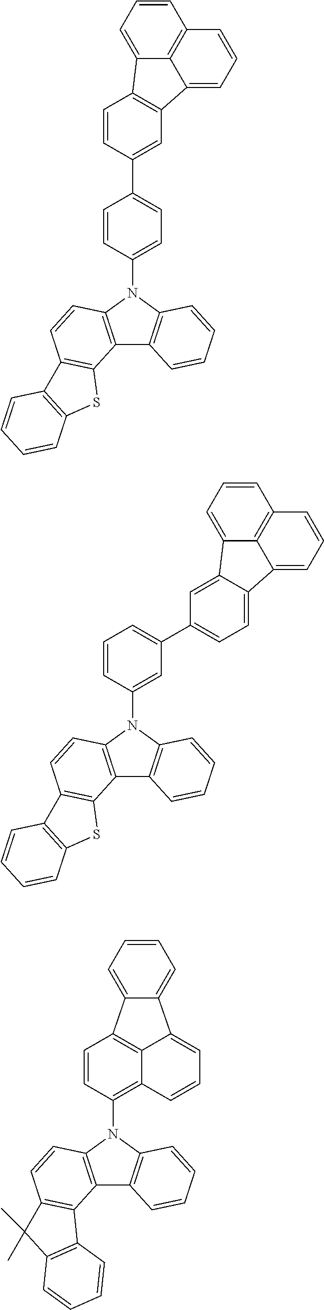 [SMILES: c1cc2c3c(cccc3c1)-c1cc(-c3ccc(-n4c5ccccc5c5c6sc7ccccc7c6ccc54)cc3)ccc1-2 ; c1cc(-c2ccc3c(c2)-c2cccc4cccc-3c24)cc(-n2c3ccccc3c3c4sc5ccccc5c4ccc32)c1 ; CC1(C)c2ccccc2-c2c1ccc1c2c2ccccc2n1-c1ccc2c3c(cccc13)-c1ccccc1-2]

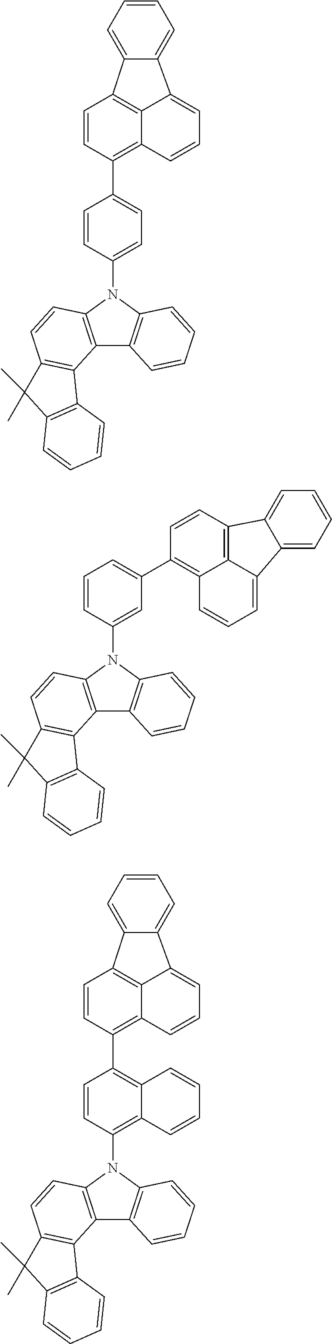 [SMILES: CC1(C)c2ccccc2-c2c1ccc1c2c2ccccc2n1-c1ccc(-c2ccc3c4c(cccc24)-c2ccccc2-3)cc1 ; CC1(C)c2ccccc2-c2c1ccc1c2c2ccccc2n1-c1cccc(-c2ccc3c4c(cccc24)-c2ccccc2-3)c1 ; CC1(C)c2ccccc2-c2c1ccc1c2c2ccccc2n1-c1ccc(-c2ccc3c4c(cccc24)-c2ccccc2-3)c2ccccc12]

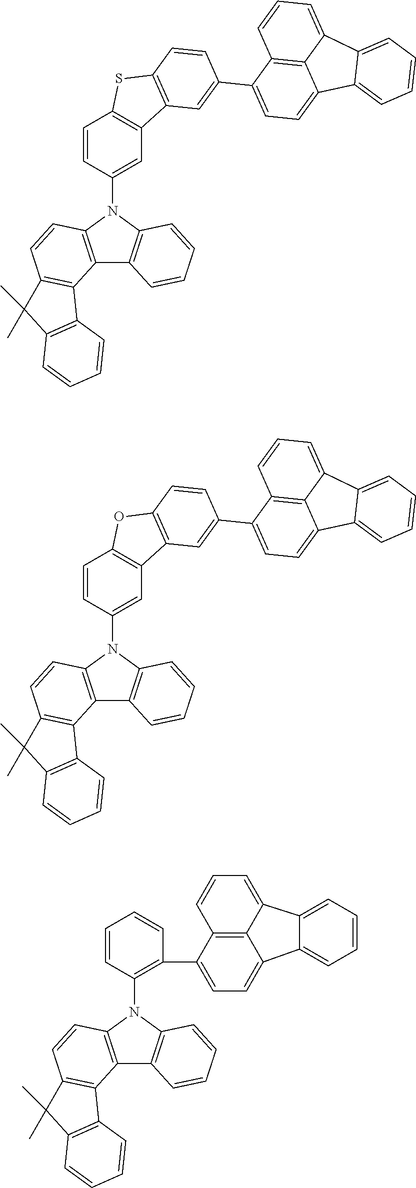 [SMILES: CC1(C)c2ccccc2-c2c1ccc1c2c2ccccc2n1-c1ccc2sc3ccc(-c4ccc5c6c(cccc46)-c4ccccc4-5)cc3c2c1 ; CC1(C)c2ccccc2-c2c1ccc1c2c2ccccc2n1-c1ccc2oc3ccc(-c4ccc5c6c(cccc46)-c4ccccc4-5)cc3c2c1 ; CC1(C)c2ccccc2-c2c1ccc1c2c2ccccc2n1-c1ccccc1-c1ccc2c3c(cccc13)-c1ccccc1-2]

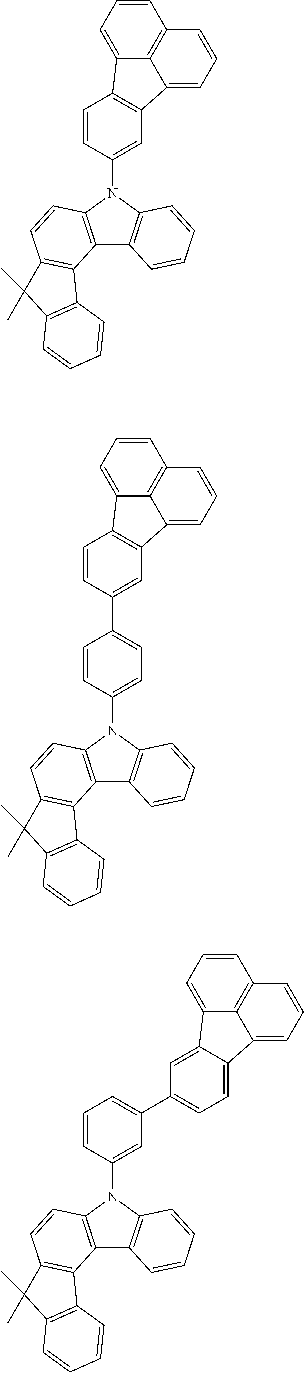 [SMILES: CC1(C)c2ccccc2-c2c1ccc1c2c2ccccc2n1-c1ccc2c(c1)-c1cccc3cccc-2c13 ; CC1(C)c2ccccc2-c2c1ccc1c2c2ccccc2n1-c1ccc(-c2ccc3c(c2)-c2cccc4cccc-3c24)cc1 ; CC1(C)c2ccccc2-c2c1ccc1c2c2ccccc2n1-c1cccc(-c2ccc3c(c2)-c2cccc4cccc-3c24)c1]

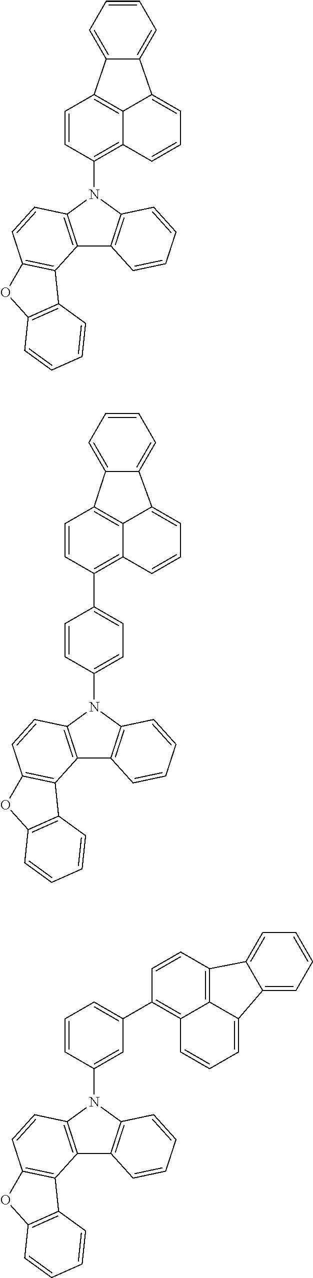 [SMILES: c1ccc2c(c1)-c1cccc3c(-n4c5ccccc5c5c6c(ccc54)oc4ccccc46)ccc-2c13 ; c1ccc2c(c1)-c1cccc3c(-c4ccc(-n5c6ccccc6c6c7c(ccc65)oc5ccccc57)cc4)ccc-2c13 ; c1cc(-c2ccc3c4c(cccc24)-c2ccccc2-3)cc(-n2c3ccccc3c3c4c(ccc32)oc2ccccc24)c1]

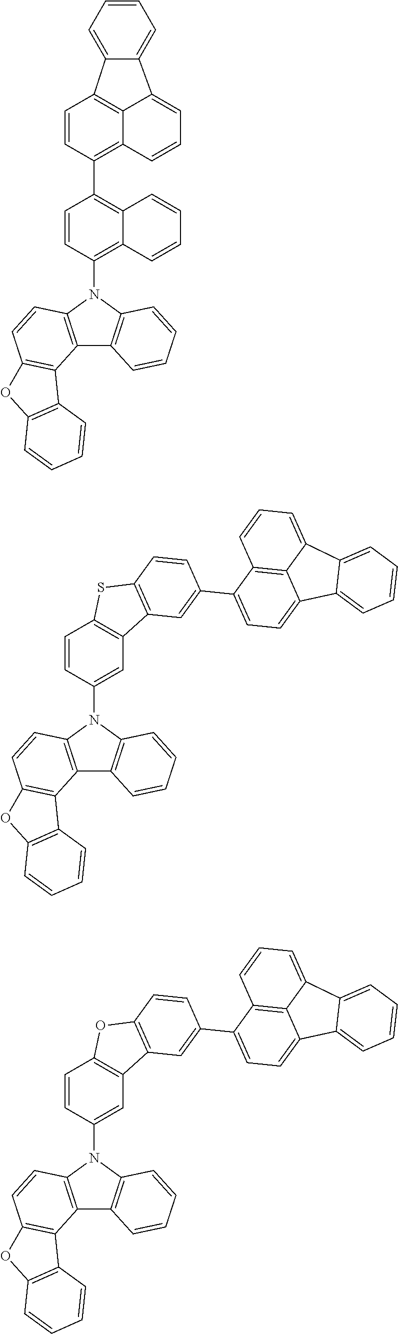 [SMILES: c1ccc2c(c1)-c1cccc3c(-c4ccc(-n5c6ccccc6c6c7c(ccc65)oc5ccccc57)c5ccccc45)ccc-2c13 ; c1ccc2c(c1)-c1cccc3c(-c4ccc5sc6ccc(-n7c8ccccc8c8c9c(ccc87)oc7ccccc79)cc6c5c4)ccc-2c13 ; c1ccc2c(c1)-c1cccc3c(-c4ccc5oc6ccc(-n7c8ccccc8c8c9c(ccc87)oc7ccccc79)cc6c5c4)ccc-2c13]

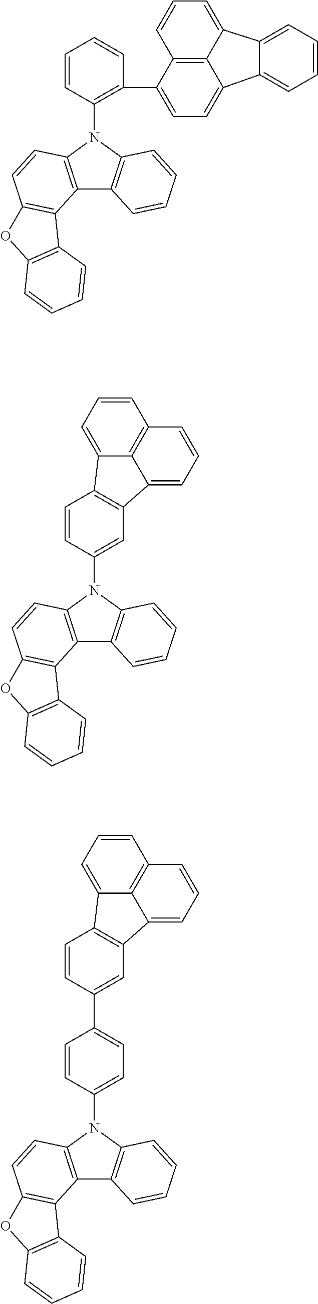 [SMILES: c1ccc2c(c1)-c1cccc3c(-c4ccccc4-n4c5ccccc5c5c6c(ccc54)oc4ccccc46)ccc-2c13 ; c1cc2c3c(cccc3c1)-c1cc(-n3c4ccccc4c4c5c(ccc43)oc3ccccc35)ccc1-2 ; c1cc2c3c(cccc3c1)-c1cc(-c3ccc(-n4c5ccccc5c5c6c(ccc54)oc4ccccc46)cc3)ccc1-2]

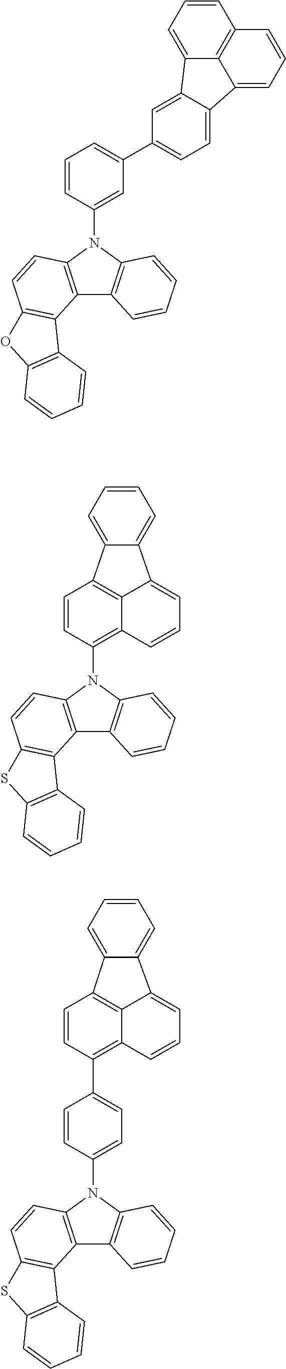 [SMILES: c1cc(-c2ccc3c(c2)-c2cccc4cccc-3c24)cc(-n2c3ccccc3c3c4c(ccc32)oc2ccccc24)c1 ; c1ccc2c(c1)-c1cccc3c(-n4c5ccccc5c5c6c(ccc54)sc4ccccc46)ccc-2c13 ; c1ccc2c(c1)-c1cccc3c(-c4ccc(-n5c6ccccc6c6c7c(ccc65)sc5ccccc57)cc4)ccc-2c13]

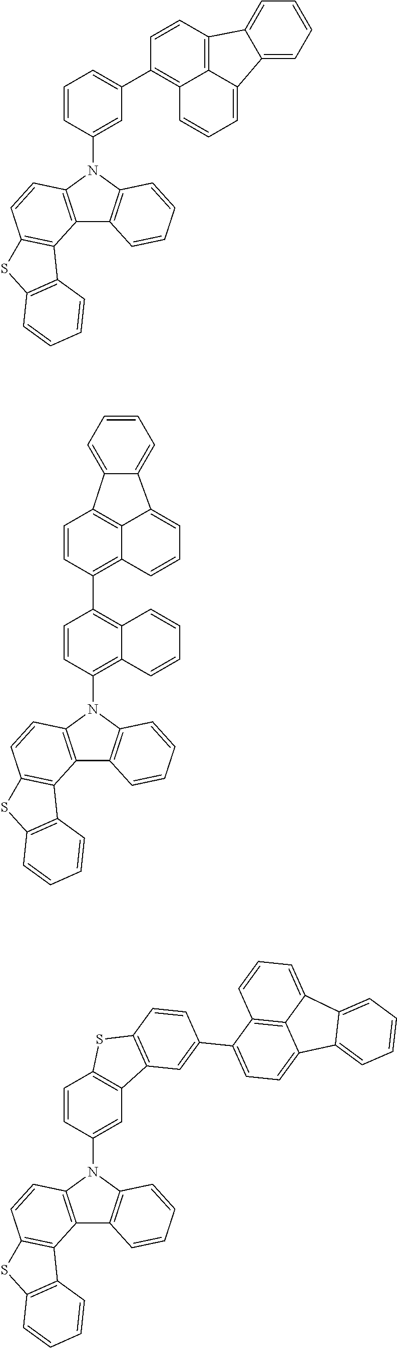 [SMILES: c1cc(-c2ccc3c4c(cccc24)-c2ccccc2-3)cc(-n2c3ccccc3c3c4c(ccc32)sc2ccccc24)c1 ; c1ccc2c(c1)-c1cccc3c(-c4ccc(-n5c6ccccc6c6c7c(ccc65)sc5ccccc57)c5ccccc45)ccc-2c13 ; c1ccc2c(c1)-c1cccc3c(-c4ccc5sc6ccc(-n7c8ccccc8c8c9c(ccc87)sc7ccccc79)cc6c5c4)ccc-2c13]

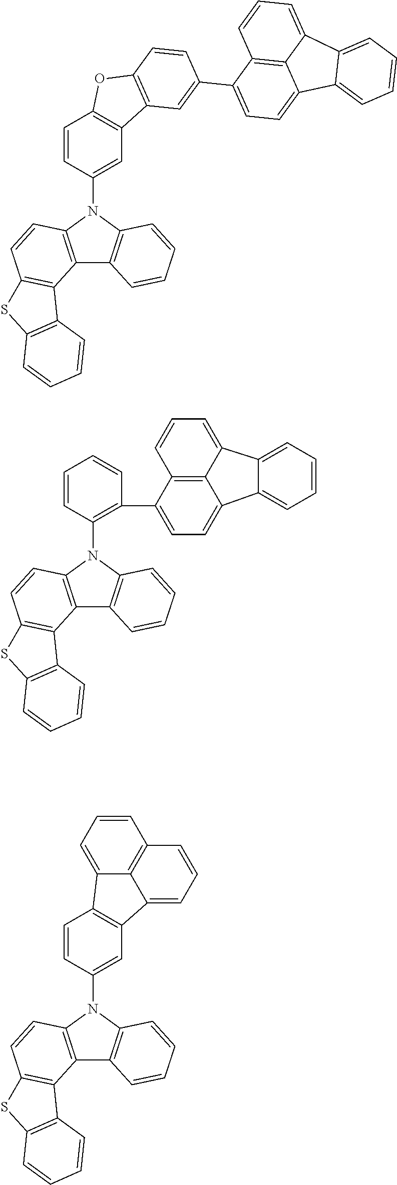 [SMILES: c1ccc2c(c1)-c1cccc3c(-c4ccc5oc6ccc(-n7c8ccccc8c8c9c(ccc87)sc7ccccc79)cc6c5c4)ccc-2c13 ; c1ccc2c(c1)-c1cccc3c(-c4ccccc4-n4c5ccccc5c5c6c(ccc54)sc4ccccc46)ccc-2c13 ; c1cc2c3c(cccc3c1)-c1cc(-n3c4ccccc4c4c5c(ccc43)sc3ccccc35)ccc1-2]

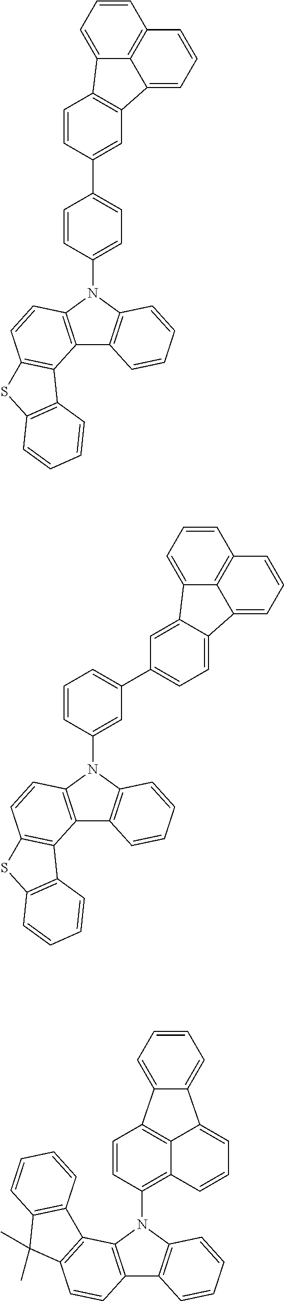 [SMILES: c1cc2c3c(cccc3c1)-c1cc(-c3ccc(-n4c5ccccc5c5c6c(ccc54)sc4ccccc46)cc3)ccc1-2 ; c1cc(-c2ccc3c(c2)-c2cccc4cccc-3c24)cc(-n2c3ccccc3c3c4c(ccc32)sc2ccccc24)c1 ; CC1(C)c2ccccc2-c2c1ccc1c3ccccc3n(-c3ccc4c5c(cccc35)-c3ccccc3-4)c21]

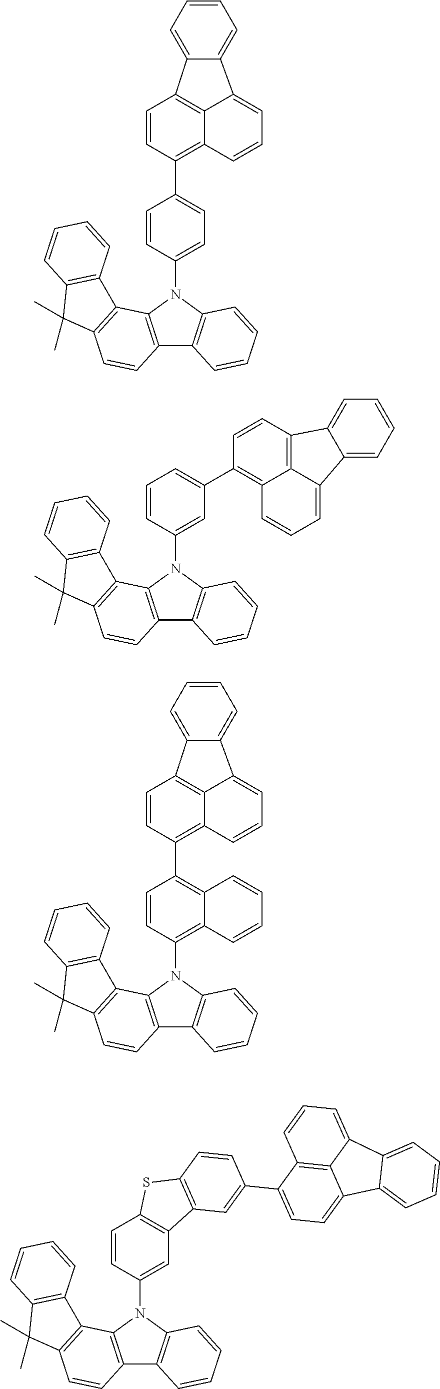

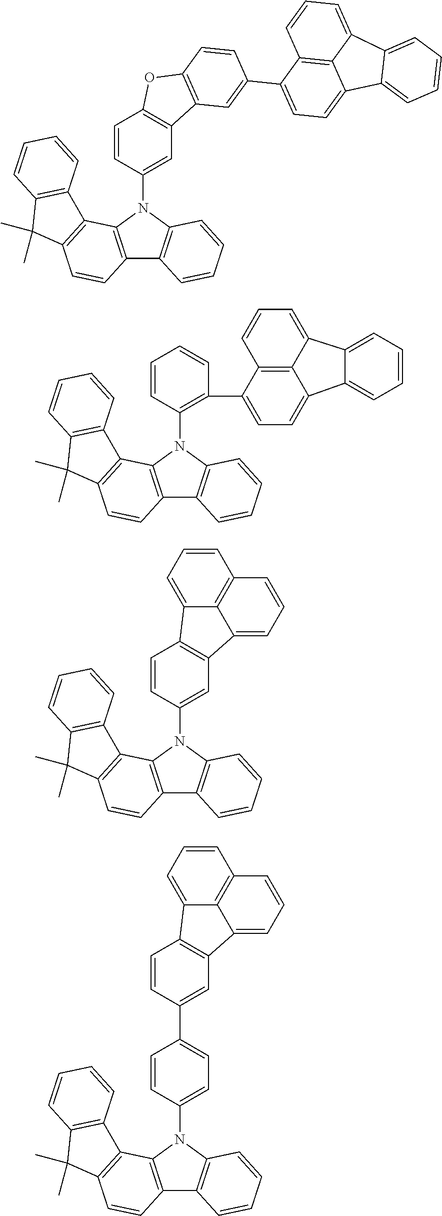

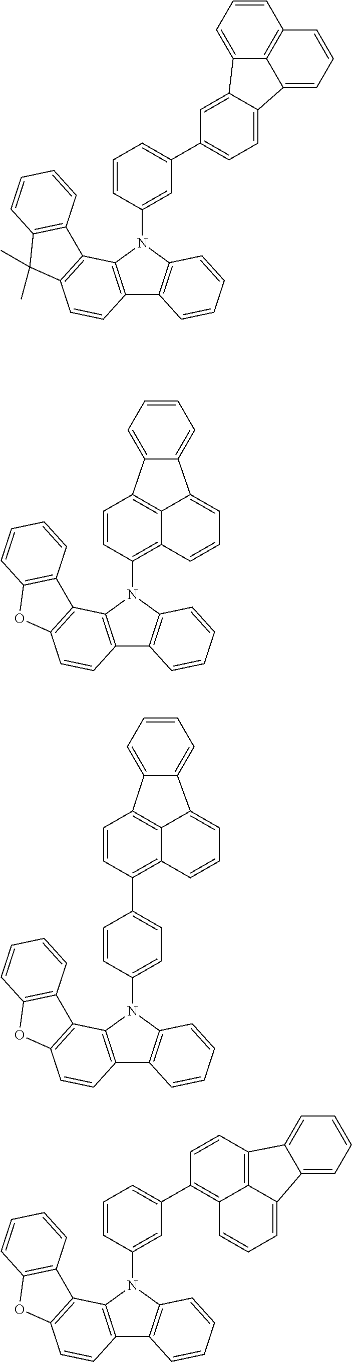 [SMILES: CC1(C)c2ccccc2-c2c1ccc1c3ccccc3n(-c3cccc(-c4ccc5c(c4)-c4cccc6cccc-5c46)c3)c21 ; c1ccc2c(c1)-c1cccc3c(-n4c5ccccc5c5ccc6oc7ccccc7c6c54)ccc-2c13 ; c1ccc2c(c1)-c1cccc3c(-c4ccc(-n5c6ccccc6c6ccc7oc8ccccc8c7c65)cc4)ccc-2c13 ; c1cc(-c2ccc3c4c(cccc24)-c2ccccc2-3)cc(-n2c3ccccc3c3ccc4oc5ccccc5c4c32)c1]

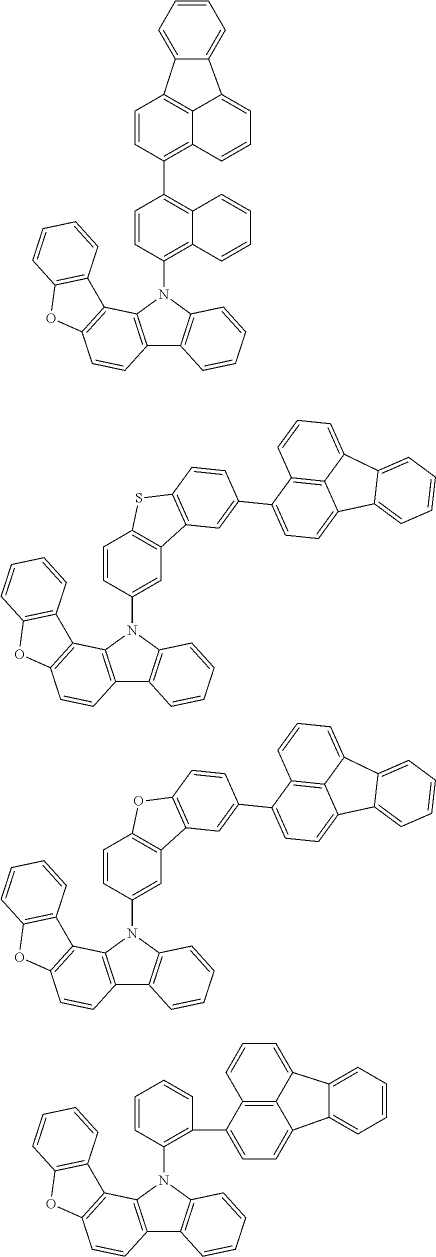 [SMILES: c1ccc2c(c1)-c1cccc3c(-c4ccc(-n5c6ccccc6c6ccc7oc8ccccc8c7c65)c5ccccc45)ccc-2c13 ; c1ccc2c(c1)-c1cccc3c(-c4ccc5sc6ccc(-n7c8ccccc8c8ccc9oc%10ccccc%10c9c87)cc6c5c4)ccc-2c13 ; c1ccc2c(c1)-c1cccc3c(-c4ccc5oc6ccc(-n7c8ccccc8c8ccc9oc%10ccccc%10c9c87)cc6c5c4)ccc-2c13 ; c1ccc2c(c1)-c1cccc3c(-c4ccccc4-n4c5ccccc5c5ccc6oc7ccccc7c6c54)ccc-2c13]

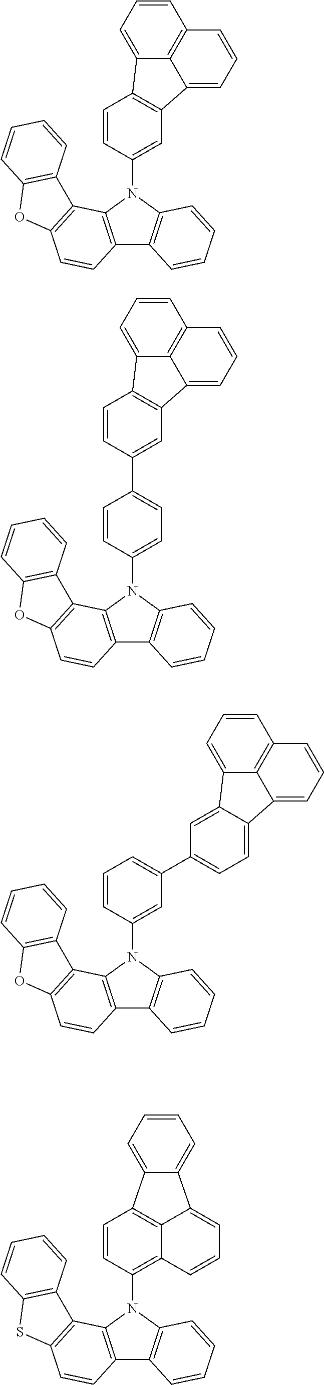

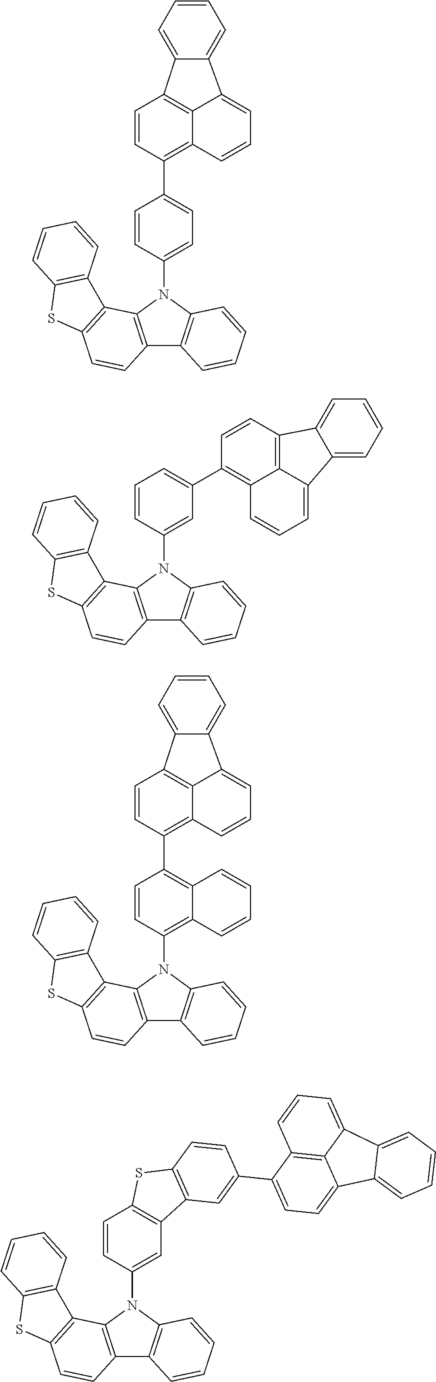

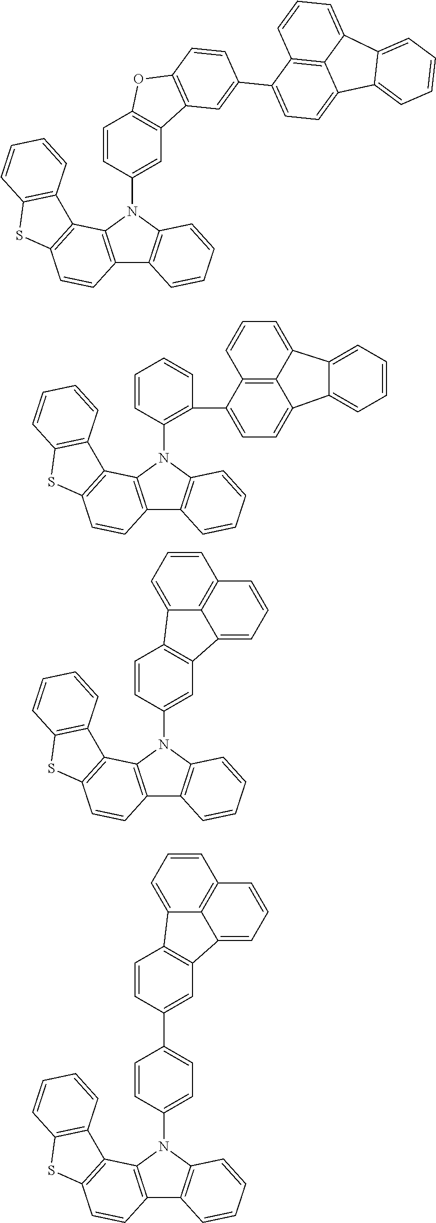

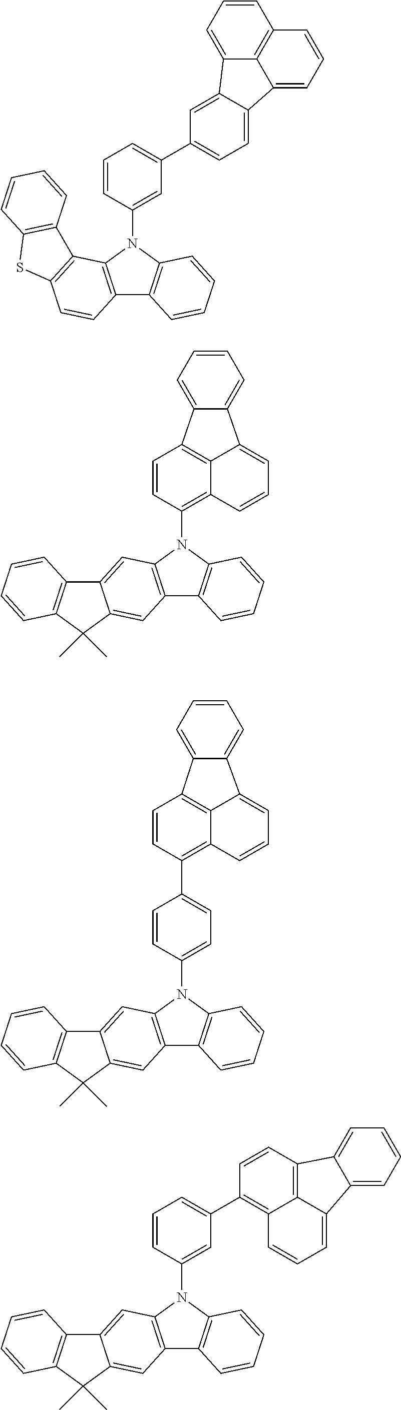

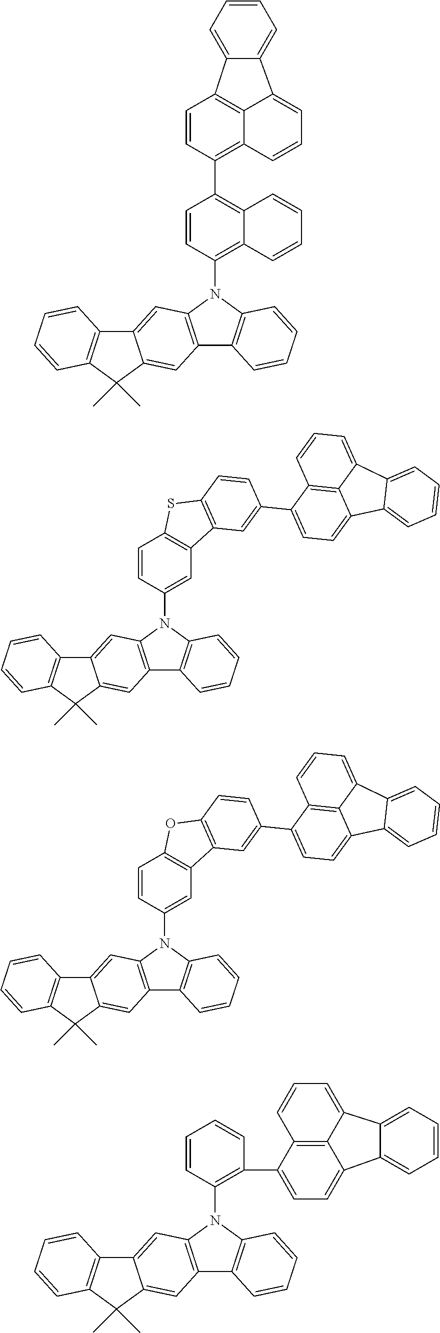 [SMILES: CC1(C)c2ccccc2-c2cc3c(cc21)c1ccccc1n3-c1ccc(-c2ccc3c4c(cccc24)-c2ccccc2-3)c2ccccc12 ; CC1(C)c2ccccc2-c2cc3c(cc21)c1ccccc1n3-c1ccc2sc3ccc(-c4ccc5c6c(cccc46)-c4ccccc4-5)cc3c2c1 ; CC1(C)c2ccccc2-c2cc3c(cc21)c1ccccc1n3-c1ccc2oc3ccc(-c4ccc5c6c(cccc46)-c4ccccc4-5)cc3c2c1 ; CC1(C)c2ccccc2-c2cc3c(cc21)c1ccccc1n3-c1ccccc1-c1ccc2c3c(cccc13)-c1ccccc1-2]

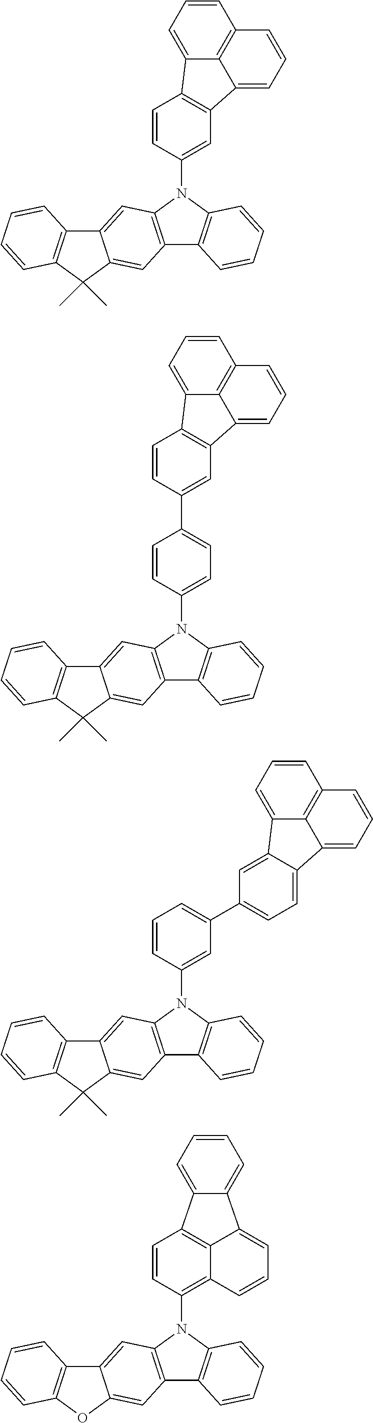 [SMILES: CC1(C)c2ccccc2-c2cc3c(cc21)c1ccccc1n3-c1ccc2c(c1)-c1cccc3cccc-2c13 ; CC1(C)c2ccccc2-c2cc3c(cc21)c1ccccc1n3-c1ccc(-c2ccc3c(c2)-c2cccc4cccc-3c24)cc1 ; CC1(C)c2ccccc2-c2cc3c(cc21)c1ccccc1n3-c1cccc(-c2ccc3c(c2)-c2cccc4cccc-3c24)c1 ; c1ccc2c(c1)-c1cccc3c(-n4c5ccccc5c5cc6oc7ccccc7c6cc54)ccc-2c13]

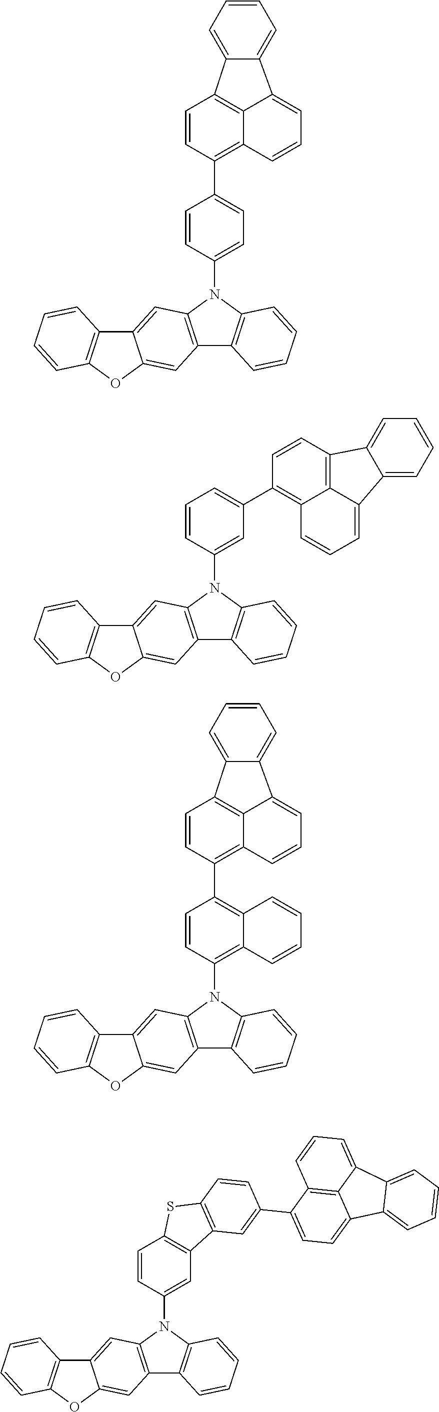

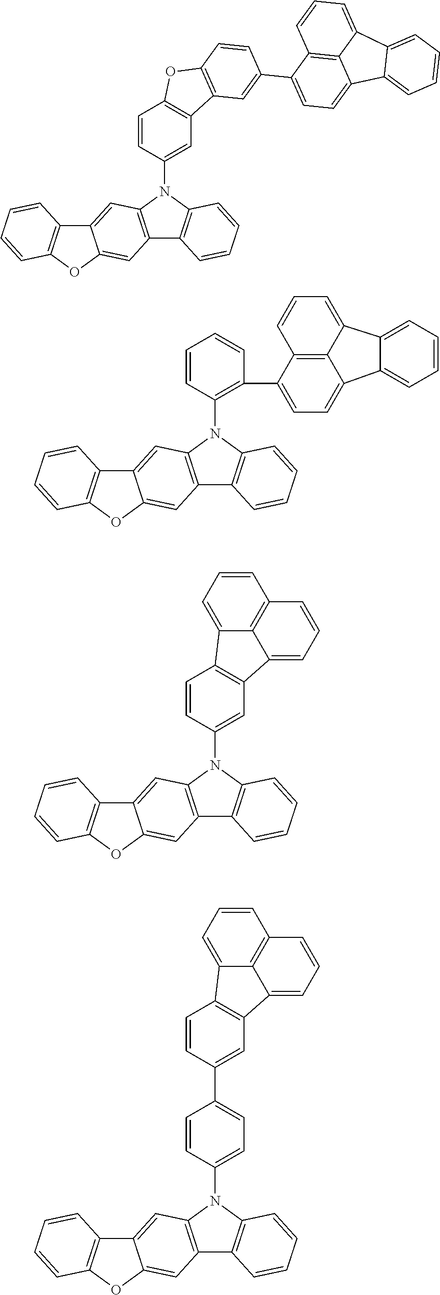 [SMILES: c1ccc2c(c1)-c1cccc3c(-c4ccc5oc6ccc(-n7c8ccccc8c8cc9oc%10ccccc%10c9cc87)cc6c5c4)ccc-2c13 ; c1ccc2c(c1)-c1cccc3c(-c4ccccc4-n4c5ccccc5c5cc6oc7ccccc7c6cc54)ccc-2c13 ; c1cc2c3c(cccc3c1)-c1cc(-n3c4ccccc4c4cc5oc6ccccc6c5cc43)ccc1-2 ; c1cc2c3c(cccc3c1)-c1cc(-c3ccc(-n4c5ccccc5c5cc6oc7ccccc7c6cc54)cc3)ccc1-2]

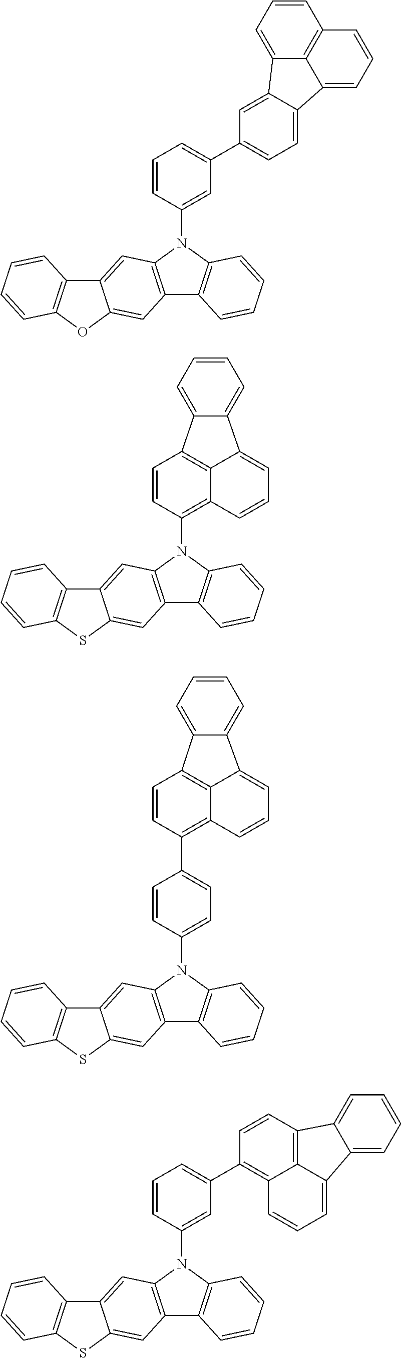

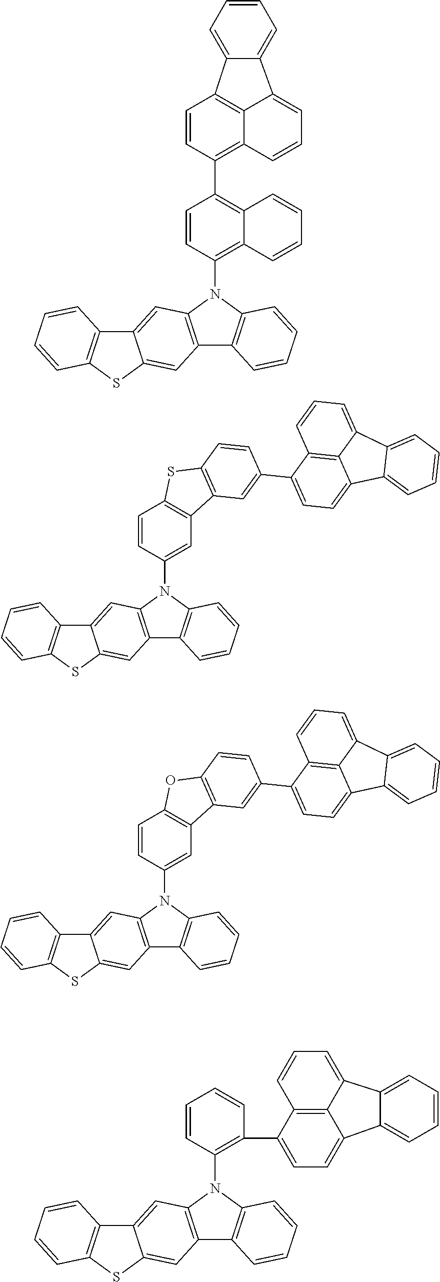 [SMILES: c1ccc2c(c1)-c1cccc3c(-c4ccc(-n5c6ccccc6c6cc7sc8ccccc8c7cc65)c5ccccc45)ccc-2c13 ; c1ccc2c(c1)-c1cccc3c(-c4ccc5sc6ccc(-n7c8ccccc8c8cc9sc%10ccccc%10c9cc87)cc6c5c4)ccc-2c13 ; c1ccc2c(c1)-c1cccc3c(-c4ccc5oc6ccc(-n7c8ccccc8c8cc9sc%10ccccc%10c9cc87)cc6c5c4)ccc-2c13 ; c1ccc2c(c1)-c1cccc3c(-c4ccccc4-n4c5ccccc5c5cc6sc7ccccc7c6cc54)ccc-2c13]

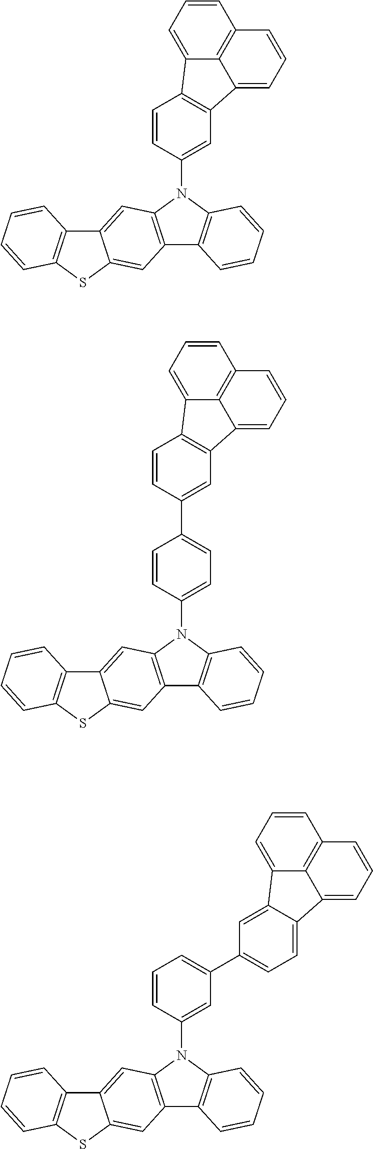 [SMILES: c1cc2c3c(cccc3c1)-c1cc(-n3c4ccccc4c4cc5sc6ccccc6c5cc43)ccc1-2 ; c1cc2c3c(cccc3c1)-c1cc(-c3ccc(-n4c5ccccc5c5cc6sc7ccccc7c6cc54)cc3)ccc1-2 ; c1cc(-c2ccc3c(c2)-c2cccc4cccc-3c24)cc(-n2c3ccccc3c3cc4sc5ccccc5c4cc32)c1]

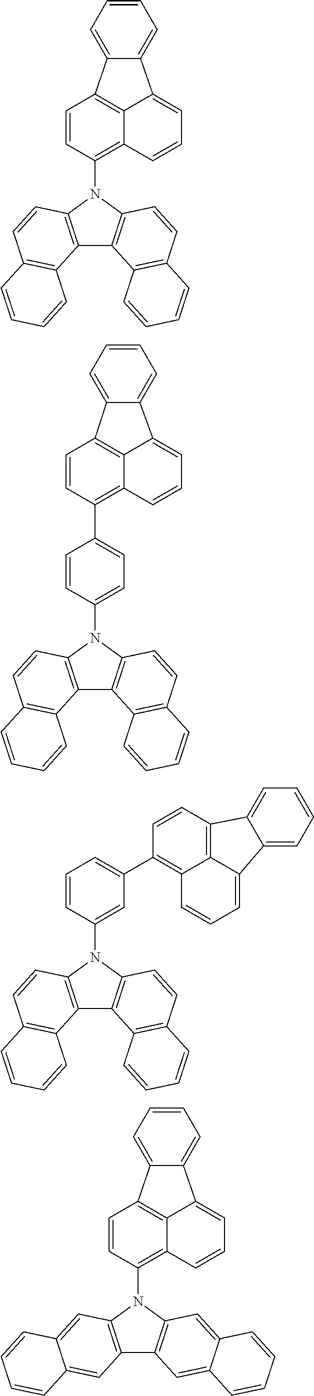

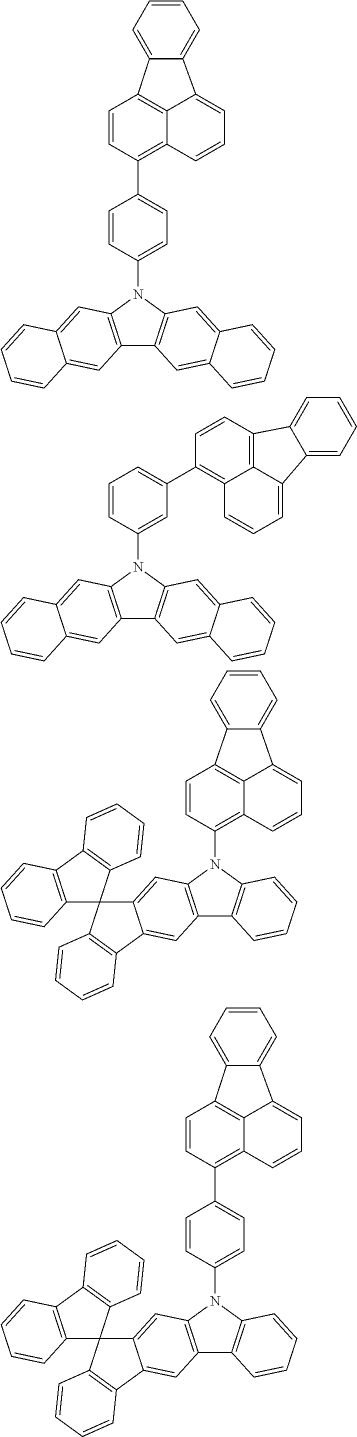

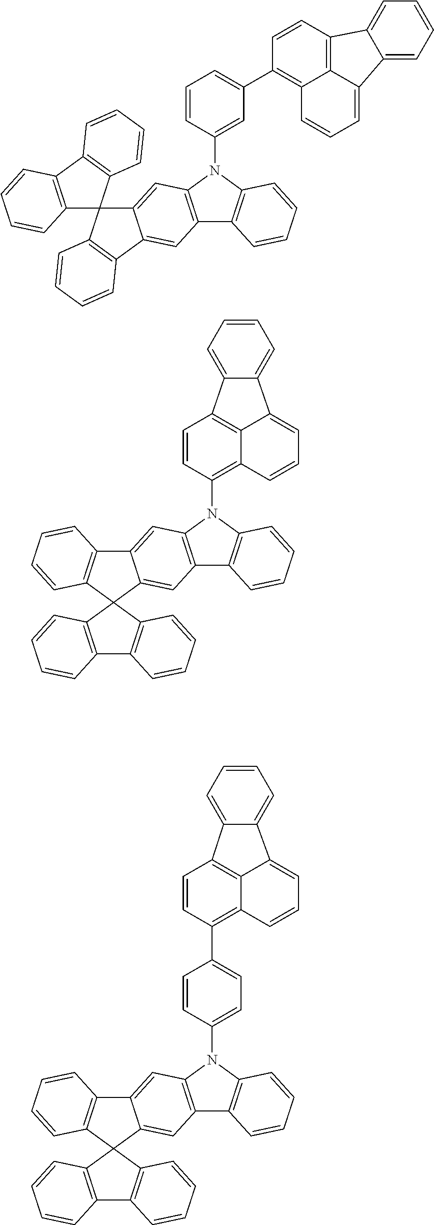 [SMILES: c1cc(-c2ccc3c4c(cccc24)-c2ccccc2-3)cc(-n2c3ccccc3c3cc4c(cc32)C2(c3ccccc3-c3ccccc32)c2ccccc2-4)c1 ; c1ccc2c(c1)-c1cccc3c(-n4c5ccccc5c5cc6c(cc54)-c4ccccc4C64c5ccccc5-c5ccccc54)ccc-2c13 ; c1ccc2c(c1)-c1cccc3c(-c4ccc(-n5c6ccccc6c6cc7c(cc65)-c5ccccc5C75c6ccccc6-c6ccccc65)cc4)ccc-2c13]

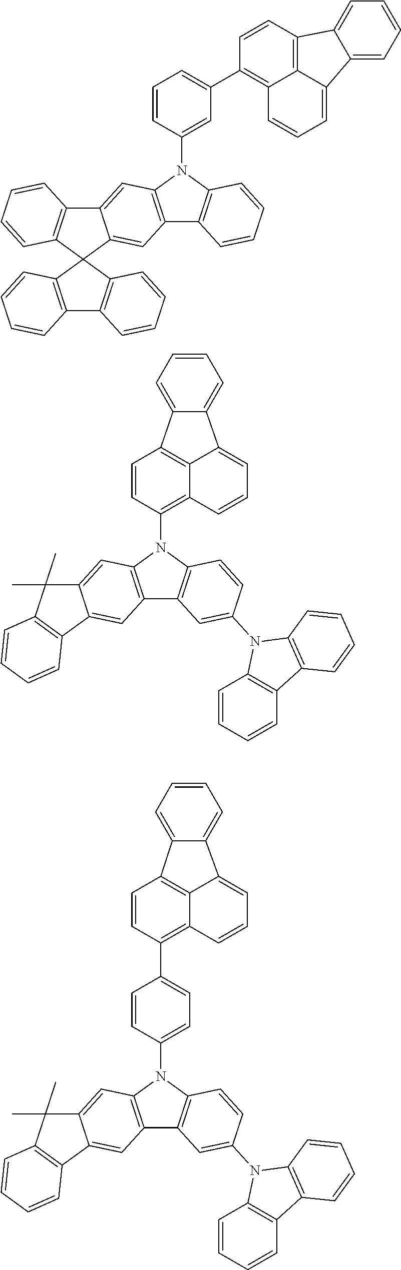

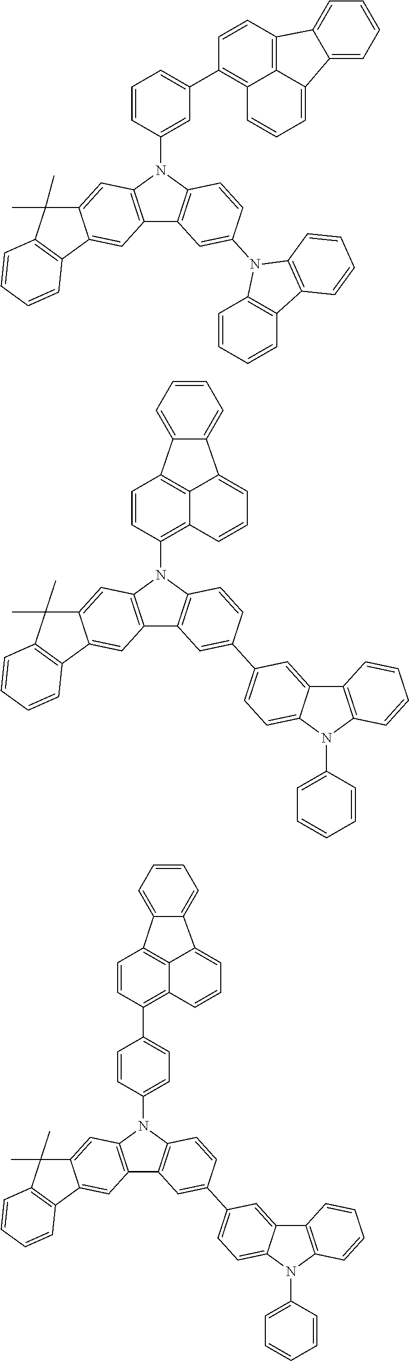

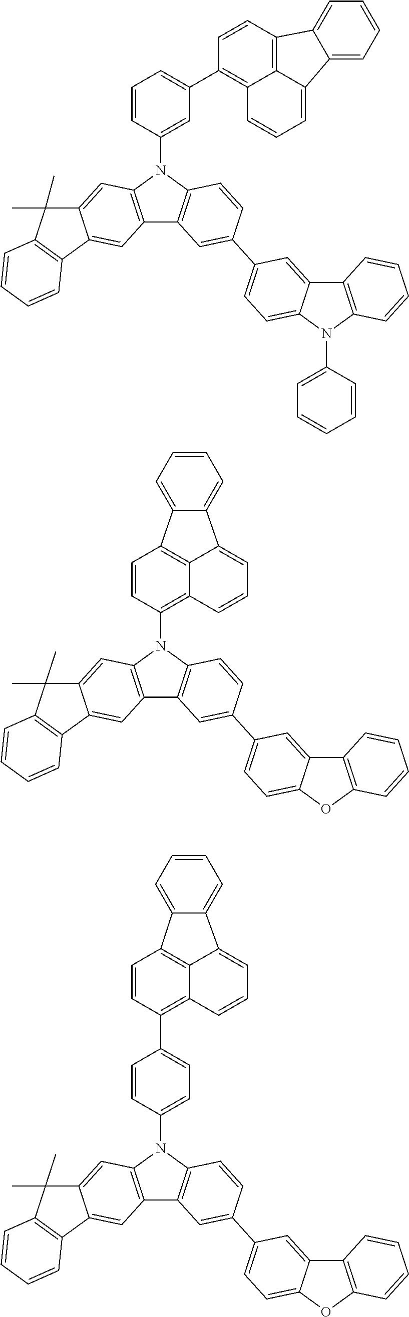 [SMILES: CC1(C)c2ccccc2-c2cc3c4cc(-c5ccc6c(c5)c5ccccc5n6-c5ccccc5)ccc4n(-c4cccc(-c5ccc6c7c(cccc57)-c5ccccc5-6)c4)c3cc21 ; CC1(C)c2ccccc2-c2cc3c4cc(-c5ccc6oc7ccccc7c6c5)ccc4n(-c4ccc5c6c(cccc46)-c4ccccc4-5)c3cc21 ; CC1(C)c2ccccc2-c2cc3c4cc(-c5ccc6oc7ccccc7c6c5)ccc4n(-c4ccc(-c5ccc6c7c(cccc57)-c5ccccc5-6)cc4)c3cc21]

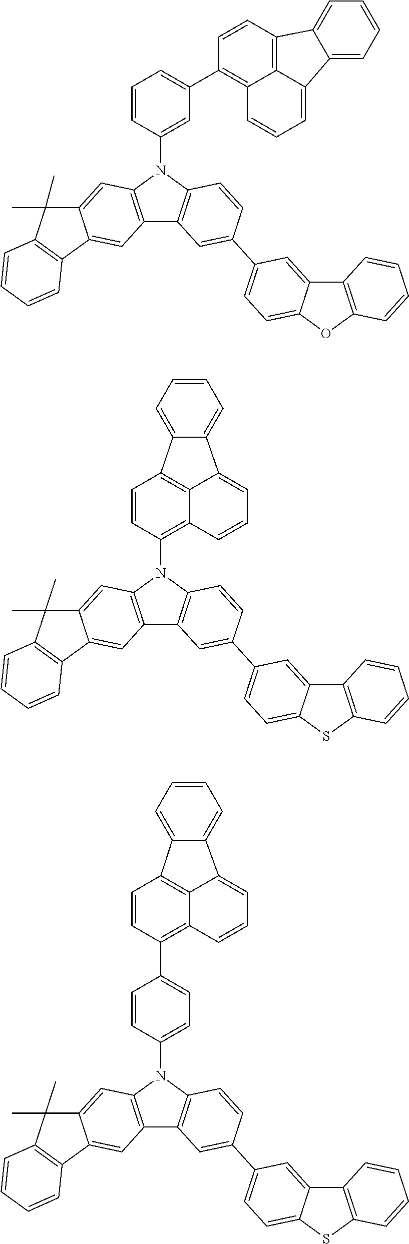 [SMILES: CC1(C)c2ccccc2-c2cc3c4cc(-c5ccc6oc7ccccc7c6c5)ccc4n(-c4cccc(-c5ccc6c7c(cccc57)-c5ccccc5-6)c4)c3cc21 ; CC1(C)c2ccccc2-c2cc3c4cc(-c5ccc6sc7ccccc7c6c5)ccc4n(-c4ccc5c6c(cccc46)-c4ccccc4-5)c3cc21 ; CC1(C)c2ccccc2-c2cc3c4cc(-c5ccc6sc7ccccc7c6c5)ccc4n(-c4ccc(-c5ccc6c7c(cccc57)-c5ccccc5-6)cc4)c3cc21]

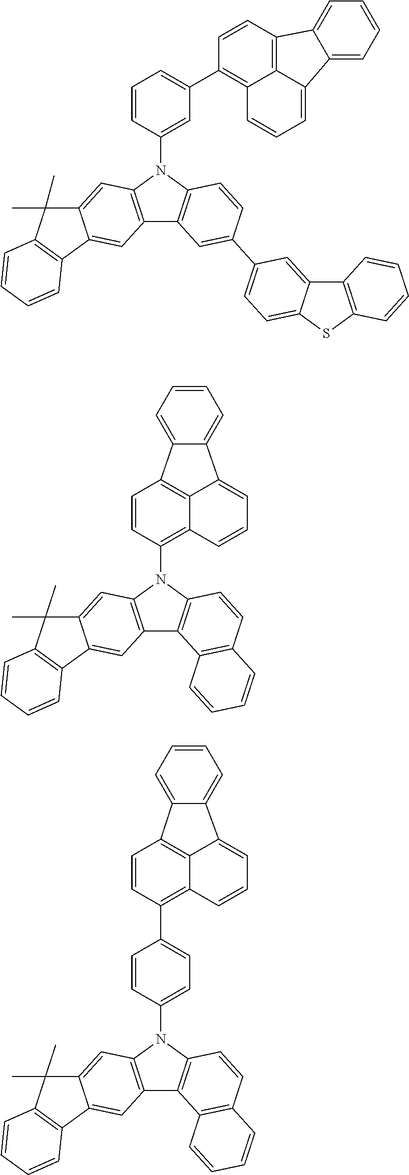

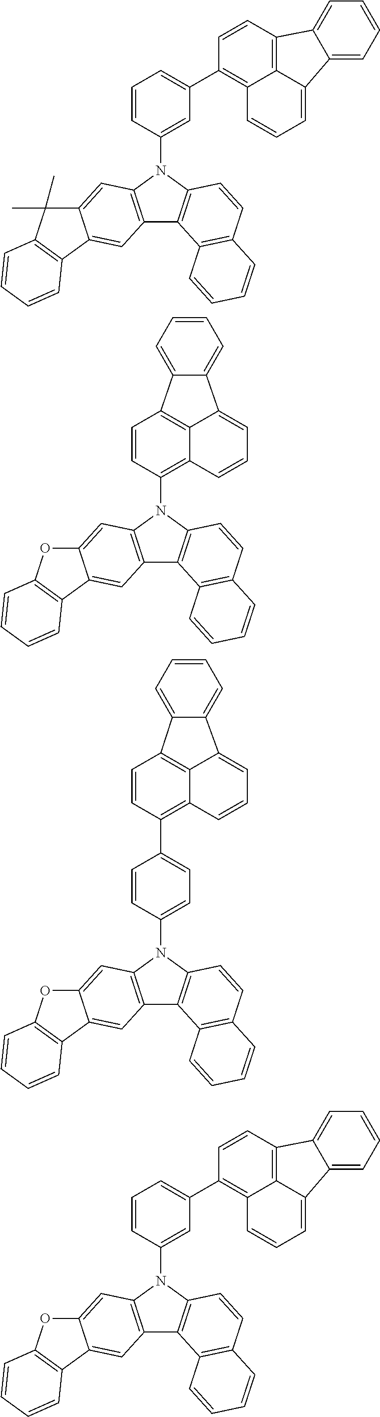

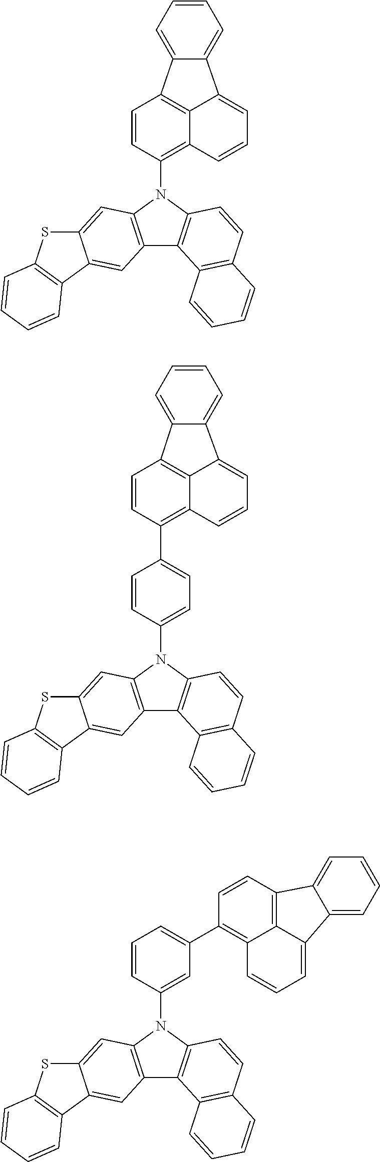

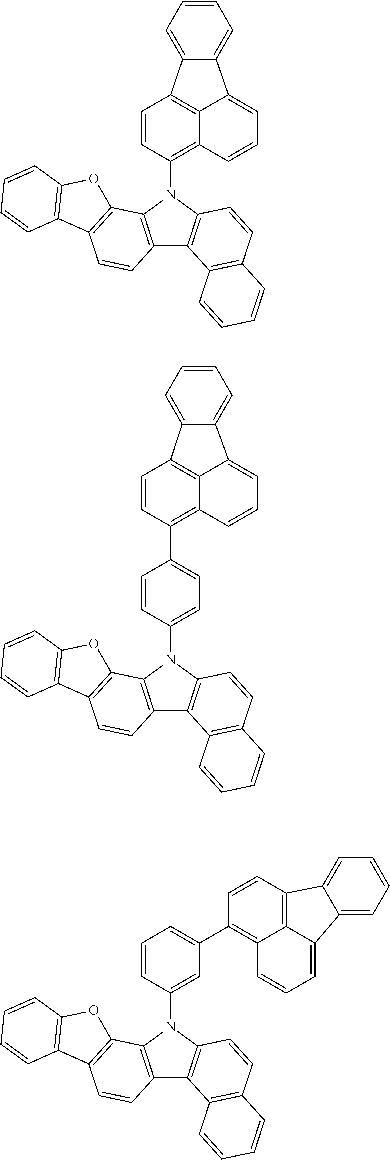

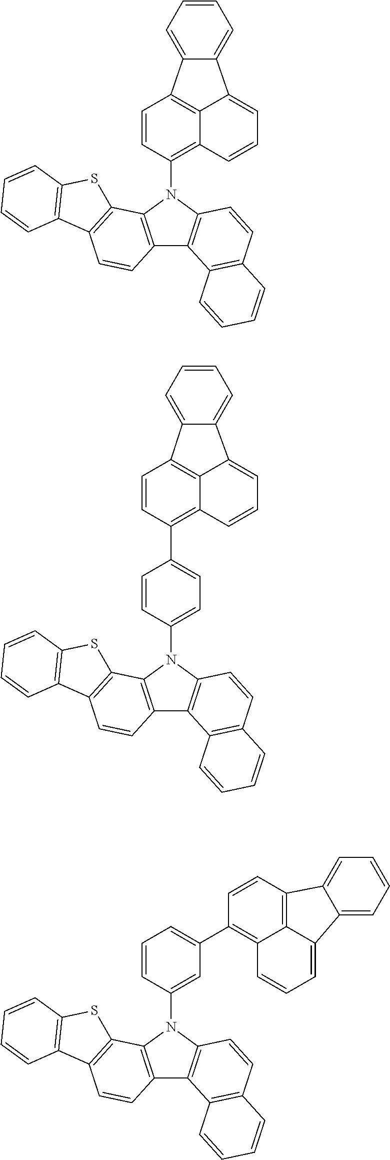

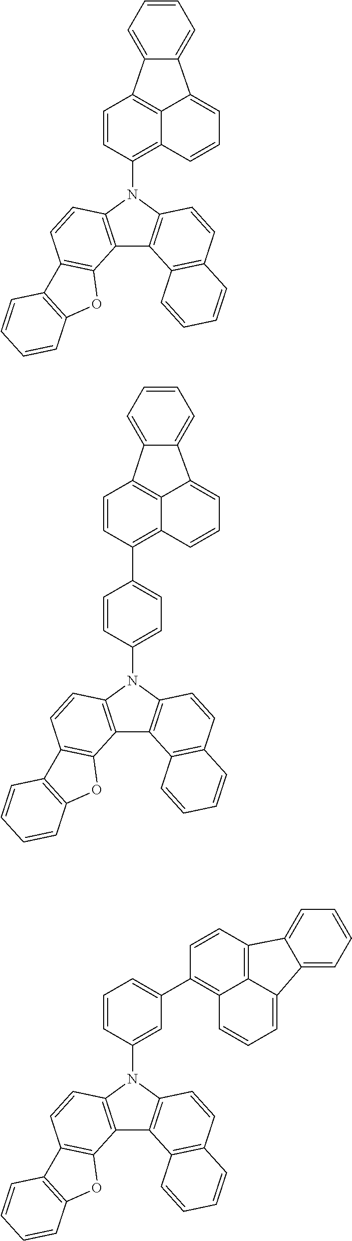 [SMILES: c1ccc2c(c1)-c1cccc3c(-n4c5ccc6ccccc6c5c5c6oc7ccccc7c6ccc54)ccc-2c13 ; c1ccc2c(c1)-c1cccc3c(-c4ccc(-n5c6ccc7ccccc7c6c6c7oc8ccccc8c7ccc65)cc4)ccc-2c13 ; c1cc(-c2ccc3c4c(cccc24)-c2ccccc2-3)cc(-n2c3ccc4ccccc4c3c3c4oc5ccccc5c4ccc32)c1]

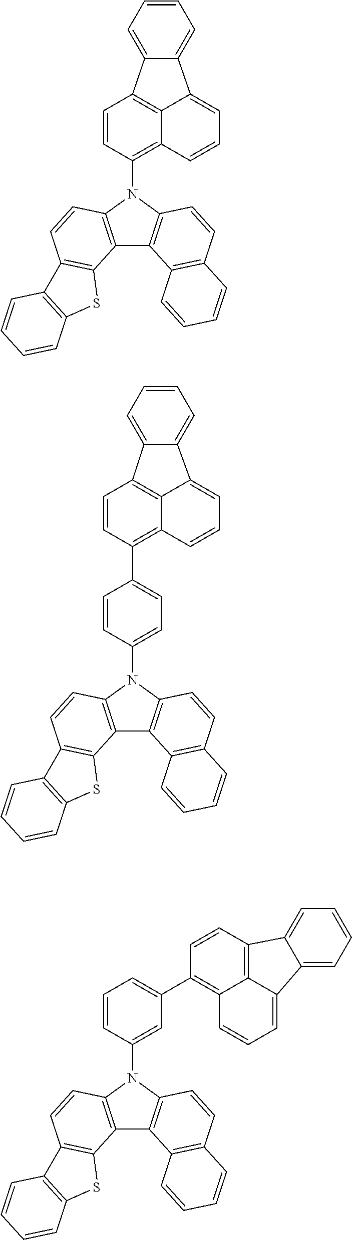 [SMILES: c1ccc2c(c1)-c1cccc3c(-n4c5ccc6ccccc6c5c5c6sc7ccccc7c6ccc54)ccc-2c13 ; c1ccc2c(c1)-c1cccc3c(-c4ccc(-n5c6ccc7ccccc7c6c6c7sc8ccccc8c7ccc65)cc4)ccc-2c13 ; c1cc(-c2ccc3c4c(cccc24)-c2ccccc2-3)cc(-n2c3ccc4ccccc4c3c3c4sc5ccccc5c4ccc32)c1]

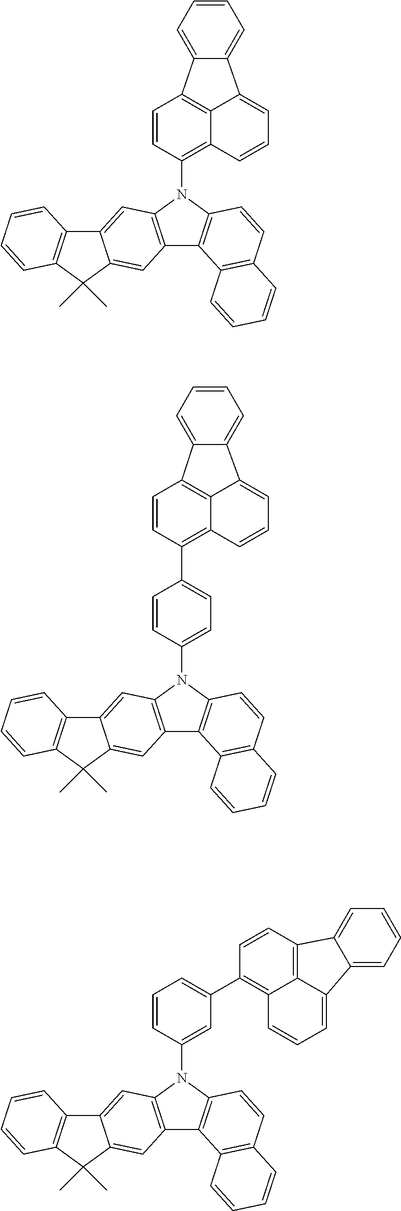 [SMILES: CC1(C)c2ccccc2-c2cc3c(cc21)c1c2ccccc2ccc1n3-c1ccc2c3c(cccc13)-c1ccccc1-2 ; CC1(C)c2ccccc2-c2cc3c(cc21)c1c2ccccc2ccc1n3-c1ccc(-c2ccc3c4c(cccc24)-c2ccccc2-3)cc1 ; CC1(C)c2ccccc2-c2cc3c(cc21)c1c2ccccc2ccc1n3-c1cccc(-c2ccc3c4c(cccc24)-c2ccccc2-3)c1]

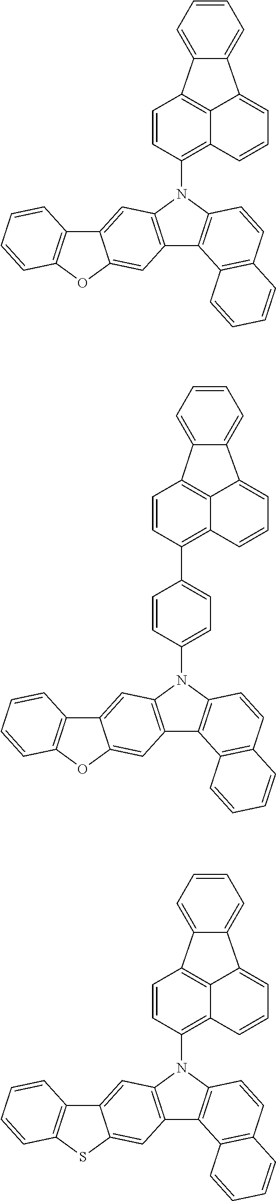 [SMILES: c1ccc2c(c1)-c1cccc3c(-n4c5cc6c(cc5c5c7ccccc7ccc54)oc4ccccc46)ccc-2c13 ; c1ccc2c(c1)-c1cccc3c(-c4ccc(-n5c6cc7c(cc6c6c8ccccc8ccc65)oc5ccccc57)cc4)ccc-2c13 ; c1ccc2c(c1)-c1cccc3c(-n4c5cc6c(cc5c5c7ccccc7ccc54)sc4ccccc46)ccc-2c13]

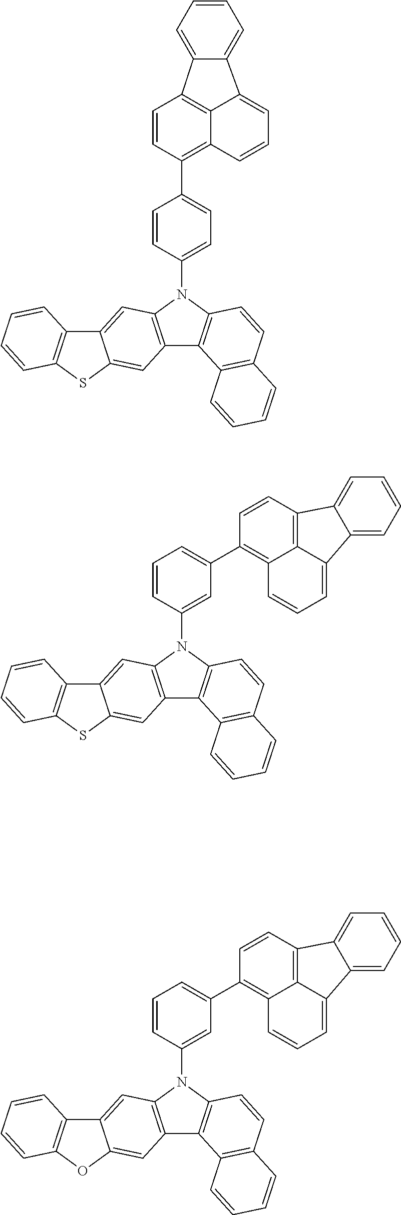 [SMILES: c1ccc2c(c1)-c1cccc3c(-c4ccc(-n5c6cc7c(cc6c6c8ccccc8ccc65)sc5ccccc57)cc4)ccc-2c13 ; c1cc(-c2ccc3c4c(cccc24)-c2ccccc2-3)cc(-n2c3cc4c(cc3c3c5ccccc5ccc32)sc2ccccc24)c1 ; c1cc(-c2ccc3c4c(cccc24)-c2ccccc2-3)cc(-n2c3cc4c(cc3c3c5ccccc5ccc32)oc2ccccc24)c1]

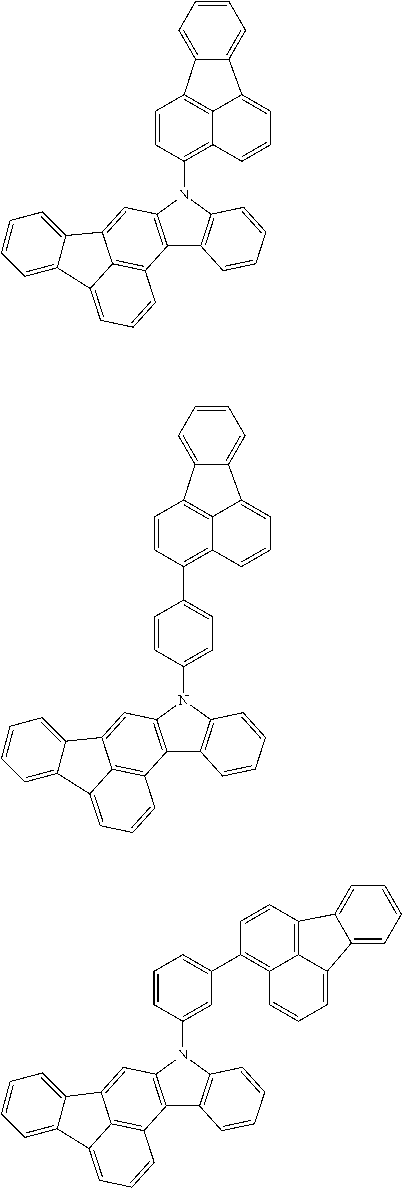 [SMILES: c1ccc2c(c1)-c1cccc3c(-n4c5ccccc5c5c6cccc7c6c(cc54)-c4ccccc4-7)ccc-2c13 ; c1ccc2c(c1)-c1cccc3c(-c4ccc(-n5c6ccccc6c6c7cccc8c7c(cc65)-c5ccccc5-8)cc4)ccc-2c13 ; c1cc(-c2ccc3c4c(cccc24)-c2ccccc2-3)cc(-n2c3ccccc3c3c4cccc5c4c(cc32)-c2ccccc2-5)c1]

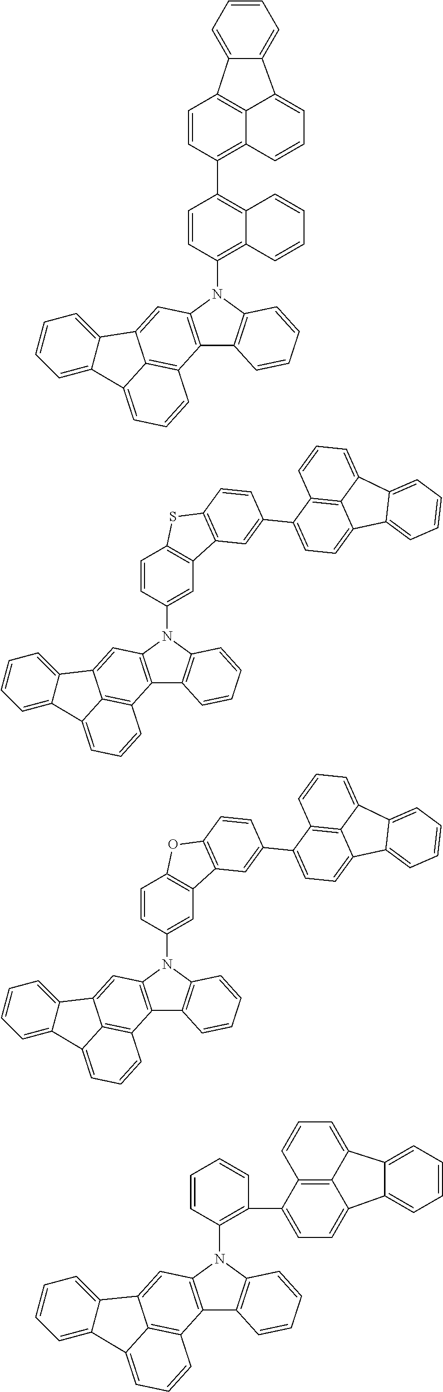 [SMILES: c1ccc2c(c1)-c1cccc3c(-c4ccc(-n5c6ccccc6c6c7cccc8c7c(cc65)-c5ccccc5-8)c5ccccc45)ccc-2c13 ; c1ccc2c(c1)-c1cccc3c(-c4ccc5sc6ccc(-n7c8ccccc8c8c9cccc%10c9c(cc87)-c7ccccc7-%10)cc6c5c4)ccc-2c13 ; c1ccc2c(c1)-c1cccc3c(-c4ccc5oc6ccc(-n7c8ccccc8c8c9cccc%10c9c(cc87)-c7ccccc7-%10)cc6c5c4)ccc-2c13 ; c1ccc2c(c1)-c1cccc3c(-c4ccccc4-n4c5ccccc5c5c6cccc7c6c(cc54)-c4ccccc4-7)ccc-2c13]

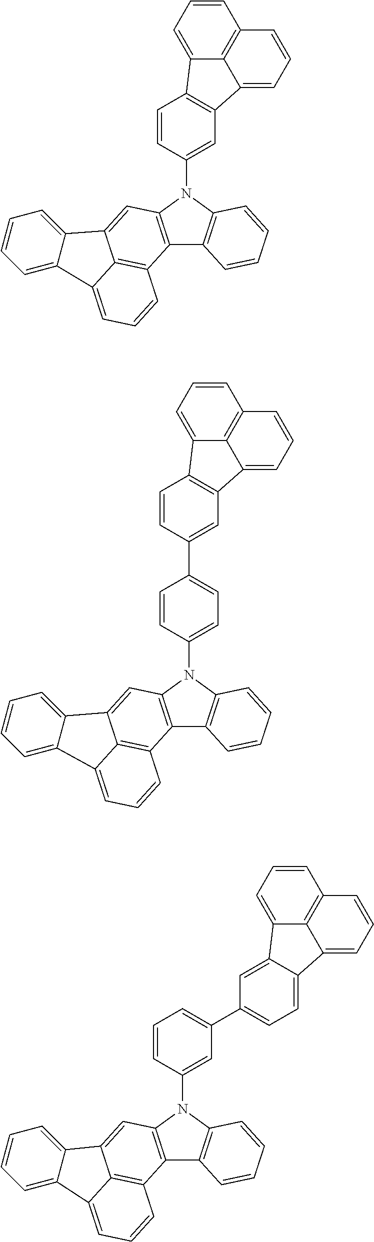 [SMILES: c1ccc2c(c1)-c1cccc3c1c-2cc1c3c2ccccc2n1-c1ccc2c(c1)-c1cccc3cccc-2c13 ; c1ccc2c(c1)-c1cccc3c1c-2cc1c3c2ccccc2n1-c1ccc(-c2ccc3c(c2)-c2cccc4cccc-3c24)cc1 ; c1cc(-c2ccc3c(c2)-c2cccc4cccc-3c24)cc(-n2c3ccccc3c3c4cccc5c4c(cc32)-c2ccccc2-5)c1]

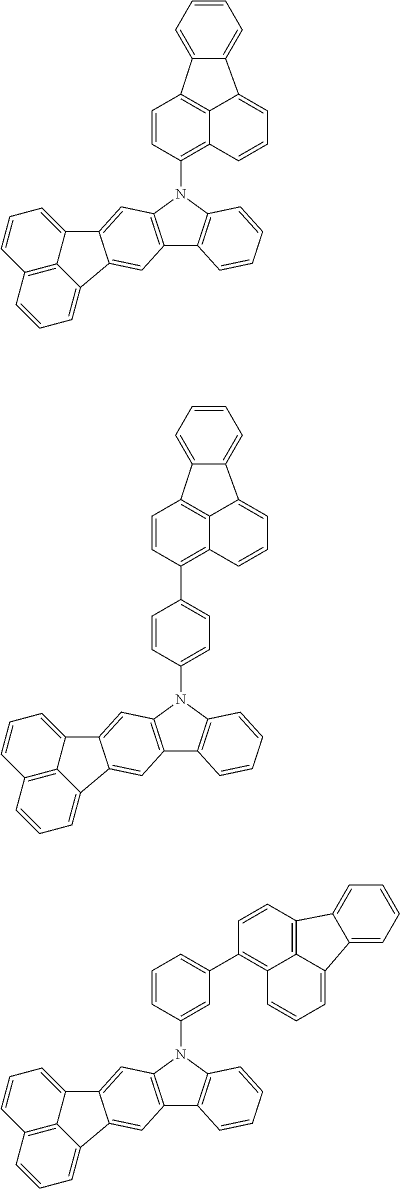 [SMILES: c1ccc2c(c1)-c1cccc3c(-n4c5ccccc5c5cc6c(cc54)-c4cccc5cccc-6c45)ccc-2c13 ; c1ccc2c(c1)-c1cccc3c(-c4ccc(-n5c6ccccc6c6cc7c(cc65)-c5cccc6cccc-7c56)cc4)ccc-2c13 ; c1cc(-c2ccc3c4c(cccc24)-c2ccccc2-3)cc(-n2c3ccccc3c3cc4c(cc32)-c2cccc3cccc-4c23)c1]

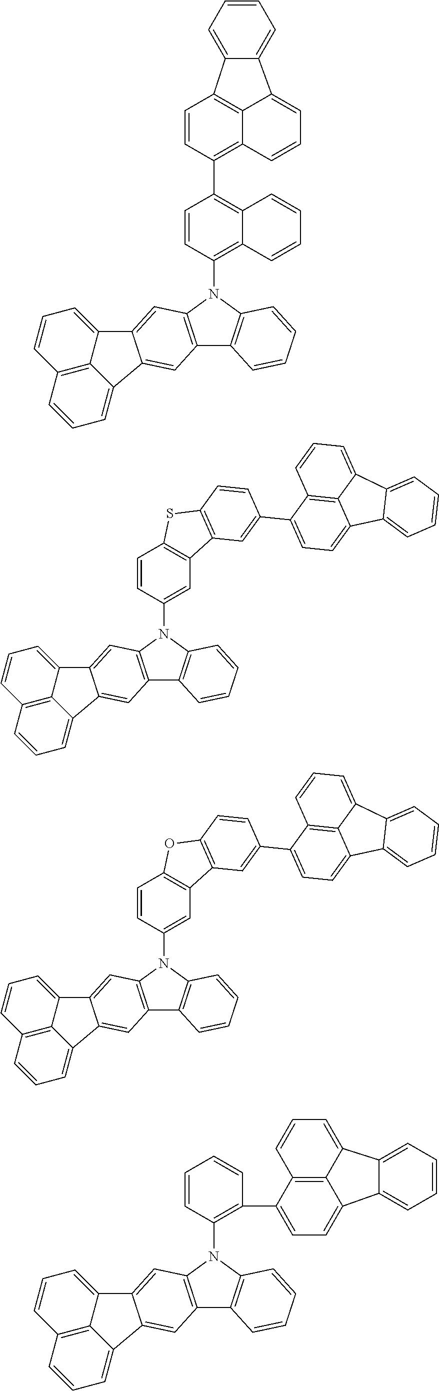 [SMILES: c1ccc2c(c1)-c1cccc3c(-c4ccc(-n5c6ccccc6c6cc7c(cc65)-c5cccc6cccc-7c56)c5ccccc45)ccc-2c13 ; c1ccc2c(c1)-c1cccc3c(-c4ccc5sc6ccc(-n7c8ccccc8c8cc9c(cc87)-c7cccc8cccc-9c78)cc6c5c4)ccc-2c13 ; c1ccc2c(c1)-c1cccc3c(-c4ccc5oc6ccc(-n7c8ccccc8c8cc9c(cc87)-c7cccc8cccc-9c78)cc6c5c4)ccc-2c13 ; c1ccc2c(c1)-c1cccc3c(-c4ccccc4-n4c5ccccc5c5cc6c(cc54)-c4cccc5cccc-6c45)ccc-2c13]

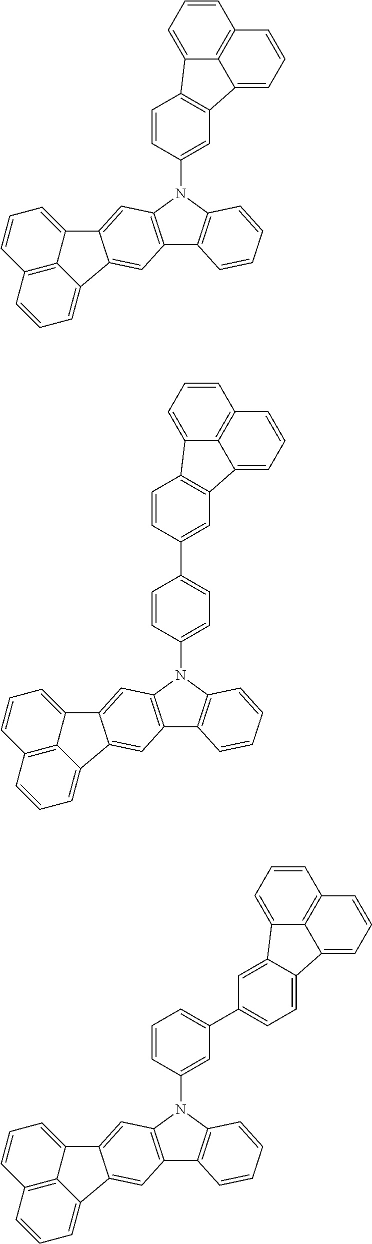 [SMILES: c1cc2c3c(cccc3c1)-c1cc(-n3c4ccccc4c4cc5c(cc43)-c3cccc4cccc-5c34)ccc1-2 ; c1cc2c3c(cccc3c1)-c1cc(-c3ccc(-n4c5ccccc5c5cc6c(cc54)-c4cccc5cccc-6c45)cc3)ccc1-2 ; c1cc(-c2ccc3c(c2)-c2cccc4cccc-3c24)cc(-n2c3ccccc3c3cc4c(cc32)-c2cccc3cccc-4c23)c1]

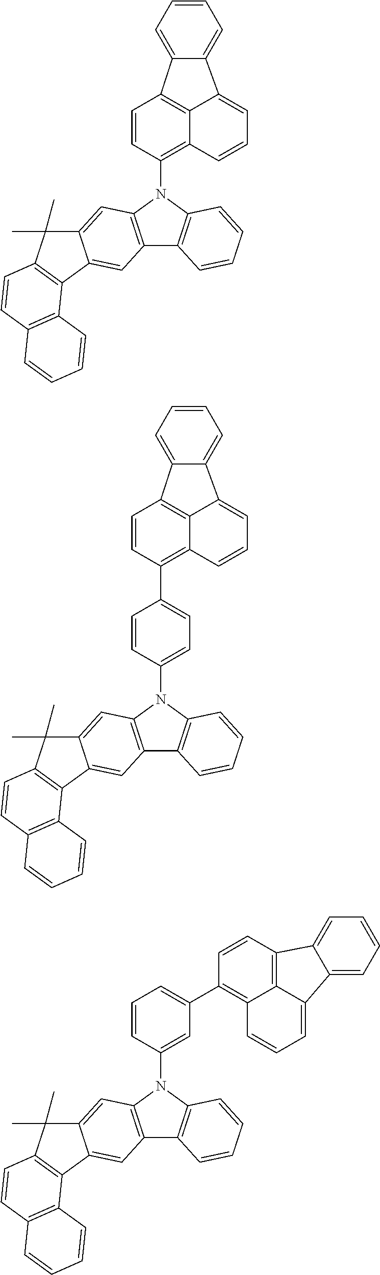 [SMILES: CC1(C)c2cc3c(cc2-c2c1ccc1ccccc21)c1ccccc1n3-c1ccc2c3c(cccc13)-c1ccccc1-2 ; CC1(C)c2cc3c(cc2-c2c1ccc1ccccc21)c1ccccc1n3-c1ccc(-c2ccc3c4c(cccc24)-c2ccccc2-3)cc1 ; CC1(C)c2cc3c(cc2-c2c1ccc1ccccc21)c1ccccc1n3-c1cccc(-c2ccc3c4c(cccc24)-c2ccccc2-3)c1]

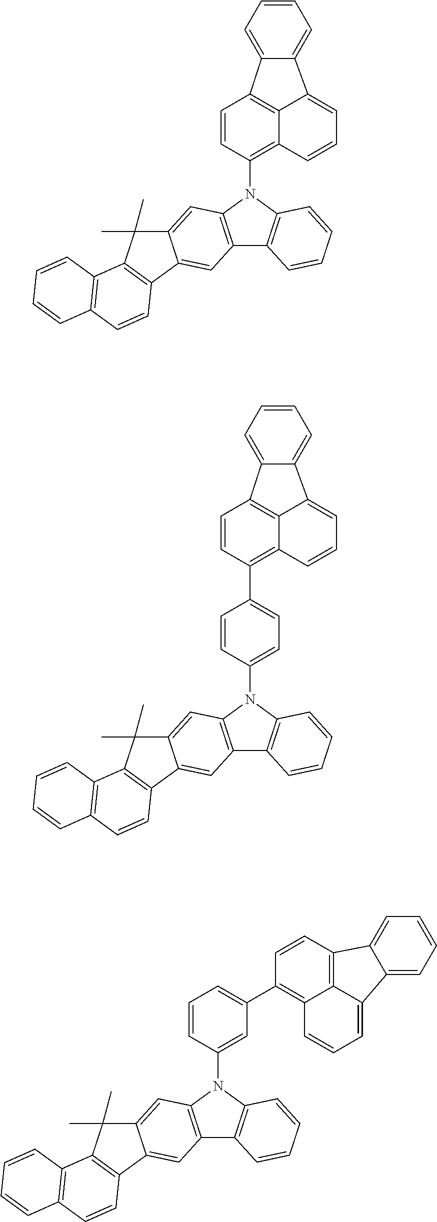 [SMILES: CC1(C)c2cc3c(cc2-c2ccc4ccccc4c21)c1ccccc1n3-c1ccc2c3c(cccc13)-c1ccccc1-2 ; CC1(C)c2cc3c(cc2-c2ccc4ccccc4c21)c1ccccc1n3-c1ccc(-c2ccc3c4c(cccc24)-c2ccccc2-3)cc1 ; CC1(C)c2cc3c(cc2-c2ccc4ccccc4c21)c1ccccc1n3-c1cccc(-c2ccc3c4c(cccc24)-c2ccccc2-3)c1]

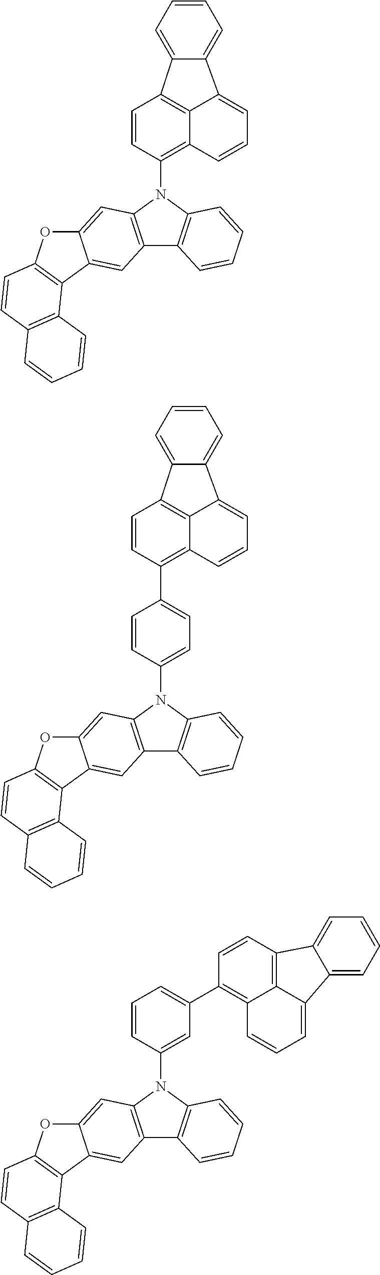 [SMILES: c1ccc2c(c1)-c1cccc3c(-n4c5ccccc5c5cc6c(cc54)oc4ccc5ccccc5c46)ccc-2c13 ; c1ccc2c(c1)-c1cccc3c(-c4ccc(-n5c6ccccc6c6cc7c(cc65)oc5ccc6ccccc6c57)cc4)ccc-2c13 ; c1cc(-c2ccc3c4c(cccc24)-c2ccccc2-3)cc(-n2c3ccccc3c3cc4c(cc32)oc2ccc3ccccc3c24)c1]

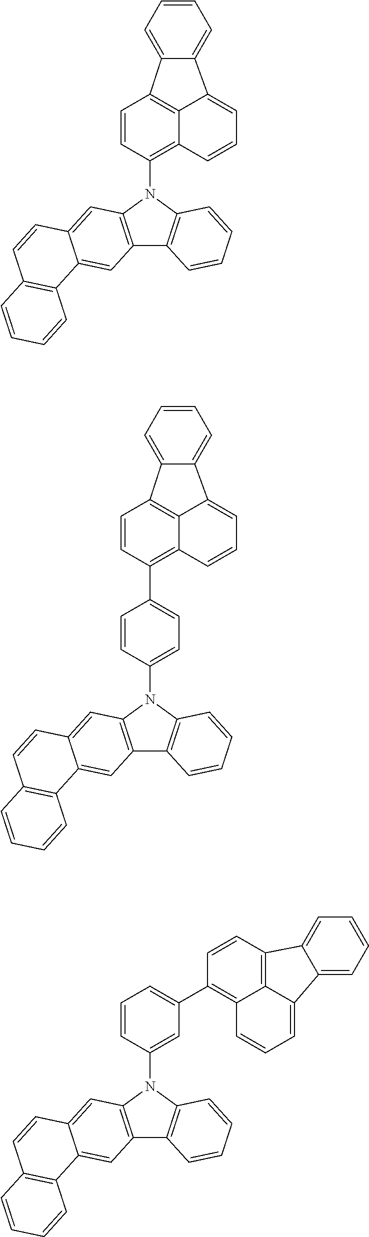 [SMILES: c1ccc2c(c1)-c1cccc3c(-n4c5ccccc5c5cc6c(ccc7ccccc76)cc54)ccc-2c13 ; c1ccc2c(c1)-c1cccc3c(-c4ccc(-n5c6ccccc6c6cc7c(ccc8ccccc87)cc65)cc4)ccc-2c13 ; c1cc(-c2ccc3c4c(cccc24)-c2ccccc2-3)cc(-n2c3ccccc3c3cc4c(ccc5ccccc54)cc32)c1]

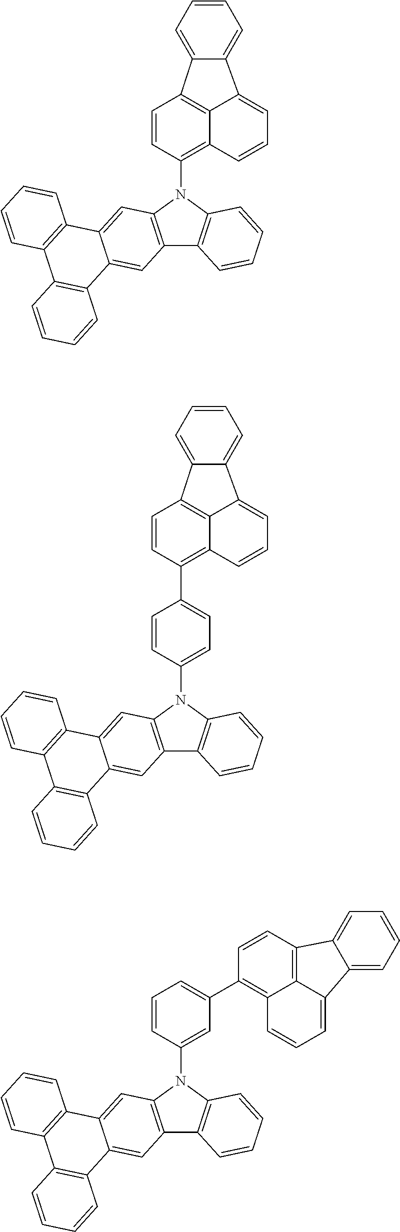 [SMILES: c1ccc2c(c1)-c1cccc3c(-n4c5ccccc5c5cc6c7ccccc7c7ccccc7c6cc54)ccc-2c13 ; c1ccc2c(c1)-c1cccc3c(-c4ccc(-n5c6ccccc6c6cc7c8ccccc8c8ccccc8c7cc65)cc4)ccc-2c13 ; c1cc(-c2ccc3c4c(cccc24)-c2ccccc2-3)cc(-n2c3ccccc3c3cc4c5ccccc5c5ccccc5c4cc32)c1]

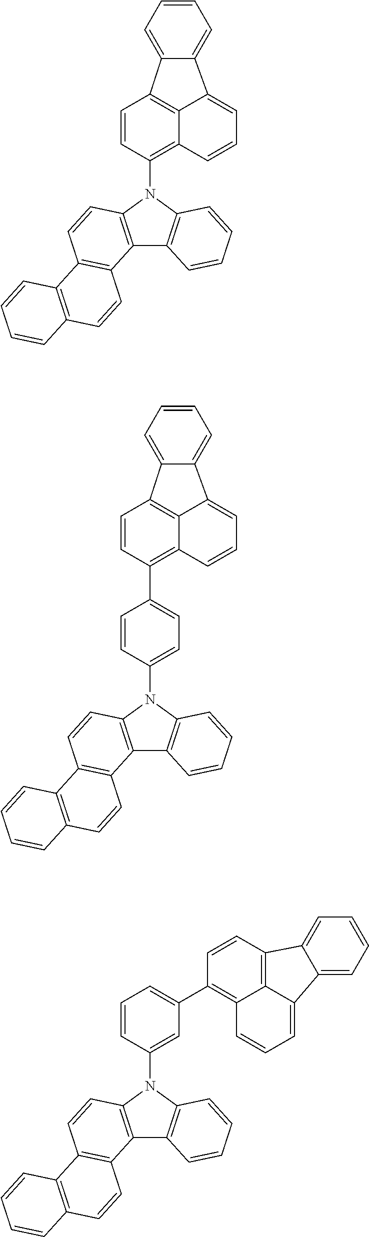 [SMILES: c1ccc2c(c1)-c1cccc3c(-n4c5ccccc5c5c6ccc7ccccc7c6ccc54)ccc-2c13 ; c1ccc2c(c1)-c1cccc3c(-c4ccc(-n5c6ccccc6c6c7ccc8ccccc8c7ccc65)cc4)ccc-2c13 ; c1cc(-c2ccc3c4c(cccc24)-c2ccccc2-3)cc(-n2c3ccccc3c3c4ccc5ccccc5c4ccc32)c1]

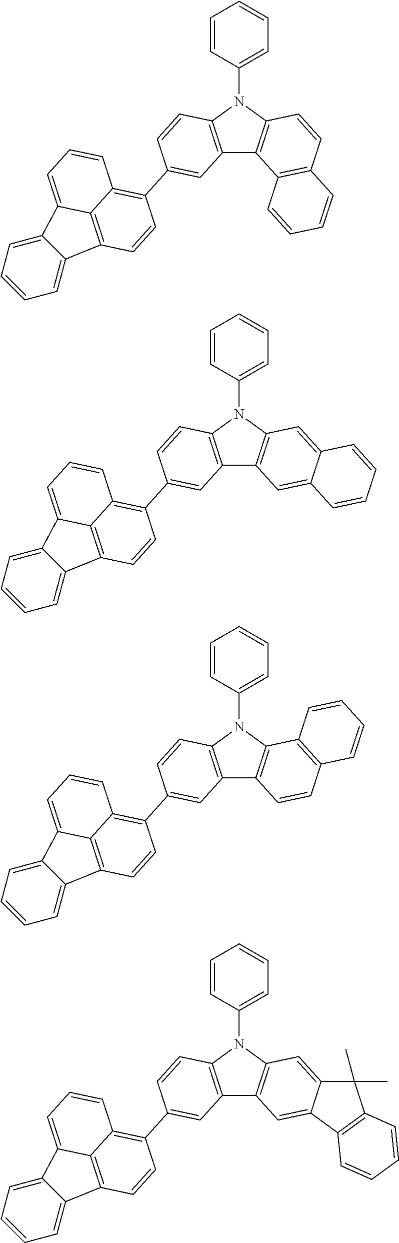 [SMILES: c1ccc(-n2c3ccc(-c4ccc5c6c(cccc46)-c4ccccc4-5)cc3c3c4ccccc4ccc32)cc1 ; c1ccc(-n2c3ccc(-c4ccc5c6c(cccc46)-c4ccccc4-5)cc3c3cc4ccccc4cc32)cc1 ; c1ccc(-n2c3ccc(-c4ccc5c6c(cccc46)-c4ccccc4-5)cc3c3ccc4ccccc4c32)cc1 ; CC1(C)c2ccccc2-c2cc3c4cc(-c5ccc6c7c(cccc57)-c5ccccc5-6)ccc4n(-c4ccccc4)c3cc21]

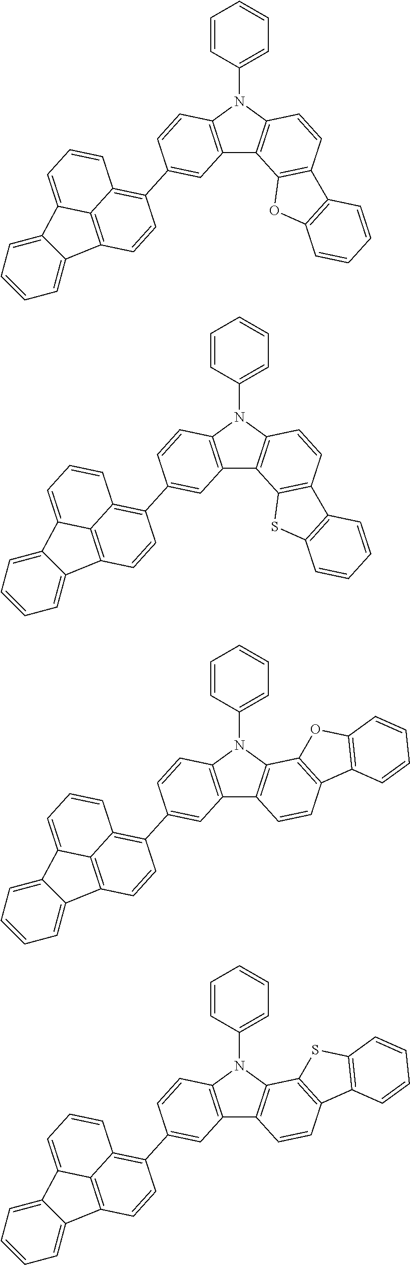 [SMILES: c1ccc(-n2c3ccc(-c4ccc5c6c(cccc46)-c4ccccc4-5)cc3c3c4oc5ccccc5c4ccc32)cc1 ; c1ccc(-n2c3ccc(-c4ccc5c6c(cccc46)-c4ccccc4-5)cc3c3c4sc5ccccc5c4ccc32)cc1 ; c1ccc(-n2c3ccc(-c4ccc5c6c(cccc46)-c4ccccc4-5)cc3c3ccc4c5ccccc5oc4c32)cc1 ; c1ccc(-n2c3ccc(-c4ccc5c6c(cccc46)-c4ccccc4-5)cc3c3ccc4c5ccccc5sc4c32)cc1]

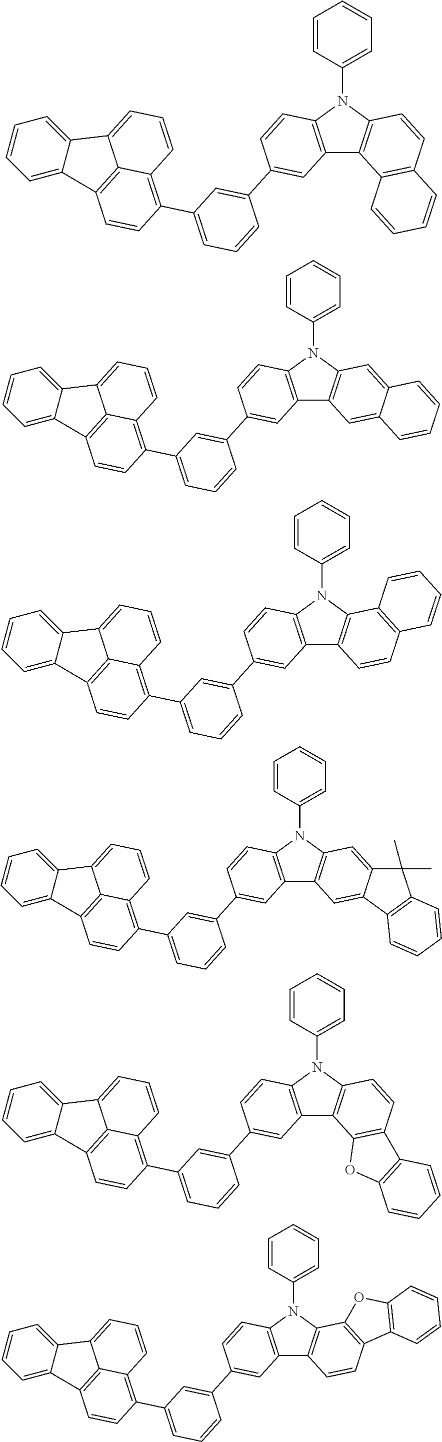

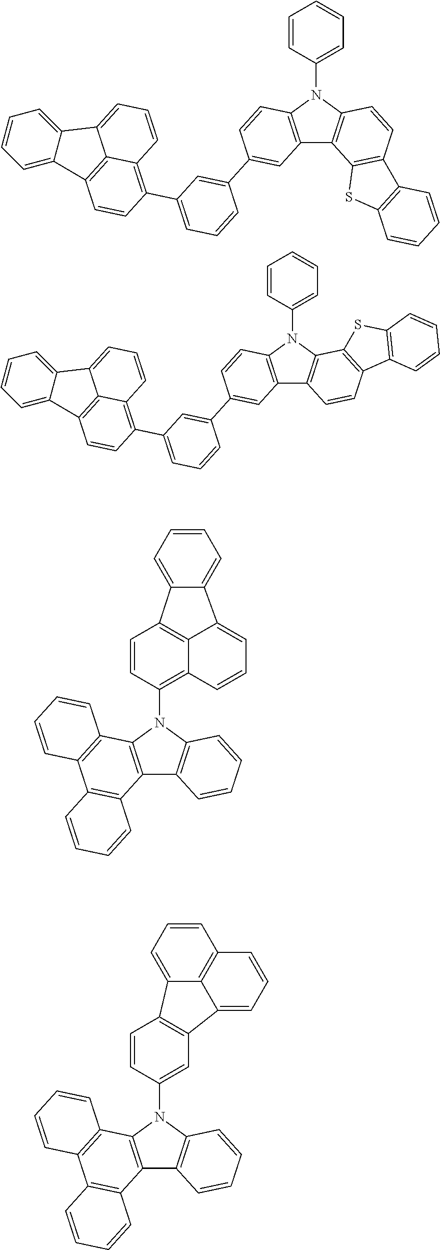 [SMILES: c1ccc(-n2c3ccc(-c4cccc(-c5ccc6c7c(cccc57)-c5ccccc5-6)c4)cc3c3c4sc5ccccc5c4ccc32)cc1 ; c1ccc(-n2c3ccc(-c4cccc(-c5ccc6c7c(cccc57)-c5ccccc5-6)c4)cc3c3ccc4c5ccccc5sc4c32)cc1 ; c1ccc2c(c1)-c1cccc3c(-n4c5ccccc5c5c6ccccc6c6ccccc6c54)ccc-2c13 ; c1cc2c3c(cccc3c1)-c1cc(-n3c4ccccc4c4c5ccccc5c5ccccc5c43)ccc1-2]

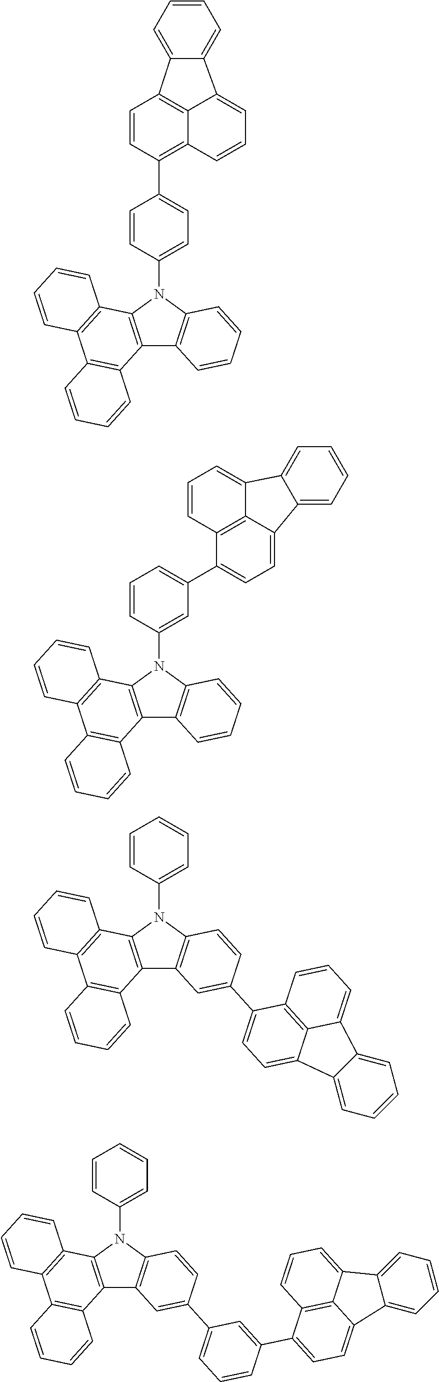

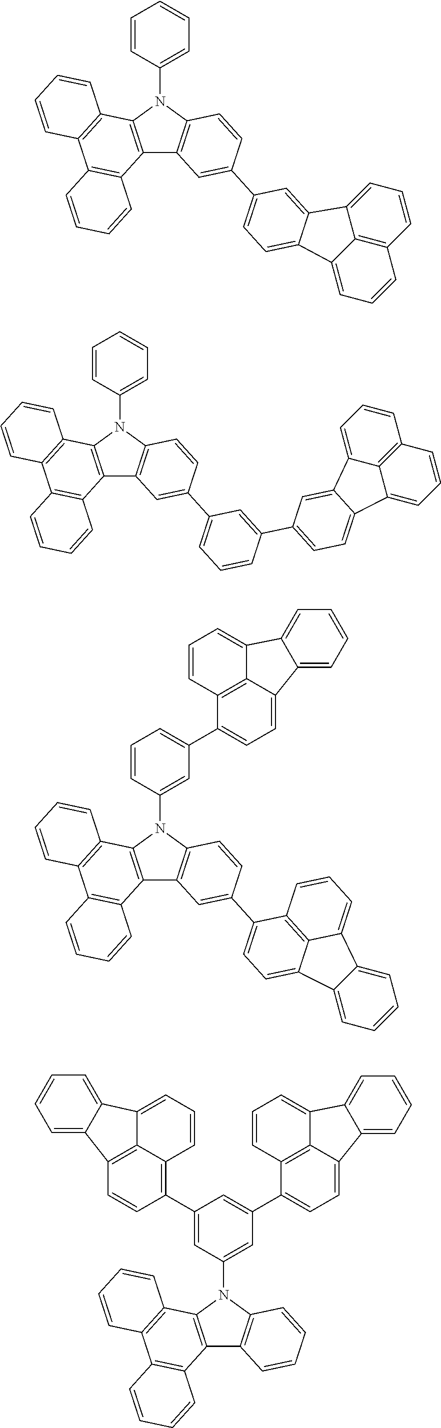 [SMILES: c1ccc(-n2c3ccc(-c4ccc5c(c4)-c4cccc6cccc-5c46)cc3c3c4ccccc4c4ccccc4c32)cc1 ; c1ccc(-n2c3ccc(-c4cccc(-c5ccc6c(c5)-c5cccc7cccc-6c57)c4)cc3c3c4ccccc4c4ccccc4c32)cc1 ; c1cc(-c2ccc3c4c(cccc24)-c2ccccc2-3)cc(-n2c3ccc(-c4ccc5c6c(cccc46)-c4ccccc4-5)cc3c3c4ccccc4c4ccccc4c32)c1 ; c1ccc2c(c1)-c1cccc3c(-c4cc(-c5ccc6c7c(cccc57)-c5ccccc5-6)cc(-n5c6ccccc6c6c7ccccc7c7ccccc7c65)c4)ccc-2c13]

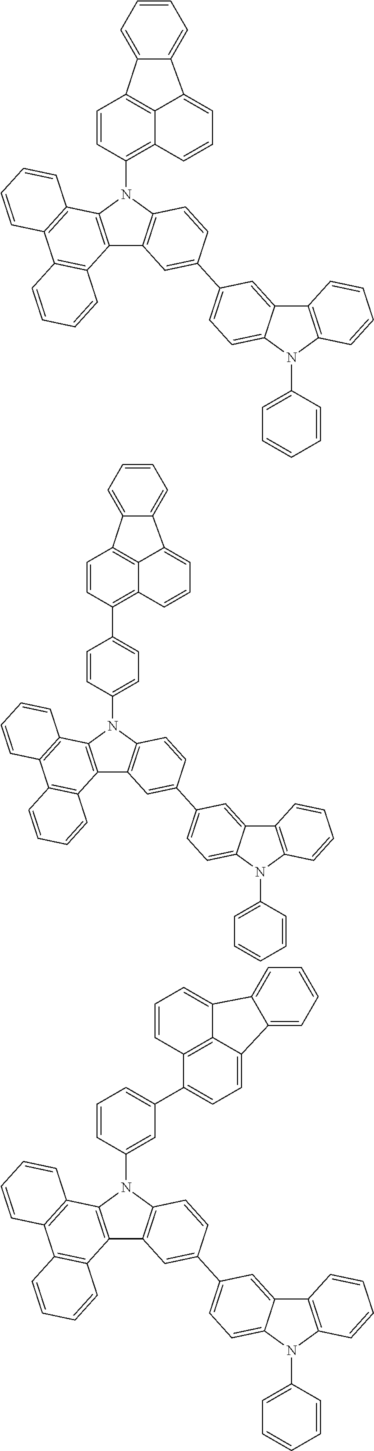

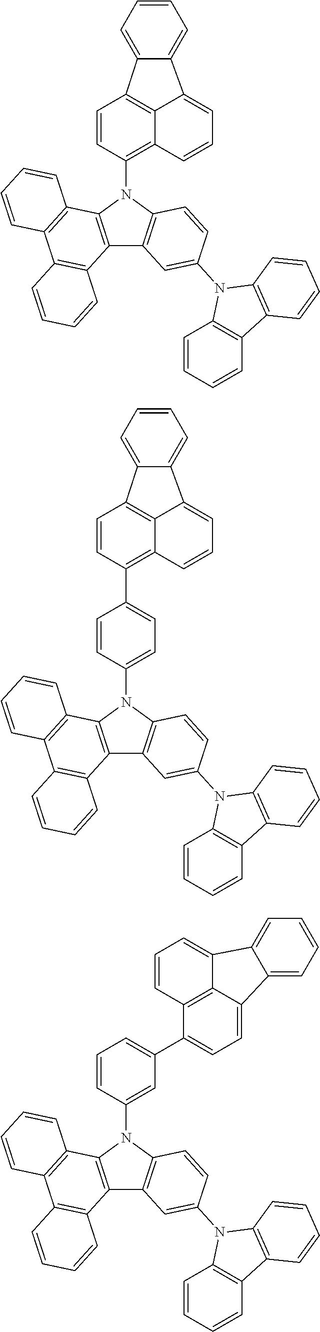

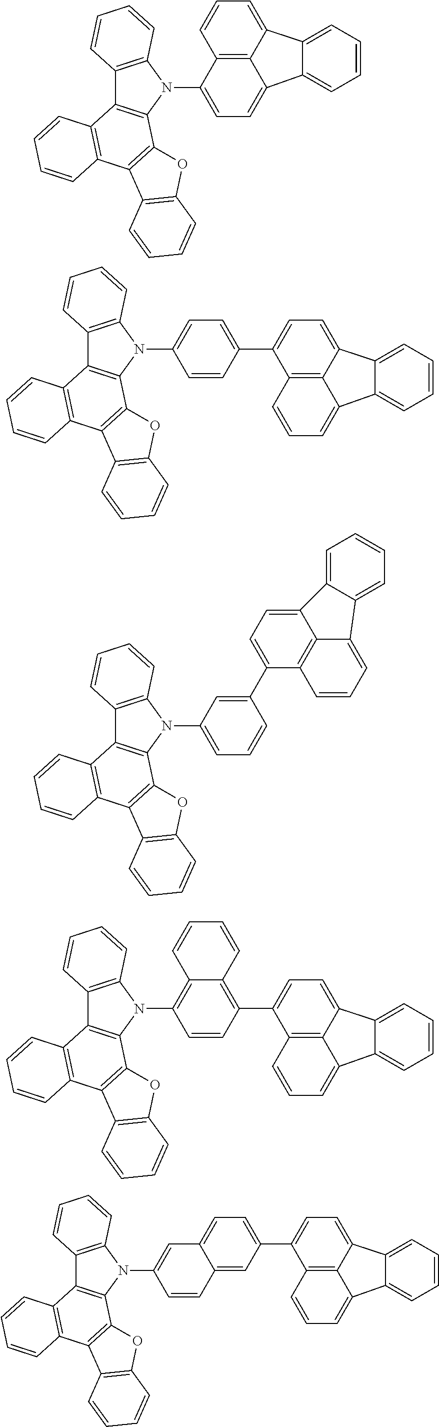

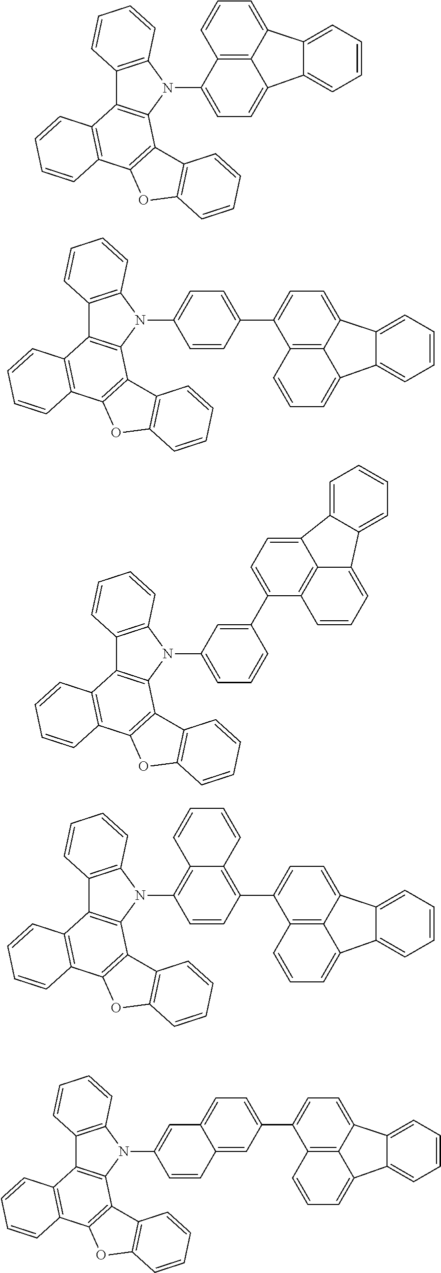

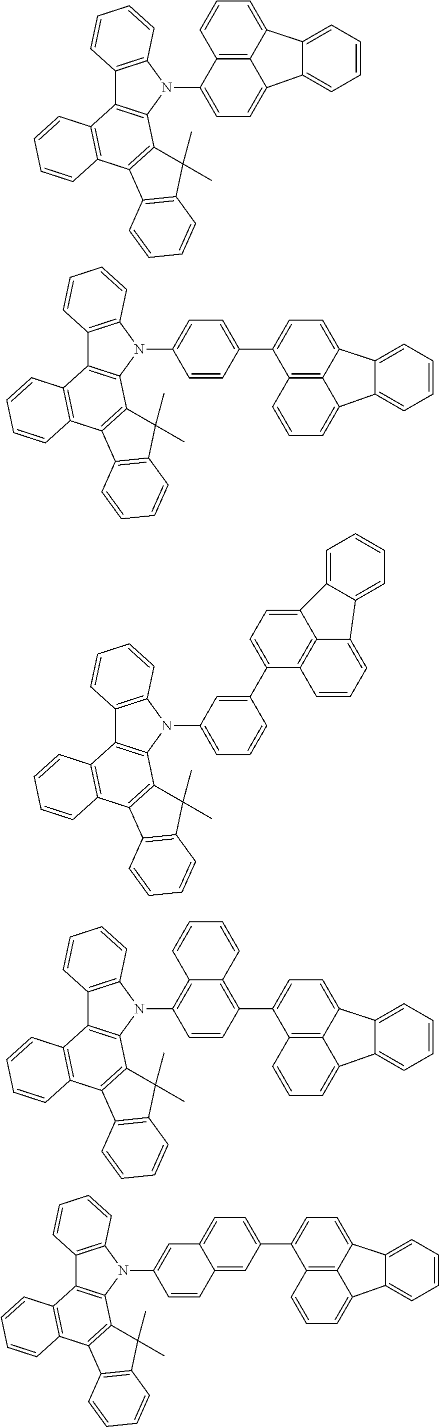

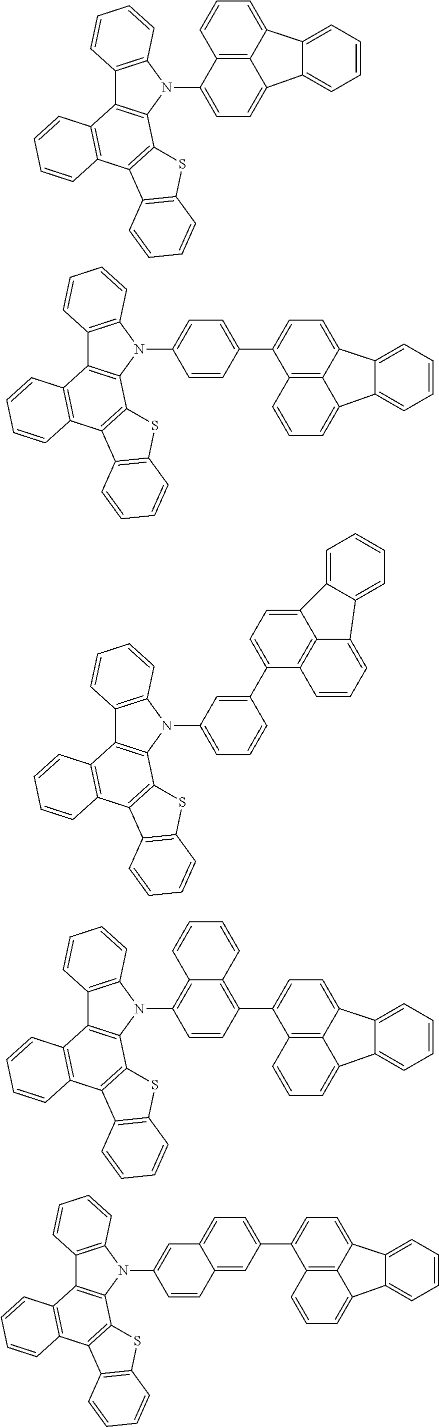

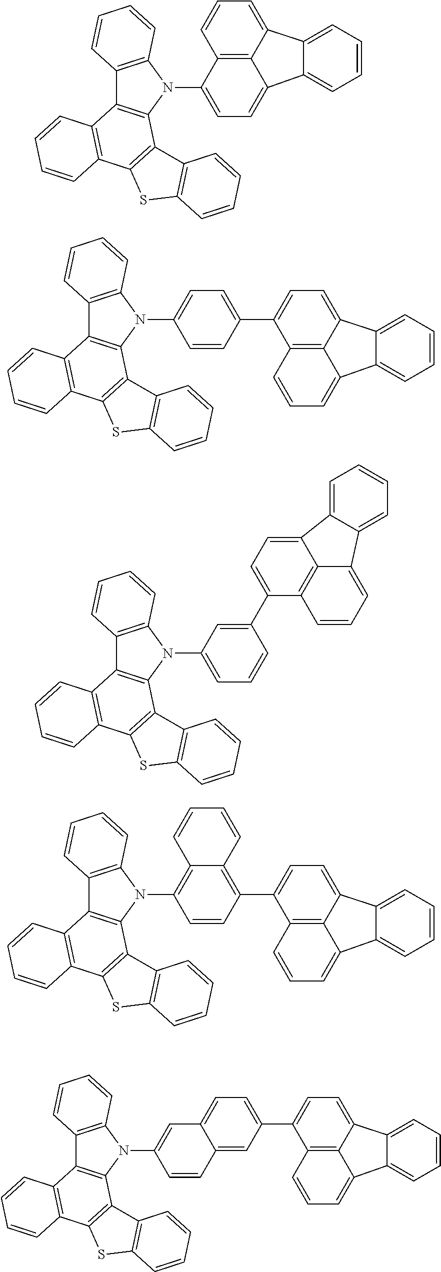

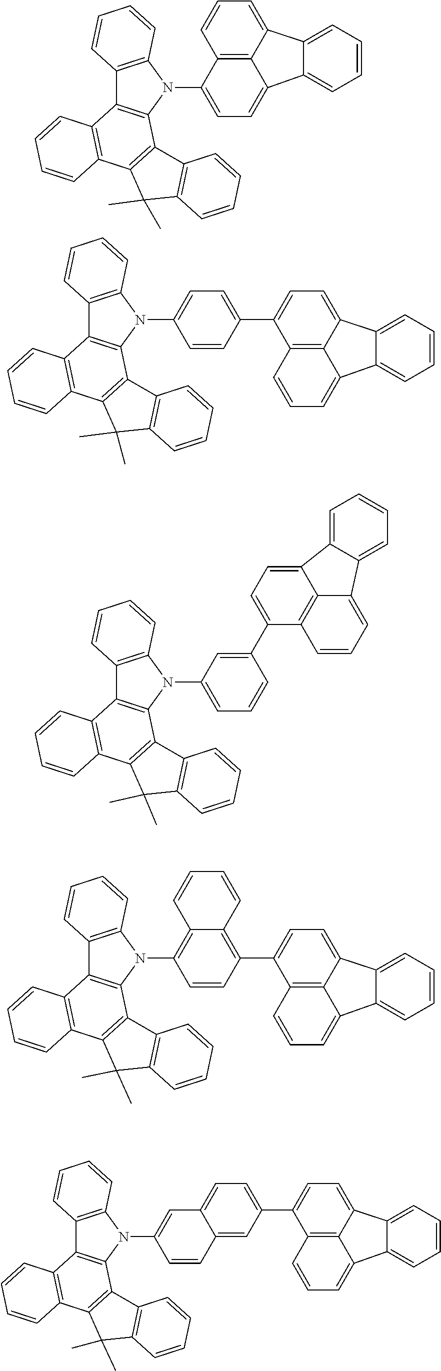

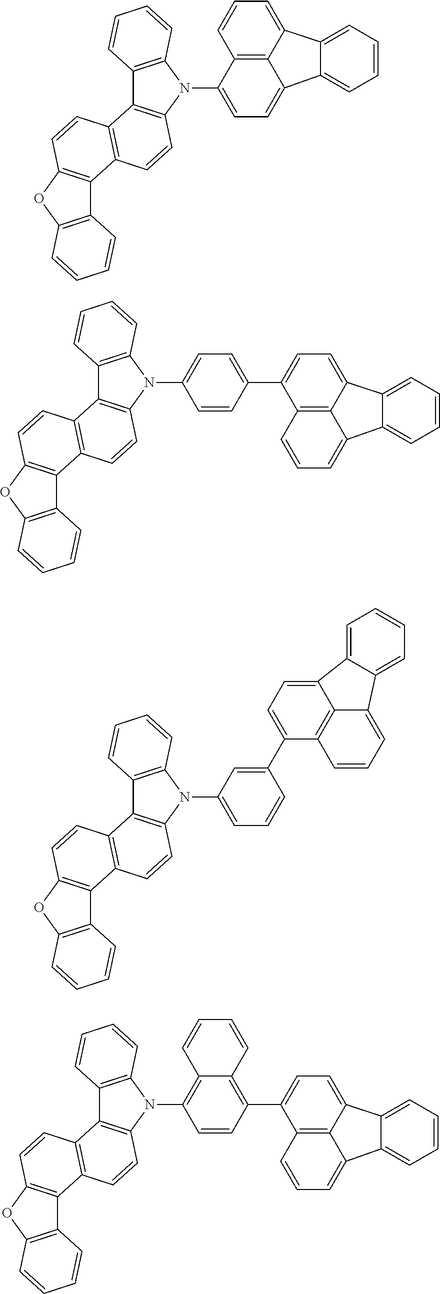

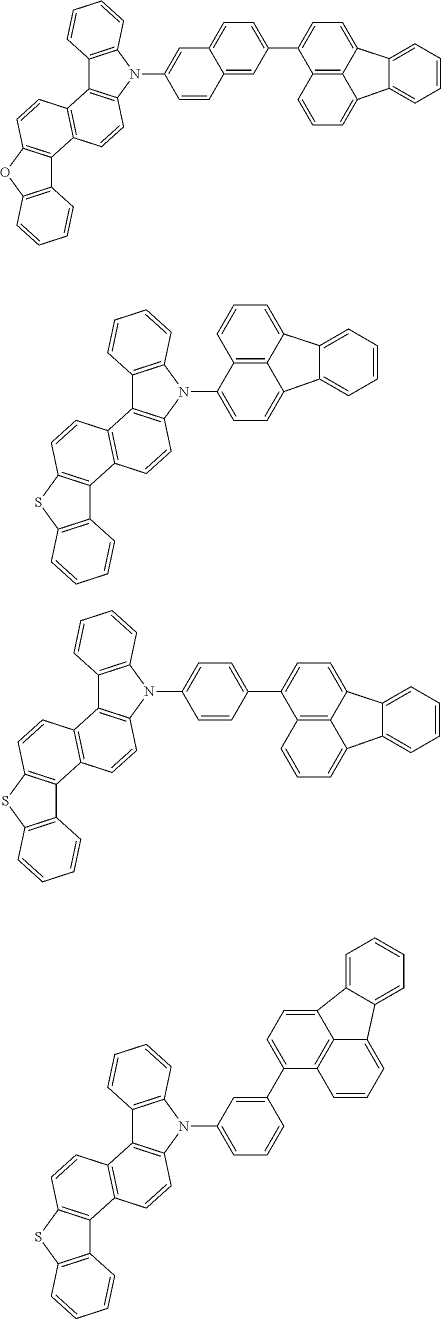

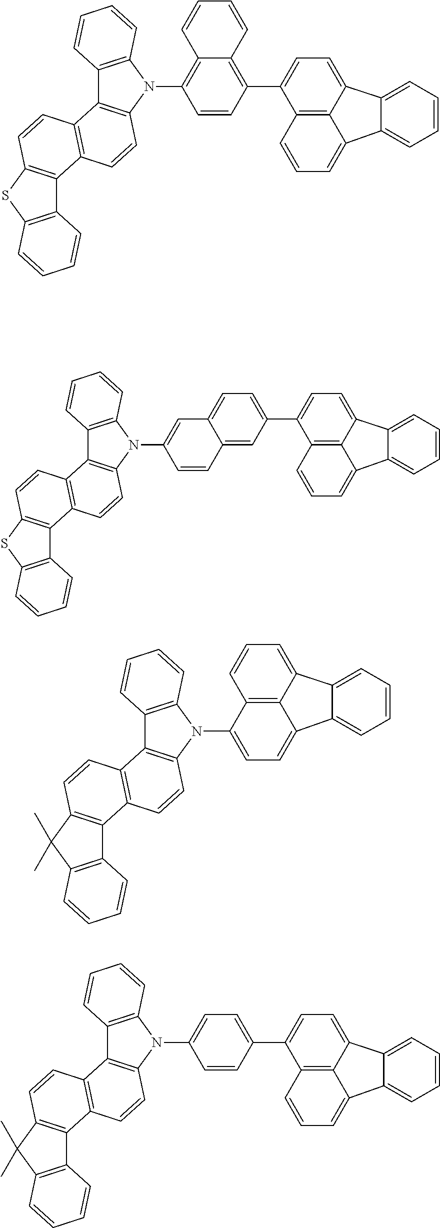 [SMILES: c1ccc2c(c1)-c1cccc3c(-c4ccc(-n5c6ccccc6c6c7ccc8sc9ccccc9c8c7ccc65)c5ccccc45)ccc-2c13 ; c1ccc2c(c1)-c1cccc3c(-c4ccc5cc(-n6c7ccccc7c7c8ccc9sc%10ccccc%10c9c8ccc76)ccc5c4)ccc-2c13 ; CC1(C)c2ccccc2-c2c1ccc1c2ccc2c1c1ccccc1n2-c1ccc2c3c(cccc13)-c1ccccc1-2 ; CC1(C)c2ccccc2-c2c1ccc1c2ccc2c1c1ccccc1n2-c1ccc(-c2ccc3c4c(cccc24)-c2ccccc2-3)cc1]

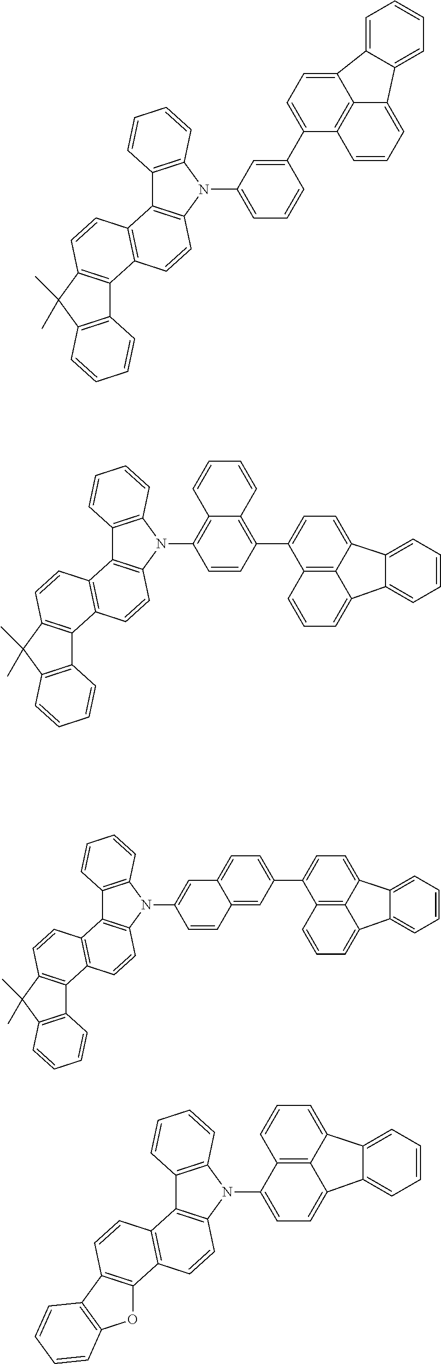 [SMILES: CC1(C)c2ccccc2-c2c1ccc1c2ccc2c1c1ccccc1n2-c1cccc(-c2ccc3c4c(cccc24)-c2ccccc2-3)c1 ; CC1(C)c2ccccc2-c2c1ccc1c2ccc2c1c1ccccc1n2-c1ccc(-c2ccc3c4c(cccc24)-c2ccccc2-3)c2ccccc12 ; CC1(C)c2ccccc2-c2c1ccc1c2ccc2c1c1ccccc1n2-c1ccc2cc(-c3ccc4c5c(cccc35)-c3ccccc3-4)ccc2c1 ; c1ccc2c(c1)-c1cccc3c(-n4c5ccccc5c5c6ccc7c8ccccc8oc7c6ccc54)ccc-2c13]

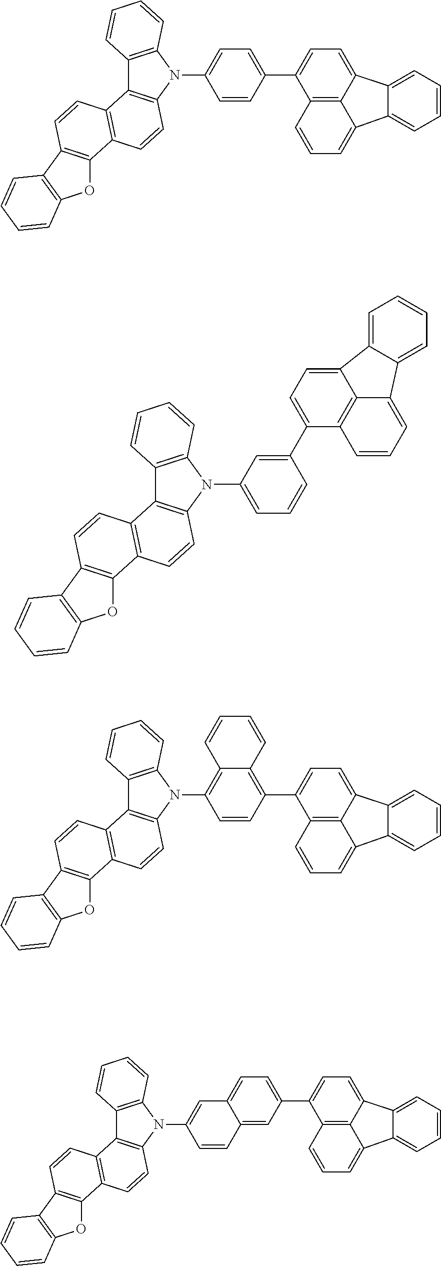 [SMILES: c1ccc2c(c1)-c1cccc3c(-c4ccc(-n5c6ccccc6c6c7ccc8c9ccccc9oc8c7ccc65)cc4)ccc-2c13 ; c1cc(-c2ccc3c4c(cccc24)-c2ccccc2-3)cc(-n2c3ccccc3c3c4ccc5c6ccccc6oc5c4ccc32)c1 ; c1ccc2c(c1)-c1cccc3c(-c4ccc(-n5c6ccccc6c6c7ccc8c9ccccc9oc8c7ccc65)c5ccccc45)ccc-2c13 ; c1ccc2c(c1)-c1cccc3c(-c4ccc5cc(-n6c7ccccc7c7c8ccc9c%10ccccc%10oc9c8ccc76)ccc5c4)ccc-2c13]

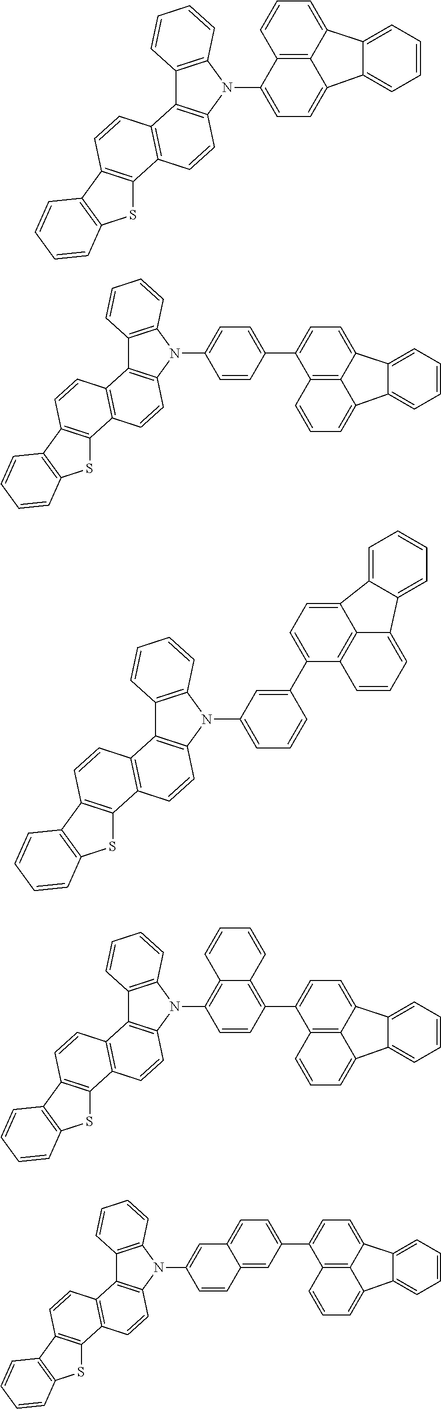

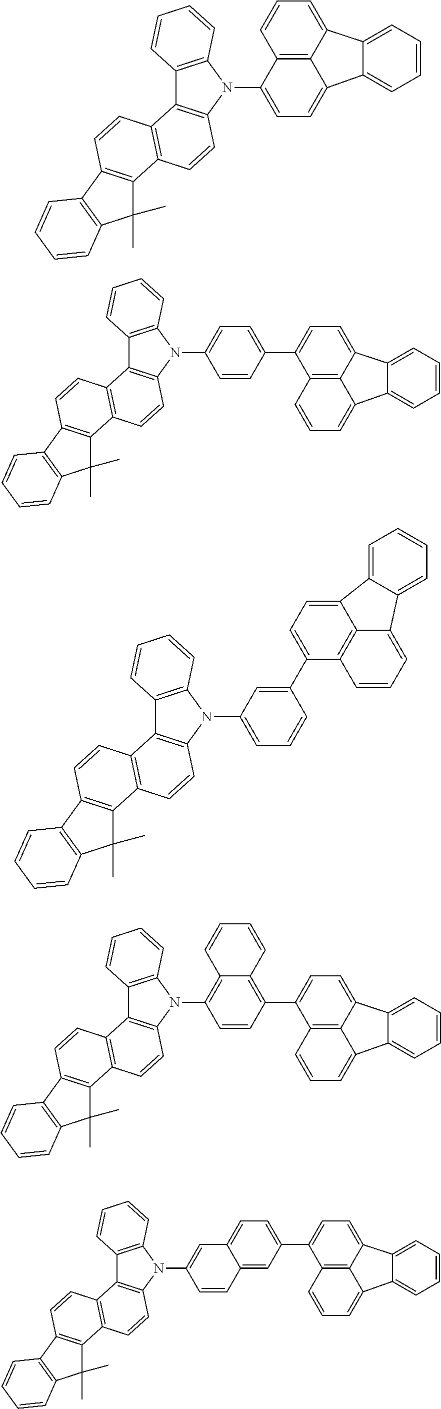

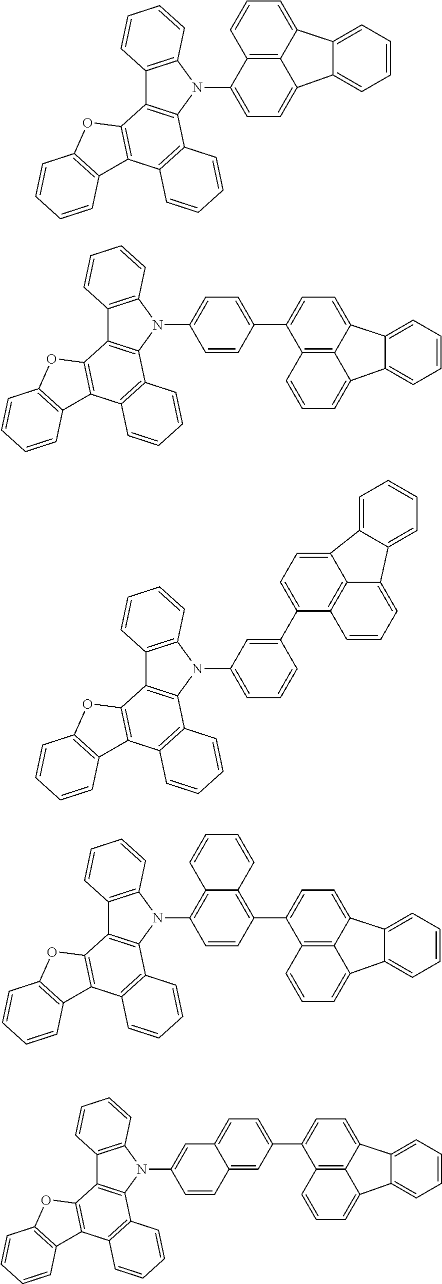

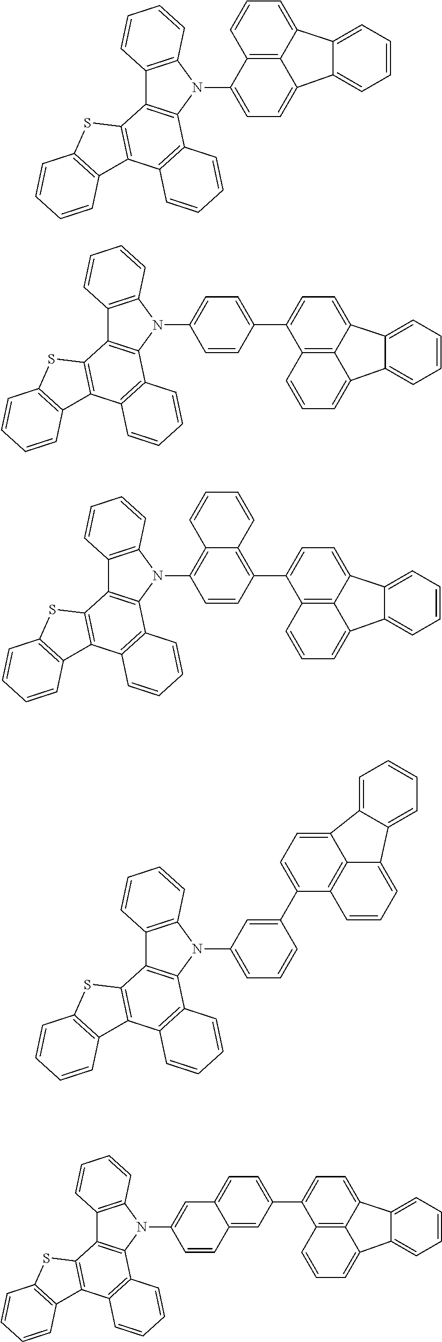 [SMILES: c1ccc2c(c1)-c1cccc3c(-n4c5ccccc5c5c6sc7ccccc7c6c6ccccc6c54)ccc-2c13 ; c1ccc2c(c1)-c1cccc3c(-c4ccc(-n5c6ccccc6c6c7sc8ccccc8c7c7ccccc7c65)cc4)ccc-2c13 ; c1ccc2c(c1)-c1cccc3c(-c4ccc(-n5c6ccccc6c6c7sc8ccccc8c7c7ccccc7c65)c5ccccc45)ccc-2c13 ; c1cc(-c2ccc3c4c(cccc24)-c2ccccc2-3)cc(-n2c3ccccc3c3c4sc5ccccc5c4c4ccccc4c32)c1 ; c1ccc2c(c1)-c1cccc3c(-c4ccc5cc(-n6c7ccccc7c7c8sc9ccccc9c8c8ccccc8c76)ccc5c4)ccc-2c13]

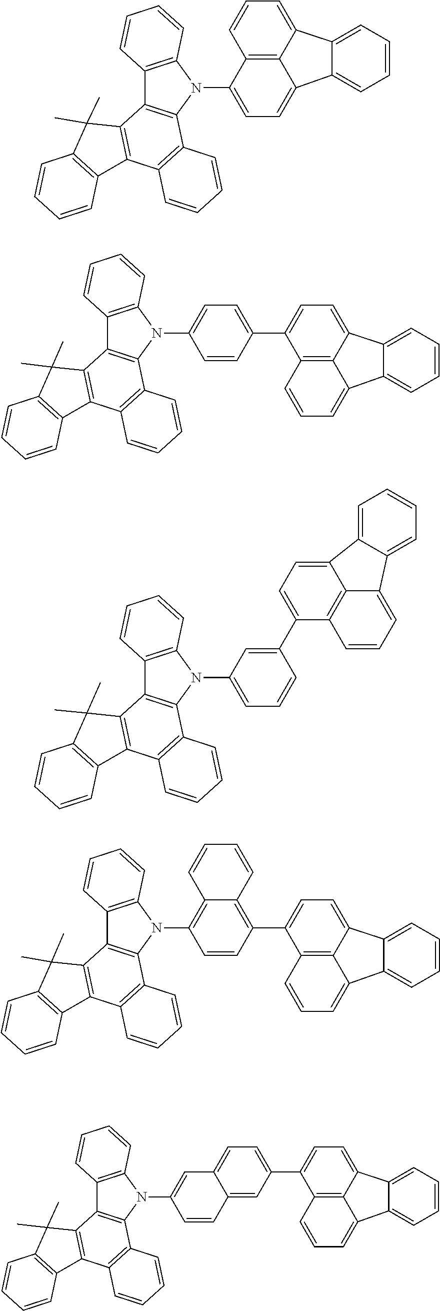

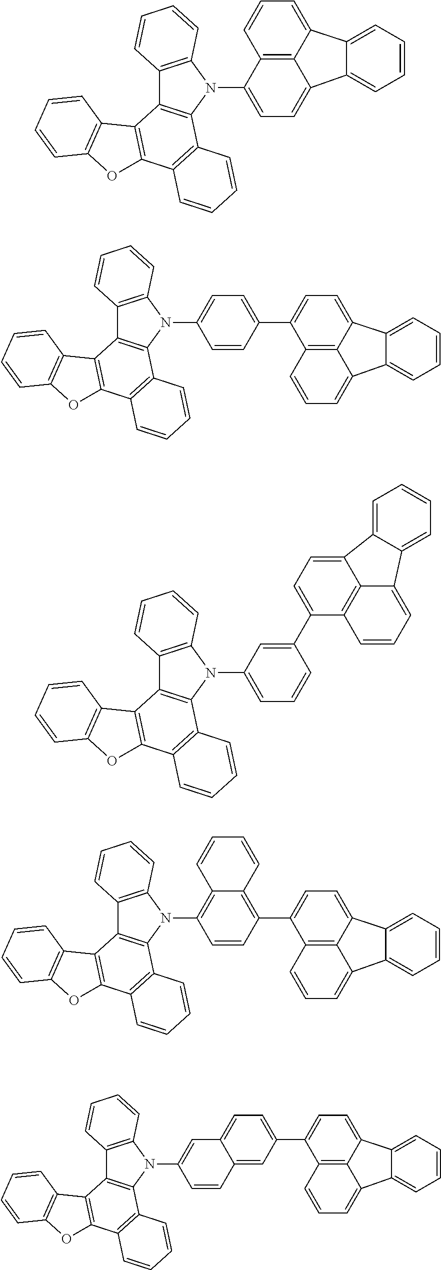

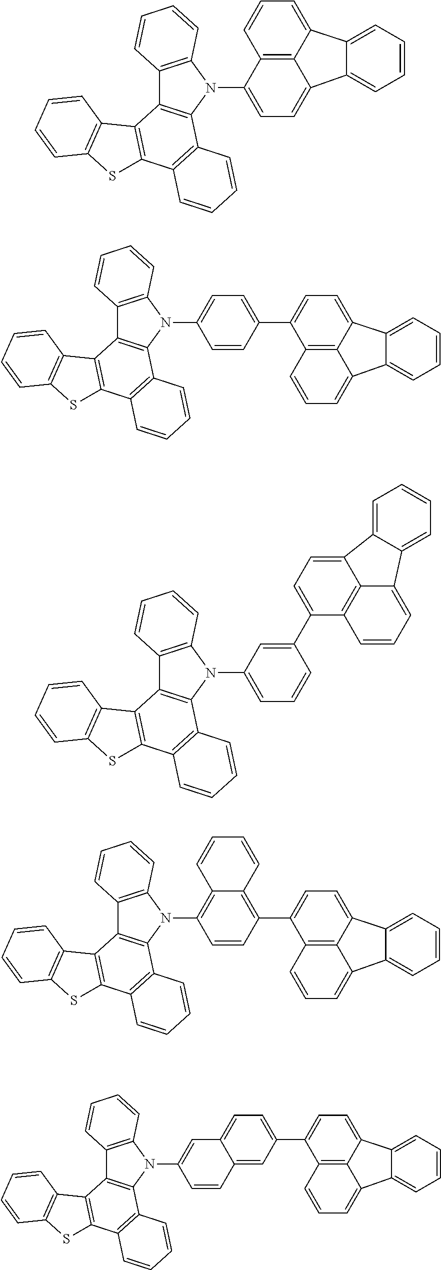

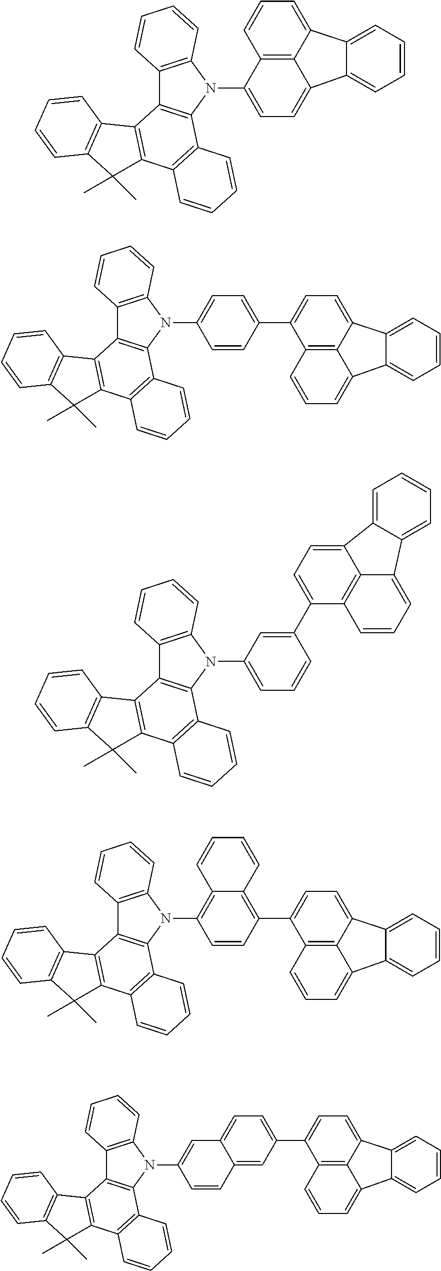 [SMILES: CC1(C)c2ccccc2-c2c1c1ccccc1c1c2c2ccccc2n1-c1ccc2c3c(cccc13)-c1ccccc1-2 ; CC1(C)c2ccccc2-c2c1c1ccccc1c1c2c2ccccc2n1-c1ccc(-c2ccc3c4c(cccc24)-c2ccccc2-3)cc1 ; CC1(C)c2ccccc2-c2c1c1ccccc1c1c2c2ccccc2n1-c1cccc(-c2ccc3c4c(cccc24)-c2ccccc2-3)c1 ; CC1(C)c2ccccc2-c2c1c1ccccc1c1c2c2ccccc2n1-c1ccc(-c2ccc3c4c(cccc24)-c2ccccc2-3)c2ccccc12 ; CC1(C)c2ccccc2-c2c1c1ccccc1c1c2c2ccccc2n1-c1ccc2cc(-c3ccc4c5c(cccc35)-c3ccccc3-4)ccc2c1]

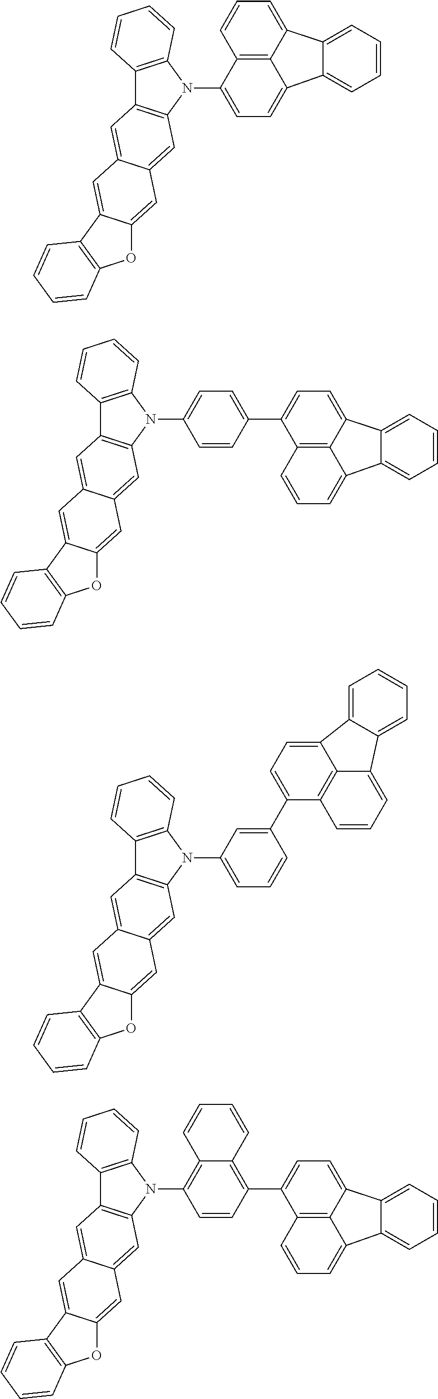 [SMILES: c1ccc2c(c1)-c1cccc3c(-n4c5ccccc5c5cc6cc7c(cc6cc54)oc4ccccc47)ccc-2c13 ; c1ccc2c(c1)-c1cccc3c(-c4ccc(-n5c6ccccc6c6cc7cc8c(cc7cc65)oc5ccccc58)cc4)ccc-2c13 ; c1cc(-c2ccc3c4c(cccc24)-c2ccccc2-3)cc(-n2c3ccccc3c3cc4cc5c(cc4cc32)oc2ccccc25)c1 ; c1ccc2c(c1)-c1cccc3c(-c4ccc(-n5c6ccccc6c6cc7cc8c(cc7cc65)oc5ccccc58)c5ccccc45)ccc-2c13]

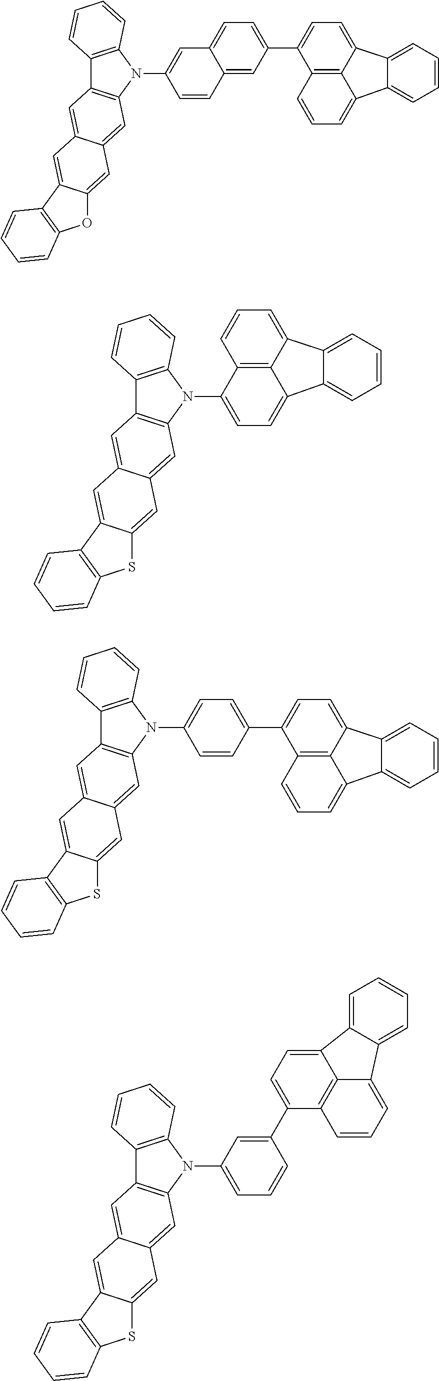 [SMILES: c1ccc2c(c1)-c1cccc3c(-c4ccc5cc(-n6c7ccccc7c7cc8cc9c(cc8cc76)oc6ccccc69)ccc5c4)ccc-2c13 ; c1ccc2c(c1)-c1cccc3c(-n4c5ccccc5c5cc6cc7c(cc6cc54)sc4ccccc47)ccc-2c13 ; c1ccc2c(c1)-c1cccc3c(-c4ccc(-n5c6ccccc6c6cc7cc8c(cc7cc65)sc5ccccc58)cc4)ccc-2c13 ; c1cc(-c2ccc3c4c(cccc24)-c2ccccc2-3)cc(-n2c3ccccc3c3cc4cc5c(cc4cc32)sc2ccccc25)c1]

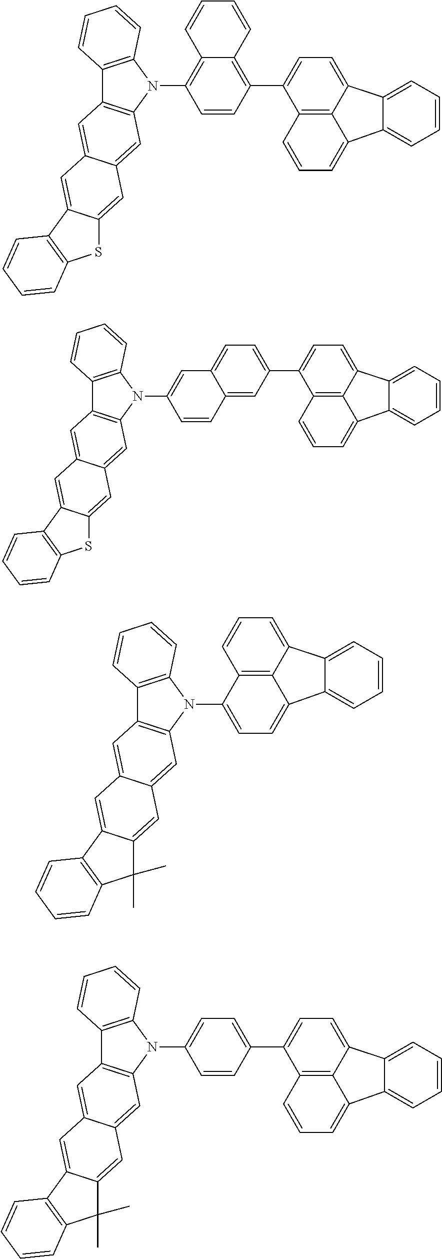 [SMILES: c1ccc2c(c1)-c1cccc3c(-c4ccc(-n5c6ccccc6c6cc7cc8c(cc7cc65)sc5ccccc58)c5ccccc45)ccc-2c13 ; c1ccc2c(c1)-c1cccc3c(-c4ccc5cc(-n6c7ccccc7c7cc8cc9c(cc8cc76)sc6ccccc69)ccc5c4)ccc-2c13 ; CC1(C)c2ccccc2-c2cc3cc4c5ccccc5n(-c5ccc6c7c(cccc57)-c5ccccc5-6)c4cc3cc21 ; CC1(C)c2ccccc2-c2cc3cc4c5ccccc5n(-c5ccc(-c6ccc7c8c(cccc68)-c6ccccc6-7)cc5)c4cc3cc21]

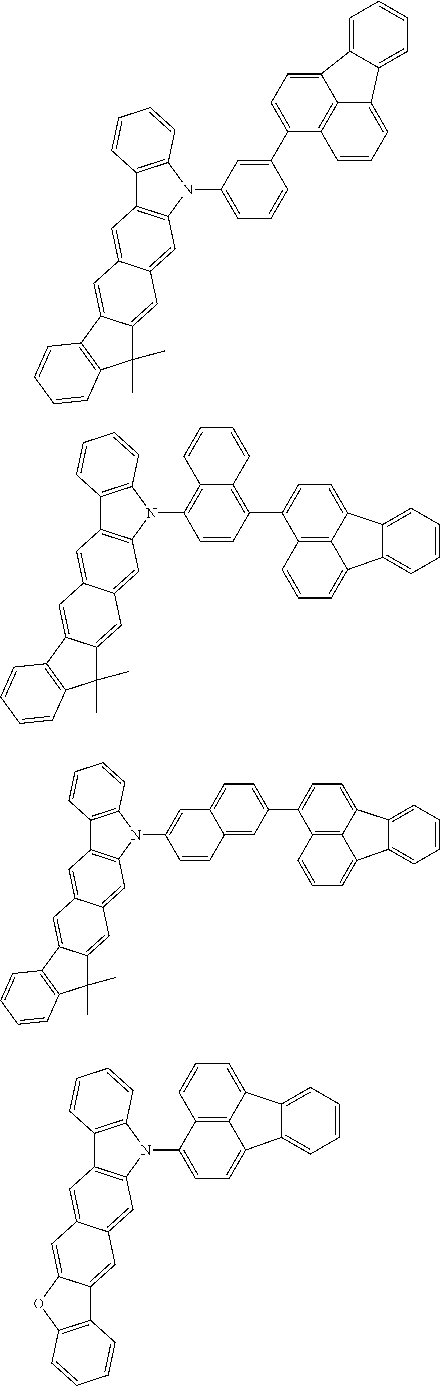

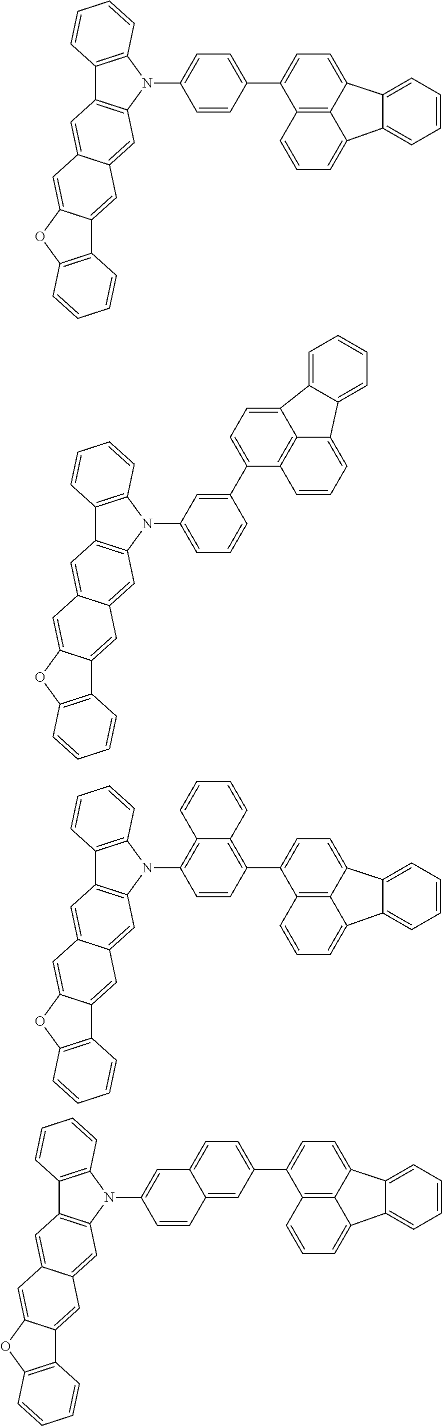

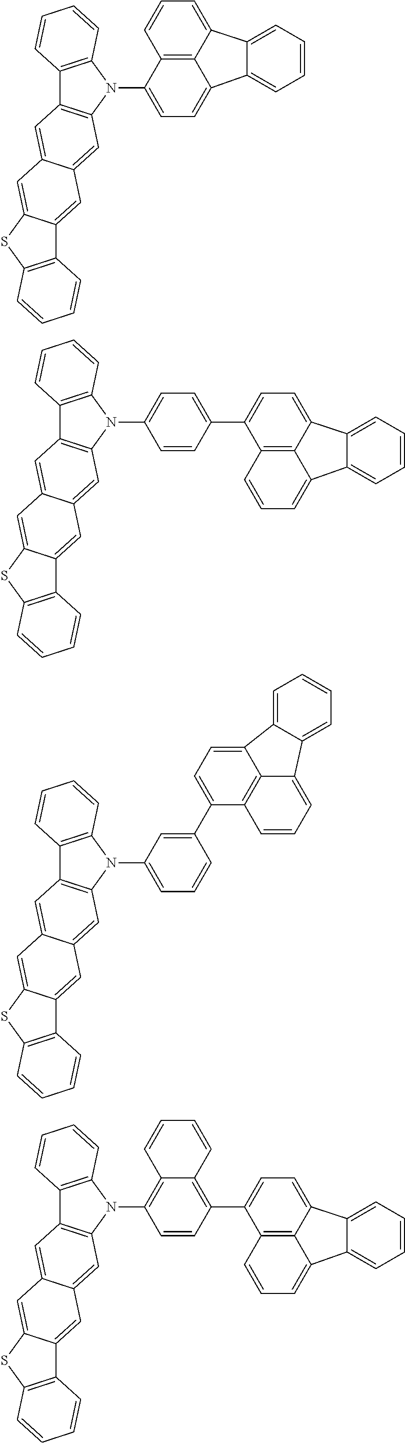

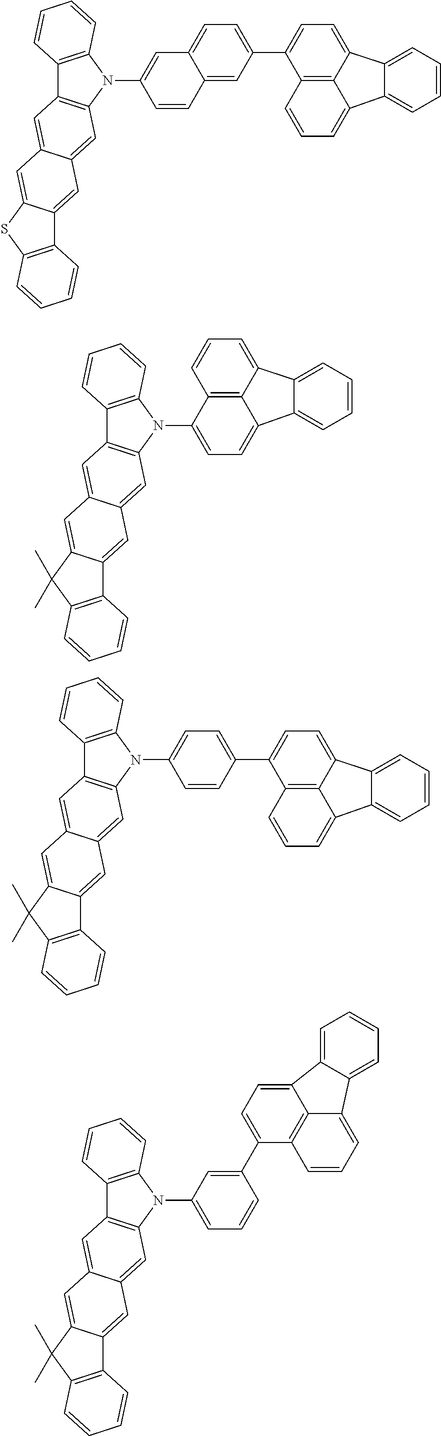

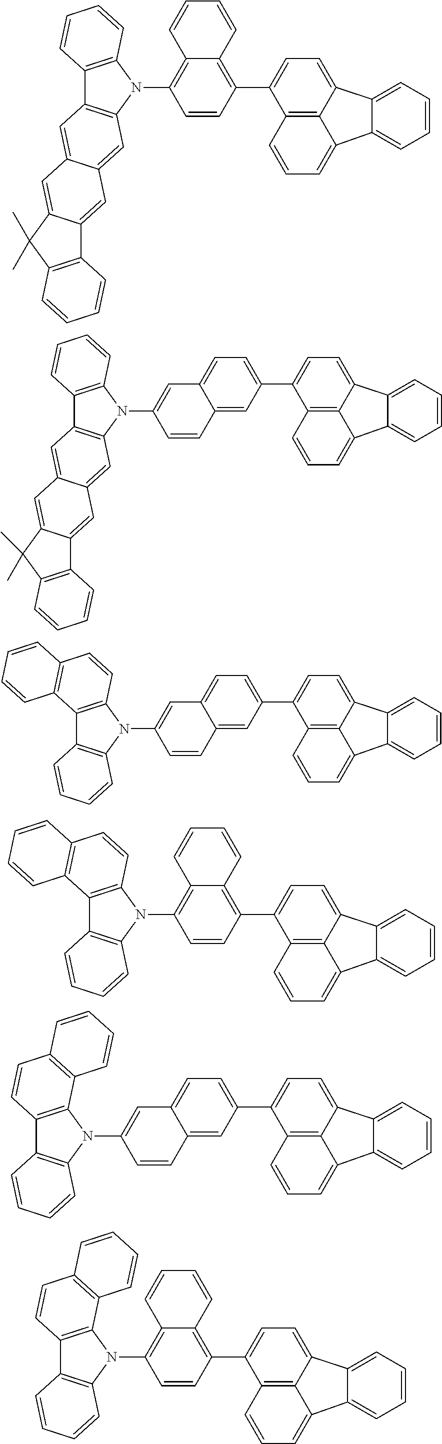

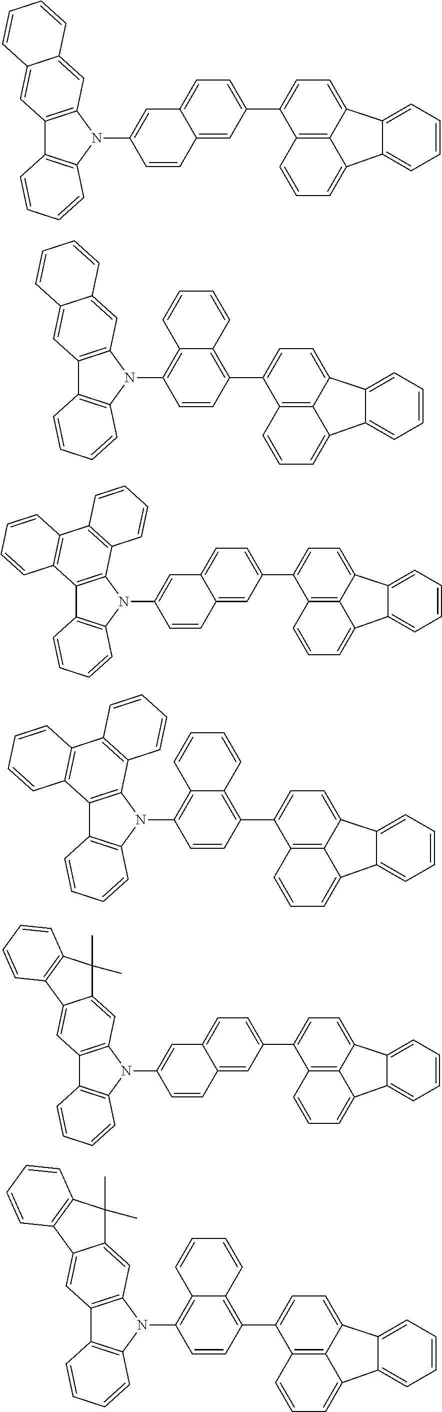

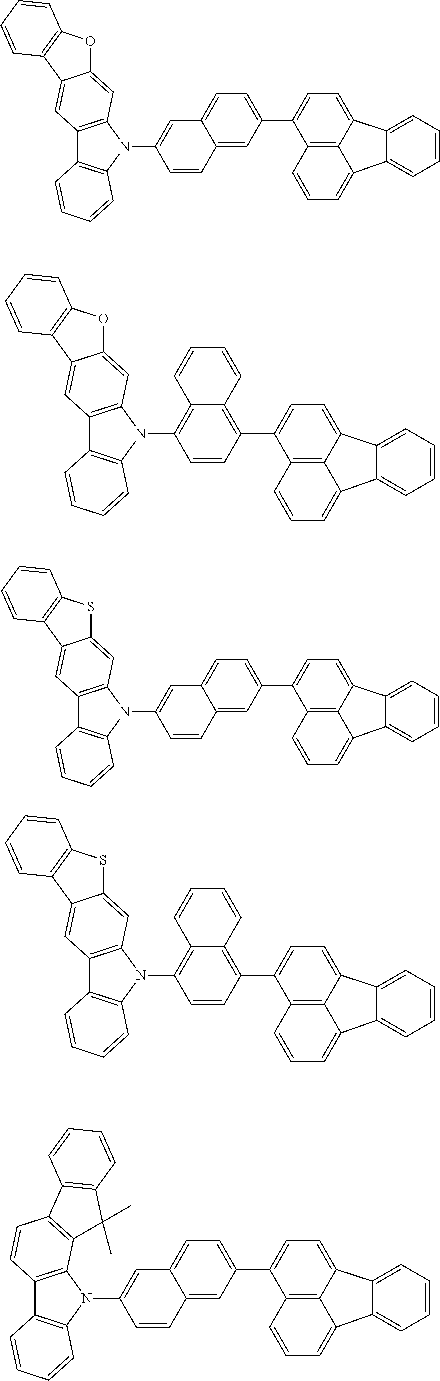

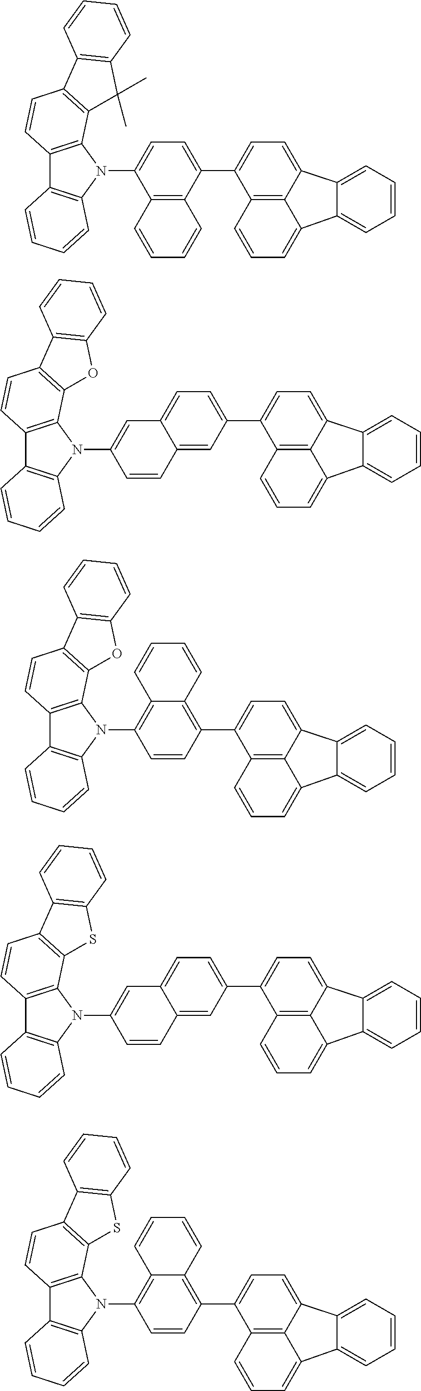

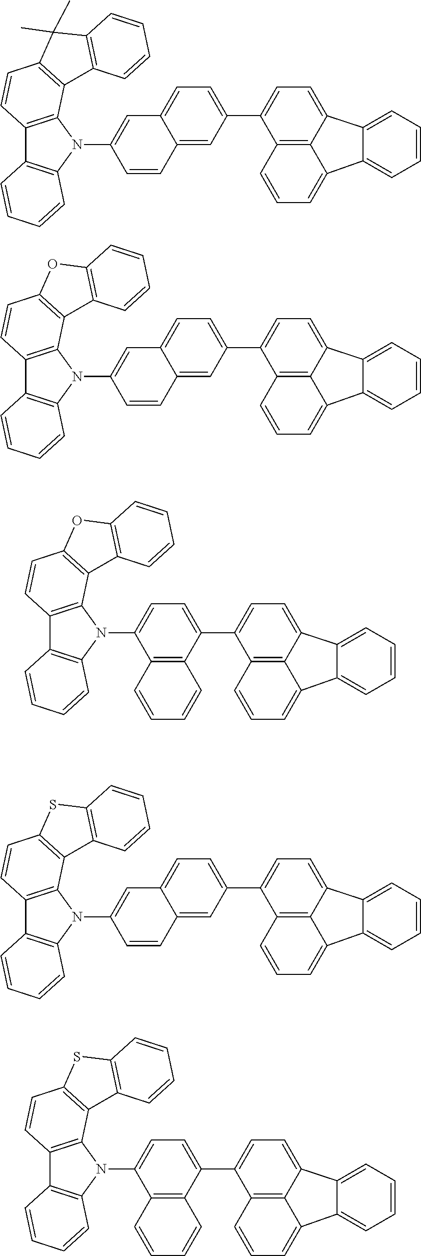

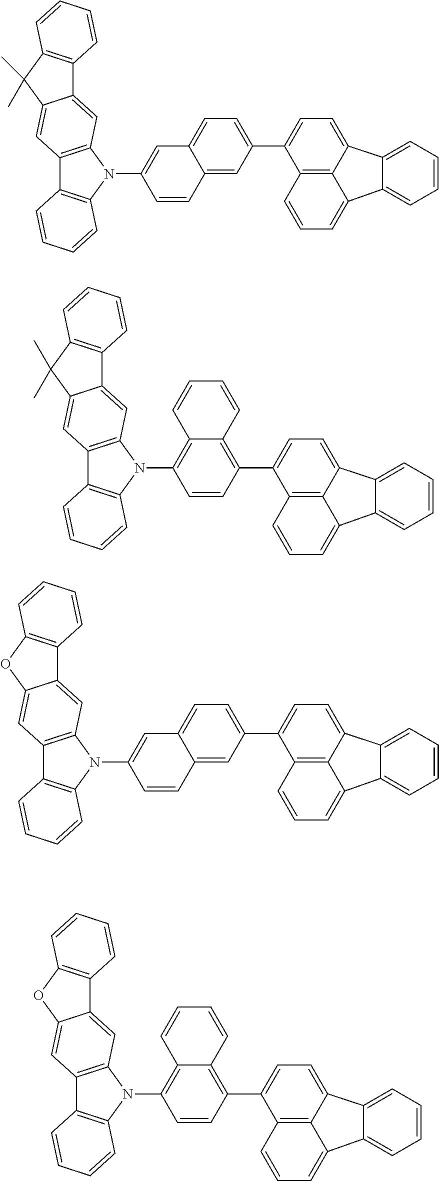

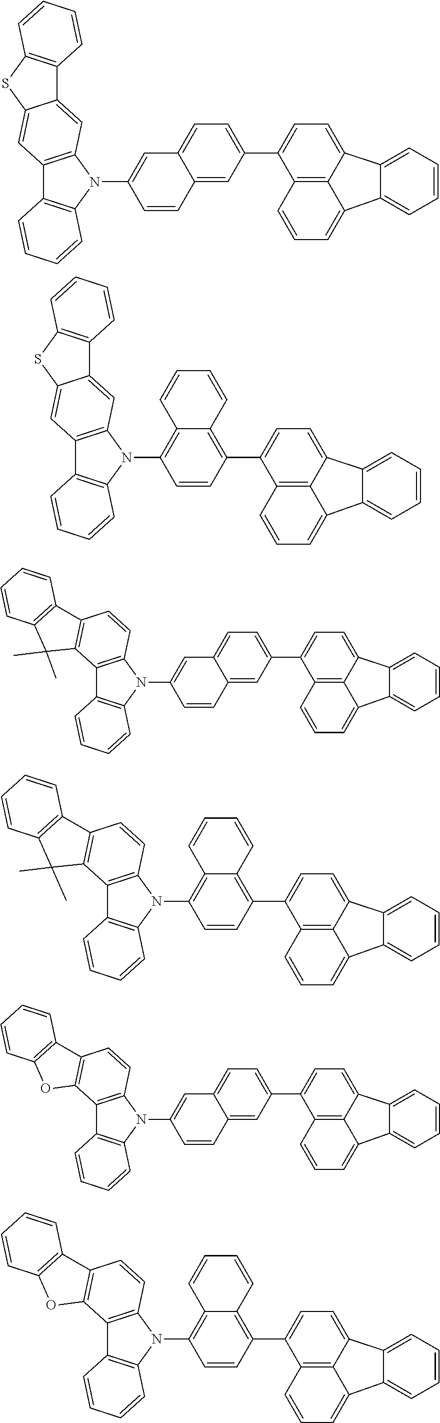

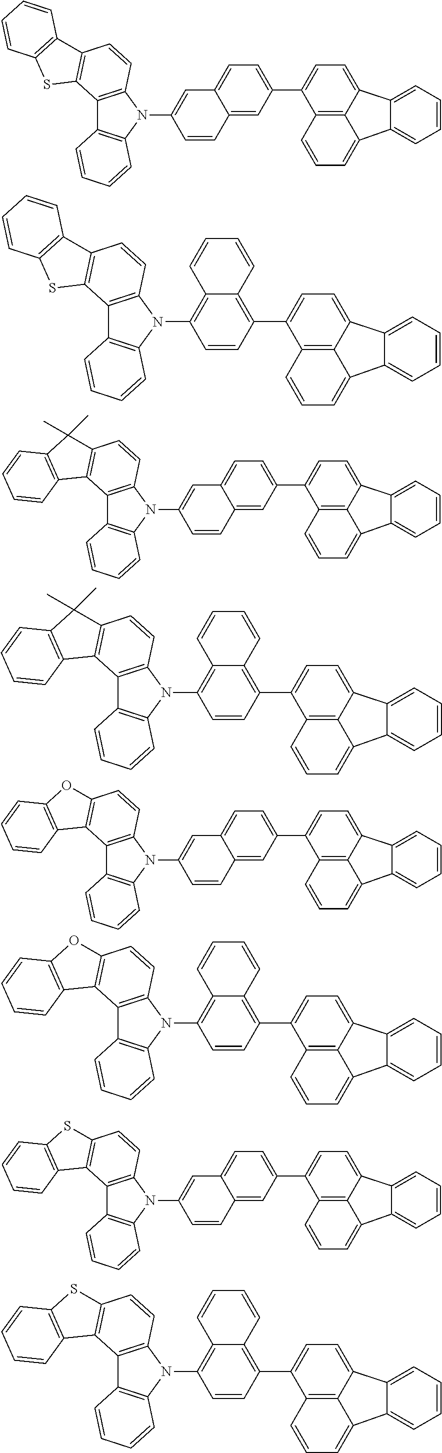

The compound of the invention is useful as a material for organic EL devices. The compound of the invention may be used as a material for organic EL devices alone or in combination of two or more. In addition, the compound of the invention may be used in combination with a known material for organic EL devices, i.e., the invention also provides a material for organic EL devices comprising the compound of the invention. The content of the compound of the invention in the material for organic EL devices is not particularly limited and may be 1% by mass or more, preferably 10% by mass or more, more preferably 50% by mass or more, still preferably 80% by mass or more, and particularly preferably 90% by mass or more.

### [Organic Electroluminescence Device]

The embodiment of the organic EL device of the invention will be described below.

The organic EL device comprises two or more organic thin film layers between a cathode and an anode. The organic thin film layers comprise a light emitting layer and at least one layer of the organic thin film layers comprises the compound of the invention (also referred to as “material for organic EL devices of the invention”), thereby providing an organic EL device with a high efficiency and a long lifetime.

Examples of the organic thin film layer in which the material for organic EL devices of the invention is usable include an anode-side organic thin film layer formed between an anode and a light emitting layer (i.e., a hole transporting layer), a cathode-side organic thin film layer formed between a cathode and a light emitting layer (i.e., an electron transporting layer), a light emitting layer, a space layer, and a blocking layer.

The material for organic EL devices of the invention is preferably used in a light emitting layer, in particular, preferably used as a host material in a light emitting layer, although not particularly limited thereto.

The light emitting layer preferably comprises a fluorescent emitting material or a phosphorescent emitting material, in particular, a phosphorescent emitting material. The material for organic EL devices of the invention is also useful for use in a blocking layer.

The organic EL device of the invention may be any of a single color emitting device of fluorescent or phosphorescent type, a white-emitting device of fluorescent-phosphorescent hybrid type, an emitting device of a simple type having a single emission unit, and an emitting device of a tandem type having two or more emission units, with the phosphorescent device being preferred. The “emission unit” referred to herein is the smallest unit for emitting light by the recombination of injected holes and injected electrons, which comprises one or more organic layers wherein at least one layer is a light emitting layer.

Representative device structures of the simple-type organic EL device are shown below.

### (1) Anode/Emission Unit/Cathode

The emission unit may be a laminate comprising two or more layers selected from a phosphorescent light emitting layer and a fluorescent light emitting layer. A space layer may be disposed between the light emitting layers to prevent the diffusion of excitons generated in the phosphorescent light emitting layer into the fluorescent light emitting layer. Representative layered structures of the emission unit are shown below.
(a) hole transporting layer/light emitting layer (/electron transporting layer);
(b) hole transporting layer/first phosphorescent light emitting layer/second phosphorescent light emitting layer (/electron transporting layer);
(c) hole transporting layer/phosphorescent light emitting layer/space layer/fluorescent light emitting layer (/electron transporting layer);
(d) hole transporting layer/first phosphorescent light emitting layer/second phosphorescent light emitting layer/space layer/fluorescent light emitting layer (/electron transporting layer);
(e) hole transporting layer/first phosphorescent light emitting layer/space layer/second phosphorescent light emitting layer/space layer/fluorescent light emitting layer (/electron transporting layer);
(f) hole transporting layer/phosphorescent light emitting layer/space layer/first fluorescent light emitting layer/second fluorescent light emitting layer (/electron transporting layer);
(g) hole transporting layer/electron blocking layer/light emitting layer (/electron transporting layer);
(h) hole transporting layer/light emitting layer/hole blocking layer (/electron transporting layer); and
(i) hole transporting layer/fluorescent light emitting layer/triplet blocking layer (/electron transporting layer).

The emission color of the phosphorescent light emitting layer and that of the fluorescent light emitting layer may be different. For example, the layered structure of the laminated light emitting layer (d) may be hole transporting layer/first phosphorescent light emitting layer (red emission)/second phosphorescent light emitting layer (green emission)/space layer/fluorescent light emitting layer (blue emission)/electron transporting layer.

An electron blocking layer may be disposed between the light emitting layer and the hole transporting layer or between the light emitting layer and the space layer, if necessary. Also, a hole blocking layer may be disposed between the light emitting layer and the electron transporting layer, if necessary. With such an electron blocking layer or a hole blocking layer, electrons and holes are confined in the light emitting layer to increase the degree of charge recombination in the light emitting layer, thereby improving the lifetime.

Representative device structure of the tandem-type organic EL device is shown below.

### (2) Anode/First Emission Unit/Intermediate Layer/Second Emission Unit/Cathode

The layered structure of the first emission unit and the second emission unit may be selected from those described above with respect to the emission unit.

Generally, the intermediate layer is also called an intermediate electrode, an intermediate conductive layer, a charge generation layer, an electron withdrawing layer, a connecting layer, or an intermediate insulating layer. The intermediate layer may be formed by known materials so as to supply electrons to the first emission unit and holes to the second emission unit.

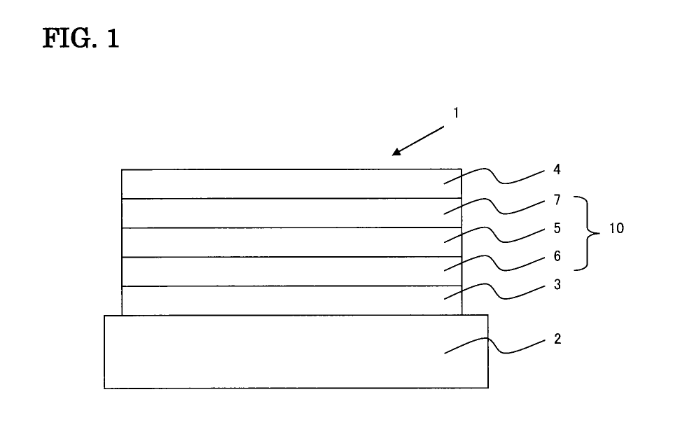

A schematic structure of an example of the organic EL device of the invention is shown in FIG. 1 wherein the organic EL device 1 comprises a substrate 2, an anode 3, a cathode 4, and an organic thin film layer 10 disposed between the anode 3 and the cathode 4. The organic thin film layer 10 comprises a light emitting layer 5 which comprises at least one phosphorescent emitting layer containing a phosphorescent host material and a phosphorescent dopant material (phosphorescent material). A hole injecting/transporting layer (an anode-side organic thin film layer) 6 may be disposed between the light emitting layer 5 and the anode 3, and an electron injecting/transporting layer (a cathode-side thin film layer) 7 may be disposed between the light emitting layer 5 and the cathode 4. An electron blocking layer may be disposed on the anode 3 side of the light emitting layer 5, and a hole blocking layer may be disposed on the cathode 4 side of the light emitting layer 5. With these blocking layers, electrons and holes are confined in the light emitting layer 5 to increase the degree of exciton generation in the light emitting layer 5.

In the present invention, a host is referred to as a fluorescent host when combinedly used with a fluorescent dopant and referred to as a phosphorescent host when combinedly used with a phosphorescent dopant. Therefore, the fluorescent host and the phosphorescent host are not distinguished from each other merely by the difference in their molecular structures. Namely, in the present invention, the term “phosphorescent host” means a material for constituting a phosphorescent emitting layer containing a phosphorescent dopant and does not mean a material that cannot be used as a material for a fluorescent emitting layer. The same applies to the fluorescent host.

### Substrate

The organic EL device in an aspect of the invention is formed on a light-transmissive substrate. The light-transmissive substrate serves as a support for the organic EL device and preferably a flat substrate having a transmittance of 50% or more to 400 to 700 nm visible light. Examples of the substrate include a glass plate and a polymer plate. The glass plate may include a plate made of soda-lime glass, barium-strontium-containing glass, lead glass, aluminosilicate glass, borosilicate glass, barium borosilicate glass, or quartz. The polymer plate may include a plate made of polycarbonate, acryl, polyethylene terephthalate, polyether sulfide, or polysulfone.

### Anode

The anode of the organic EL device injects holes to the hole transporting layer or the light emitting layer, and an anode having a work function of 4.5 eV or more is effective. Examples of the material for anode include indium tin oxide alloy (ITO), tin oxide (NESA), indium zinc oxide alloy, gold, silver, platinum, and cupper. The anode is formed by making the electrode material into a thin film by a method, such as a vapor deposition method or a sputtering method. When getting the light emitted from the light emitting layer through the anode, the transmittance of anode to visible light is preferably 10% or more. The sheet resistance of anode is preferably several hundreds Ω/□ or less. The film thickness of anode depends upon the kind of material and generally 10 nm to 1 μm, preferably 10 to 200 nm.

### Cathode

The cathode injects electrons to the electron injecting layer, the electron transporting layer or the light emitting layer, and formed preferably by a material having a small work function. Examples of the material for cathode include, but not limited to, indium, aluminum, magnesium, magnesium-indium alloy, magnesium-aluminum alloy, aluminum-lithium alloy, aluminum-scandium-lithium alloy, and magnesium-silver alloy. Like the anode, the cathode is formed by making the material into a thin film by a method, such as the vapor deposition method and the sputtering method. The emitted light may be taken through the cathode, if necessary.

### Light Emitting Layer

The light emitting layer is an organic layer having a light emitting function and contains a host material and a dopant material when a doping system is employed. The major function of the host material is to promote the recombination of electrons and holes and confine excitons in the light emitting layer. The dopant material causes the excitons generated by recombination to emit light efficiently.

In case of a phosphorescent device, the major function of the host material is to confine the excitons generated on the dopant in the light emitting layer.

To control the carrier balance in the light emitting layer, the light emitting layer may be made into a double host (host/co-host) layer, for example, by combinedly using an electron transporting host and a hole transporting host. In a preferred embodiment, the light emitting layer comprises a first host material and a second host material, and the first host material is the material for organic EL devices of the invention.

The light emitting layer may be made into a double dopant layer, in which two or more kinds of dopant materials having high quantum yield are combinedly used and each dopant material emits light with its own color. For example, to obtain a yellow emission, a light emitting layer formed by co-depositing a host, a red-emitting dopant and a green-emitting dopant is used.

In a laminate of two or more light emitting layers, electrons and holes are accumulated in the interface between the light emitting layers, and therefore, the recombination region is localized in the interface between the light emitting layers, to improve the quantum efficiency.

The easiness of hole injection to the light emitting layer and the easiness of electron injection to the light emitting layer may be different from each other. Also, the hole transporting ability and the electron transporting ability each being expressed by mobility of holes and electrons in the light emitting layer may be different from each other.

The light emitting layer is formed, for example, by a known method, such as a vapor deposition method, a spin coating method, and LB method (Langmuir Blodgett method). The light emitting layer can be formed also by making a solution of a binder, such as resin, and the material for the light emitting layer in a solvent into a thin film by a method such as spin coating.

The light emitting layer is preferably a molecular deposit film. The molecular deposit film is a thin film formed by depositing a vaporized material or a film formed by solidifying a material in the state of solution or liquid. The molecular deposit film can be distinguished from a thin film formed by LB method (molecular build-up film) by the differences in the assembly structures and higher order structures and the functional difference due to the structural differences.

The dopant material is selected from known fluorescent dopants and phosphorescent dopants.

The fluorescent dopant is selected from, for example, a fluoranthene derivative, a pyrene derivative, an arylacetylene derivative, a fluorene derivative, a boron complex, a perylene derivative, an oxadiazole derivative, an anthracene derivative, and a chrysene derivative, and preferably selected from a fluoranthene derivative, a pyrene derivative and a boron complex.

The phosphorescent dopant (phosphorescent material) used in the light emitting layer is a compound which emits light by releasing the energy of excited triplet state and preferably a organometallic complex comprising at least one metal selected from Ir, Pt, Os, Au, Cu, Re, and Ru and a ligand, although not particularly limited thereto as long as emitting light by releasing the energy of excited triplet state. The ligand is preferably ortho-metallated. In view of obtaining a high phosphorescent quantum yield and further improving the external quantum efficiency of luminescent device, a metal complex comprising a metal selected from Ir, Os, and Pt is preferred, with a metal complex, such as an iridium complex, an osmium complex and a platinum complex, particularly an ortho-metallated complex being more preferred, an iridium complex and a platinum complex being still more preferred, and an ortho-metallated iridium complex being particularly preferred.

The content of the phosphorescent dopant in the light emitting layer is not particularly limited and selected according to the use of the device, and preferably 0.1 to 70% by mass, and more preferably 1 to 30% by mass. If being 0.1% by mass or more, the amount of light emission is sufficient. If being 70% by mass or less, the concentration quenching can be avoided.

Preferred examples of the organometallic complex for the phosphorescent dopant are shown below.

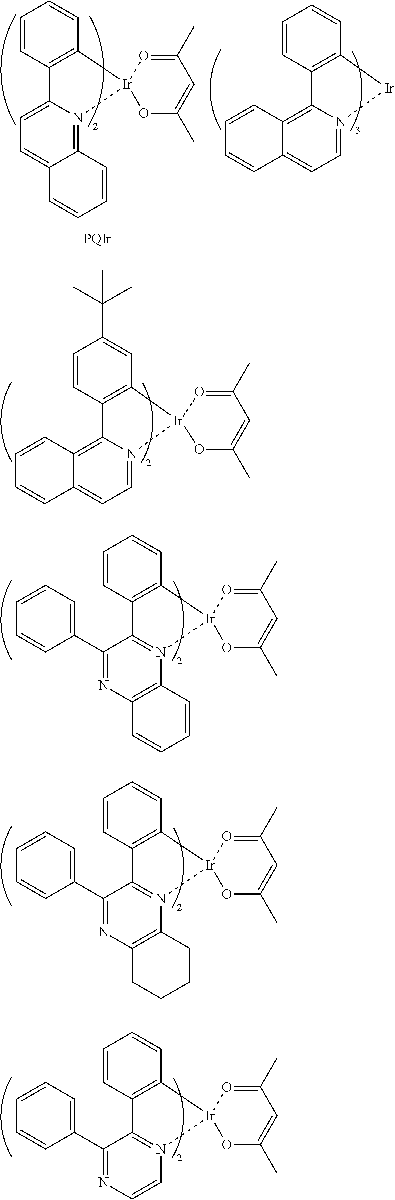

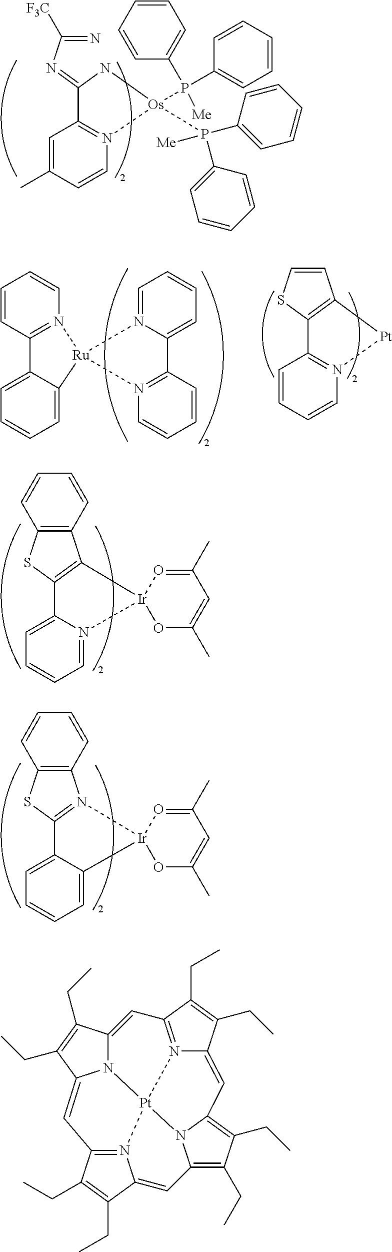

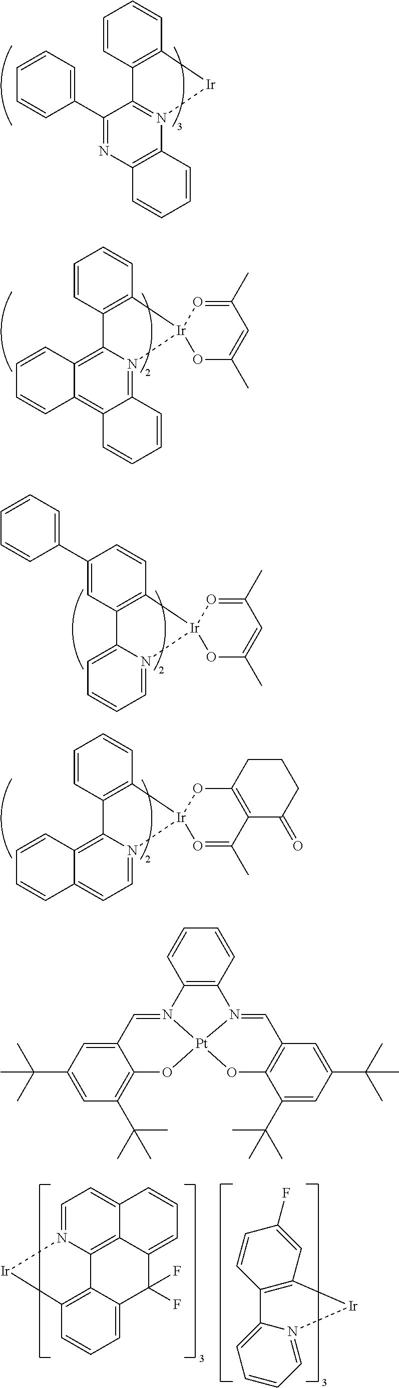

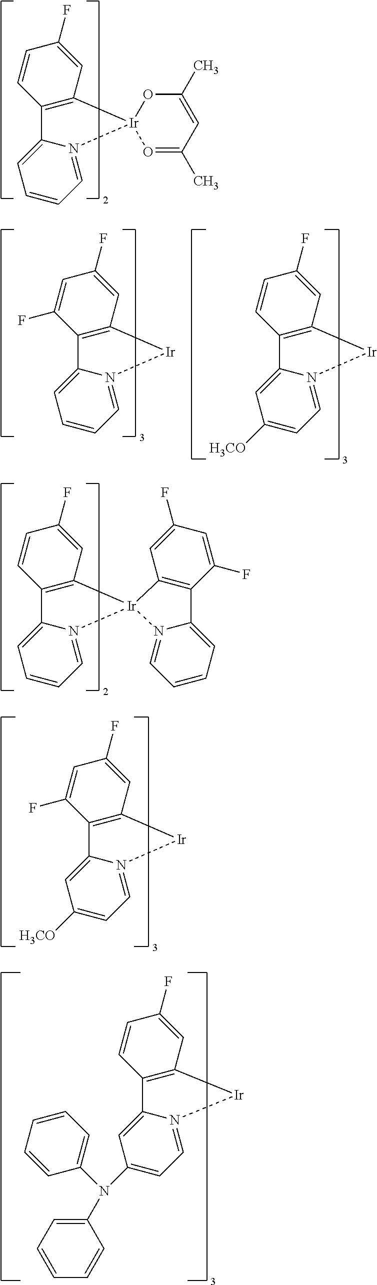

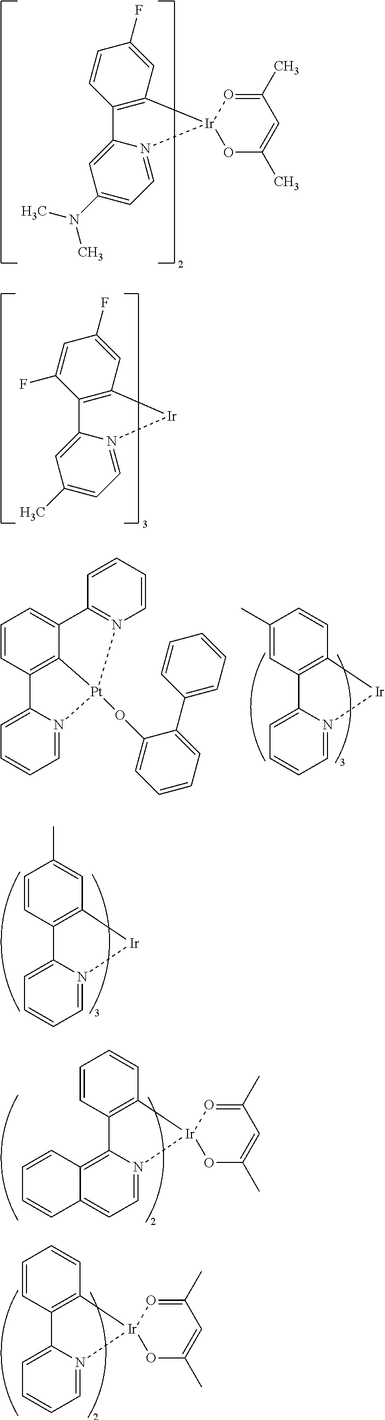

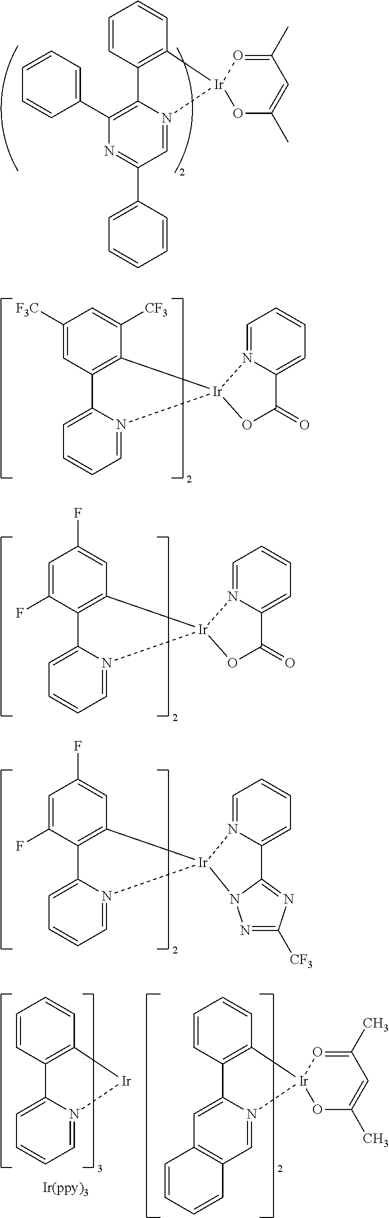

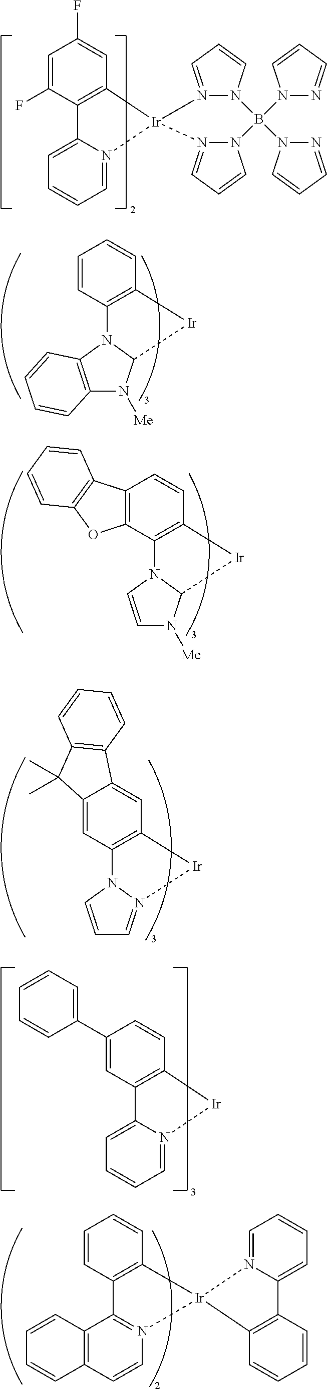

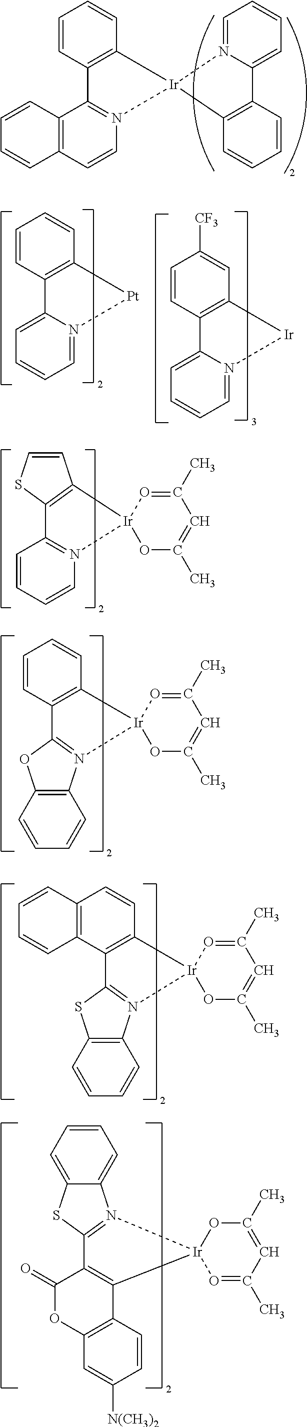

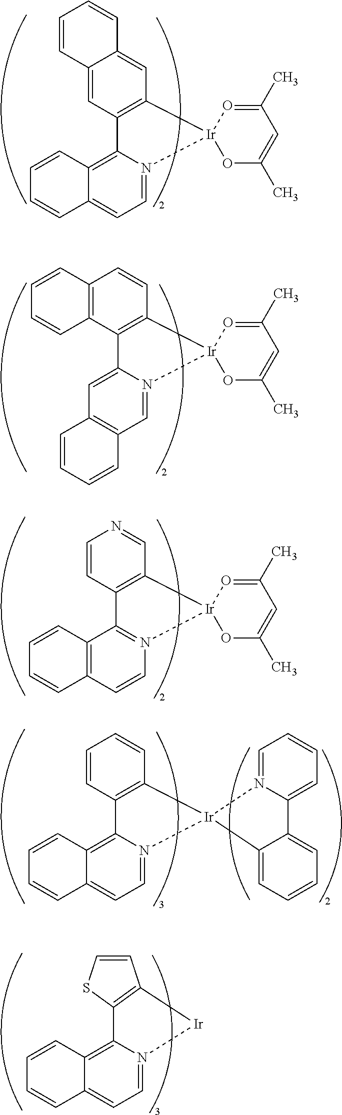

The phosphorescent host is a compound which confines the triplet energy of the phosphorescent dopant efficiently in the light emitting layer to cause the phosphorescent dopant to emit light efficiently. The material for organic EL devices of the invention is suitable as a phosphorescent host. The light emitting layer may comprise the material for organic EL devices singly or in combination of two or more.

When the material for organic EL devices is used in a light emitting layer as a host material, the emission wavelength of a phosphorescent dopant material in the light emitting layer is not particularly limited. In a preferred embodiment, at least one of the phosphorescent dopant materials in a light emitting layer emits light with a peak wavelength of preferably 490 nm or more and 700 nm or less and more preferably 490 nm or more and 650 nm or less. The emission color is preferably red, yellow or green. By using a light emitting layer constituted of the compound of the invention as a host material doped with a phosphorescent dopant material having such an emission wavelength, the life time of an organic EL device is prolonged.

A compound other than the material for organic EL devices of the invention may be suitably used in the organic EL device of the invention as a phosphorescent host according to the purpose.

The material for organic EL devices of the invention and a compound other than it may be combinedly used in the same light emitting layer as the phosphorescent host materials. If two or more light emitting layers are formed, the material for organic EL devices can be used in one of the light emitting layers as a phosphorescent host material and a compound other than the material for organic EL devices can be used in another light emitting layer as a phosphorescent host material. The material for organic EL devices may be used in an organic layer other than the light emitting layer. In this case, a compound other than the material for organic EL devices may be used as a phosphorescent host of the light emitting layer.

Examples of the preferred phosphorescent host other than the material for organic EL device in an aspect of the invention include a carbazole derivative, a triazole derivative, a oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styrylanthracene derivative, a fluorenone derivative, a hydrazone derivative, a stilbene derivative, a silazane derivative, an aromatic tertiary amine compound, a styrylamine compound, an aromatic methylidene compound, a porphyrin compound, an anthraquinodimethane derivative, an anthrone derivative, a diphenylquinone derivative, a thiopyran dioxide derivative, a carbodiimide derivative, a fluorenylidenemethane derivative, a distyrylpyrazine derivative, a tetracarboxylic anhydride of fused ring such as naphthalene and perylene, a phthalocyanine derivative, a metal complex of 8-quinolinol derivative, metal phthalocyanine, metal complexes having a ligand such as benzoxazole and benzothiazole, an electroconductive oligomer, such as a polysilane compound, a poly(N-vinylcarbazole) derivative, an aniline copolymer, thiophene oligomer, and a polythiophene, and a polymer such as a polythiophene derivative, a polyphenylene derivative, a polyphenylenevinylene derivative, and a polyfluorene derivative. These phosphorescent hosts may be used alone or in combination of two or more. Examples thereof are shown below.

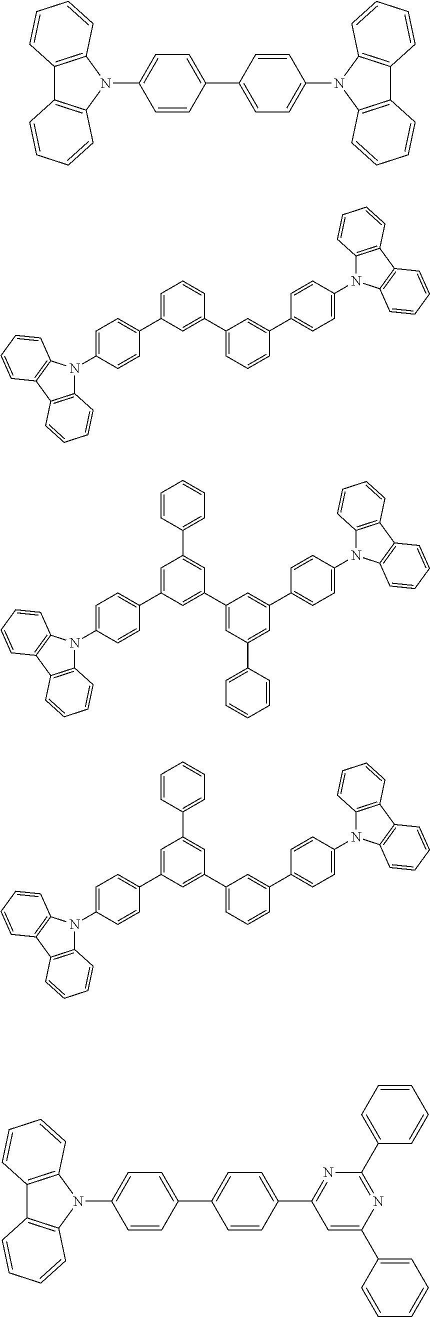 [SMILES: c1ccc2c(c1)c1ccccc1n2-c1ccc(-c2ccc(-n3c4ccccc4c4ccccc43)cc2)cc1 ; c1cc(-c2ccc(-n3c4ccccc4c4ccccc43)cc2)cc(-c2cccc(-c3ccc(-n4c5ccccc5c5ccccc54)cc3)c2)c1 ; c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1 ; c1ccc(-c2cc(-c3ccc(-c4ccc(-n5c6ccccc6c6ccccc65)cc4)cc3)nc(-c3ccccc3)n2)cc1]

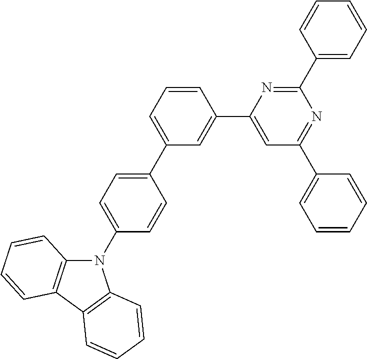 [SMILES: c1ccc(-c2cc(-c3cccc(-c4ccc(-n5c6ccccc6c6ccccc65)cc4)c3)nc(-c3ccccc3)n2)cc1]

The light emitting layer may comprise a first host material and a second host material, in which the material for organic EL devices of the invention is used as the first host material and a compound other than the material for organic EL devices of the invention is used as the second host material. The term “first host material” and the term “second host material” are distinguished by the difference in their chemical structures and not by the contents thereof in the light emitting layer.

The second host material is a compound other than the material for organic EL devices of the invention and selected from those mentioned above as the compounds suitable as the phosphorescent host, although not particularly limited thereto. The second host material is preferably a compound free from cyano group, and also preferred are a carbazole derivative, an arylamine derivative, a fluorenone derivative, and an aromatic tertiary amine compound.

The thickness of the light emitting layer is preferably 5 to 50 nm, more preferably 7 to 50 nm and still more preferably 10 to 50 nm. If 5 nm or more, the light emitting layer is easily formed. If 50 nm or less, the increase in the driving voltage can be avoided.

### Electron-Donating Dopant

The organic EL device in an aspect of the invention preferably comprises an electron-donating dopant at an interfacial region between the cathode and the emitting unit. With such a construction, the organic EL device has an improved luminance and an elongated lifetime. The electron-donating dopant comprises a metal having a work function of 3.8 eV or less and examples thereof include at least one selected from alkali metal, alkali metal complex, alkali metal compound, alkaline earth metal, alkaline earth metal complex, alkaline earth metal compound, rare earth metal, rare earth metal complex, and rare earth metal compound.

Examples of the alkali metal include Na (work function: 2.36 eV), K (work function: 2.28 eV), Rb (work function: 2.16 eV), and Cs (work function: 1.95 eV), with those having a work function of 2.9 eV or less being particularly preferred. Of the above, preferred are K, Rb, and Cs, more preferred are Rb and Cs, and most preferred is Cs. Examples of the alkaline earth metal include Ca (work function: 2.9 eV), Sr (work function: 2.0 to 2.5 eV), and Ba (work function: 2.52 eV), with those having a work function of 2.9 eV or less being particularly preferred. Examples of the rare earth metal include Sc, Y, Ce, Tb, and Yb, with those having a work function of 2.9 eV or less being particularly preferred.

Examples of the alkali metal compound include alkali oxide, such as Li₂O, Cs₂O, K₂O, and alkali halide, such as LiF, NaF, CsF, and KF, with LiF, Li₂O, and NaF being preferred. Examples of the alkaline earth metal compound include BaO, SrO, CaO, and mixture thereof, such as BaₓSr₁₋ₓO (0<x<1) and BaₓCA¹₋ₓO (0<x<1), with BaO, SrO, and CaO being preferred. Examples of the rare earth metal compound include YbF₃, ScF₃, ScO₃, Y₂O₃, Ce₂O₃, GdF₃, and TbF₃, with YbF₃, ScF₃, and TbF₃ being preferred.

Examples of the alkali metal complex, alkaline earth metal complex, and rare earth metal are not particularly limited as long as containing at least one metal ion selected from alkali metal ions, alkaline earth metal ions, and rare earth metal ions, respectively. The ligand is preferably, but not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfulborane, bipyridyl, phenanthroline, phthalocyanine, porphyrin, cyclopentadiene, β-diketones, azomethines, and derivative thereof.

The electron-donating dopant is added to the interfacial region preferably into a form of layer or island. The electron-donating dopant is added preferably by co-depositing the electron-donating dopant with the organic compound (light emitting material, electron injecting material) for forming the interfacial region by a resistance heating deposition method, thereby dispersing the electron-donating dopant into the organic material. The disperse concentration expressed by the molar ratio of the organic material and the electron-donating dopant is 100:1 to 1:100 and preferably 5:1 to 1:5.

When the electron-donating dopant is formed into a form of layer, a light emitting material or an electron injecting material is made into a layer which serves as an organic layer in the interface, and then, the electron-donating dopant alone is deposited by a resistance heating deposition method into a layer having a thickness preferably 0.1 to 15 nm. When the electron-donating dopant is formed into a form of island, a light emitting material or an electron injecting material is made into a form of island which serves as an organic layer in the interface, and then, the electron-donating dopant alone is deposited by a resistance heating deposition method into a form of island having a thickness preferably 0.05 to 1 nm.

The molar ratio of the main component and the electron-donating dopant in the organic electroluminescence device in an aspect of the invention is preferably 5:1 to 1:5 and more preferably 2:1 to 1:2.

### Electron Transporting Layer

The electron transporting layer is an organic layer disposed between the light emitting layer and the cathode and transports electrons from the cathode to the light emitting layer. If two or more electron transporting layers are provided, the organic layer closer to the cathode may be called an electron injecting layer in some cases. The electron injecting layer injects electrons from the cathode to the organic layer unit efficiently.

An aromatic heterocyclic compound having one or more heteroatoms in a molecule thereof is preferably used as an electron transporting material used in the electron transporting layer, and a nitrogen-containing ring derivative is particularly preferred. In addition, the nitrogen-containing ring derivative is preferably an aromatic ring compound having a nitrogen-containing, 6- or 5-membered ring, or a fused aromatic ring compound having a nitrogen-containing, 6- or 5-membered ring.

The nitrogen-containing ring derivative is preferably, for example, a metal chelate complex of a nitrogen-containing ring represented by formula (A):

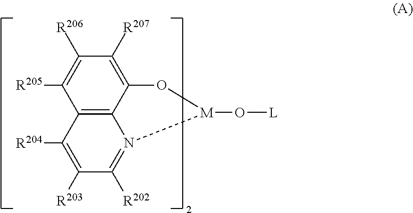

wherein each of R²⁰² to R²⁰⁷ independently represents a hydrogen atom, a heavy hydrogen atom, a halogen atom, a hydroxyl group, an amino group, a hydrocarbon group having 1 to 40 carbon atoms, an alkoxy group having 1 to 40 carbon atoms, an aryloxy group having 6 to 50 carbon atoms, an alkoxycarbonyl group, or an aromatic heterocyclic group having 5 to 50 ring carbon atoms, each being optionally substituted.

The halogen atom may include fluorine, chlorine, bromine, and iodine.

The substituted amino group may include an alkylamino group, an arylamino group, and an aralkylamino group.

The alkylamino group and the aralkylamino group are represented by —NQ¹Q². Each of Q¹ and Q² independently represents an alkyl group having 1 to 20 carbon atoms or an aralkyl group having 1 to 20 carbon atoms. One of Q¹ and Q² may be a hydrogen atom or a heavy hydrogen atom.

The arylamino group is represented by —NAr¹⁰¹Ar¹⁰², wherein each of Ar¹⁰¹ and Ar¹⁰² independently represents a non-fused aromatic hydrocarbon groups or a fused aromatic hydrocarbon groups, each having 6 to 50 carbon atoms. One of Ar¹⁰¹ and Ar¹⁰² may be a hydrogen atom or a heavy hydrogen atom.

Examples of the hydrocarbon group having 1 to 40 carbon atoms include an alkyl group, an alkenyl group, a cycloalkyl group, an aryl group, and an aralkyl group.

The alkoxycarbonyl group is represented by —COOY′, wherein Y′ is an alkyl group having 1 to 20 carbon atoms.

M is aluminum (Al), gallium (Ga), or indium (In), with In being preferred.

L is a group represented by formula (A′) or (A″):

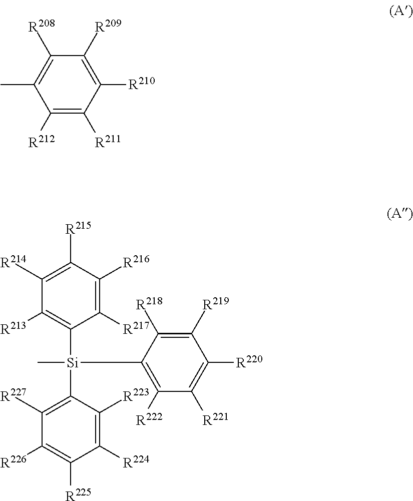

wherein each of R²⁰⁸ to R²¹² independently represents a hydrogen atom, a heavy hydrogen atom or a substituted or unsubstituted hydrocarbon group having 1 to 40 carbon atoms; two neighboring groups may form a ring structure; each of R²¹³ to R²²⁷ independently represents a hydrogen atom, a heavy hydrogen atom or a substituted or unsubstituted hydrocarbon group having 1 to 40 carbon atoms; and two neighboring groups may form a ring structure.

Examples of the hydrocarbon group having 1 to 40 carbon atoms for R²⁰⁸ to R²¹² and R²¹³ to R²²⁷ in formulae (A′) and (A″) are the same as those described above with respect to R²⁰² to R²⁰⁷ of formula (A). Examples of the divalent group formed by two neighboring groups of R²⁰⁸ to R²¹² and R²¹³ to R²²⁷ which completes the ring structure include a tetramethylene group, a pentamethylene group, a hexamethylene group, a diphenylmethane-2,2′-diyl group, a diphenylethane-3,3′-diyl group, and a diphenylpropane-4,4′-diyl group.

The electron transporting compound for use in the electron transporting layer is preferably a metal complex including 8-hydroxyquinoline or its derivative, an oxadiazole derivative, or a nitrogen-containing heterocyclic derivative. Examples of the metal complex including 8-hydroxyquinoline or its derivative include a metal chelate oxinoid including a chelated oxine (generally, 8-quinolinol or 8-hydroxyquinoline), for example, tris(8-quinolinol)aluminum. Examples of the oxadiazole derivative are shown below:

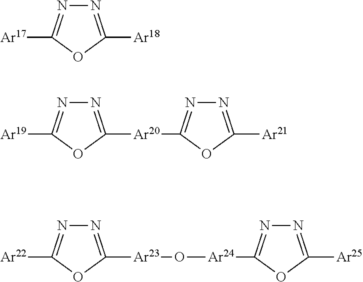

In the above formulae, each of Ar¹⁷, Ar¹⁸, Ar¹⁹, Ar²¹, Ar²², and Ar²⁵ is a substituted or unsubstituted aromatic hydrocarbon group or a substituted or unsubstituted fused aromatic hydrocarbon group each having 6 to 50, preferably 6 to 30, more preferably 6 to 20, and still more preferably 6 to 12 carbon atoms, and Ar¹⁷ and Ar¹⁸, Ar¹⁹ and Ar²¹, and Ar²² and Ar²⁵ may be the same or different. Examples of the aromatic hydrocarbon group and the fused aromatic hydrocarbon group include a phenyl group, a naphthyl group, a biphenyl group, an anthranyl group, a perylenyl group, and a pyrenyl group. The optional substituent may be an alkyl group having 1 to 10 carbon atoms, an alkoxyl group having 1 to 10 carbon atoms or a cyano group.

Each of Ar²⁰, Ar²³, and Ar²⁴ is a substituted or unsubstituted bivalent aromatic hydrocarbon group or a substituted or unsubstituted bivalent fused aromatic hydrocarbon group each having 6 to 50, preferably 6 to 30, more preferably 6 to 20, and still more preferably 6 to 12 carbon atoms, and Ar²³ and Ar²⁴ may be the same or different. Examples of the bivalent aromatic hydrocarbon group or the bivalent fused aromatic hydrocarbon group include a phenylene group, a naphthylene group, a biphenylene group, an anthranylene group, a perylenylene group, and a pyrenylene group. The optional substituent may be an alkyl group having 1 to 10 carbon atoms, an alkoxyl group having 1 to 10 carbon atoms or a cyano group.

Electron transporting compounds which have a good thin film-forming property are preferably used. Examples of the electron transporting compound are shown below.

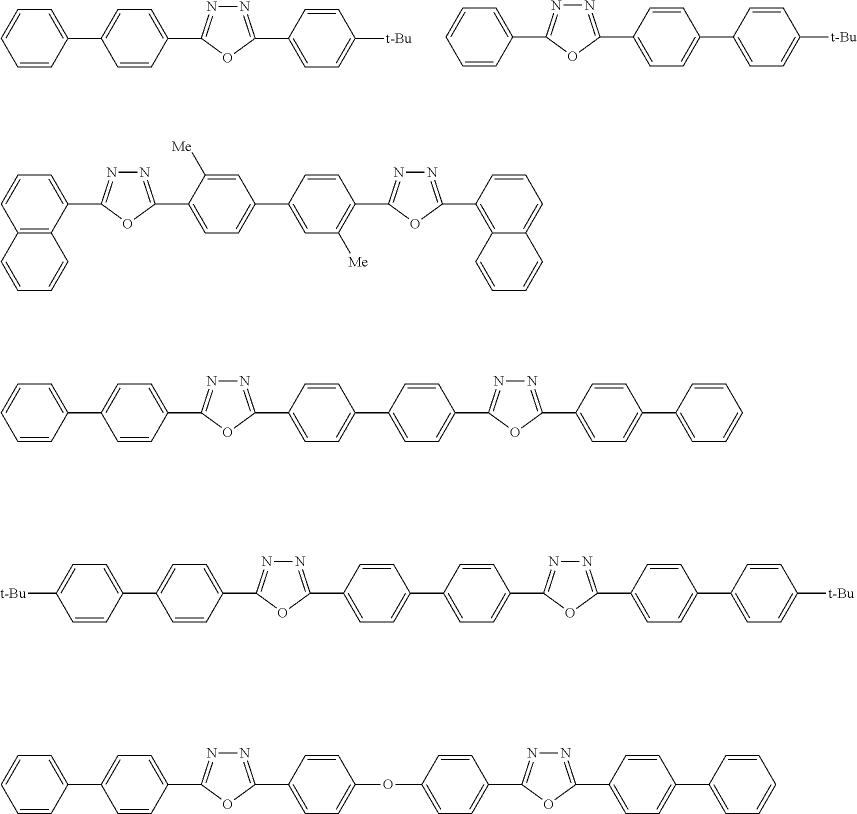 [SMILES: CC(C)(C)c1ccc(-c2nnc(-c3ccc(-c4ccccc4)cc3)o2)cc1 ; CC(C)(C)c1ccc(-c2ccc(-c3nnc(-c4ccccc4)o3)cc2)cc1 ; Cc1cc(-c2ccc(-c3nnc(-c4cccc5ccccc45)o3)c(C)c2)ccc1-c1nnc(-c2cccc3ccccc23)o1 ; c1ccc(-c2ccc(-c3nnc(-c4ccc(-c5ccc(-c6nnc(-c7ccc(-c8ccccc8)cc7)o6)cc5)cc4)o3)cc2)cc1 ; CC(C)(C)c1ccc(-c2ccc(-c3nnc(-c4ccc(-c5ccc(-c6nnc(-c7ccc(-c8ccc(C(C)(C)C)cc8)cc7)o6)cc5)cc4)o3)cc2)cc1 ; c1ccc(-c2ccc(-c3nnc(-c4ccc(Oc5ccc(-c6nnc(-c7ccc(-c8ccccc8)cc7)o6)cc5)cc4)o3)cc2)cc1]

Examples of the nitrogen-containing heterocyclic derivative for use as the electron transporting compound include a nitrogen-containing heterocyclic derivative having the following formulae but exclusive of metal complex, for example, a compound having a 5- or 6-membered ring which has the skeleton represented by formula (B) or having the structure represented by formula (C):

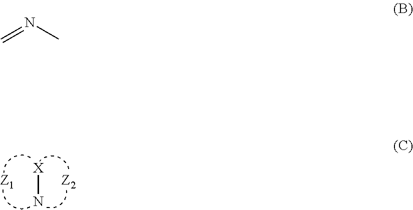

wherein X is a carbon atom or a nitrogen atom and each of Z₁ and Z₂ independently represents a group of atoms for completing the nitrogen-containing heterocyclic ring.

The nitrogen-containing heterocyclic derivative is more preferably an organic compound which has a nitrogen-containing aromatic polycyclic ring comprising a 5-membered ring or a 6-membered ring. If two or more nitrogen atoms are included, the nitrogen-containing aromatic polycyclic compound preferably has a skeleton of a combination of (B) and (C) or a combination of (B) and (D):

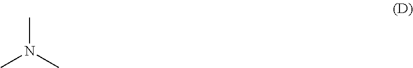

The nitrogen-containing group of the nitrogen-containing aromatic polycyclic compound is selected, for example, from the nitrogen-containing heterocyclic groups shown below:

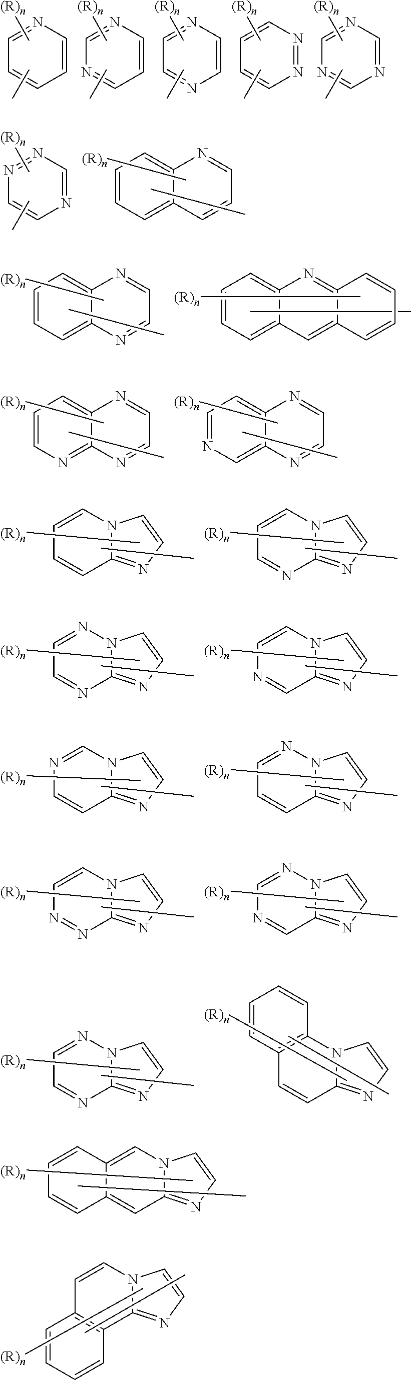

wherein R is an aromatic hydrocarbon group or a fused aromatic hydrocarbon group each having 6 to 40, preferably 6 to 30, more preferably 6 to 20, and still more preferably 6 to 12 carbon atoms, an aromatic heterocyclic group or a fused aromatic heterocyclic group each having 5 to 40, preferably 5 to 20, and more preferably 5 to 12 ring atoms, an alkyl group having 1 to 20, preferably 1 to 10, and more preferably 1 to 6 carbon atoms, or an alkoxy group having 1 to 20, preferably 1 to 10, and more preferably 1 to 6 carbon atoms; and n is an integer of 0 to 5. If n is an integer of 2 or more, groups R may be the same or different.

More preferred is a nitrogen-containing heterocyclic derivative represented by formula (D1);

HAr-L¹¹-Ar¹—Ar² (D1)

wherein HAr is a substituted or unsubstituted nitrogen-containing heterocyclic group having 5 to 40, preferably 5 to 30, more preferably 5 to 20, and still more preferably 5 to 12 ring atoms; L¹¹ is a single bond, a substituted or unsubstituted aromatic hydrocarbon group or fused aromatic hydrocarbon group each having 6 to 40, preferably 6 to 30, more preferably 6 to 20, and still more preferably 6 to 12 ring carbon atoms, or a substituted or unsubstituted aromatic heterocyclic group or fused aromatic heterocyclic group each having 5 to 40 ring atoms; Ar¹ is a substituted or unsubstituted divalent aromatic hydrocarbon group having 6 to 40, preferably 6 to 30, more preferably 6 to 20, and still more preferably 6 to 12 ring carbon atoms; and Ar² is a substituted or unsubstituted aromatic hydrocarbon group or fused aromatic hydrocarbon group each having 6 to 40, preferably 6 to 30, more preferably 6 to 20, and still more preferably 6 to 12 ring carbon atoms or a substituted or unsubstituted aromatic heterocyclic group or fused aromatic heterocyclic group each having 5 to 40, preferably 5 to 30, more preferably 5 to 20, and still more preferably 5 to 12 ring atoms.

HAr is selected, for example, from the following groups:

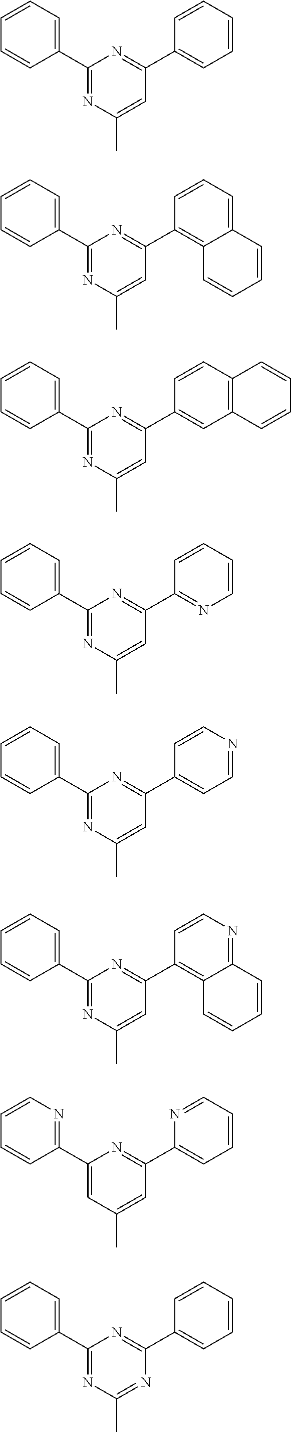

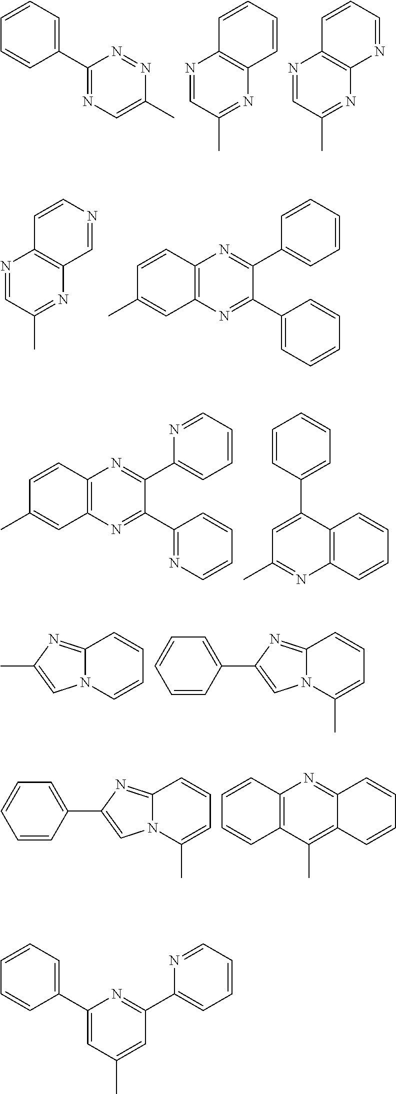

L¹¹ is selected, for example, from the following groups:

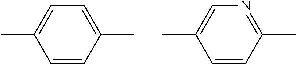

Ar¹ is selected, for example, from the anthracenediyl groups represented by formula (D2) or (D3);

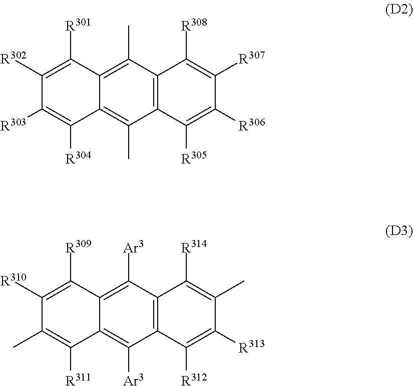

wherein R³⁰¹ to R³¹⁴ are each independently a hydrogen atom, a halogen atom, an alkyl group having 1 to 20, preferably 1 to 10, and more preferably 1 to 6 carbon atoms, an alkoxy group having 1 to 20, preferably 1 to 10, and more preferably 1 to 6 carbon atoms, an aryloxy group having 6 to 40, preferably 6 to 30, more preferably 6 to 20, and still more preferably 6 to 12 ring carbon atoms, a substituted or unsubstituted aromatic hydrocarbon group or a fused aromatic hydrocarbon group each having 6 to 40, preferably 6 to 30, more preferably 6 to 20, and still more preferably 6 to 12 ring carbon atoms, or a substituted or unsubstituted aromatic heterocyclic group or a fused aromatic heterocyclic group each having 5 to 40 ring atoms; and Ar³ is a substituted or unsubstituted aromatic hydrocarbon group or a fused aromatic hydrocarbon group each having 6 to 40, preferably 6 to 30, more preferably 6 to 20, and still more preferably 6 to 12 ring carbon atoms or a substituted or unsubstituted aromatic heterocyclic group or a fused aromatic heterocyclic group each having 5 to 40, preferably 5 to 30, more preferably 5 to 20, and still more preferably 5 to 12 ring atoms. R³⁰¹ to R³⁰⁸ may be all hydrogen atoms.

Ar² is selected, for example, from the following groups:

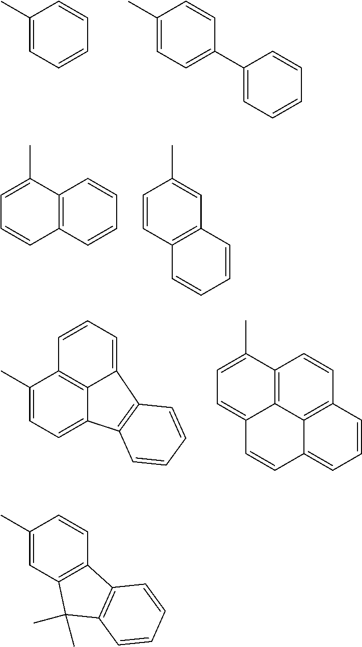

In addition, the following compound is preferably used as the nitrogen-containing aromatic polycyclic compound for use as the electron transporting compound:

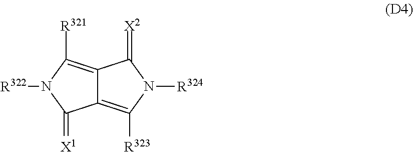

wherein R³²¹ to R³²⁴ each independently represent a hydrogen atom, a substituted or unsubstituted aliphatic group having 1 to 20 carbon atoms, a substituted or unsubstituted alicyclic group having 3 to 20, preferably 3 to 10, and more preferably 5 to 8 ring carbon atoms, a substituted or unsubstituted aromatic ring group having 6 to 50, preferably 6 to 30, more preferably 6 to 20, and still more preferably 6 to 12 ring carbon atoms, or a substituted or unsubstituted heterocyclic group having 5 to 50, preferably 5 to 30, more preferably 5 to 20, and still more preferably 5 to 12 ring atoms; and X¹ and X² each independently represent an oxygen atom, a sulfur atom, or a dicyanomethylene group.

Further, the following compound is also suitable as the electron transporting compound:

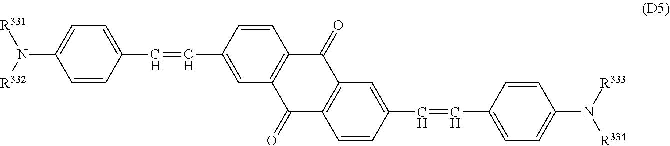

wherein R³³¹ to R³³⁴ may be the same or different and each represents an aromatic hydrocarbon group or a fused aromatic hydrocarbon group each represented by formula (D6):

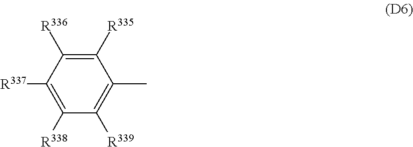

wherein R³³⁵ to R³³⁹ may be the same or different and each represents a hydrogen atom, a heavy hydrogen atom, a saturated or unsaturated alkoxyl group having 1 to 20 carbon atoms, a saturated or unsaturated alkyl group having 1 to 20, preferably 1 to 10, and more preferably 1 to 6 carbon atoms, an amino group, or an alkylamino group having 1 to 20, preferably 1 to 10, and more preferably 1 to 6 carbon atoms. At least one of R³³⁵ to R³³⁹ represents a group other than a hydrogen atom and a heavy hydrogen atom.

Further, a polymer including the above nitrogen-containing heterocyclic group or the above nitrogen-containing heterocyclic derivative is also usable as the electron transporting compound.

In a particularly preferred embodiment of the invention, the electron transporting layer of the organic EL device comprises at least one compound selected from the nitrogen-containing heterocyclic derivatives represented by formulae (E) to (G):

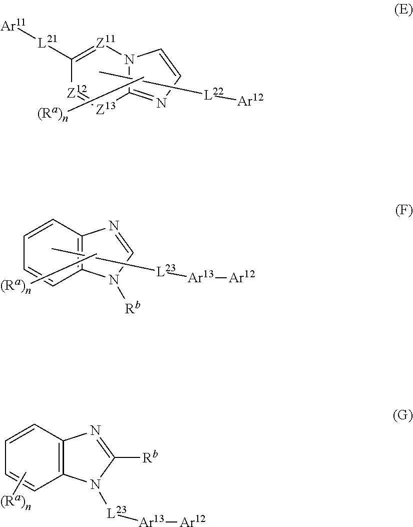

wherein Z¹¹, Z¹², and Z¹³ each independently represent a nitrogen atom or a carbon atom;

R^{a} and R^{b} each independently represent a substituted or unsubstituted aryl group having 6 to 50, preferably 6 to 30, more preferably 6 to 20, and still more preferably 6 to 12 ring carbon atoms, a substituted or unsubstituted heteroaryl group having 5 to 50, preferably 5 to 30, more preferably 5 to 20, and still more preferably 5 to 12 ring atoms, a substituted or unsubstituted alkyl group having 1 to 20, preferably 1 to 10, and more preferably 1 to 6 carbon atoms, a substituted or unsubstituted haloalkyl group having 1 to 20, preferably 1 to 10, and more preferably 1 to 6 carbon atoms, or a substituted or unsubstituted alkoxyl group having 1 to 20, preferably 1 to 10, and more preferably 1 to 6 carbon atoms, wherein R^{b} is preferably a substituted or unsubstituted alkyl group having 1 to 20, preferably 1 to 10, and more preferably 1 to 6 carbon atoms and more preferably a methyl group or an ethyl group;

n is an integer of 0 to 5, when n is an integer of 2 or more, groups R^{a} may be the same or different, and adjacent two groups R^{a} may bond to each other to form a substituted or unsubstituted hydrocarbon ring;

Ar¹¹ represents a substituted or unsubstituted aryl group having 6 to 50, preferably 6 to 30, more preferably 6 to 20, and still more preferably 6 to 12 ring carbon atoms or a substituted or unsubstituted heteroaryl group having 5 to 50 ring atoms;

Ar¹² represents a hydrogen atom, a substituted or unsubstituted alkyl group having 1 to 20, preferably 1 to 10, and more preferably 1 to 6 carbon atoms, a substituted or unsubstituted haloalkyl group having 1 to 20, preferably 1 to 10, and more preferably 1 to 6 carbon atoms, a substituted or unsubstituted alkoxyl group having 1 to 20, preferably 1 to 10, and more preferably 1 to 6 carbon atoms, a substituted or unsubstituted aryl group having 6 to 50, preferably 6 to 30, more preferably 6 to 20, and still more preferably 6 to 12 ring carbon atoms, or a substituted or unsubstituted heteroaryl group having 5 to 50, preferably 5 to 30, more preferably 5 to 20, and still more preferably 5 to 12 ring atoms, wherein Ar¹² is preferably a substituted or unsubstituted aryl group having 6 to 50, preferably 6 to 30, more preferably 6 to 20, and still more preferably 6 to 12 ring carbon atoms and more preferably a phenyl group;

provided that one of Ar¹¹ and Ar¹² is a substituted or unsubstituted fused aromatic hydrocarbon ring group having 10 to 50, preferably 10 to 30, more preferably 10 to 20, and still more preferably 10 to 14 ring carbon atoms or a substituted or unsubstituted fused aromatic heterocyclic group having 9 to 50, preferably 9 to 30, more preferably 9 to 20, and still more preferably 9 to 14 ring atoms, wherein the fused aromatic hydrocarbon ring for the fused aromatic hydrocarbon ring group is preferably an anthracene ring;

Ar¹³ represents a substituted or unsubstituted arylene group having 6 to 50, preferably 6 to 30, more preferably 6 to 20, and still more preferably 6 to 14 ring carbon atoms or a substituted or unsubstituted heteroarylene group having 5 to 50, preferably 5 to 30, more preferably 5 to 20, and still more preferably 5 to 14 ring atoms; and

L²¹, L²², and L²³ each independently represent a single bond, a substituted or unsubstituted arylene group having 6 to 50, preferably 6 to 30, more preferably 6 to 20, and still more preferably 6 to 12 ring carbon atoms or a substituted or unsubstituted, divalent fused aromatic heterocyclic group having 9 to 50, preferably 9 to 30, more preferably 9 to 20, and still more preferably 9 to 14 ring atoms, wherein each of L²¹, L²², and L²³ is preferably a substituted or unsubstituted arylene group having 6 to 50, preferably 6 to 30, more preferably 6 to 20, and still more preferably 6 to 12 ring carbon atoms and more preferably a phenylene group.

Examples of the aryl group having 6 to 50 ring carbon atoms include a phenyl group, a naphthyl group, an anthryl group, a phenanthryl group, a naphthacenyl group, a chrysenyl group, pyrenyl group, a biphenyl group, a terphenyl group, a tolyl group, a fluoranthenyl group, and a fluorenyl group.

Examples of the heteroaryl group having 5 to 50 ring atoms include a pyrrolyl group, a furyl group, a thiophenyl group, a silolyl group, a pyridyl group, a quinolyl group, an isoquinolyl group, a benzofuryl group, an imidazolyl group, a pyrimidyl group, a carbazolyl group, a selenophenyl group, an oxadiazolyl group, a triazolyl group, a pyrazinyl group, a pyridazinyl group, a triazinyl group, a quinoxalinyl group, an acridinyl group, an imidazo[1,2-a]pyridinyl group, and an imidazo[1,2-a]pyrimidinyl.

Examples of the alkyl group having 1 to 20 carbon atoms include a methyl group, an ethyl group, a propyl group, a butyl group, a pentyl group, and a hexyl group.

Examples of the haloalkyl group having 1 to 20 carbon atoms include the groups obtained by replacing one or more hydrogen atoms of the alkyl group mentioned above with at least one halogen atom selected from fluorine, chlorine, iodine, and bromine.

Examples of the alkyl moiety of the alkoxyl group having 1 to 20 carbon atoms include the alkyl group mentioned above.

Examples of the arylene groups include the groups obtained by removing one hydrogen atom from the aryl group mentioned above.

Examples of the divalent fused aromatic heterocyclic group having 9 to 50 ring atoms include the groups obtained by removing one hydrogen atom from the fused aromatic heterocyclic group mentioned above as the heteroaryl group.

Of formulae (E) to (G), preferred is formula (G).

The thickness of the electron transporting layer is preferably, but not particularly limited to, 1 to 100 nm.

Preferred examples of the material for an electron injecting layer optionally formed adjacent to the electron transporting layer include, in addition to the nitrogen-containing ring derivative, an inorganic compound, such as an insulating material and a semiconductor. The electron injecting layer containing the insulating material or the semiconductor effectively prevents the leak of electric current to enhance the electron injecting properties.

The insulating material is preferably at least one metal compound selected from the group consisting of alkali metal chalcogenides, alkaline earth metal chalcogenides, alkali metal halides and alkaline earth metal halides. The alkali metal chalcogenide, etc. mentioned above are preferred because the electron injecting properties of the electron injecting layer are further enhanced. Examples of preferred alkali metal chalcogenide include Li₂O, K₂O, Na₂S, Na₂Se and Na₂O, and examples of preferred alkaline earth metal chalcogenide include CaO, BaO, SrO, BeO, BaS and CaSe. Examples of preferred alkali metal halide include LiF, NaF, KF, LiCl, KCl and NaCl. Examples of the alkaline earth metal halide include fluorides, such as CaF₂, BaF₂, SrF₂, MgF₂ and BeF₂, and halides other than fluorides.

Examples of the semiconductor include oxides, nitrides or oxynitrides of at least one element selected from the group consisting of Ba, Ca, Sr, Yb, Al, Ga, In, Li, Na, Cd, Mg, Si, Ta, Sb and Zn. The semiconductor may be used alone or in combination of two or more. The inorganic compound included in the electron injecting layer preferably forms a microcrystalline or amorphous insulating thin film. If the electron injecting layer is formed from such an insulating thin film, the pixel defects, such as dark spots, can be decreased because a more uniform thin film is formed. Examples of such inorganic compound include the alkali metal chalcogenide, the alkaline earth metal chalcogenide, the alkali metal halide and the alkaline earth metal halide.

When using the insulating material or the semiconductor, the thickness of its layer is preferably about 0.1 to 15 nm. The electron injecting layer may contain the electron-donating dopant mentioned above.

### Hole Transporting Layer

The hole transporting layer is an organic layer formed between the light emitting layer and the anode and has a function of transporting holes from the anode to the light emitting layer. When the hole transporting layer is formed into two or more layers, the layer closer to the anode may be defined as a hole injecting layer in some cases. The hole injecting layer has a function of efficiently injecting holes from the anode to the organic layer unit.

Another preferred material for the hole transporting layer may include an aromatic amine compound, for example, an aromatic amine derivative represented by formula (H):

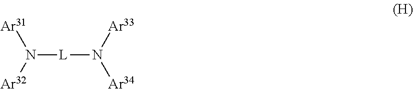

wherein each of Ar³¹ to Ar³⁴ represents a substituted or unsubstituted aromatic hydrocarbon group or a fused aromatic hydrocarbon group each having 6 to 50, preferably 6 to 30, more preferably 6 to 20, and still more preferably 6 to 12 ring carbon atoms, a substituted or unsubstituted aromatic heterocyclic group or a fused aromatic heterocyclic group each having 5 to 50, preferably 5 to 30, more preferably 5 to 20, and still more preferably 5 to 12 ring atoms, or a group wherein the aromatic hydrocarbon group or the fused aromatic hydrocarbon group is bonded to the aromatic heterocyclic group or the fused aromatic heterocyclic group; and

L represents a substituted or unsubstituted aromatic hydrocarbon group or a fused aromatic hydrocarbon group each having 6 to 50, preferably 6 to 30, more preferably 6 to 20, and still more preferably 6 to 12 ring carbon atoms or a substituted or unsubstituted aromatic heterocyclic group or a fused aromatic heterocyclic group each having 5 to 50, preferably 5 to 30, more preferably 5 to 20, and still more preferably 5 to 12 ring atoms.

Examples of the compound represented by formula (H) are shown below.

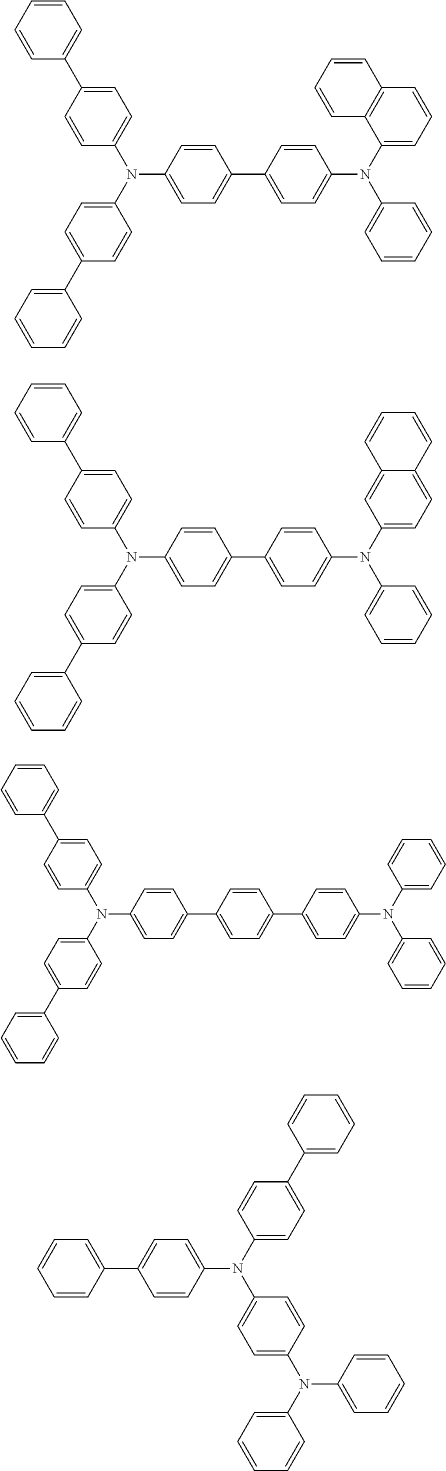

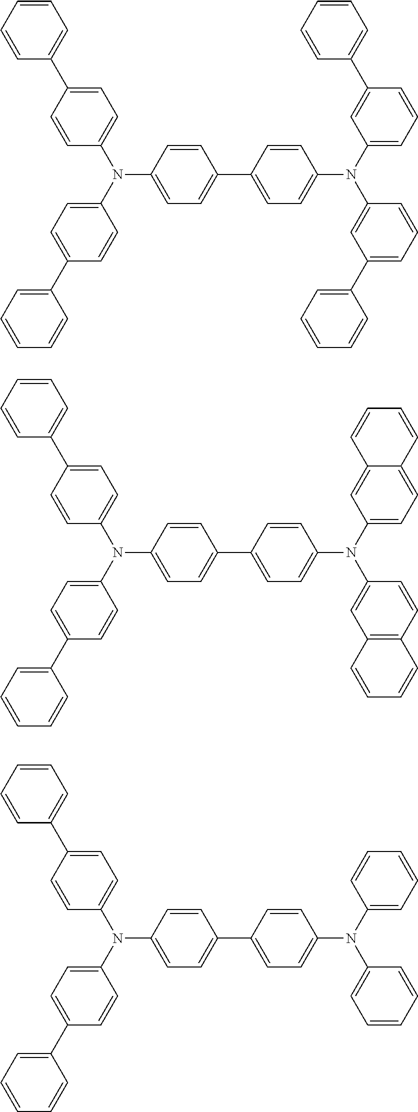

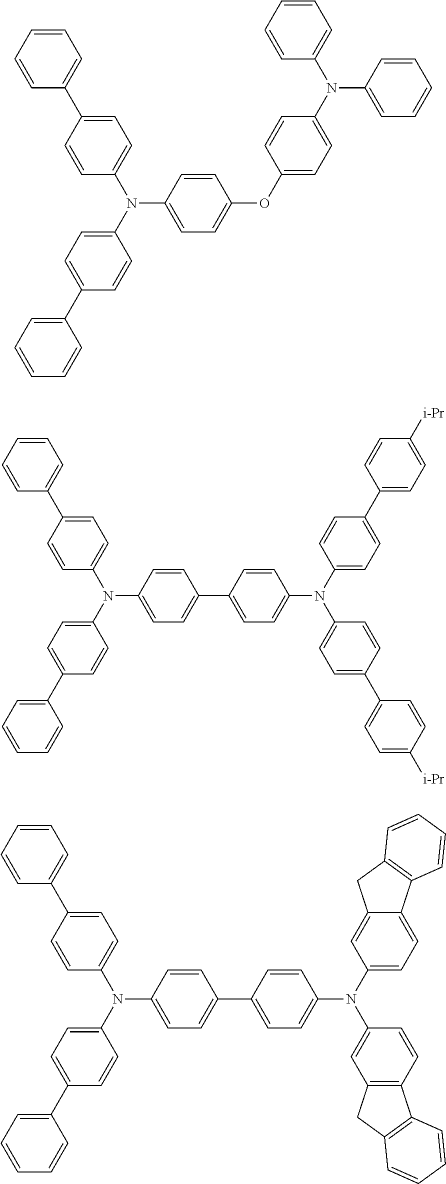

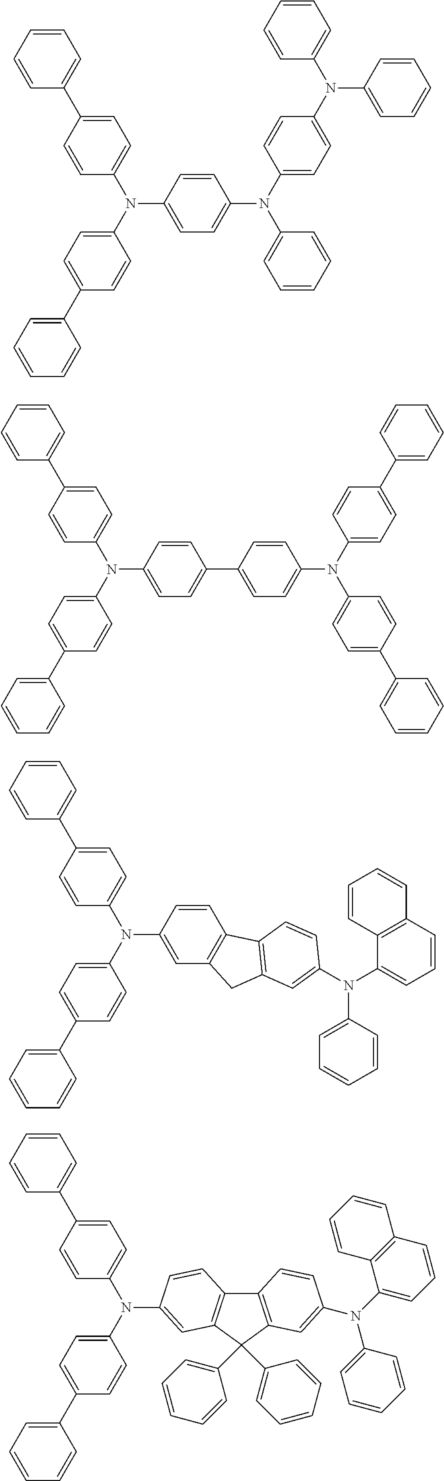

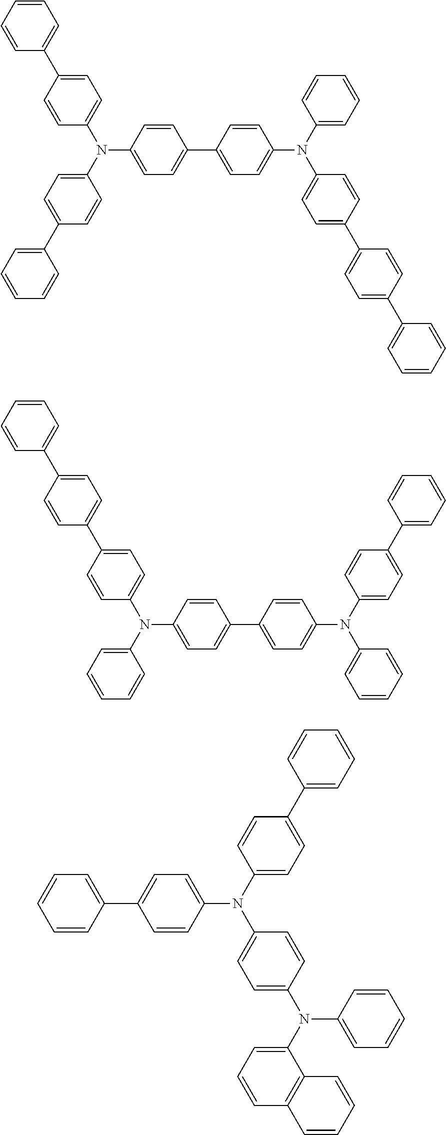

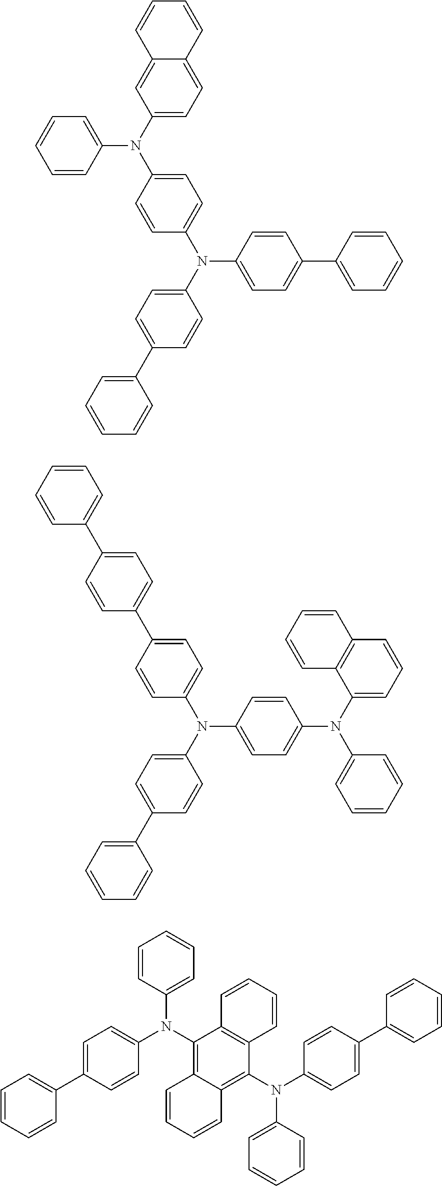

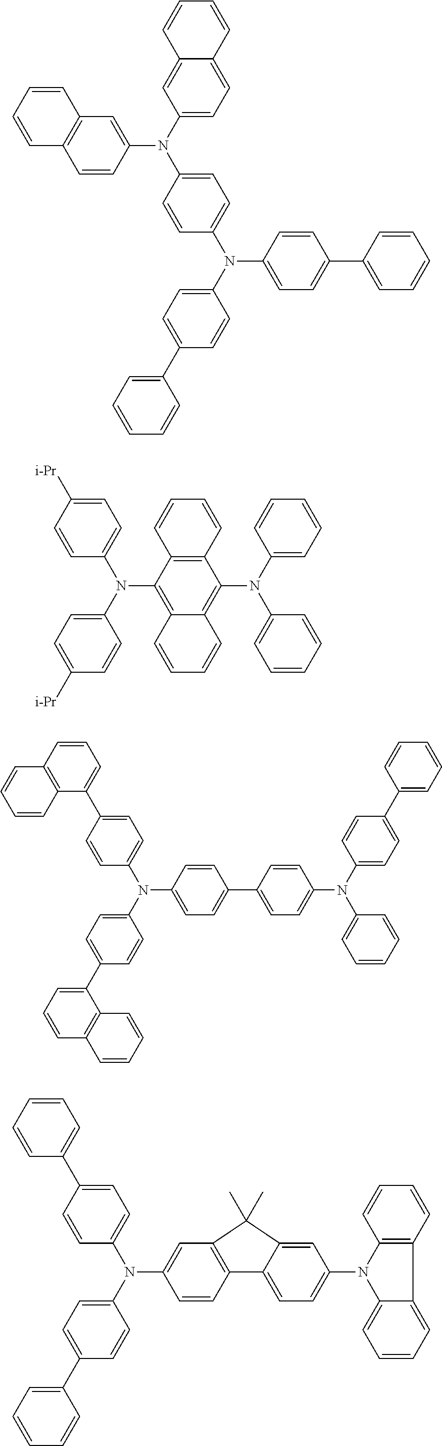

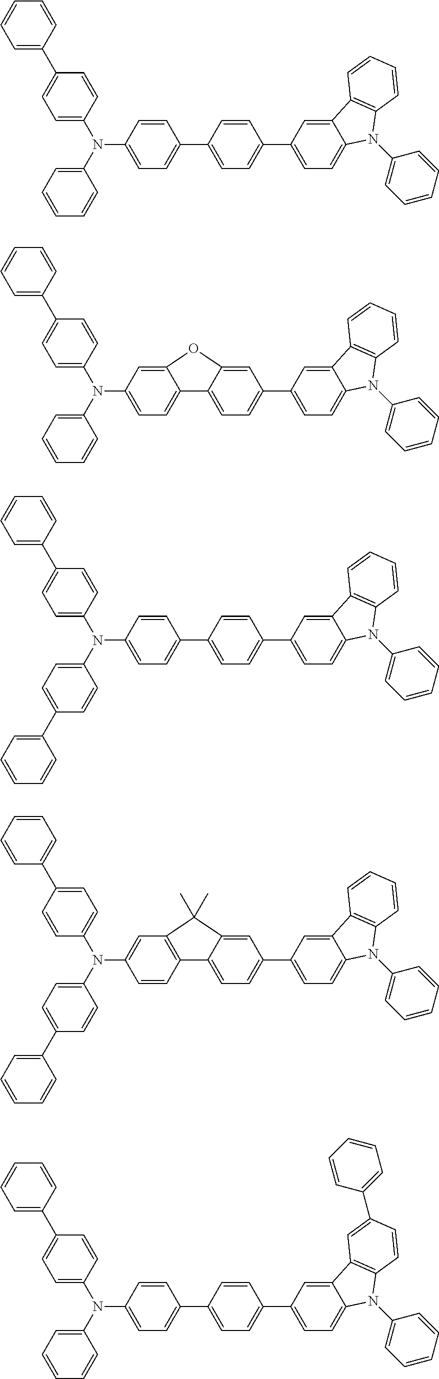

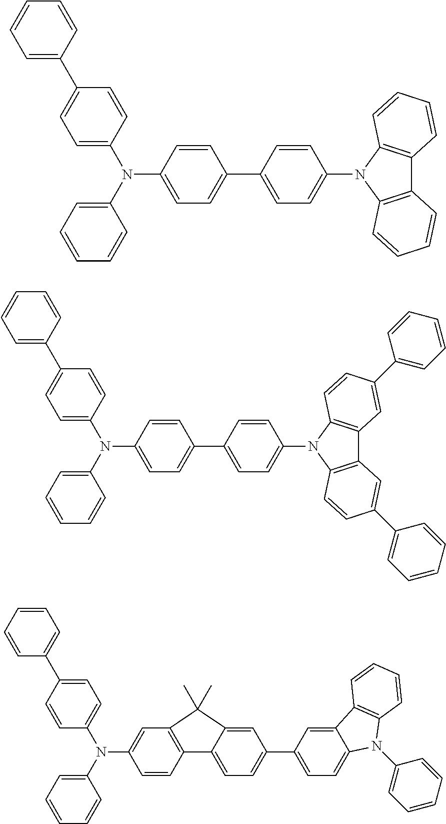 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccccc3)c3ccc(-c4ccc(-n5c6ccc(-c7ccccc7)cc6c6cc(-c7ccccc7)ccc65)cc4)cc3)cc2)cc1 ; CC1(C)c2cc(-c3ccc4c(c3)c3ccccc3n4-c3ccccc3)ccc2-c2ccc(N(c3ccccc3)c3ccc(-c4ccccc4)cc3)cc21]

An aromatic amine represented by formula (J) is also preferably used to form the hole transporting layer:

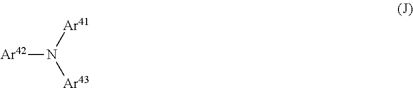

wherein Ar⁴¹ to Ar⁴³ and preferred examples thereof are as defined above with respect to Ar³¹ to Ar³⁴ of formula (H). Examples of the compound represented by formula (J) are shown below, although not limited thereto.

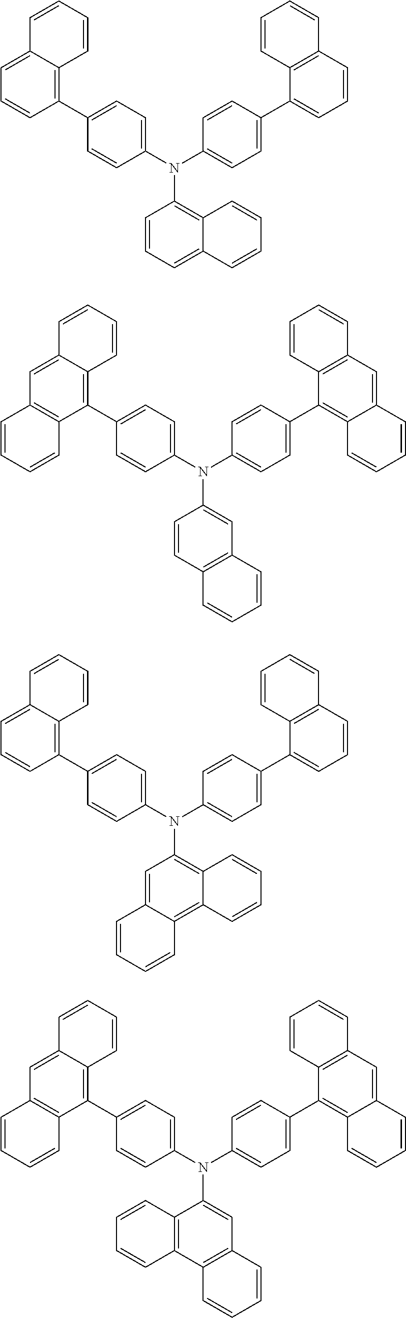 [SMILES: c1ccc2c(-c3ccc(N(c4ccc(-c5cccc6ccccc56)cc4)c4cccc5ccccc45)cc3)cccc2c1 ; c1ccc2cc(N(c3ccc(-c4c5ccccc5cc5ccccc45)cc3)c3ccc(-c4c5ccccc5cc5ccccc45)cc3)ccc2c1 ; c1ccc2c(-c3ccc(N(c4ccc(-c5cccc6ccccc56)cc4)c4cc5ccccc5c5ccccc45)cc3)cccc2c1 ; c1ccc2c(-c3ccc(N(c4ccc(-c5c6ccccc6cc6ccccc56)cc4)c4cc5ccccc5c5ccccc45)cc3)c3ccccc3cc2c1]

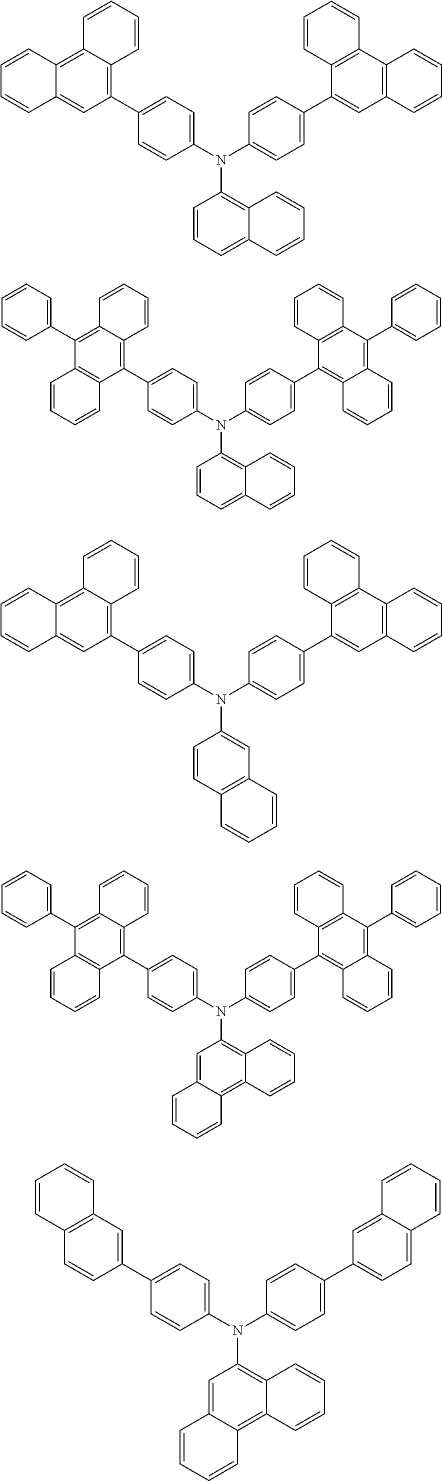 [SMILES: c1ccc2c(N(c3ccc(-c4cc5ccccc5c5ccccc45)cc3)c3ccc(-c4cc5ccccc5c5ccccc45)cc3)cccc2c1 ; c1ccc(-c2c3ccccc3c(-c3ccc(N(c4ccc(-c5c6ccccc6c(-c6ccccc6)c6ccccc56)cc4)c4cccc5ccccc45)cc3)c3ccccc23)cc1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2c3ccccc3c(-c3ccc(N(c4ccc(-c5c6ccccc6c(-c6ccccc6)c6ccccc56)cc4)c4cc5ccccc5c5ccccc45)cc3)c3ccccc23)cc1 ; c1ccc2cc(-c3ccc(N(c4ccc(-c5ccc6ccccc6c5)cc4)c4cc5ccccc5c5ccccc45)cc3)ccc2c1]

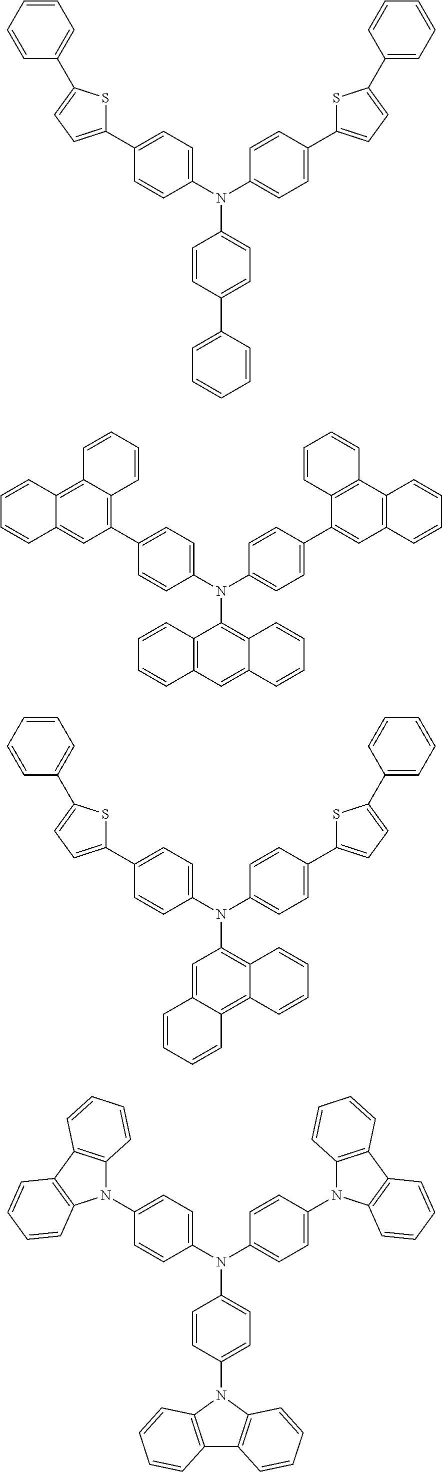 [SMILES: c1ccc(-c2ccc(N(c3ccc(-c4ccc(-c5ccccc5)s4)cc3)c3ccc(-c4ccc(-c5ccccc5)s4)cc3)cc2)cc1 ; c1ccc2c(N(c3ccc(-c4cc5ccccc5c5ccccc45)cc3)c3ccc(-c4cc5ccccc5c5ccccc45)cc3)c3ccccc3cc2c1 ; c1ccc(-c2ccc(-c3ccc(N(c4ccc(-c5ccc(-c6ccccc6)s5)cc4)c4cc5ccccc5c5ccccc45)cc3)s2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

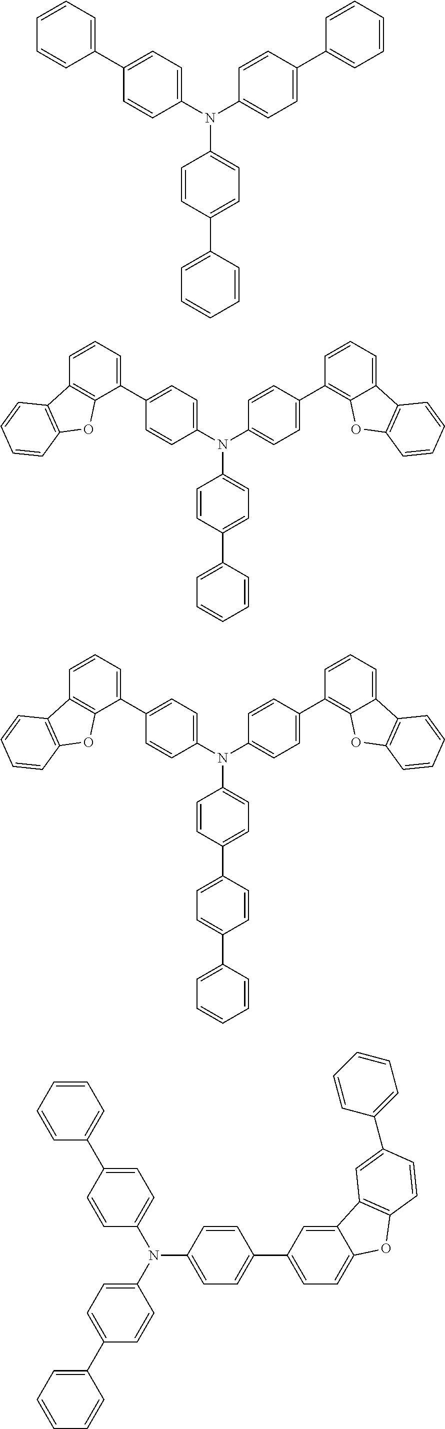 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4ccccc4)cc3)c3ccc(-c4ccc5oc6ccc(-c7ccccc7)cc6c5c4)cc3)cc2)cc1]

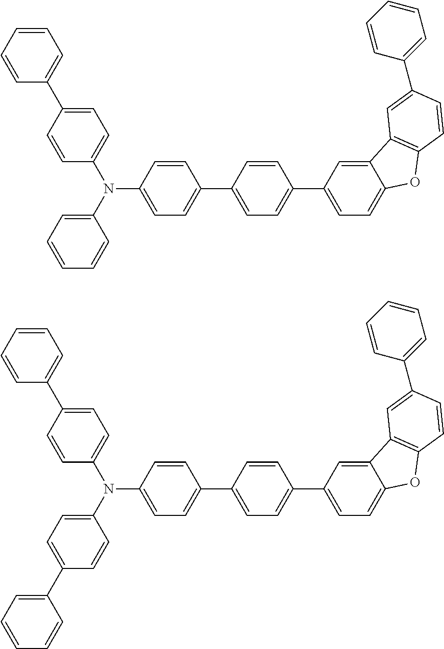 [SMILES: c1ccc(-c2ccc(N(c3ccccc3)c3ccc(-c4ccc(-c5ccc6oc7ccc(-c8ccccc8)cc7c6c5)cc4)cc3)cc2)cc1 ; c1ccc(-c2ccc(N(c3ccc(-c4ccccc4)cc3)c3ccc(-c4ccc(-c5ccc6oc7ccc(-c8ccccc8)cc7c6c5)cc4)cc3)cc2)cc1]

The hole transporting layer may be made into two-layered structure of a first hole transporting layer (anode side) and a second hole transporting layer (cathode side).

The thickness of the hole transporting layer is preferably 10 to 200 nm, although not particularly limited thereto.

The organic EL device of the invention may comprise a layer comprising an acceptor material, which is disposed in contact with the anode side of the hole transporting layer or the first hole transporting layer. With such a layer, it is expected that the driving voltage is lowered and the production cost is reduced.

The acceptor material is preferably a compound represented by formula (K):

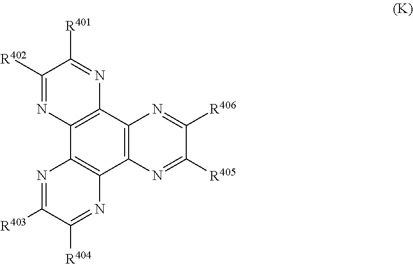

wherein R⁴⁰¹ to R⁴⁰⁶ each independently represent a cyano group, —CONH₂, a carboxyl group, or —COO R⁴⁰⁷ wherein R⁴⁰⁷ represents an alkyl group having 1 to 20 carbon atoms, or R⁴⁰¹ and R⁴⁰², R⁴⁰³ and R⁴⁰⁴, or R⁴⁰⁵ and R⁴⁰⁶ are bonded to each other to form a group represented by —CO—O—CO—.

Examples of the alkyl group for R⁴⁰⁷ include a methyl group, an ethyl group, a n-propyl group, an isopropyl group, a n-butyl group, an isobutyl group, a t-butyl group, a cyclopentyl group, and a cyclohexyl group.

The thickness of the layer comprising the acceptor material is preferably 5 to 20 nm, although not particularly limited thereto.

### N/P Doping

The carrier injecting properties of the hole transporting layer and the electron transporting layer can be controlled, as described in JP 3695714B, by the doping (n) with a donor material or the doping (p) with an acceptor material.

A typical example of the n-doping is an electron transporting material doped with a metal, such as Li and Cs, and a typical example of the p-doping is a hole transporting material doped with an acceptor material, such as 2,3,5,6-tetrafluoro-7,7,8,8-tetracyanoquinodimethane (F₄TCNQ).

### Space Layer

For example, in an organic EL device wherein a fluorescent light emitting layer and a phosphorescent light emitting layer are laminated, a space layer is disposed between the fluorescent light emitting layer and the phosphorescent light emitting layer to prevent the diffusion of excitons generated in the phosphorescent light emitting layer to the fluorescent light emitting layer or to control the carrier balance. The space layer may be disposed between two or more phosphorescent light emitting layers.

Since the space layer is disposed between the light emitting layers, a material combining the electron transporting ability and the hole transporting ability is preferably used for forming the space layer. To prevent the diffusion of triplet energy in the adjacent phosphorescent light emitting layer, the triplet energy of the material for the space layer is preferably 2.6 eV or more. The materials described above with respect to the hole transporting layer are usable as the material for the space layer.

### Blocking Layer

The organic EL device of the invention preferably has a blocking layer, such as an electron blocking layer, a hole blocking layer, and a triplet blocking layer, which is disposed adjacent to the light emitting layer. The electron blocking layer is a layer which prevents the diffusion of electrons from the light emitting layer to the hole transporting layer. The hole blocking layer is a layer which prevents the diffusion of holes from the light emitting layer to the electron transporting layer.

The triplet blocking layer prevents the diffusion of triplet excitons generated in the light emitting layer to adjacent layers and has a function of confining the triplet excitons in the light emitting layer, thereby preventing the deactivation of energy on molecules other than the emitting dopant of triplet excitons, for example, on molecules in the electron transporting layer.

If a phosphorescent device having a triplet blocking layer satisfies the following energy relationship:

E^{T}_{d}<E^{T}_{TB}

wherein E^{T}_{d} is the triplet energy of the phosphorescent dopant in the light emitting layer and E^{T}_{TB} is the triplet energy of the compound forming the triplet blocking layer,
the triplet excitons of phosphorescent dopant are confined (not diffuse to other molecules). Therefore, the energy deactivation process other than the emission on the phosphorescent dopant may be prevented to cause the emission with high efficiency. However, even in case of satisfying the relationship of E^{T}_{d}<E^{T}_{TB}, the triplet excitons may move into other molecules if the energy difference (ΔE^{T}=E^{T}_{TB}−E^{T}_{d}) is small, because the energy difference ΔE^{T} may be overcome by the absorption of ambient heat energy when driving a device at around room temperature as generally employed in practical drive of device. As compared with the fluorescent emission, the phosphorescent emission is relatively likely to be affected by the diffusion of excitons due to the heat absorption because the lifetime of triplet excitons is longer. Therefore, as for the energy difference ΔE^{T}, the larger as compared with the heat energy of room temperature, the better. The energy difference ΔE^{T} is more preferably 0.1 eV or more and particularly preferably 0.2 eV or more. In fluorescent devices, the material for organic EL device in an aspect of the invention is usable as the material for triplet blocking layer of the TTF device described in WO 2010/134350A1.

The electron mobility of the material for the triplet blocking layer is preferably 10⁻⁶ cm²/Vs or more at an electric field strength of 0.04 to 0.5 MV/cm. There are several methods for measuring the electron mobility of organic material, for example, Time of Flight method. In the present invention, the electron mobility is determined by impedance spectroscopy.

The electron mobility of the electron injecting layer is preferably 10⁻⁶ cm²/Vs or more at an electric field strength of 0.04 to 0.5 MV/cm. Within the above range, the injection of electrons from the cathode to the electron transporting layer is promoted and the injection of electrons to the adjacent blocking layer and light emitting layer is also promoted, thereby enabling to drive a device at lower voltage.

The organic EL devices employing the compound of the invention are further improved in the emission efficiency and the lifetime, and some of them can be driven at a low voltage. Therefore, the organic EL device of the invention is usable in electronic equipment, for example, as display parts, such as organic EL panel module, display devices of television sets, mobile phones, personal computer, etc., and light emitting sources of lighting equipment and vehicle lighting equipment.

### EXAMPLES

The present invention will be described below in more detail with reference to the examples and comparative examples. However, it should be noted that the scope of the invention is not limited thereto.

### Example 1

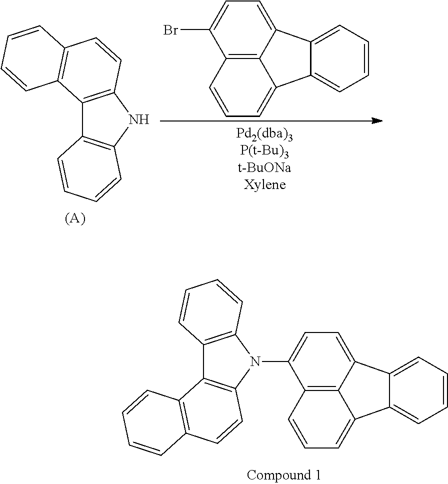

In an argon atmosphere, 2.17 g of a starting compound (A), 3.10 g of 3-bromofluoranthene synthesized by a known method, 0.18 g of trisdibenzylideneacetone dipalladium(0), 0.23 g of tri-t-butylphosphine tetrafluorohydroborate, 1.30 g of sodium t-butoxide, and 100 mL of dehydrated xylene were charged in a flask, and the resultant mixture was heat-refluxed for 8 h under stirring.

After cooling to room temperature, the reaction solution was extracted with toluene and filtered through celite. The filtrate was concentrated, and the residue was purified by silica gel column chromatography to obtain 2.71 g of a yellow solid, which was identified as the target compound (compound 1) by a result of mass spectrometric analysis which showed m/e=417 to the molecular weight of 417.15.

### Example 2

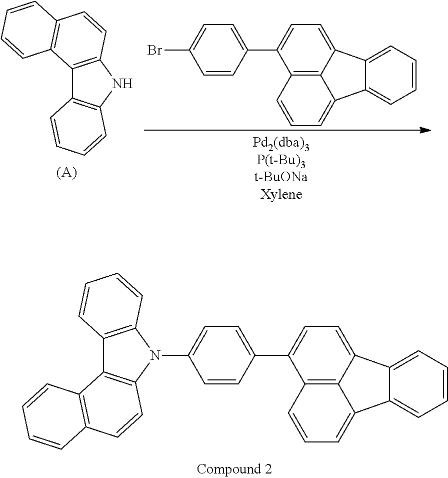

A compound was synthesized in the same manner as in Example 1 except for using 3-(4-bromophenyl)fluoranthene synthesized by a known method in place of 3-bromofluoranthene. The obtained compound was identified as the target compound (compound 2) by a result of mass spectrometric analysis which showed m/e=493 to the molecular weight of 493.18.

### Example 3

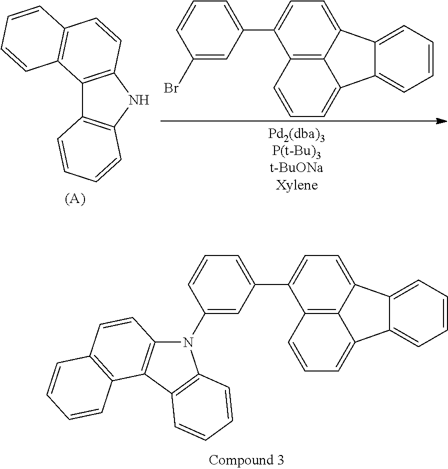

A compound was synthesized in the same manner as in Example 1 except for using 3-(3-bromophenyl)fluoranthene synthesized by a known method in place of 3-bromofluoranthene. The obtained compound was identified as the target compound (compound 3) by a result of mass spectrometric analysis which showed m/e=493 to the molecular weight of 493.18.

### Example 4

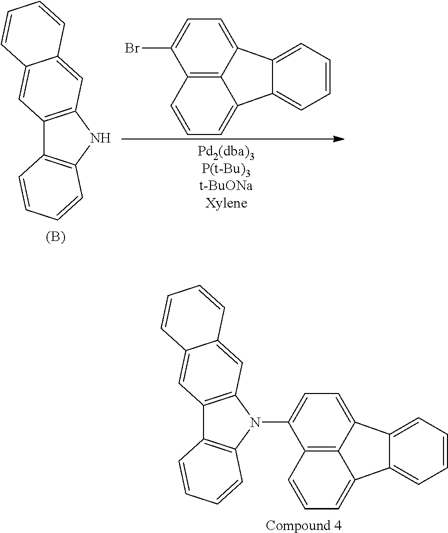

A compound was synthesized in the same manner as in Example 1 except for using a starting compound (B) synthesized by a known method in place of the starting compound (A). The obtained compound was identified as the target compound (compound 4) by a result of mass spectrometric analysis which showed m/e=417 to the molecular weight of 417.15.

### Example 5

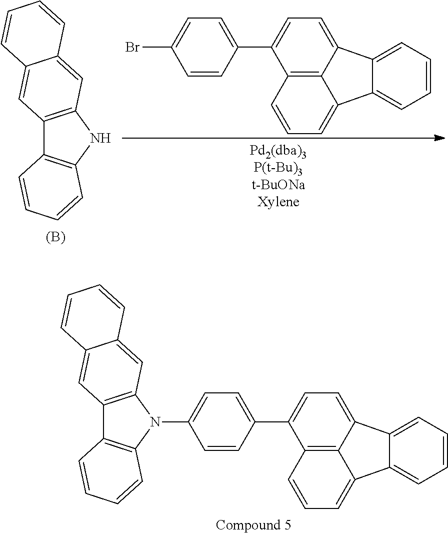

A compound was synthesized in the same manner as in Example 1 except for using the starting compound (B) synthesized by a known method in place of the starting compound (A) and using 3-(4-bromophenyl)fluoranthene synthesized by a known method in place of 3-bromofluoranthene. The obtained compound was identified as the target compound (compound 5) by a result of mass spectrometric analysis which showed m/e=493 to the molecular weight of 493.18.

### Example 6

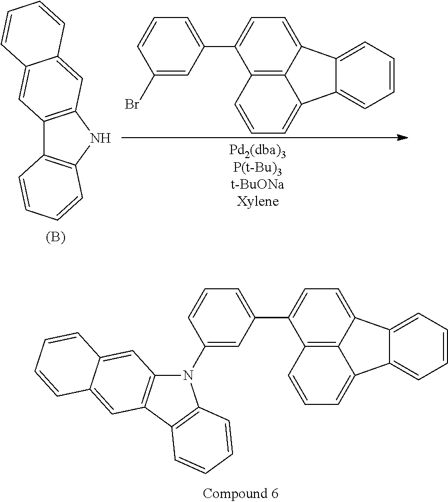

A compound was synthesized in the same manner as in Example 1 except for using the starting compound (B) synthesized by a known method in place of the starting compound (A) and using 3-(3-bromophenyl)fluoranthene synthesized by a known method in place of 3-bromofluoranthene. The obtained compound was identified as the target compound (compound 6) by a result of mass spectrometric analysis which showed m/e=493 to the molecular weight of 493.18.

### Example 7

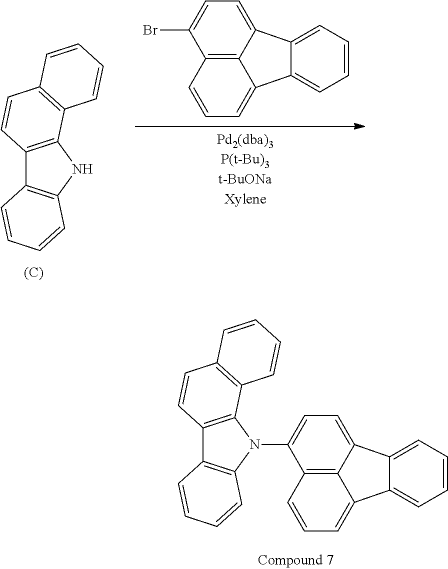

A compound was synthesized in the same manner as in Example 1 except for using a starting compound (C) synthesized by a known method in place of the starting compound (A). The obtained compound was identified as the target compound (compound 7) by a result of mass spectrometric analysis which showed m/e=417 to the molecular weight of 417.15.

### Example 8

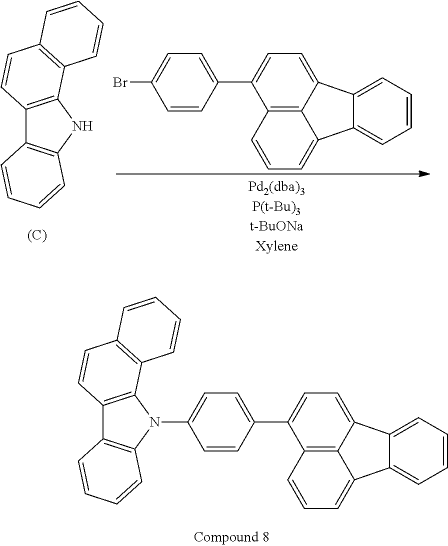

A compound was synthesized in the same manner as in Example 1 except for using the starting compound (C) synthesized by a known method in place of the starting compound (A) and using 3-(4-bromophenyl)fluoranthene synthesized by a known method in place of 3-bromofluoranthene. The obtained compound was identified as the target compound (compound 8) by a result of mass spectrometric analysis which showed m/e=493 to the molecular weight of 493.18.

### Example 9

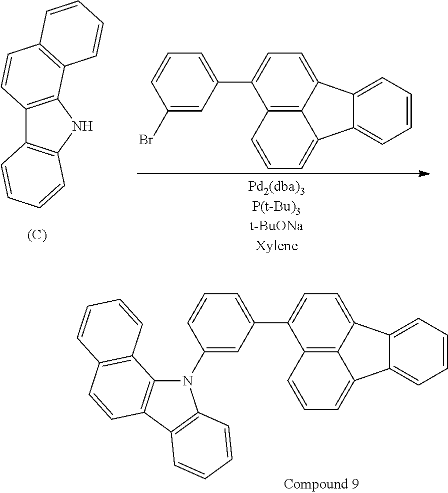

A compound was synthesized in the same manner as in Example 1 except for using the starting compound (C) synthesized by a known method in place of the starting compound (A) and using 3-(3-bromophenyl)fluoranthene synthesized by a known method in place of 3-bromofluoranthene. The obtained compound was identified as the target compound (compound 9) by a result of mass spectrometric analysis which showed m/e=493 to the molecular weight of 493.18.

### Example 10

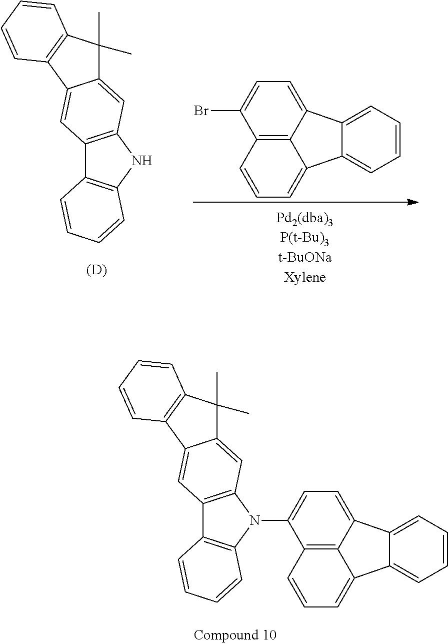

A compound was synthesized in the same manner as in Example 1 except for using a starting compound (D) synthesized by a known method in place of the starting compound (A). The obtained compound was identified as the target compound (compound 10) by a result of mass spectrometric analysis which showed m/e=483 to the molecular weight of 483.20.

### Example 11

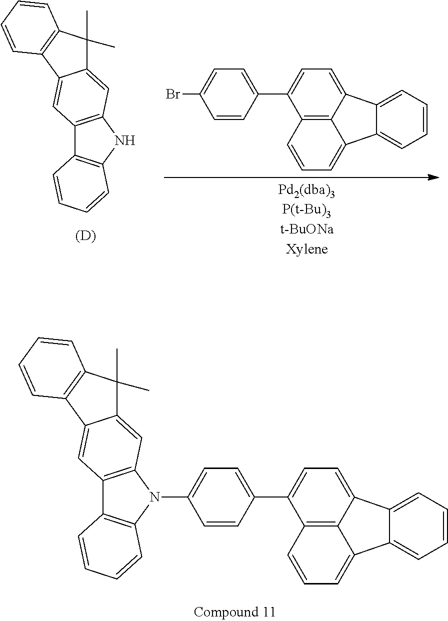

A compound was synthesized in the same manner as in Example 1 except for using the starting compound (D) synthesized by a known method in place of the starting compound (A) and using 3-(4-bromophenyl)fluoranthene synthesized by a known method in place of 3-bromofluoranthene. The obtained compound was identified as the target compound (compound 11) by a result of mass spectrometric analysis which showed m/e=559 to the molecular weight of 559.23.

### Example 12

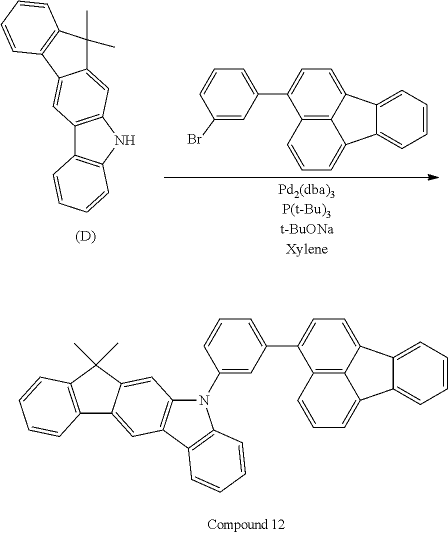

A compound was synthesized in the same manner as in Example 1 except for using the starting compound (D) synthesized by a known method in place of the starting compound (A) and using 3-(3-bromophenyl)fluoranthene synthesized by a known method in place of 3-bromofluoranthene. The obtained compound was identified as the target compound (compound 12) by a result of mass spectrometric analysis which showed m/e=559 to the molecular weight of 559.23.

### Example 13

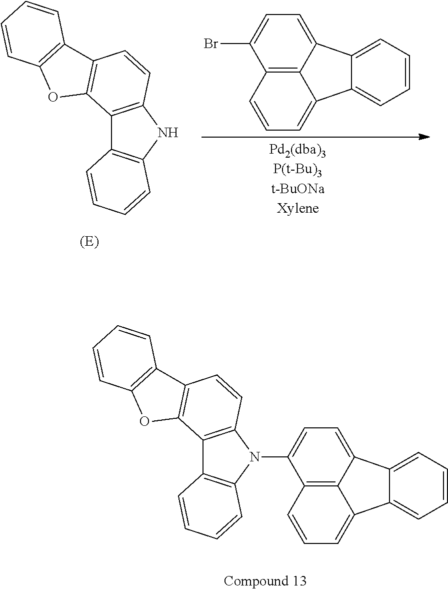

A compound was synthesized in the same manner as in Example 1 except for using a starting compound (E) synthesized by a known method in place of the starting compound (A). The obtained compound was identified as the target compound (compound 13) by a result of mass spectrometric analysis which showed m/e=457 to the molecular weight of 457.15.

### Example 14

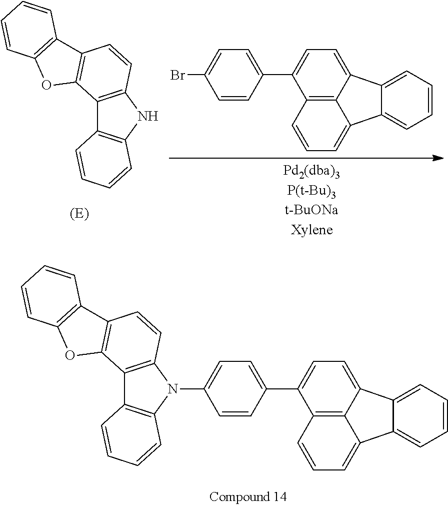

A compound was synthesized in the same manner as in Example 1 except for using the starting compound (E) synthesized by a known method in place of the starting compound (A) and using 3-(4-bromophenyl)fluoranthene synthesized by a known method in place of 3-bromofluoranthene. The obtained compound was identified as the target compound (compound 14) by a result of mass spectrometric analysis which showed m/e=559 to the molecular weight of 559.23.

### Example 15

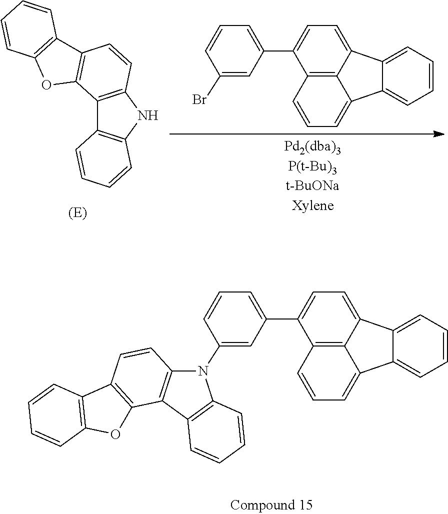

A compound was synthesized in the same manner as in Example 1 except for using the starting compound (F) synthesized by a known method in place of the starting compound (A) and using 3-(3-bromophenyl)fluoranthene synthesized by a known method in place of 3-bromofluoranthene. The obtained compound was identified as the target compound (compound 15) by a result of mass spectrometric analysis which showed m/e=559 to the molecular weight of 559.23.

### Example 16

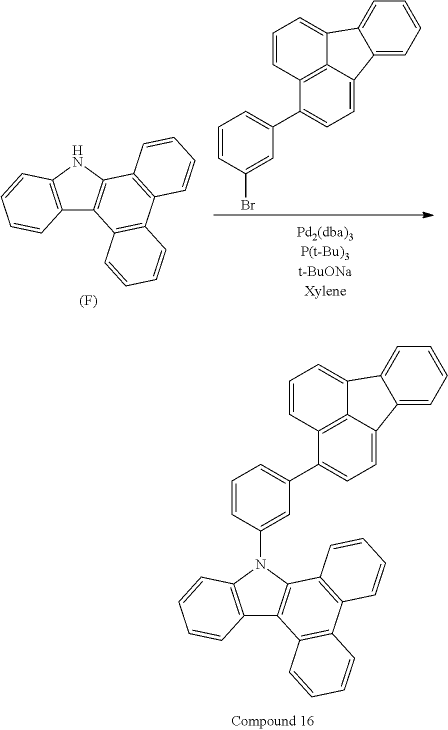

A compound was synthesized in the same manner as in Example 1 except for using a starting compound (F) synthesized by a known method in place of the starting compound (A) and using 3-(3-bromophenyl)fluoranthene synthesized by a known method in place of 3-bromofluoranthene. The obtained compound was identified as the target compound (compound 16) by a result of mass spectrometric analysis which showed m/e=543 to the molecular weight of 543.20.

### Example 17

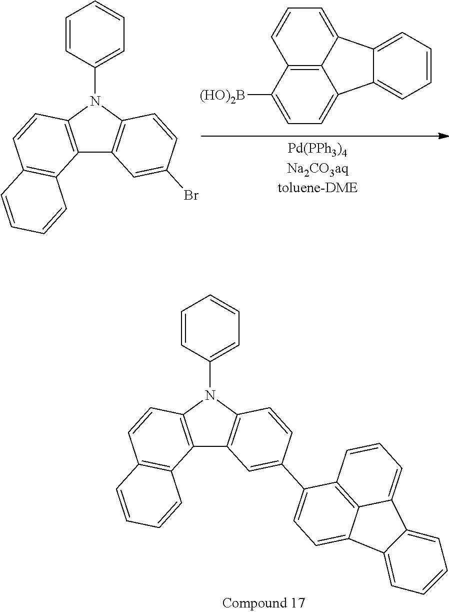 [SMILES: Brc1ccc2c(c1)c1c3ccccc3ccc1n2-c1ccccc1 ; OB(O)c1ccc2c3c(cccc13)-c1ccccc1-2 ; c1ccc(-n2c3ccc(-c4ccc5c6c(cccc46)-c4ccccc4-5)cc3c3c4ccccc4ccc32)cc1]

In an argon atmosphere, 2.95 g of 3-fluoranthene boronic acid, 3.72 g of 10-bromo-7-phenylbenzo[c]carbazole synthesized by a known method, 0.231 g of tetrakis(triphenylphosphine) palladium(0), 20 mL of 1,2-dimethoxyethane, 20 mL of toluene, and 20 mL of a 2 M aqueous solution of sodium carbonate were charged in a flask, and the resultant mixture was heat-refluxed for 8 h under stirring.

After cooling to room temperature, the reaction solution was extracted with toluene and then the aqueous layer was removed. The organic layer was washed with a saturated saline, dried over magnesium sulfate, and concentrated. The residue was purified by silica gel column chromatography to obtain 3.2 g of a yellow solid, which was identified as the target compound (compound 17) by a result of mass spectrometric analysis which showed m/e=493 to the molecular weight of 493.18.

### Example 18

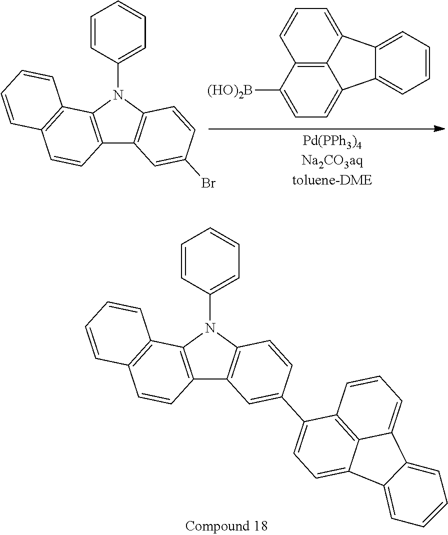

A compound was synthesized in the same manner as in Example 17 except for using 8-bromo-11-phenylbenzo[a]carbazole in place of 10-bromo-7-phenylbenzo[c]carbazole. The obtained compound was identified as the target compound (compound 18) by a result of mass spectrometric analysis which showed m/e=493 to the molecular weight of 493.18.

### Example 19

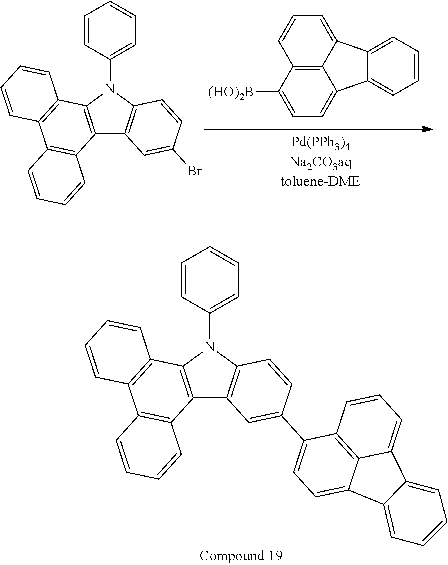 [SMILES: Brc1ccc2c(c1)c1c3ccccc3c3ccccc3c1n2-c1ccccc1 ; OB(O)c1ccc2c3c(cccc13)-c1ccccc1-2 ; c1ccc(-n2c3ccc(-c4ccc5c6c(cccc46)-c4ccccc4-5)cc3c3c4ccccc4c4ccccc4c32)cc1]

A compound was synthesized in the same manner as in Example 17 except for using 12-bromo-9-phenyldibenzo[a,c]carbazole in place of 10-bromo-7-phenylbenzo[c]carbazole. The obtained compound was identified as the target compound (compound 19) by a result of mass spectrometric analysis which showed m/e=543 to the molecular weight of 543.20.

### Example 20

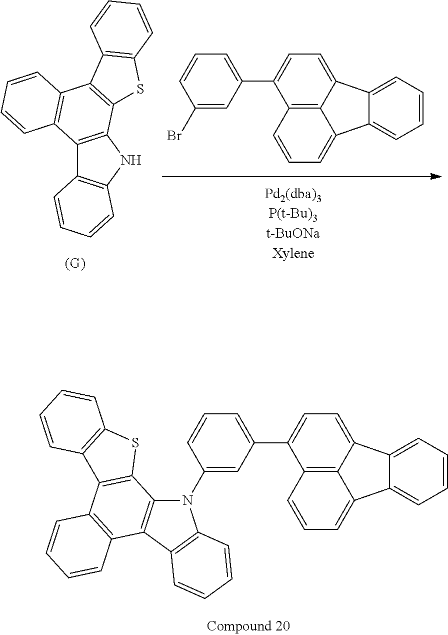

A compound was synthesized in the same manner as in Example 1 except for using 3-(3-bromophenyl)fluoranthene synthesized by a known method in place of 3-bromofluoranthene and using an intermediate (G) synthesized by a known method in place of the intermediate (A). The obtained compound was identified as the target compound (compound 20) by a result of mass spectrometric analysis which showed m/e=599 to the molecular weight of 599.17.

Other compounds within the claimed scope can be synthesized according to the reactions mentioned above, while using a known reaction and a known starting materials according to the target compound.

### [Production of Organic El Device and Evaluation of Emission Performance]

### Examples 21-35 and Comparative Example 1

A glass substrate of 25 mm×75 mm×1.1 mm thickness having an ITO transparent electrode (product of Geomatec Company) was cleaned by ultrasonic cleaning in isopropyl alcohol for 5 min and then UV ozone cleaning for 30 min.

The cleaned glass substrate having a transparent electrode line was mounted to a substrate holder of a vacuum vapor deposition apparatus. The following compound HT-1 was vapor-deposited so as to cover the transparent electrode to form a first hole transporting layer (anode-side organic thin film layer) with a thickness of 45 nm. Successively after forming the first hole transporting layer, the following compound HT-2 was vapor-deposited to form a second hole transporting layer (anode-side organic thin film layer) with a thickness of 10 nm.

On the second hole transporting layer, each compound (host material) shown in Table 1 and the following compound RD-1 (phosphorescent material) were vapor co-deposited to form a phosphorescent light emitting layer with a thickness of 40 nm. The concentration of the compound RD-1 in the light emitting layer was 5.0% by mass. The co-deposited film works as a light emitting layer.

Successively after forming the light emitting layer, the following compound ET-1 was vapor-deposited into a film with a thickness of 40 nm. The film of the compound ET-1 works as an electron transporting layer (cathode-side organic thin film layer).

Then, LiF was vapor-deposited into a film with a thickness of 1 nm at a film-forming speed of 0.1 Å/min to form an electron injecting electrode (cathode). On the LiF film, metallic Al was vapor-deposited to form a metallic cathode with a thickness of 80 nm, thereby obtaining an organic EL device.

The compounds used in the examples and comparative example are shown below.

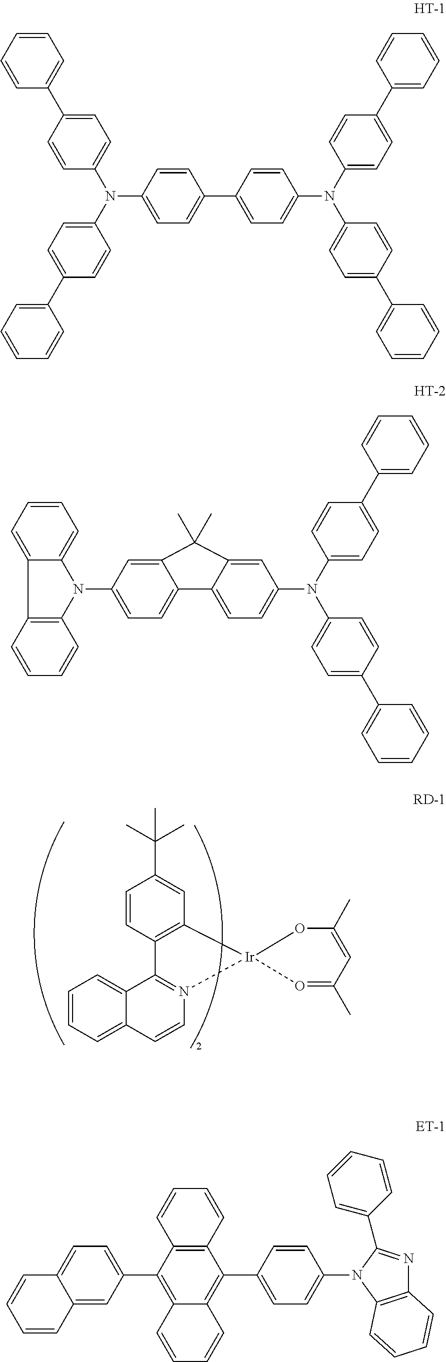

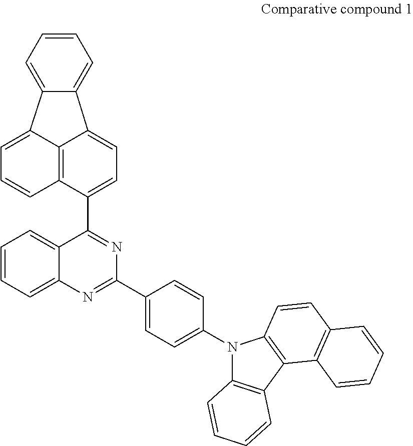 [SMILES: c1ccc2c(c1)-c1cccc3c(-c4nc(-c5ccc(-n6c7ccccc7c7c8ccccc8ccc76)cc5)nc5ccccc45)ccc-2c13]

### (Evaluation of Organic EL Device)

Each of the organic EL devices thus obtained was driven at a current density of 50 mA/cm² and measured for the lifetime (the time taken until the luminance was reduced to 90% of the initial luminance when driving at a constant current) by using a luminance meter (spectroradiometer “CS-1000” manufactured by Minolta). In addition, by driving each organic EL device at room temperature by a constant direct current (current density: 10 mA/cm²), the emission efficiency was measured by using a luminance meter (spectroradiometer “CS-1000” manufactured by Minolta). The results are shown in Table 1.

TABLE 1

| | | Results | |
|---|---|---|---|
| | Light emitting layer | emission | |
| | host material | efficiency (%) | lifetime (h) |
| Examples 21 | compound 1 | 16.2 | 100 |
| Examples 22 | compound 2 | 16.1 | 100 |
| Examples 23 | compound 3 | 16.3 | 110 |
| Examples 24 | compound 4 | 15.9 | 130 |
| Examples 25 | compound 5 | 16.3 | 120 |
| Examples 26 | compound 6 | 16.1 | 140 |
| Examples 27 | compound 7 | 15.8 | 130 |
| Examples 28 | compound 8 | 15.9 | 120 |
| Examples 29 | compound 9 | 15.7 | 120 |
| Examples 30 | compound 10 | 16.2 | 140 |
| Examples 31 | compound 11 | 16.4 | 130 |
| Examples 32 | compound 12 | 16.3 | 150 |
| Examples 33 | compound 13 | 16.5 | 140 |
| Examples 34 | compound 14 | 16.4 | 130 |
| Examples 35 | compound 15 | 16.6 | 120 |
| Comparative | comparative | 14.5 | 20 |
| Example 1 | compound 1 | | |

As seen from Table 1, the organic EL device having a light emitting layer in which each of the compounds 1 to 15 is used exhibits an improved emission efficiency and a drastically improved device life, as compared with the organic EL device having a light emitting layer comprising the comparative compound 1 wherein a fluoranthene ring and a benzocarbazole are bonded to each other via a nitrogen-containing heterocyclic derivative (disclosed in WO 2012/030145).

### [Production of Organic El Device and Evaluation of Emission Performance]

### Examples 36-39

A glass substrate of 25 mm×75 mm×1.1 mm thickness having an ITO transparent electrode (product of Geomatec Company) was cleaned by ultrasonic cleaning in isopropyl alcohol for 5 min and then UV ozone cleaning for 30 min.

The cleaned glass substrate having a transparent electrode line was mounted to a substrate holder of a vacuum vapor deposition apparatus. The following compound K-1 was vapor-deposited so as to cover the transparent electrode to form an acceptor layer with a thickness of 10 nm. Successively after forming the acceptor layer, the following compound HT-3 and compound HT-4 were vapor-deposited in this order to form a first hole transporting layer with a thickness of 20 nm and a second hole transporting layer with a thickness of 10 nm (both anode-side organic thin film layers).

On the second hole transporting layer, each compound (host material) shown in Table 2 and the following compound RD-1 (phosphorescent material) were vapor co-deposited to form a phosphorescent light emitting layer with a thickness of 40 nm. The concentration of the compound RD-1 in the light emitting layer was 5.0% by mass. The co-deposited film works as a light emitting layer.

Successively after forming the light emitting layer, the following compound ET-2 was vapor-deposited to form an electron transporting layer (cathode-side organic thin film layer) with a thickness of 45 nm.

Then, LiF was vapor-deposited into a film with a thickness of 1 nm at a film-forming speed of 0.1 Å/min to form an electron injecting electrode (cathode). On the LiF film, metallic Al was vapor-deposited to form a metallic cathode with a thickness of 80 nm, thereby obtaining an organic EL device.

The compounds used in the examples are shown below.

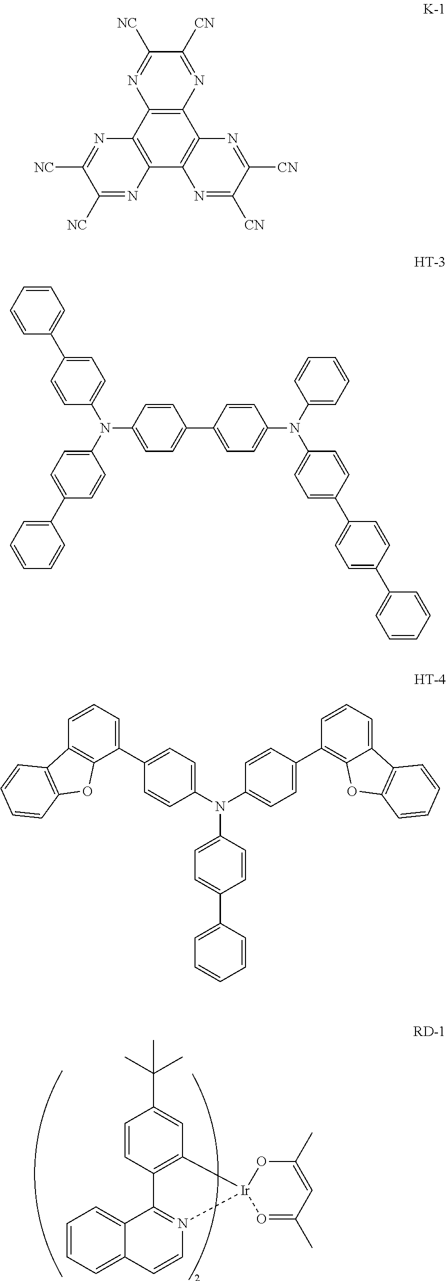

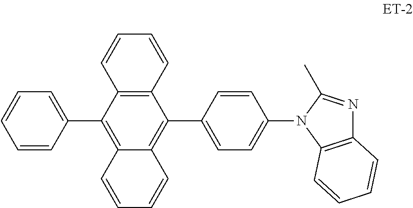 [SMILES: Cc1nc2ccccc2n1-c1ccc(-c2c3ccccc3c(-c3ccccc3)c3ccccc23)cc1]

### (Evaluation of Organic EL Device)

A voltage was applied to each of the organic EL devices thus obtained so as to control the current density to 10 mA/cm², and the external quantum efficiency (EQE) was determided. In addition, the time taken until the luminance was reduced to 80% of the initial luminance (LT80) was measured by using a luminance meter (spectroradiometer “CS-1000” manufactured by Minolta) while driving the device at a current density of 50 mA/cm². The results are shown in Table 2.

TABLE 2

Results

| | Light emitting layer | EQE | |
|---|---|---|---|
| | host material | (%) | lifetime (h) |
| Example 36 | compound 3 | 21.5 | 500 |
| Example 37 | compound 13 | 20.5 | 450 |
| Example 38 | compound 14 | 18.2 | 500 |
| Example 39 | compound 16 | 20.7 | 900 |

It can be seen from Table 2 that the organic EL device having a light emitting layer comprising the compound 3, 13, 14 or 16 exhibits a high external quantum yield (EQE) and a device life drastically improved.

The driving voltages at 10 mA/cm² of the organic EL devices obtained in Examples 36 to 39 are shown in Table 3. As compared with the compound 3 in which one ring is fused to the carbazole, the compounds 13, 14 and 16 in which two or more rings are fused to the carbazole are effective for further reducing the driving voltage and contribute much more to the reduction of power consumption of organic EL devices.

TABLE 3

| | Light emitting layer | Results |
|---|---|---|
| | host material | driving voltage (V) |
| Example 36 | compound 3 | 4.3 |
| Example 37 | compound 13 | 3.6 |
| Example 38 | compound 14 | 3.5 |
| Example 39 | compound 16 | 3.4 |

### [Production of Organic El Device and Evaluation of Emission Performance]

### Examples 40-42 and Comparative Example 2

A glass substrate of 25 mm×75 mm×1.1 mm thickness having an ITO transparent electrode (product of Geomatec Company) was cleaned by ultrasonic cleaning in isopropyl alcohol for 5 min and then UV ozone cleaning for 30 min.

The cleaned glass substrate having a transparent electrode line was mounted to a substrate holder of a vacuum vapor deposition apparatus. The following compound K-1 was vapor-deposited so as to cover the transparent electrode to form an acceptor layer with a thickness of 10 nm. Successively after forming the acceptor layer, the following compound HT-3 and compound HT-5 were vapor deposited in this order to form a first hole transporting layer with a thickness of 20 nm and a second hole transporting layer with a thickness of 10 nm (both anode-side organic thin film layers).

On the second hole transporting layer, each compound (host material) shown in Table 4 and the following compound RD-1 (phosphorescent material) were vapor co-deposited to form a phosphorescent light emitting layer with a thickness of 40 nm. The concentration of the compound RD-1 in the light emitting layer was 5.0% by mass. The co-deposited film works as a light emitting layer.

Successively after forming the light emitting layer, the following compound ET-3 was vapor-deposited to form an electron transporting layer (cathode-side organic thin film layer) with a thickness of 45 nm.

Then, LiF was vapor-deposited into a film with a thickness of 1 nm at a film-forming speed of 0.1 Å/min to form an electron injecting electrode (cathode). On the LiF film, metallic Al was vapor deposited to form a metallic cathode with a thickness of 80 nm, thereby obtaining an organic EL device.

The compounds used in the examples and the comparative example are shown below.

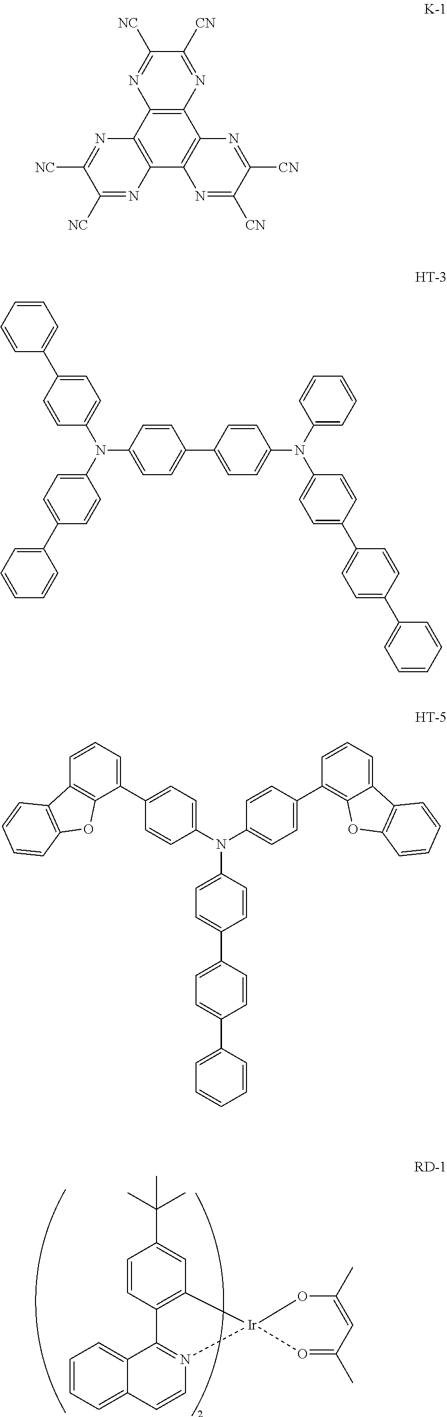

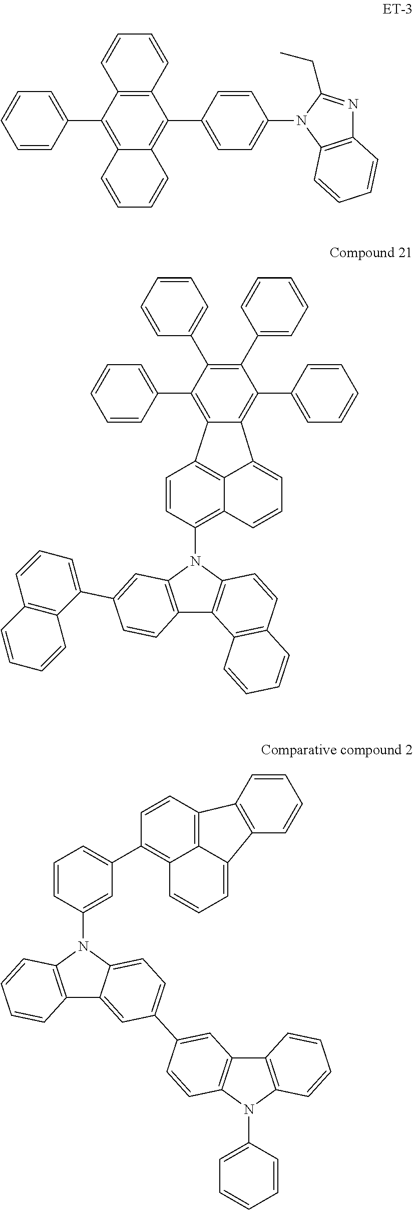 [SMILES: CCc1nc2ccccc2n1-c1ccc(-c2c3ccccc3c(-c3ccccc3)c3ccccc23)cc1 ; c1ccc(-c2c(-c3ccccc3)c(-c3ccccc3)c3c(c2-c2ccccc2)-c2cccc4c(-n5c6cc(-c7cccc8ccccc78)ccc6c6c7ccccc7ccc65)ccc-3c24)cc1 ; c1ccc(-n2c3ccccc3c3cc(-c4ccc5c(c4)c4ccccc4n5-c4cccc(-c5ccc6c7c(cccc57)-c5ccccc5-6)c4)ccc32)cc1]

### (Evaluation of Organic EL Device)

Each of the organic EL devices thus obtained was measured for the driving voltage (V) at a current density of 10 mA/cm² to evaluate the external quantum efficiency (EQE). In addition, the time taken until the luminance was reduced to 80% of the initial luminance (LT80) when driving at a current density of 50 mA/cm² was measured by using a luminance meter (spectroradiometer “CS-200” manufactured by Minolta). The results are shown in Table 4.

TABLE 4

| | | Results | | |
|---|---|---|---|---|
| | Light emitting layer | driving voltage | EQE | lifetime |
| | host material | (V) | (%) | (h) |
| Example 40 | compound 15 | 3.50 | 19.2 | 600 |
| Example 41 | compound 16 | 3.40 | 19.8 | 700 |
| Example 42 | compound 21 | 3.90 | 18.4 | 550 |
| Comparative | comparative | 4.10 | 18.1 | 500 |
| example 2 | compound 2 | | | |

It can be seen from Table 4 that the organic EL device having a light emitting layer which comprises any of the compounds 15, 16 and 21 is capable of driving at a low voltage and exhibits a high external quantum efficiency (EQE) and an improved device life. These effects are remarkable particularly in the organic EL device having a light emitting layer comprising the compound 15 or 16 (Examples 40 and 41).

### REFERENCE NUMERALS

1: Organic electroluminescence device
3: Anode
4: Cathode
5: Light emitting layer
6: Anode-side organic thin film layer
7: Cathode-side organic thin film layer
10: Organic thin film layer

1. A compound of formula (1): 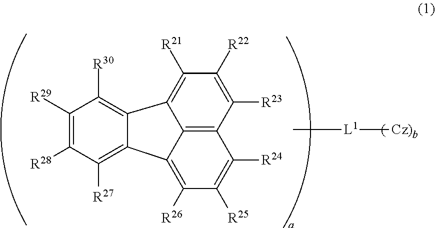
wherein each of R²¹ to R³⁰ independently represents a hydrogen atom or a substituent, wherein one of R²¹ to R³⁰ represents a direct bond to L¹ or Cz; L¹ represents a direct bond, a substituted or unsubstituted, divalent aromatic hydrocarbon group having 6 to 60 ring carbon atoms, a substituted or unsubstituted, divalent oxygen-containing heterocyclic group having 5 to 60 ring atoms, or a substituted or unsubstituted, divalent sulfur-containing heterocyclic group having 5 to 60 ring atoms; Cz represents a structure of formula (2); and each of a and b independently represents an integer of 1 to 3, wherein when at least one of a and b is 2 or 3, L¹ is not a direct bond, 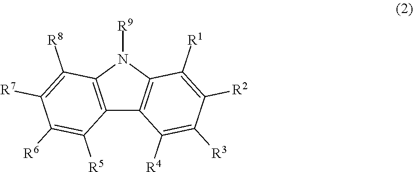
wherein each of R¹ to R⁹ independently represents a hydrogen atom or a substituent; and adjacent groups in R¹ to R⁸ may be bonded to each other to form a ring structure, wherein at least one adjacent pair of R¹ to R⁸ are bonded to each other to form a ring structure of formula (3) or (4): 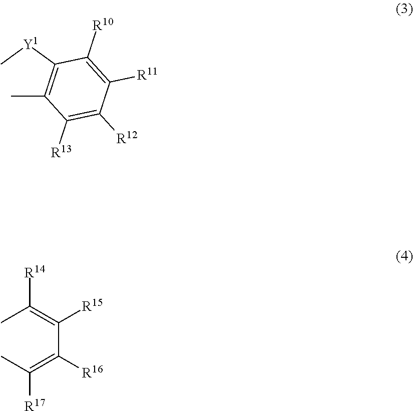
wherein each of R¹⁰ to R¹⁷ independently represents a hydrogen atom or a substituent; Y¹ represents an oxygen atom, a sulfur atom, or —CR³¹R³²—, wherein each of R³¹ and R³² independently represents a hydrogen atom or a substituent; adjacent groups in R¹⁰ to R¹³ may be bonded to each other to form a ring structure; Y¹ and R¹⁰ may be bonded to each other to form a ring structure; and adjacent groups in R¹⁴ to R¹⁷ may be bonded to each other to form a ring structure;
wherein one of R¹ to R¹⁷, R³¹, and R³² represents a direct bond to L¹ or one of R²¹ to R³⁰.

2. The compound according to claim 1, wherein Cz represents a structure of any one of formulae (5) to (14): 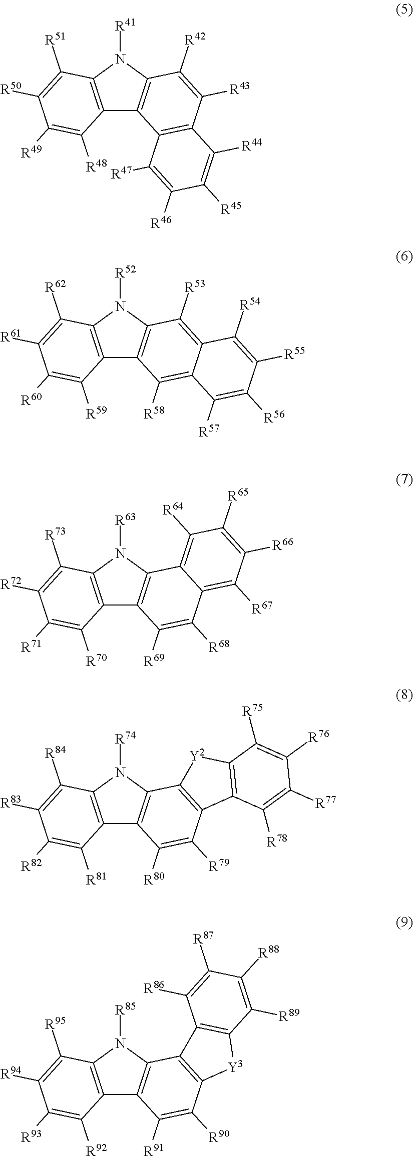 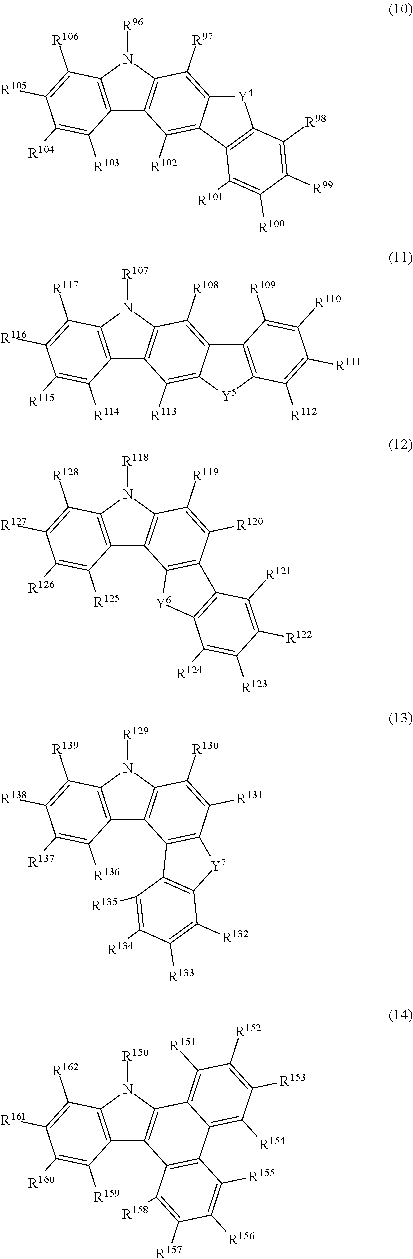
wherein, each of R⁴¹ to R¹³⁹ and R¹⁵⁰ to R¹⁶² independently represents a hydrogen atom or a substituent; adjacent groups in R⁴² to R⁵¹, R⁵³ to R⁶², R⁶⁴ to R⁷³, R⁷⁵, to R⁸⁴, R⁸⁶to R⁹⁵, R⁹⁸ to R¹⁰⁶, R¹⁰⁸ to R¹¹⁷, R¹¹⁹ to R¹²⁸, R¹³⁰ to R¹³⁹, and R¹⁵¹ to R¹⁶² may be bonded to each other to form a ring structure; each of Y² to Y⁷ represents an oxygen atom, a sulfur atom, or —CR¹⁴⁰R¹⁴¹—, wherein each of R¹⁴⁰ and R¹⁴¹ independently represents a hydrogen atom or a substituent;
one of R⁴¹ to R⁵¹, one of R⁵² to R⁶², one of R⁶³ to R⁷³, one of R⁷⁴ to R⁸⁴, one of R⁸⁵ to R⁹⁵, one of R⁹⁶ to R¹⁰⁶, one of R¹⁰⁷ to R¹¹⁷, one of R¹¹⁸ to R¹²⁸, one of R¹²⁹ to R¹³⁹, and one of R¹⁵⁰ to R¹⁶² each represent a direct bond to L¹ or one of R²¹ to R³⁰.

3. The compound according to claim 1, wherein R⁹ in formula (2) represents a direct bond to L¹ or one of R²¹ to R³⁰.

4. The compound according to claim 1, wherein a and b are both 1.

5. The compound according to claim 4, wherein the compound is represented by any of formulae (1-5) to (1-7) and (1-14): 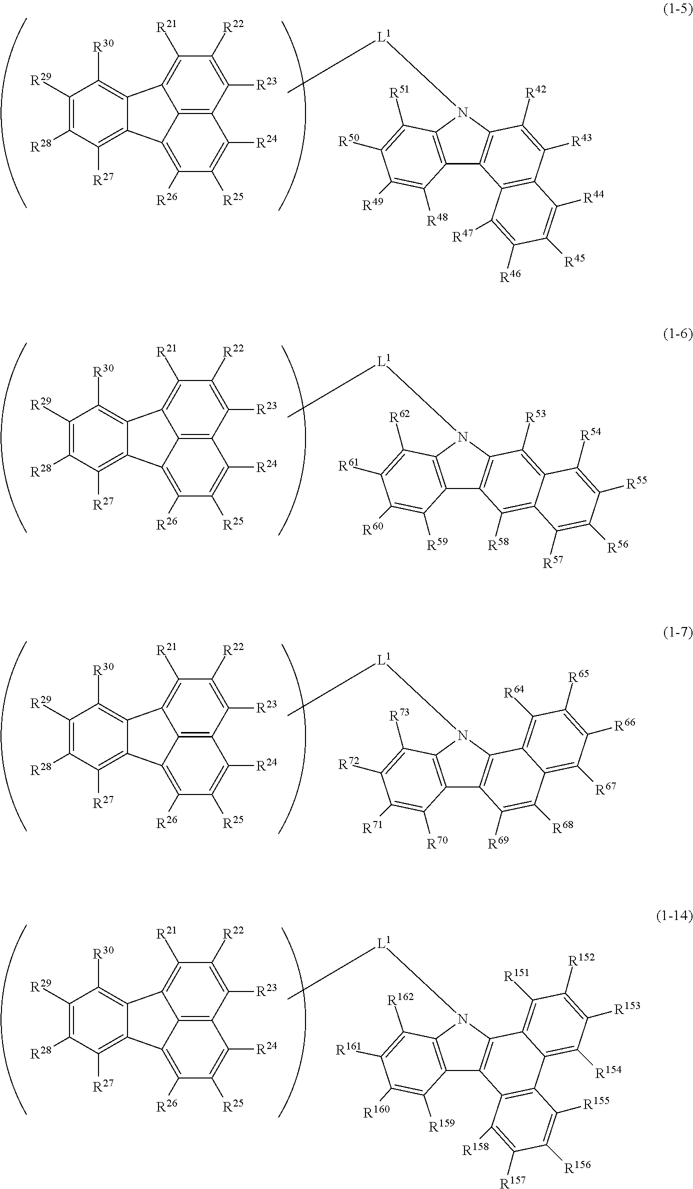
wherein each of R⁴² to R⁵¹, R⁵² to R⁶², and R⁶⁴ to R⁷³ independently represents a hydrogen atom or a substituent; and adjacent groups in R⁴² to R⁵¹, R⁵³ to R⁶², R⁶⁴ to R⁷³, and R¹⁵¹ to R¹⁶² may be bonded to each other to form a ring structure.

6. The compound according to claim 4, wherein the compound is represented by any of formulae (1-8) to (1-10): 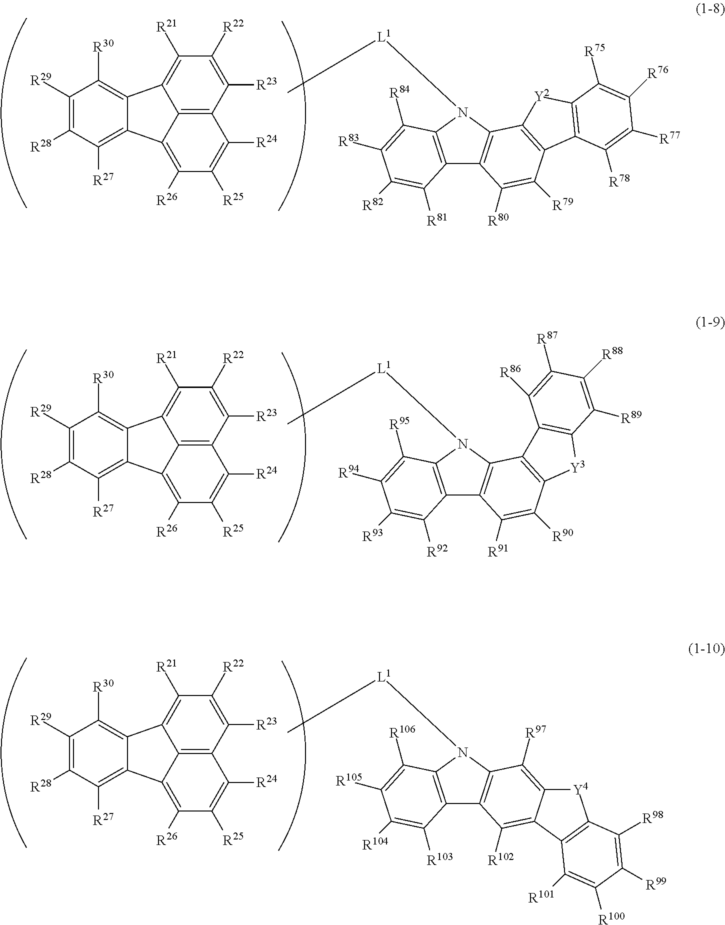
wherein each of R⁷⁵ to R⁸⁴, R⁸⁶ to R⁹⁵, and R⁹⁷ to R¹⁰⁶ independently represents a hydrogen atom or a substituent; adjacent groups in R⁷⁵ to R⁸⁴, R⁸⁶ to R⁹⁵, and R⁹⁸ to R¹⁰⁶ may be bonded to each other to form a ring structure; and each of Y² to Y⁴ represents an oxygen atom, a sulfur atom, or —CR¹⁴⁰R¹⁴¹—, wherein each of R¹⁴⁰ and R¹⁴¹ independently represents a hydrogen atom or a substituent.

7. The compound according to claim 4, wherein the compound is represented by any of formulae (1-11) to (1-13): 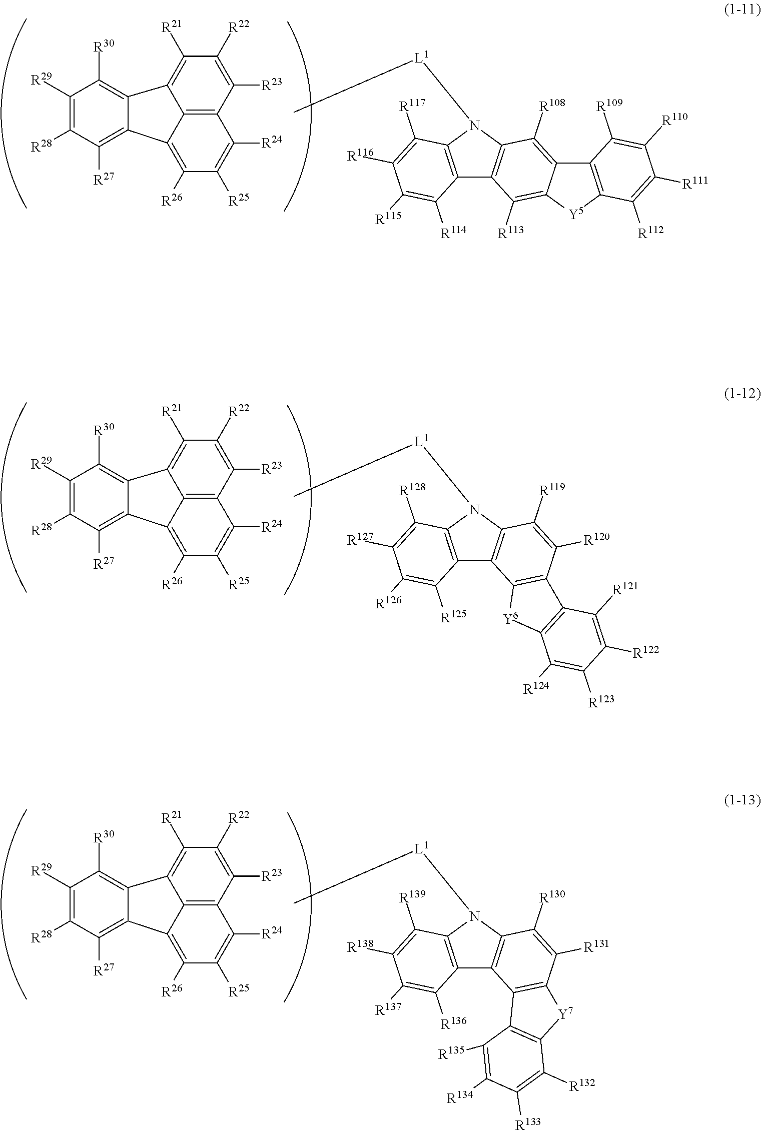
wherein each of R¹⁰⁸ to R¹¹⁷, R¹¹⁹ to R¹²⁸, and R¹³⁰ to R¹³⁹ independently represents a hydrogen atom or a substituent; adjacent groups in R¹⁰⁸ to R¹¹⁷, R¹¹⁹ to R¹²⁸, and R¹³⁰ to R¹³⁹ may be bonded to each other to form a ring structure; and each of Y⁵ to Y⁷ represents an oxygen atom, a sulfur atom, or —CR¹⁴⁰R¹⁴¹—, wherein each of R¹⁴⁰ and R¹⁴¹ independently represents a hydrogen atom or a substituent.

8. The compound according to claim 1, wherein the compound is represented by formula (1′):

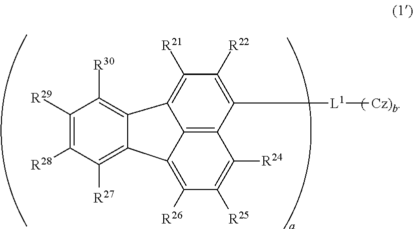

9. The compound according to claim 1, wherein the compound is represented by formula (1″):

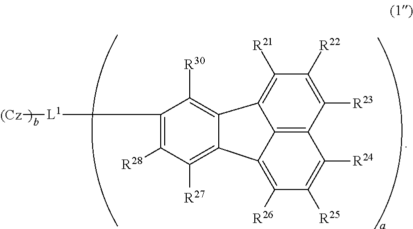

10. The compound according to claim 1, wherein the substituent in each occurrence represents a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted aralkyl group having 7 to 51 carbon atoms, an amino group, a mono- or di-substituted amino group wherein the substituent is selected from a substituted or unsubstituted alkyl group having 1 to 50 carbon atoms and a substituted or unsubstituted aryl group having 6 to 50 ring carbon atoms, a substituted or unsubstituted alkoxy group having 1 to 50 carbon atoms, a substituted or unsubstituted aryloxy group having 6 to 50 ring carbon atoms, a mono-, di- or tri-substituted silyl group wherein the substituent is selected from a substituted or unsubstituted alkyl group having 1 to 50 carbon atoms and a substituted or unsubstituted aryl group having 6 to 50 ring carbon atoms, a substituted or unsubstituted heteroaryl group having 5 to 50 ring atoms, a substituted or unsubstituted haloalkyl group having 1 to 50 carbon atoms, a halogen atom, a cyano group, a nitro group, or a sulfonyl group substituted by a substituent selected from a substituted or unsubstituted alkyl group having 1 to 50 carbon atoms and a substituted or unsubstituted aryl group having 6 to 50 ring carbon atoms.

11. The compound according to claim 10, wherein the substituent in each occurrence represents a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, a mono- or di-substituted amino group wherein the substituent is selected from a substituted or unsubstituted alkyl group having 1 to 50 carbon atoms and a substituted or unsubstituted aryl group having 6 to 50 ring carbon atoms, a substituted or unsubstituted heteroaryl group having 5 to 50 ring atoms, a halogen atom, or a cyano group.

12. The compound according to claim 1, wherein R²¹ to R³⁰ except for one which represents a direct bond to L¹ or Cz are all hydrogen atoms.

13. A material for organic electroluminescence devices comprising the compound according to claim 1.

14. An organic electroluminescence device comprising two or more organic thin film layers between a cathode and an anode, wherein the thin film layers comprise a light emitting layer and at least one layer of the thin film layers comprises the compound according to claim 1.

15. The organic electroluminescence device according to claim 14, wherein the light emitting layer comprises the compound of formula (1).

16. The organic electroluminescence device according to claim 14, wherein the light emitting layer comprises a phosphorescent emitting material.

17. The organic electroluminescence device according to claim 16, wherein the phosphorescent emitting material is an ortho-metallated complex comprising a metal selected from iridium (Ir), osmium (Os), and platinum (Pt).

18. The organic electroluminescence device according to claim 14, wherein the organic electroluminescence device comprises a cathode-side organic thin film layer between the cathode and the light emitting layer, and the cathode-side organic thin film layer comprises the compound of formula (1).

19. The organic electroluminescence device according to claim 14, wherein the organic electroluminescence device comprises an anode-side organic thin film layer between the anode and the light emitting layer, and the anode-side organic thin film layer comprises the compound of formula (1).

20. An electronic equipment comprising the organic electroluminescence device according to claim 14.
